(19) United States
(12) Patent Application Publication
(10) Pub. No.: US 2024/0115734 A1
(43) Pub. Date: Apr. 11, 2024

(54) RECOMBINANT AAVS WITH IMPROVED TROPISM AND SPECIFICITY

(71) Applicant: AFFINIA THERAPEUTICS INC., Waltham, MA (US)

(72) Inventors: Christopher TIPPER, Cambridge, MA (US); Lisa STANEX, Natick, MA (US); Keven OLIVIERI, Winchester, MA (US)

(21) Appl. No.: 18/264,919

(22) PCT Filed: Feb. 9, 2022
(86) PCT No.: PCT/US2022/015842

§ 371 Date: Aug. 9, 2023

Related U.S. Application Data
(60) Provisional application No. 63/147,701, filed on Feb. 9, 2021.
(60) Provisional application No. 63/173,998, filed on Apr. 12, 2021.
(60) Provisional application No. 63/186,641, filed on May 10, 2021.
(60) Provisional application No. 63/290,517, filed on Dec. 16, 2021.

(51) Int. Cl. A61K 48/00; A61P 21/00; C07K 14/005; C12N 9/16; C12N 15/86

(52) U.S. Cl. CPC A61K 48/005; A61P 21/00; C07K 14/005; C12N 9/16; C12N 15/86; C12N 2750/14122; C12N 2750/14143; C12N 2750/14145; C12Y 301/03

(57) ABSTRACT

The present disclosure provides a modified AAV capsid protein comprising a targeting peptide, optionally further comprising a liver-toggle mutation. The modified AAV capsid protein can form an rAAV, which has a preferred tropism, specificity or biodistribution in vivo or in vitro. The rAAV of the present disclosure can be used for gene therapies targeted at a specific tissue. The present disclosure also provides rAAV compositions comprising MTM1 coding sequences and their use to treat subjects suffering from X-linked myotubular myopathy (XLMTM).

### 1. CROSS-REFERENCE TO RELATED APPLICATIONS

This application claims the benefit of U.S. Provisional Application No. 63/147,701, filed Feb. 9, 2021, U.S. Provisional Application No. 63/173,998 filed Apr. 12, 2021, U.S. Provisional Application No. 63/186,641 filed May 10, 2021 and U.S. Provisional Application No. 63/290,517 filed Dec. 16, 2021, which are incorporated by reference in their entireties herein.

### 2. SEQUENCE LISTING

The instant application contains a Sequence Listing.

### 3. BACKGROUND

Adeno-associated virus (AAV) has become the vector system of choice for in vivo gene therapy. A growing variety of recombinant AAVs (rAAVs) engineered to deliver therapeutic nucleic acids have been developed and tested in nonhuman primates and humans, and the FDA has recently approved two rAAV gene therapy products for commercialization.

Although AAV vectors are safer and less inflammatory than other viruses, toxicities have occurred following administration of high doses of rAAVs for gene therapy. Thus, local administration of rAAVs to a target tissue or organ has been used to improve targeting and reduce systemic toxicity. Further, various natural and synthetic AAV variants have been tested to develop an AAV vector with desired tropism and specificity.

In general, the capsid is thought to be the primary determinant of infectivity and host-vector related properties such as adaptive immune responses, tropism, specificity, potency, and bio-distribution. Indeed, several of these properties are known to vary between natural serotypes and engineered AAV variants. Over the last decade, novel synthetic AAV variants have been developed by using a variety of capsid engineering techniques, one of which is the insertion of small, 7 amino acid-long, peptides into an exposed loop of the capsid protein, called variable region VIII (VRVIII). In some circumstances, the insertion of a novel peptide into a wild type capsid changes the tropism of the variant. For example, insertion of a peptide having the sequence RGDLGLS (SEQ ID NO: 156) into the capsid of AAV9 was found to increase infection of astrocytes (see PhD thesis of Eike Kienle, Ruprecht-Karls-Universitat Heidelberg, 2014) and primary breast cancer cells (Michelfelder et al. (2009)).

To date, however, there is little understanding as to how these changes on the capsid functionally alter these properties. Additionally, AAV vectors with a desired tropism and specificity to common therapeutic targets, such as muscles, have not yet been available.

For example, X-linked myotubular myopathy (XLMTM; OMIM 310400) is a fatal monogenic disease of skeletal muscle. XLMTM results from loss-of-function mutations in Myotubularin 1 (MTM1), which encodes one of a family of 3-phosphoinositide phosphatases acting on the second messengers phosphatidylinositol 3-monophosphate [PI(3)P] and phosphatidylinositol 3,5-bisphosphate [PI(3,5)P2] (see, e.g., Miyagoe-Suzuki and Takeda, 2010, Exp Cell Res 316(18):3087-92). Although myotubularin is expressed ubiquitously, loss of this enzyme primarily affects skeletal muscle.

A recent clinical trial evaluated a recombinant adeno-associated virus (rAAV) as a gene therapy for XLMTM. Unfortunately, the rAAV, which was an rAAV serotype 8 vector carrying the MTM1 gene under the control of the muscle-specific desmin promoter, reported three deaths from the high-dose limb of the trial resulting from liver dysfunction (Mendell et al., 2021, Mol Ther. 29(2):464-488. doi: 10.1016/j.ymthe.2020.12.007. Epub 2020 Dec. 10. PMID: 33309881; PMCID: PMC7854298).

Thus, there remains a need in the art for gene therapies for muscular disorders, such as XLMTM, with improved safety profiles.

### 4. SUMMARY OF THE INVENTION

The present disclosure provides a modified AAV capsid protein that can form an rAAV having a preferred tropism and specificity to a therapeutic target. Specifically, a modified AAV capsid protein comprising a targeting peptide, RGDLLLS (SEQ ID NO: 1), in the VR VIII region is provided. The rAAVs containing the modified AAV capsid protein demonstrated better targeting with more specific expression of a transgene in the target tissue, e.g., muscles, when systemically administered to a mammalian subject.

Additionally, it was demonstrated that the specific targeting of the rAAV can be enhanced by introducing a liver-toggle mutation together with a targeting peptide to the capsid protein. Applicant previously demonstrated that the liver-toggle mutation is associated with liver-on or liver-off tropism. Applicant now reports that the liver-toggle mutation provides synergistic effects to the specific targeting of an rAAV to a target tissue when combined with a targeting peptide.

The use of AAV for gene therapy for muscular disorders (e.g., XLMTM) has been limited because of liver toxicity. Modified AAV capsid proteins provided herein provide an improved way to treat the diseases with better safety. The modified AAV capsid proteins could deliver a construct encoding a therapeutic gene (e.g., MTM1) with reduced liver tropism and/or improved muscle tropism. Additionally, the construct could drive higher and more specific MTM1 expression at the target by virtue of appropriate expression regulatory elements (ERE) (e.g., promoter sequences) and/or codon optimized coding sequences.

Accordingly, one aspect of the present disclosure provides a modified adeno-associated virus (AAV) capsid protein, comprising: (i) a reference AAV capsid protein, and (ii) a 7-mer peptide having the sequence RGDLLLS (SEQ ID NO: 1) inserted into a site within VR VIII of the reference AAV capsid protein.

In some embodiments, the AAV capsid protein is selected from one or more of VP1, VP2 and VP3. In some embodiments, the reference AAV capsid protein is a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI. In some embodiments, the reference AAV capsid protein is a capsid protein having a sequence selected from SEQ ID Nos: 54-152 or a fragment thereof.

In some embodiments, the 7-mer peptide is inserted into an amino acid position between 565 and 595 of the reference AAV capsid protein. In some embodiments, (i) the reference AAV capsid protein is a capsid protein of AAV1 and the 7-mer peptide is inserted between D590 and P591 or between S588 and T589 of the capsid protein; (ii) the reference AAV capsid protein is a capsid protein of AAV2 and the 7-mer peptide is inserted between R588 and Q589 or between N587 and R588 of the capsid protein; (iii) the reference AAV capsid protein is a capsid protein of AAV3b and the 7-mer peptide is inserted between S586 and S587 or between N588 and T589 of the capsid protein; (iv) the reference AAV capsid protein is a capsid protein of AAV4 and the 7-mer peptide is inserted between S584 and N585 or between S586 and N587 of the capsid protein; (v) the reference AAV capsid protein is a capsid protein of AAV5 and the 7-mer peptide is inserted between S575 and S576 or between T577 and T578 of the capsid protein; (vi) the reference AAV capsid protein is a capsid protein of AAV6 and the 7-mer peptide is inserted between D590 and P591 or S588 and T589 of the capsid protein; (vii) the reference AAV capsid protein is a capsid protein of AAV7 and the 7-mer peptide is inserted between N589 and T590 of the capsid protein; (viii) the reference AAV capsid protein is a capsid protein of AAV8 and the 7-mer peptide is inserted between N590 and T591 of the capsid protein; (ix) the reference AAV capsid protein is a capsid protein of AAV9 and the 7-mer peptide is inserted between Q588 and A589 of the capsid protein; (x) the reference AAV capsid protein is a capsid protein of AAVrh10 and the 7-mer peptide is inserted between N590 and A591 of the capsid protein; (xi) the reference AAV capsid protein is a capsid protein of AAVpo.1 and the 7-mer peptide is inserted between N567 and S568 or between N569 and T570 of the capsid protein; or (xii) the reference AAV capsid protein is a capsid protein of AAV12 and the 7-mer peptide is inserted between N592 and A593 or between T594 and T595 of the capsid protein.

In some embodiments, the modified AAV capsid protein has a sequence of SEQ ID NO: 158.

In some embodiments, the reference AAV capsid protein is a liver-toggle mutant of a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI. In some embodiments, the reference AAV capsid protein is a liver-toggle mutant of a capsid protein having a sequence selected from SEQ ID Nos: 54-152 or a fragment thereof.

In some embodiments, the modified AAV capsid protein comprises an alanine (A) amino acid residue at an amino acid position corresponding to position 266 in Anc80; or a lysine (K) amino acid residue at an amino acid position corresponding to position 168 in Anc80.

In some embodiments, the modified AAV capsid protein comprises a glycine (G) amino acid residue at an amino acid position corresponding to position 266 in Anc80; or an arginine (R) amino acid residue at an amino acid position corresponding to position 168 in Anc80.

In some embodiments, the reference AAV capsid protein is a liver toggle mutant of a capsid protein of AAV9 comprising an alanine (A) amino acid residue at an amino acid position 267 and a threonine (T) amino acid residue at an amino acid position 269. In some embodiments, the modified AAV capsid protein comprises the sequence of SEQ ID NO: 159.

In some embodiments, the modified AAV capsid protein comprises a glycine (G) amino acid residue at an amino acid position corresponding to position 266 in Anc80; or a lysine (K) amino acid residue at an amino acid position corresponding to position 168 in Anc80.

In another aspect, the present disclosure provides a modified adeno-associated virus (AAV) capsid protein, comprising: (i) a liver-toggle mutant of a reference AAV capsid protein, comprising a) an alanine (A) or glycine (G) amino acid residue at an amino acid position corresponding to position 266 in Anc80; or b) a lysine (K) or arginine (R) amino acid residue at an amino acid position corresponding to position 168 in Anc80; and (ii) a targeting peptide inserted into a site within VR VIII of the liver-toggle mutant.

In some embodiments, the liver-toggle mutant comprises: a) an alanine (A) amino acid residue at an amino acid position corresponding to position 266 in Anc80; or b) a lysine (K) amino acid residue at an amino acid position corresponding to position 168 in Anc80. In some embodiments, the liver-toggle mutant comprises: a) an alanine (A) amino acid residue at an amino acid position corresponding to position 266 in Anc80; and b) a lysine (K) amino acid residue at an amino acid position corresponding to position 168 in Anc80.

In some embodiments, the liver-toggle mutant comprises: a) a glycine (G) amino acid residue at an amino acid position corresponding to position 266 in Anc80; or b) an arginine (R) amino acid residue at an amino acid position corresponding to position 168 in Anc80. In some embodiments, the liver-toggle mutant comprises: a) a glycine (G) amino acid residue at an amino acid position corresponding to position 266 in Anc80; and b) an arginine (R) amino acid residue at an amino acid position corresponding to position 168 in Anc80.

In some embodiments, the targeting peptide is 7-mer peptide having the sequence RGDX¹X²X³X⁴ (SEQ ID NO: 52), wherein X¹ to X⁴ are independently selected amino acid residues. In some embodiments, X¹, X², and X³ are independently selected from L, G, V, and A; and X⁴ is selected from S, V, A, G, and L. In some embodiments, X¹, X², and X³ are independently selected from L, V, and A; and at least two of X¹, X², and X³ are independently L. In some embodiments, X² is L. In some embodiments, 7-mer peptide has a sequence of RGDLLLS (SEQ ID NO: 1).

In some embodiments, the targeting peptide is the 7-mer peptide TLAVPFK (SEQ ID NO: 53). In some embodiments, the targeting peptide has a sequence selected from SEQ ID Nos: 2-51 and 53.

In some embodiments, the reference AAV capsid protein is a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-13; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc8l; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI. In some embodiments, the reference AAV capsid protein is a capsid protein having a sequence selected from SEQ ID Nos: 54-152 or a fragment thereof.

In some embodiments, the reference AAV capsid polypeptide is an AAV9 capsid protein.

In some embodiments, the liver-toggle mutant comprises an alanine (A) amino acid residue at position 267. In some embodiments, the liver-toggle mutant comprises a threonine (T) amino acid residue at position 269. In some embodiments, the liver-toggle mutant comprises an alanine (A) amino acid residue at position 267 and a threonine (T) amino acid residue at position 269.

In some embodiments, the targeting peptide is inserted into an amino acid position between 565 and 595 of the liver toggle mutant. In some embodiments, (i) the reference AAV capsid protein is a capsid protein of AAV1 and the targeting peptide is inserted between D590 and P591 or between S588 and T589 of the liver-toggle mutant; (ii) the reference AAV capsid protein is a capsid protein of AAV2 and the targeting peptide is inserted between R588 and Q589 or between N587 and R588 of the liver-toggle mutant; (iii) the reference AAV capsid protein is a capsid protein of AAV3b and the targeting peptide is inserted between S586 and S587 or between N588 and T589 of the liver-toggle mutant; (iv) the reference AAV capsid protein is a capsid protein of AAV4 and the targeting peptide is inserted between S584 and N585 or between S586 and N587 of the liver-toggle mutant; (v) the reference AAV capsid protein is a capsid protein of AAV5 and the targeting peptide is inserted between S575 and S576 or between T577 and T578 of the liver-toggle mutant; (vi) the reference AAV capsid protein is a capsid protein of AAV6 and the targeting peptide is inserted between D590 and P591 or S588 and T589 of the liver-toggle mutant; (vii) the reference AAV capsid protein is a capsid protein of AAV7 and the targeting peptide is inserted between N589 and T590 of the liver-toggle mutant; (viii) the reference AAV capsid protein is a capsid protein of AAV8 and the targeting peptide is inserted between N590 and T591 of the liver-toggle mutant; (ix) the reference AAV capsid protein is a capsid protein of AAV9 and the targeting peptide is inserted between Q588 and A589 of the liver-toggle mutant; (x) the reference AAV capsid protein is a capsid protein of AAVrh10 and the targeting peptide is inserted between N590 and A591 of the liver-toggle mutant; (xi) the reference AAV capsid protein is a capsid protein of AAVpo.1 and the targeting peptide is inserted between N567 and S568 or between N569 and T570 of the liver-toggle mutant; or (xii) the reference AAV capsid protein is a capsid protein of AAV12 and the targeting peptide is inserted between N592 and A593 or between T594 and T595 of the liver-toggle mutant.

In some embodiments, the liver-toggle mutant comprises a sequence selected from NSTSGASS (SEQ ID NO: 160), NSTSGGST (SEQ ID NO: 161) and NSTSGAST (SEQ ID NO: 162).

In some embodiments, the liver-toggle mutant of a reference AAV capsid protein, comprises a) an alanine (A) amino acid residue at an amino acid position corresponding to position 266 in Anc80; and b) a lysine (K) amino acid residue at an amino acid position corresponding to position 168 in Anc80

In some embodiments, the liver-toggle mutant of a reference AAV capsid protein, comprises a) an alanine (A) amino acid residue at an amino acid position corresponding to position 267 in AAV9; and b) a threonine (T) amino acid residue at an amino acid position corresponding to position 269 in AAV9.

In some embodiments, the liver-toggle mutant further comprises a) an alanine (A) amino acid residue at an amino acid position corresponding to position 504 in AAV9; and b) an alanine (A) amino acid residue at an amino acid position corresponding to position 505 in AAV9.

In some embodiments, the modified AAV capsid protein comprises a sequence of SEQ ID NO: 159.

In yet another aspect, the present disclosure provides a polynucleotide encoding the modified AAV capsid protein disclosed herein. In one aspect, the present disclosure relates to a vector comprising the polynucleotide. In some embodiments, the vector further comprises a promoter operably linked to the polynucleotide. Further disclosed herein includes a host cell comprising the modified AAV capsid protein, the polynucleotide, or the vector.

One aspect of the present disclosure provides a recombinant AAV virion (rAAV) comprising the modified AAV capsid protein disclosed herein. In some embodiments, the rAAV virion further comprises an exogenous polynucleotide. In some embodiments, the exogenous polynucleotide comprises a template for homology directed repair. In some embodiments, the exogenous polynucleotide comprises an expressible polynucleotide encoding a therapeutic tRNA, miRNA, gene editing guide RNA, or RNA-editing guide RNA. In some embodiments, the exogenous polynucleotide comprises an expressible polynucleotide encoding a therapeutic protein.

Another aspect of the present disclosure provides a pharmaceutical composition comprising the modified AAV capsid protein or the AAV virion.

It further discloses a method for treating or ameliorating or preventing a disease or condition in a subject, comprising administering a therapeutically effective amount of the AAV virion or the pharmaceutical composition of the present disclosure. In some embodiments, the disease is a muscular disease and/or the condition is muscle degeneration. In some embodiments, said muscle is a striated muscle, preferably heart or a skeletal muscle or diaphragm. In some embodiments, said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

In one aspect, the present disclosure provides a modified adeno-associated virus (AAV) capsid protein for use in treating and/or preventing a muscular disease and/or muscle degeneration. It further discloses an AAV virion comprising the modified AAV capsid protein for use in treating and/or preventing a muscular disease and/or in muscle regeneration. It also discloses a pharmaceutical composition comprising the modified AAV capsid protein, and/or the AAV virion for use in treating and/or preventing a muscular disease and/or in muscle regeneration. Additionally, provided herein includes use of the AAV capsid polypeptide, and/or the AAV virion for transferring an active compound into a muscle cell. In some embodiments, said use is a non-therapeutic use, preferably wherein said use is an in vitro use.

In one aspect, the present disclosure provides a method of transferring an exogenous polynucleotide into a muscle cell, comprising the step of administering the AAV virion of the present disclosure to a subject. In some embodiments, the administration results in transfer of the exogenous polynucleotide in the muscle cell, at a muscle:liver infection ratio of greater than 1 when measured by genome copies of the AAV virion. In some embodiments, the muscle:liver infection ratio ranges from 1 to 100. In some embodiments, the muscle:liver infection ration ranges from 1 to 10. In some embodiments, the muscle:liver infection ratio ranges from 2 to 8.

In some embodiments, the administration results in expression of the exogenous polynucleotide in the muscle cell, at a muscle:liver expression ratio of greater than 10. In some embodiments, the muscle:liver expression ratio ranges from 10 to 100. In some embodiments, the muscle:liver expression ratio ranges from 20 to 80. In some embodiments, the muscle:liver expression ratio ranges from 50 to 80 when measured by mRNA transcript expression. In some embodiments, the muscle:liver expression ratio ranges from 10 to 50 when measured by protein expression.

In some embodiments, the muscle cell is selected from triceps surae, biceps, heart and quadricep.

In another aspect, the present disclosure provides an rAAV whose genome comprises an MTM1 coding sequence operably linked to an expression regulatory element (ERE); and one, two or all three of the following features: (a) the ERE is a hybrid expression regulatory element (ERE) comprising a CMV enhancer and a chicken beta actin promoter operably linked to the MTM1 coding sequence; and/or (b) the rAAV comprises a modified AAV capsid protein comprising at least one liver-toggle mutation and/or one muscle-targeting element; and/or (c) the MTM1 coding sequence is codon optimized for expression in human cells, optionally wherein the coding sequence has at least 90%, at least 95%, at least 98% or at least 99% sequence identity to any one of SEQ ID NOS:167 to 170.

In some embodiments, the MTM1 sequence encodes a protein comprising an amino acid sequence having at least 95% sequence identity to the amino acid sequence of SEQ ID NO: 164. In some embodiments, the MTM1 protein comprises an amino acid sequence having at least 98% or at least 99% sequence identity to the amino acid sequence of SEQ ID NO:164. In some embodiments, the MTM1 protein comprises an amino acid sequence having 100% sequence identity to the amino acid sequence of SEQ ID NO:164.

In some embodiments, the MTM1 sequence encodes a protein comprising an amino acid sequence having at least 95% sequence identity to the amino acid sequence of SEQ ID NO:165. In some embodiments, the MTM1 protein comprises an amino acid sequence having at least 98% sequence identity to the amino acid sequence of SEQ ID NO:165. In some embodiments, the MTM1 protein comprises an amino acid sequence having at least 99% sequence identity to the amino acid sequence of SEQ ID NO:165. In some embodiments, the MTM1 protein comprises an amino acid sequence having 100% sequence identity to the amino acid sequence of SEQ ID NO:165.

In some embodiments, the MTM1 coding sequence comprises a nucleotide sequence having at least 90% sequence identity to SEQ ID NO 166. In some embodiments, the MTM1 coding sequence comprises a nucleotide sequence having at least 95% sequence identity to SEQ ID NO:166. In some embodiments, the MTM1 coding sequence comprises a nucleotide sequence having at least 98% sequence identity to SEQ ID NO:166. In some embodiments, the MTM1 coding sequence comprises a nucleotide sequence having at least 99% sequence identity to SEQ ID NO:166. In some embodiments, the MTM1 coding sequence comprises a nucleotide sequence having 100% sequence identity to SEQ ID NO:166.

In some embodiments, the MTM1 coding sequence is codon optimized for expression in human cells. In some embodiments, the MTM1 coding sequence comprises a nucleotide sequence having at least 90% sequence identity to any one of SEQ ID NOS:167 to 170. In some embodiments, the MTM1 coding sequence comprises a nucleotide sequence having at least 95% sequence identity to any one of SEQ ID NOS: 167 to 170. In some embodiments, the MTM1 coding sequence comprises a nucleotide sequence having at least 98% sequence identity to any one of SEQ ID NOS: 167 to 170. In some embodiments, the MTM1 coding sequence comprises a nucleotide sequence having at least 99% sequence identity to any one of SEQ ID NOS:167 to 170. In some embodiments, the MTM1 coding sequence comprises a nucleotide sequence having 100% sequence identity to any one of SEQ ID NOS:167 to 170. In some embodiments, the sequence identity is to SEQ ID NO:167. In some embodiments, the sequence identity is to SEQ ID NO: 168. In some embodiments, the sequence identity is to SEQ ID NO:169, In some embodiments, the sequence identity is to SEQ ID NO:169.

In some embodiments, the rAAV comprises a hybrid expression regulatory element (ERE) comprising a CMV enhancer and a chicken beta actin promoter operably linked to the MTM1 coding sequence.

In some embodiments, the ERE comprises (a) a nucleotide sequence having at least 90% sequence identity to SEQ ID NO: 171 and a nucleotide sequence having at least 90% sequence identity to SEQ ID NO:172 or (b) a nucleotide sequence having at least 90% sequence identity to SEQ ID NO:173. In some embodiments, the ERE comprises (a) a nucleotide sequence having at least 95% sequence identity to SEQ ID NO:171 and a nucleotide sequence having at least 95% sequence identity to SEQ ID NO: 172 or (b) a nucleotide sequence having at least 95% sequence identity to SEQ ID NO:173. In some embodiments, the ERE comprises (a) a nucleotide sequence having at least 98% sequence identity to SEQ ID NO: 171 and a nucleotide sequence having at least 98% sequence identity to SEQ ID NO: 172 or (b) a nucleotide sequence having at least 98% sequence identity to SEQ ID NO:173. In some embodiments, the ERE comprises (a) a nucleotide sequence having at least 99% sequence identity to SEQ ID NO:171 and a nucleotide sequence having at least 99% sequence identity to SEQ ID NO:172 or (b) a nucleotide sequence having at least 99% sequence identity to SEQ ID NO:173. In some embodiments, the ERE comprises (a) a nucleotide sequence having 100% sequence identity to SEQ ID NO:171 and a nucleotide sequence having 100% sequence identity to SEQ ID NO:172 or (b) a nucleotide sequence having 100% sequence identity to SEQ ID NO: 173.

In some embodiments, the rAAV further comprises a chimeric intron formed from intron sequences derived from chicken beta actin and/or human betaherpes virus and/or human beta globin and/or operably linked to the MTM1 coding sequence.

In some embodiments, the chimeric intron comprises a nucleotide sequence derived from human beta globin, which optionally comprises a nucleotide sequence having at least 90% sequence identity to SEQ ID NO:174. In some embodiments, the chimeric intron comprises a nucleotide sequence derived from human beta globin comprises SEQ ID NO: 174.

In some embodiments, the chimeric intron comprises a nucleotide sequence derived from human beta herpes virus, which optionally comprises a nucleotide sequence having at least 90% sequence identity to SEQ ID NO:175. In some embodiments, the nucleotide sequence is derived from human beta herpes virus comprises SEQ ID NO:175.

In some embodiments, the chimeric intron is formed from introns from human beta herpes virus and rabbit beta globin. In some embodiments, the chimeric intron comprises a nucleotide sequence having at least 90% sequence identity to SEQ ID NO:176. In some embodiments, the chimeric intron comprises a nucleotide sequence having at least 95% sequence identity to SEQ ID NO:176. In some embodiments, the chimeric intron comprises a nucleotide sequence having at least 98% sequence identity to SEQ ID NO: 176. In some embodiments, the chimeric intron comprises a nucleotide sequence having at least 99% sequence identity to SEQ ID NO:176. In some embodiments, the chimeric intron comprises a nucleotide sequence having 100% sequence identity to SEQ ID NO:176. In some embodiments, the chimeric intron comprises the nucleotide sequence of SEQ ID NO: 176.

In some embodiments, the rAAV comprises an unmodified or modified AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI capsid protein.

In some embodiments, the rAAV comprises an unmodified or modified rAAV9 capsid protein. In some embodiments, the rAAV comprises a VP1, VP2 and/or VP3 capsid protein comprising an amino acid sequence having at least 90% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

In some embodiments, the rAAV comprises a VP1, VP2 and/or VP3 capsid protein comprising an amino acid sequence having at least 95% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

In some embodiments, the rAAV comprises a VP1, VP2 and/or VP3 capsid protein comprising an amino acid sequence having at least 98% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

In some embodiments, the rAAV comprises a VP1, VP2 and/or VP3 capsid protein comprising an amino acid sequence having at least 99% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

In some embodiments, the rAAV comprises a VP1, VP2 and/or VP3 capsid protein comprising an amino acid sequence having 100% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

In some embodiments, the rAAV comprises a modified AAV capsid protein comprising at least one liver-toggle mutation as compared to a reference capsid protein.

In some embodiments, the reference capsid protein is a VP1, VP2 and/or VP3 protein. In some embodiments, the reference AAV capsid protein is a capsid protein having any one of SEQ ID NOs:54-152 or a fragment thereof.

In some embodiments, the at least one liver-toggle mutation comprises: an alanine (A) or glycine (G) amino acid residue at an amino acid position corresponding to position 266 in Anc80; and/or a lysine (K) or arginine (R) amino acid residue at an amino acid position corresponding to position 168 in Anc80.

In some embodiments, the at least one liver-toggle mutation comprises: an alanine (A) amino acid residue at an amino acid position corresponding to position 266 in Anc80; and/or a lysine (K) amino acid residue at an amino acid position corresponding to position 168 in Anc80.

In some embodiments, the at least one liver-toggle mutation comprises: an alanine (A) amino acid residue at an amino acid position corresponding to position 266 in Anc80; and/or an arginine (R) amino acid residue at an amino acid position corresponding to position 168 in Anc80.

In some embodiments, the at least one liver-toggle mutation comprises: a glycine (G) amino acid residue at an amino acid position corresponding to position 266 in Anc80; and/or a lysine (K) amino acid residue at an amino acid position corresponding to position 168 in Anc80.

In some embodiments, the at least one liver-toggle mutation comprises: a glycine (G) amino acid residue at an amino acid position corresponding to position 266 in Anc80; and/or an arginine (R) amino acid residue at an amino acid position corresponding to position 168 in Anc80.

In some embodiments, the at least one liver-toggle mutation comprises an alanine (A) at an amino acid position corresponding to position 267 in AAV9. In some embodiments, the at least one liver-toggle mutation comprises a threonine (T) at an amino acid position corresponding to position 269 in AAV9.

In some embodiments, the capsid protein is a modified AAV9 capsid protein, optionally wherein the capsid protein is a modified AAV9 VP1 capsid protein.

In some embodiments, the liver-toggle mutation comprises: an alanine (A) amino acid residue at an amino acid position corresponding to position 267 in AAV9; and a threonine (T) amino acid residue at an amino acid position corresponding to position 269 in AAV9.

In some embodiments, the liver-toggle mutation further comprises an alanine (A) amino acid residue at an amino acid position corresponding to position 504 in AAV9; and/or an Alanine (A) amino acid residue at an amino acid position corresponding to position 505 in AAV9.

In some embodiments, the liver-toggle mutant comprises the sequence NSTSGASS (SEQ ID NO: 160), NSTSGGST (SEQ ID NO: 161) or NSTSGAST (SEQ ID NO:162). In some embodiments, the rAAV capsid protein has the sequence of SEQ ID NO:159. In some embodiments, the rAAV capsid protein has the sequence of SEQ ID NO:163.

In some embodiments, the one or more liver toggle mutations comprise one or more amino acid substitutions at one or more of Q263, S264, G265, A266, S267, N268, H271, N382, G383, S384, Q385, S446, R471, W502, T503, D528, D529, Q589, K706, and V708 as compared to an AAV2 reference capsid protein (SEQ ID NO:1 of WO2021/050614, which is incorporated by reference herein).

In some embodiments, the one or more liver toggle mutations comprise the amino acid substitution S446R as compared to a reference capsid protein. In some embodiments, the one or more liver toggle mutations comprise the amino acid substitution R471A as compared to a reference capsid protein. In some embodiments, the one or more liver toggle mutations comprise the amino acid substitution V708T or V708A as compared to a reference capsid protein.

In some embodiments, the rAAV comprises a modified AAV capsid protein comprising at least one muscle-targeting element as compared to a reference capsid protein. In some embodiments, the reference capsid protein is a VP1, VP2 and/or VP³ protein.

In some embodiments, the muscle targeting element is 7-mer peptide having the sequence RGDX1X2X3X4 (SEQ ID NO:52), wherein X1 to X4 are independently selected amino acid residues. In some embodiments, X1, X2, and X3 are independently selected from L, G, V, and A; and X4 is selected from S, V, A, G, and L. In some embodiments, X1, X2, and X3 are independently selected from L, V, and A; and at least two of X1, X2, and X3 are independently L. In some embodiments, X2 is L.

In some embodiments, 7-mer peptide has a sequence of RGDLLLS (SEQ ID NO: 1). In some embodiments, the targeting peptide is the 7-mer peptide TLAVPFK (SEQ ID NO:53). In some embodiments, the targeting peptide is a peptide having any one of SEQ ID NOs:2-51 and 53.

In some embodiments, the muscle-targeting element consists of a 7-mer peptide having the sequence RGDLLLS (SEQ ID NO:1) inserted into a site within VR VIII of the AAV capsid protein. In some embodiments, the 7-mer peptide is inserted into an amino acid position between 565 and 595 of the reference AAV capsid protein.

In some embodiments, the reference AAV capsid protein is a capsid protein of AAV1 and a 7-mer muscle-targeting peptide is inserted between D590 and P591 or between S588 and T589 of the capsid protein; the reference AAV capsid protein is a capsid protein of AAV2 and the 7-mer muscle-targeting peptide is inserted between R588 and Q589 or between N587 and R588 of the capsid protein; the reference AAV capsid protein is a capsid protein of AAV3b and the 7-mer muscle-targeting peptide is inserted between S586 and S587 or between N588 and T589 of the capsid protein; the reference AAV capsid protein is a capsid protein of AAV4 and the 7-mer muscle-targeting peptide is inserted between S584 and N585 or between S586 and N587 of the capsid protein; the reference AAV capsid protein is a capsid protein of AAV5 and the 7-mer muscle-targeting peptide is inserted between S575 and S576 or between T577 and T578 of the capsid protein; the reference AAV capsid protein is a capsid protein of AAV6 and the 7-mer muscle-targeting peptide is inserted between D590 and P591 or S588 and T589 of the capsid protein; the reference AAV capsid protein is a capsid protein of AAV7 and the 7-mer muscle-targeting peptide is inserted between N589 and T590 of the capsid protein; the reference AAV capsid protein is a capsid protein of AAV8 and the 7-mer muscle-targeting peptide is inserted between N590 and T591 of the capsid protein; the reference AAV capsid protein is a capsid protein of AAV9 and the 7-mer muscle-targeting peptide is inserted between Q588 and A589 of the capsid protein; the reference AAV capsid protein is a capsid protein of AAVrh10 and the 7-mer muscle-targeting peptide is inserted between N590 and A591 of the capsid protein; the reference AAV capsid protein is a capsid protein of AAVpo.1 and the 7-mer muscle-targeting peptide is inserted between N567 and S568 or between N569 and T570 of the capsid protein; or the reference AAV capsid protein is a capsid protein of AAV12 and the 7-mer muscle-targeting peptide is inserted between N592 and A593 or between T594 and T595 of the capsid protein.

In some embodiments, the muscle targeting peptide is inserted into a site within VR VIII of a liver-toggle mutant capsid, optionally a liver-toggle mutant capsid as described in any one of embodiments 49 to 62. In some embodiments, the muscle targeting peptide is inserted into an amino acid position between 565 and 595 of the liver toggle mutant.

In some embodiments, the reference AAV capsid protein is a capsid protein of AAV1 and the targeting peptide is inserted between D590 and P591 or between S588 and T589 of the liver-toggle mutant; the reference AAV capsid protein is a capsid protein of AAV2 and the targeting peptide is inserted between R588 and Q589 or between N587 and R588 of the liver-toggle mutant; the reference AAV capsid protein is a capsid protein of AAV3b and the targeting peptide is inserted between S586 and S587 or between N588 and T589 of the liver-toggle mutant; the reference AAV capsid protein is a capsid protein of AAV4 and the targeting peptide is inserted between S584 and N585 or between S586 and N587 of the liver-toggle mutant; the reference AAV capsid protein is a capsid protein of AAV5 and the targeting peptide is inserted between S575 and S576 or between T577 and T578 of the liver-toggle mutant; the reference AAV capsid protein is a capsid protein of AAV6 and the targeting peptide is inserted between D590 and P591 or S588 and T589 of the liver-toggle mutant; the reference AAV capsid protein is a capsid protein of AAV7 and the targeting peptide is inserted between N589 and T590 of the liver-toggle mutant; the reference AAV capsid protein is a capsid protein of AAV8 and the targeting peptide is inserted between N590 and T591 of the liver-toggle mutant; the reference AAV capsid protein is a capsid protein of AAV9 and the targeting peptide is inserted between Q588 and A589 of the liver-toggle mutant; the reference AAV capsid protein is a capsid protein of AAVrh10 and the targeting peptide is inserted between N590 and A591 of the liver-toggle mutant; the reference AAV capsid protein is a capsid protein of AAVpo.1 and the targeting peptide is inserted between N567 and 5568 or between N569 and T570 of the liver-toggle mutant; or the reference AAV capsid protein is a capsid protein of AAV12 and the targeting peptide is inserted between N592 and A593 or between T594 and T595 of the liver-toggle mutant.

In some embodiments, the capsid protein has the sequence of SEQ ID NO: 158. In some embodiments, the rAAV capsid protein has the sequence of SEQ ID NO:159.

In some embodiments, the ERE comprises a constitutive promoter. In some embodiments, the constitutive promoter is the Rous sarcoma virus (RSV) LTR promoter (optionally with the RSV enhancer), the cytomegalovirus (CMV) promoter (optionally with the CMV enhancer), the SV40 promoter, the dihydrofolate reductase (DHFR) promoter, the β-actin promoter, the phosphoglycerol kinase 1 (PGK1) promoter (optionally the minimal PGK1 promoter), or the EF1 alpha promoter (optionally with intron).

In some embodiments, the ERE comprises an inducible promoter. In some embodiments, the inducible promoter is a tetracycline or rapamycin inducible promoter. In some embodiments, the ERE comprises a muscle-specific promoter. In some embodiments, the muscle specific promoter is a desmin promoter (which is optionally a CpG depleted desmin promoter), a CKM promoter derivative or an MTM1 promoter. In some embodiments, the promoter is a human promoter.

In some embodiments, the rAAV comprises a rabbit globin poly A sequence 3′ to the MTM1 coding sequence, optionally wherein the rabbit globin poly A sequence has at least 90% sequence identity to SEQ ID NO:177. In some embodiments, the rabbit globin poly A sequence has at least 95% sequence identity to SEQ ID NO:177. In some embodiments, the rabbit globin poly A sequence has at least 98% sequence identity to SEQ ID NO:177. In some embodiments, the rabbit globin poly A sequence has at least 99% sequence identity to SEQ ID NO:177. In some embodiments, the rabbit globin poly A sequence has 100% sequence identity to SEQ ID NO:177.

In some embodiments, the genome of the rAAV comprises AAV-derived inverted terminal repeat sequences (ITRs). In some embodiments, the ITRs are derived from AAV serotype 2. In some embodiments, the rAAV comprises a first ITR having at least 90% sequence identity to SEQ ID NO: 178 and a second ITR having at least 90% sequence identity to SEQ ID NO: 179. In some embodiments, the first ITR has at least 95% sequence identity to SEQ ID NO:178 and the second ITR has at least 95% sequence identity to SEQ ID NO: 179. In some embodiments, the first JTR has at least 98% sequence identity to SEQ ID NO:178 and the second ITR has at least 98% sequence identity to SEQ ID NO: 179. In some embodiments, the first JTR has at least 99% sequence identity to SEQ ID NO:178 and the second ITR has at least 99% sequence identity to SEQ ID NO:179. In some embodiments, the first ITR 100% sequence identity to SEQ ID NO: 178 and the second ITR has 100% sequence identity to SEQ ID NO:179.

In some embodiments, the rAAV comprises a heterologous splice acceptor sequence 5′ to the MTM1 coding sequence. In some embodiments, the heterologous splice acceptor sequence is derived from human beta globin exon 3. In some embodiments, the heterologous splice acceptor sequence comprises the nucleotide sequence of SEQ ID NO: 180.

In one aspect, the present disclosure provides an rAAV comprising: modified AAV capsid protein comprising at least one liver-toggle mutation and/or one muscle-targeting element, optionally wherein the modified capsid protein comprises the amino acid sequence of SEQ ID NO:158, SEQ ID NO:159, or SEQ ID NO:163, and a genome comprising: a first ITR sequence; a hybrid expression regulatory element (ERE) comprising a CMV enhancer and a chicken beta actin promoter, optionally wherein the ERE comprises the nucleotide sequence of SEQ ID NO: 173; an MTM1 coding sequence operably linked to the ERE; and a second ITR sequence.

In some embodiments, the rAAV further comprises a chimeric intron between the ERE and the MTM1 coding sequence, optionally wherein the chimeric intron comprises the nucleotide sequence of SEQ ID NO:176. In some embodiments, the rAAV further comprises a splice acceptor site 5′ to the MTM1 coding sequence, optionally wherein the splice acceptor site comprises the nucleotide sequence of SEQ ID NO:180. In some embodiments, the rAAV further comprises a polyadenylation sequence 3′ to the MTM1 coding sequence, optionally wherein the polyadenylation sequence comprises the nucleotide sequence of SEQ ID NO: 177.

In some embodiments, the MTM1 coding sequence is codon optimized for expression in human cells, optionally wherein the MTM1 coding sequence comprises the nucleotide sequence of SEQ ID NO:167, SEQ ID NO:168, SEQ ID NO:169 or SEQ ID NO:170.

In some embodiments, the rAAV has a genome which is self-complementary, optionally wherein the genome is fully self-complementary.

The present disclosure further provides a pharmaceutical composition comprising the rAAV described herein and a pharmaceutically acceptable carrier. In some embodiments, the pharmaceutical composition is in the form of a unit dose.

In some embodiments, the pharmaceutical composition comprises 1×10¹⁰ to 1×10¹⁶ genome copy numbers (GC) of the rAAV and/or in which the rAAV concentration is 1×10¹⁰ vg/ml to 1×10¹⁶ vg/ml.

In some embodiments, the pharmaceutical composition is formulated for parenteral administration, for example systemic (e.g., intravenous), intramuscular or subcutaneous administration.

The present disclosure further discloses a host cell engineered to produce the rAAV described herein. In some embodiments, the host cell comprises a polynucleotide expressing one or more capsid proteins of the rAAV, a functional rep gene, and a recombinant nucleic acid vector comprising AAV ITRs and the MTM coding sequence operably linked to an expression regulatory element (ERE), optionally wherein the ERE is a hybrid ERE comprising a CMV enhancer and a chicken beta actin promoter.

In another aspect, the present disclosure provides a method for treating or ameliorating or preventing X-linked myotubular myopathy in a subject, comprising administering a therapeutically effective amount of the rAAV or the pharmaceutical composition described herein. In some embodiments, the effective dose comprises 1×10¹⁰ to 1×10¹⁶ genome copy numbers (GC) of the rAAV. In some embodiments, the effective dose is 1×10¹⁵ GC or less. In some embodiments, the effective dose is 5×10¹⁴ GC or less. In some embodiments, the effective dose is 1×10¹⁴ GC or less. In some embodiments, the effective dose is 5×10¹³ GC or less. In some embodiments, the effective dose is 1×10¹³ GC or less.

In some embodiments, the administration is parenteral. In some embodiments, the administration is systemic (e.g., intravenous). In some embodiments, the administration is intramuscular. In some embodiments, the administration is subcutaneous.

In yet another aspect, the present disclosure provides the rAAV or the pharmaceutical composition described herein for use in treating and/or preventing X-linked myotubular myopathy. In some embodiments, the rAAV or the pharmaceutical composition is for use in expressing myotubularin in a muscle cell.

Without being bound by theory, it is believed that the rAAVs of the disclosure have improved therapeutics indices due to higher MTM1 expression levels per viral genome administered and/or reduce off-target (e.g., liver) tropism or expression per viral genome administered as compared to a control rAAV whose genome comprises the MTM1 coding sequence under the control of the desmin promoter and/or includes an unmodified capsid protein.

### 5. BRIEF DESCRIPTION OF THE SEVERAL VIEWS OF THE DRAWINGS

These and other features, aspects, and advantages of the present invention will become better understood with regard to the following description, and accompanying drawings where.

FIG. 1 illustrates the structure of an AAV VP1 protein with certain variable regions (VR I, VR III, VR IV) highlighted. The location of the liver toggle (mut1) in VR I and the peptide insertion (deco1) in VR VIII are indicated.

FIGS. 2A-2C provide the sequence alignment of VP1 sequences of certain AAV variants using AAV2 VP1 as a reference. The location of residue 168, the liver toggle site, mut1 (FIG. 2A), and the site of targeting peptide, deco1, insertion (FIG. 2B), are indicated.

FIGS. 3A-3D provide the sequence alignment of VP1 sequences of ancestral AAVs using AAV2 as a reference. The location of the liver toggle sites, residue 168 (FIG. 3A), and residue 266 (FIG. 3B), and the insertion site of a targeting peptide (FIG. 3C), are indicated. One or more representative member sequences for each of the Anc80, Anc81, Anc82, Anc83, Anc84, Anc94, Ac110, Anc113, Anc126 and Anc127 libraries were used for the alignment.

FIGS. 4A-4J shows immunohistochemistry data obtained from the experiment described in Example 2 below in the Example section. Anti-GFP immunohistochemistry was performed on liver with vehicle (FIG. 4A), AAV9 (FIG. 4B), AAV^{mut1} (FIG. 4C), AAV^{deco1} (FIG. 4D), or AAV^{mut1-deco1} (FIG. 4E); and skeletal muscle (quadriceps) tissue cross-sections of mice injected with vehicle (FIG. 4F), AAV9 (FIG. 4G), AAV^{mut1} (FIG. 4I), AAV^{deco1} (FIG. 4I), or AAV^{mut1-deco1} (FIG. 4J).

FIGS. 5A-5B show mRNA expression in various tissues of C57BL/6 mice treated with different AAV vectors, as measure by RT-ddPCR. Y-axis represents the ratio of copies of eGFP mRNA transcripts over RPP30 mRNA and x-axis represents AAV vectors and the dose injected into the experimental animals. Each graph shows eGFP expression in liver (FIG. 5A) and quadriceps (FIG. 5B).

FIGS. 6A-6E show eGFP mRNA expression in various tissues of C57BL/6 mice treated with different AAV vectors, as measure by RT-ddPCR. Y-axis represents the ratio of copies of eGFP over RPP30 mRNA and x-axis represents AAV vectors and the dose injected into the experimental animals. Each graph shows eGFP expression in liver (FIG. 6A), heart (FIG. 6B), triceps surae (FIG. 6C), quadriceps (FIG. 6D), or diaphragm (FIG. 6E).

FIGS. 7A-7D show eGFP vector genome (DNA) and eGFP expression (mRNA) in liver and quad tissues of C57BL/6 mice treated with vehicle, AAV^{Mut1} and AAV^{Mut1-deco1} AAV vectors. DNA data is shown in FIGS. 7A and 7B with eGFP genomic copies as measured by RT-ddPCR plotted at 14 and 28 days, respectively. Y-axis represents vector genome (copies per DPG) and x-axis represents vehicle and AAV vectors. mRNA data is shown in FIGS. 7C and 7D with eGFP expression as measured by RT-ddPCR plotted at 14 and 28 days, respectively. Y-axis represents the ratio of copies of eGFP over RPP30 mRNA and x-axis represents AAV vectors.

FIG. 8 shows eGFP mRNA expression in various tissues of BalbC mice treated with vehicle, AAV^{mut1} and AAV^{mut1-deco1} AAV vectors, as measured by RT-ddPCR. Y-axis represents the ratio of copies of eGFP over RPP30 mRNA and x-axis represents AAV vectors and the dose injected into the experimental animals. The graph shows eGFP expression in liver (left) and quadriceps (right).

FIGS. 9A and 9B show exemplary IHC tissue analysis obtained from of Run 1 samples from NHPs. Liver tissue is shown in FIG. 9A, the left side shows tissue obtained from an AAV9 vector treated NHP and the right side shows tissue obtained from an AAV^{mut1_deco1} vector treated NHP; exemplary IHC quadriceps tissue is shown in FIG. 9B, obtained from AAV9 vector treated NHP on left and AAV^{mut1_deco1} vector treated NHP on the right.

FIG. 10 shows the % GFP positive cells in the liver tissue (right and left side of the organ) and quadriceps tissue (right and left leg) in slides obtained from Run 1 from NHPs administered vehicle, AAV9 or AAV^{mut1-deco1} vector.

FIG. 11 shows the % GFP positive cells in various skeletal muscle and liver tissue (average from Runs 1 and 2) in slides obtained from NHPs administered vehicle, AAV9 or AAV^{mut1_deco1} vector.

FIG. 12 shows the % GFP positive cells per animal in various skeletal muscle and liver tissue (average from Runs 1 and 2) in slides obtained from NHPs administered vehicle, AAV9 or AAV^{mut1_deco1} vector.

FIG. 13 shows the average combined quantification of % GFP positive cells per animal in various skeletal muscle and liver tissue (average from Runs 1 and 2) obtained from NHPs administered vehicle, AAV9 or AAV^{mut1_deco1} vector.

FIG. 14 shows the % GFP positive cells in various cardiac tissues (average from Runs 1 and 2) obtained from NHPs administered vehicle, AAV9 or AAV^{mut1_deco1} vector.

FIG. 15 shows the % GFP positive cells per animal in various cardiac muscle (average from Runs 1 and 2) obtained from NHPs administered vehicle, AAV9 or AAV^{mut1_deco1} vector.

FIG. 16 shows the average % GFP positive cells per animal in ventricle wall, atria, inter ventr septum slides (average from Runs 1 and 2) obtained from NHPs administered vehicle, AAV9 or AAV^{mut1_deco1} vectors.

FIGS. 17A-17C shows the average % GFP positive cells per NHP animal in various tissues (average from Runs 1 and 2) administered vehicle and AAV9 and AAV^{mut1_deco1} vectors. FIG. 17A shows average % GFP positive cells per animal in liver tissue. FIG. 17B shows average % GFP positive cells per animal in various skeletal muscle tissue. FIG. 17C shows average % GFP positive cells per animal in various cardiac tissue.

FIGS. 18A-18D show the results of DNA samples analyzed for biodistribution of vector genomes in the liver and quadriceps tissue using a duplexed ddPCR method targeting the transgene (eGFP) and a reference gene (RPP30). The results are shown in FIGS. 18A (liver), 18B (quadriceps), 18C (biceps), 18D (heart) where the x-axis represents AAV vectors (wild type AAV9 on the left and AAV^{mut1deco1} on the right of each plot) and indicating whether the sample was taken from the left or right side of the organ/animal.

FIGS. 19A-19D show the results of mRNA transcript analysis measured by eGFP copies of eGFP over RPP30 mRNA. FIGS. 19A (liver), 19B (quadriceps), 19C (biceps), 19D (heart), are illustrated where the x-axis represents AAV vectors (wild type AAV9 on the left and AAV^{mut1deco1} on the right) and indicating whether the sample was taken from the left or right side of the organ/animal.

FIG. 20 shows human MTM1 protein expression in RD cells. The expression level of human MTM protein was determined by automated JESS-ProteinSimple instrument. Each bar represents by peak area values of JESS, either before (blue) or after (orange) being normalized to total protein load. Data were obtained from one run using the 1:4 dilution as described in the western protocol.

### 6. DETAILED DESCRIPTION OF THE INVENTION

### 6.1. Definitions

The term “reference AAV capsid protein” as used herein refers to a VP1, VP2, or VP3 capsid protein of a naturally occurring AAV variant or a non-naturally occurring VP1, VP2, or VP3 capsid protein that is known in the art.

The term “liver-toggle mutant” or “liver-toggle mutant of a reference AAV capsid protein” as used herein refers to a capsid protein comprising a sequence different from the reference AAV capsid protein by having (i) an alanine (A) or glycine (G) amino acid residue at an amino acid position corresponding to position 266 in Anc80 VP1 and/or b) a lysine (K) or arginine (R) amino acid residue at an amino acid position corresponding to position 168 in Anc80 VP1. In some embodiments, a liver-toggle mutant of a reference AAV capsid protein is a capsid protein comprising a sequence different from the reference AAV capsid protein by having an alanine (A) amino acid residue at an amino acid position corresponding to position 267 in AAV9 VP1 protein and a threonine (T) amino acid residue at an amino acid position corresponding to position 269 in AAV9 VP1. The liver toggle mutant can have tropism, specificity or distribution in a liver different from the reference AAV capsid protein when administered to a mammalian subject. The mammalian subject can be a human, non-human primate (NHP), mice, rats, birds, rabbits, guinea pigs, hamsters, farm animals (including pigs and sheep), dogs, or cats.

The term “targeting peptide” as used herein refers to a peptide capable of directing AAV to a target cell, tissue or organ in vivo. An AAV comprising a capsid protein with a targeting peptide has an increased localization in a target cell, tissue or organ compared to the AAV with a capsid protein without the target peptide.

The term “amino acid position” as here herein refers to a position of an amino acid residue in an AAV VP1 protein sequence, counted from the first amino acid in the N terminal.

For the avoidance of doubt, as used herein, the indication that an insertion site is at amino acid position X means that the targeting peptide is inserted between amino acids X and X+1, i.e., the targeting peptide is inserted after the indicated amino acid.

The term “liver off” is used herein to describe an AAV having a lower tropism to liver or less biodistribution in liver when administered to a mammalian subject compared to other AAV variants. The term “liver off” is also used to describe a modification in the AAV capsid protein that reduces the tropism to liver or biodistribution in liver when administered to a mammalian subject.

The term “liver on” is used herein to describe an AAV having a higher tropism to liver or more biodistribution in liver when administered to a mammalian subject compared to other AAV variants. The term “liver on” is also used to describe a modification in the AAV capsid protein that increases the tropism to liver or biodistribution in liver when administered to a mammalian subject.

“AAV” is adeno-associated virus and may be used to refer to the virus itself or derivatives thereof. The term covers all subtypes, serotypes and pseudotypes, and both naturally occurring and recombinant forms, except where required otherwise.

The term “AAV capsid protein” or simply “capsid protein” refers to a VP1, VP2, or VP3 capsid protein. The AAV capsid protein may be naturally occurring or synthetic/artificial (e.g., ancestral) capsid protein or a capsid protein that is modified as compared to such naturally occurring or synthetic/artificial capsid protein, referred to as a “modified AAV capsid protein” or simply “modified capsid protein”. The naturally occurring or synthetic capsid protein against which a modified AAV capsid protein is referred to herein as a “reference” capsid protein. In some embodiments, the AAV capsid protein is a wild type or modified capsid protein of AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-13; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI. In some embodiments, the modified capsid protein is a modified VP1 capsid protein.

The term “amino acid position” as here herein refers to a position of an amino acid residue in an AAV VP1 protein sequence, counted from the first amino acid in the N terminal.

The term “CAG” when used in relation to a promoter or ERE refers to a promoter or ERE with chicken beta actin promoter and CMV enhancer sequences.

The term “constitutive” promoter or ERE as used herein refers to a nucleotide sequence which, when operably linked with a polynucleotide which encodes or specifies a gene product, causes the gene product to be produced in a cell under most or all physiological conditions of the cell.

The term “expression regulatory element” or “ERE” as used herein in the context of the rAAV of the disclosure refers to a nucleic acid sequence which is required for expression of the MTM1 coding sequence operably linked to the ERE. In some instances, an ERE sequence may be the core promoter sequence and in other instances, this sequence may also include an enhancer sequence and other regulatory elements which are required for expression of the gene product, for example exon sequences.

The term “functional fragment” in the context of the myotubularin or MTM1 refers to a biologically functional fragment of myotubularin or MTM1. As would be understood in the art, a biologically functional fragment is a portion or portions of a full length sequence that retain a biological function of the full length sequence. An exemplary functional fragment corresponds to amino acids 29-486 of SEQ ID NO:165 (and is disclosed herein as SEQ ID NO:164). Biological functions of MTM1 include the ability cleave or hydrolyze an endogenous phosphoinositide substrate known in the art, or an artificial phosphoinositide substrate for in vitro assays (i.e., a phosphoinositide phosphatase activity), to recruit and/or associate with other proteins such as, for example, the GTPase Rab5, the PI 3-kinase Vps34 or Vps15 (i.e., proper localization), or treat myotubular myopathy.

The term “functional variant” in the context of the myotubularin or MTM1 refers to various splicing isoforms, variants, fusion proteins, and modified forms of the wildtype MTM1 polypeptide or a functional fragment thereof. Such isoforms, bioactive fragments or variants, fusion proteins, and modified forms of the MTM1 polypeptides retain at least one biological function of the full length MTM1 protein (e.g., a protein of SEQ ID NO 165).

The term “inducible” promoter or ERE as used herein refers to a nucleotide sequence which, when operably linked with a polynucleotide which encodes or specifies a gene product, causes the gene product to be produced in a cell substantially only when an inducer which corresponds to the promoter is present in the cell.

As used herein, the term “internalizing moiety” refers to a moiety capable of interacting with a target tissue or a cell type to effect delivery of the attached molecule into the cell (i.e., penetrate desired cell; transport across a cellular membrane). In certain embodiments, an MTM1 polypeptide encoded by the rAAV of the disclosure can be a fusion protein comprising an internalizing moiety. In some embodiments, the internalizing moiety selectively, although not necessarily exclusively, targets and penetrates muscle cells. In certain embodiments, the internalizing moiety has limited cross-reactivity, and thus preferentially targets a particular cell or tissue type. In certain embodiments, suitable internalizing moieties include, for example, antibodies, monoclonal antibodies, or derivatives or analogs thereof. Other internalizing moieties include for example, homing peptides, receptors, and ligands. In certain embodiments, the internalizing moiety mediates transit across cellular membranes via an ENT2 transporter. Exemplary internalizing moieties are disclosed in U.S. Pat. No. 9,447,394 B2, the contents of which are incorporated by reference herein.

The term “inverted terminal repeat” (or “ITR”) refers to a polynucleotide sequence found at the ends of AAV genomes that form a hairpin, which contributes to the genome's ability to self-prime (allowing for primase-independent synthesis of the complementary second DNA strand) and provides for encapsidation of the genome into an AAV particle. An ITR can be a wild-type ITR or a variant thereof.

The terms “liver-toggle mutant”, “liver-toggle mutant of a reference AAV capsid protein” and the like, as used herein, refers to a capsid protein comprising a sequence different from a reference AAV capsid protein by having one or more mutations (e.g., amino acid substitutions) that alter tropism, specificity or distribution in a liver as compared to the reference AAV capsid protein when administered to a mammalian subject (such a sequence difference referred to herein as a “liver toggle mutation”). The mammalian subject can be a human, non-human primate (NHP), mice, rats, birds, rabbits, guinea pigs, hamsters, farm animals (including pigs and sheep), dogs, or cats. Exemplary liver toggle mutations are disclosed in WO2019/217911 and WO2021/050614, incorporated by reference in their entireties herein. In some embodiments, the liver toggle mutations comprise (i) an alanine (A) or guanine (G) amino acid residue at an amino acid position corresponding to position 266 in Anc80 VP1 and/or b) a lysine (K) or arginine (R) amino acid residue at an amino acid position corresponding to position 168 in Anc80 VP1. In other embodiments, a liver-toggle mutant of a reference AAV capsid protein is a capsid protein comprising a sequence different from the reference AAV capsid protein by having an alanine (A) amino acid residue at an amino acid position corresponding to position 267 in AAV9 VP1 protein and a threonine (T) amino acid residue at an amino acid position corresponding to position 269 in AAV9 VP1. In yet further embodiments, the liver toggle mutations comprise a sequence different from the reference AAV capsid protein by having any combination of (i) an arginine (R) instead of serine (S) at position 446; (ii) an alanine (A) instead of an arginine (R) at position 471; and (iii) a threonine (T) or alanine (A) instead of a valine (V) at position 708, in each case numbered according to an AAV2 reference capsid protein (SEQ ID NO:1 of WO2021/050614, which is incorporated by reference herein).

The term “liver off” is used herein to describe an AAV having a lower tropism to liver or less biodistribution in liver when administered to a mammalian subject compared to other AAV variants. The term “liver off” is also used to describe a modification in the AAV capsid protein that reduces the tropism to liver or biodistribution in liver when administered to a mammalian subject.

The term “liver on” is used herein to describe an AAV having a higher tropism to liver or more biodistribution in liver when administered to a mammalian subject compared to other AAV variants. The term “liver on” is also used to describe a modification in the AAV capsid protein that increases the tropism to liver or biodistribution in liver when administered to a mammalian subject.

The term “MTM1 coding sequence” is used herein to refer to a specific sequence of nucleotides in a polynucleotide, such as an rAAV genome or mRNA produced thereby, that encodes an MTM1 polypeptide.

The term “MTM1 polypeptide” refers to a polypeptide comprising an amino acid sequence having at least 90%, at least 95%, at least 98%, at least 99% or 100% identity to human MTM1 (SEQ ID NO:165) or a functional fragment (e.g., SEQ ID NO:164) or functional variant thereof.

The terms “operably linked” and “operatively linked” refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA.

The term “parenteral” administration of a composition includes, e.g., subcutaneous (s.c.), intravenous (i.v.), intramuscular (i.m.), or intrasternal injection, or infusion techniques.

The terms “peptide”, “polypeptide” and “protein” are used interchangeably herein to refer to a polymer of amino acid residues.

The term “pharmaceutically acceptable carrier” includes any of the standard pharmaceutical carriers, excipients, stabilizers and adjuvants. For examples of carriers, excipients, stabilizers and adjuvants, see Remington: The Science and Practice of Pharmacy, 22nd Revised Ed., Pharmaceutical Press, 2012.

The abbreviation “rAAV” refers to a recombinant adeno-associated viral particle composed of at least one AAV capsid protein and an encapsidated polynucleotide, sometimes referred to herein as a “genome”. rAAV can include a genome that comprises a heterologous polynucleotide (i.e., a polynucleotide other than a wild-type AAV genome), such as a heterologous polynucleotide encoding a gene delivered to a mammalian cell such as the MTM1 gene. The heterologous nucleotide is sometimes referred to as a transgene.

The term “self-complementary” rAAV vector or genome as used herein means a fully or partially self-complementary rAAV vector or genome, respectively. A “fully self-complementary” rAAV vector refers to a vector containing a genome generated by the absence of a terminal resolution site (TR) from one of the ITRs of the rAAV. The absence of a TR prevents the initiation of replication at the vector terminus where the TR is not present. In general, fully self-complementary rAAV vectors generate single-stranded, inverted repeat genomes, with a wild-type (wt) AAV TR at each end and a mutated TR (mTR) in the middle. Thus, a fully self-complementary rAAV genome is typically a single stranded polynucleotide having, in the 5′ to 3′ direction, a first ITR sequence, a heterologous sequence (e.g., MTM1 coding sequence and/or ERE), a second ITR sequence, a second heterologous sequence that is complementary to the first heterologous sequence, and a third ITR sequence. A “partially self-complementary” rAAV genome refers to a single stranded polynucleotide having, in the 5′ to 3′ direction or the 3′ to 5′ direction, a first ITR sequence, a heterologous sequence (e.g., MTM1 coding sequence and/or ERE), a second ITR sequence, and a self-complementary region that is complementary to a portion of the heterologous sequence and has a length that is less than the entire length the heterologous sequence.

The term “targeting peptide” as used herein refers to a peptide capable of directing AAV to a target cell, tissue or organ in vivo. An AAV comprising a capsid protein with a target peptide has an increased localization in a target cell, tissue or organ compared to the AAV with a capsid protein without the target peptide.

For the avoidance of doubt, as used herein, the indication that an insertion site is at amino acid position X means that the targeting peptide is inserted between amino acids X and X+l, i.e., the targeting peptide is inserted after the indicated amino acid.

The term “tissue-specific” promoter or ERE as used herein refers to a nucleotide sequence which, when operably linked with a polynucleotide encodes or specified by a gene, causes the gene product to be produced in a cell substantially only if the cell is a cell of the tissue type corresponding to the promoter.

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The terms “vector”, “AAV vector” and “rAAV vector” refer to an rAAV that comprises a heterologous polynucleotide, e.g., a transgene.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### 6.2. Modified AAV Capsid Proteins

One aspect of the present disclosure provides a modified adeno-associated virus (AAV) capsid protein, comprising: (i) a reference AAV capsid protein, and (ii) a targeting peptide inserted into an insertion site of the reference AAV capsid protein. In some embodiments, the targeting peptide is a 7-mer peptide having the sequence RGDLLLS (SEQ ID NO: 1).

In some embodiments, the modified AAV capsid protein further includes a liver-toggle mutation relative to a reference AAV capsid protein. The liver-toggle mutant can comprise (1) an alanine (A) or glycine (G) amino acid residue at an amino acid position corresponding to position 266 in Anc80 VP1; and/or (2) a lysine (K) or arginine (R) amino acid residue at an amino acid position corresponding to position 168 in Anc80 VP1.

6.2.1. Reference AAV Capsid Proteins

The reference AAV capsid protein used in various embodiments of the present disclosure is a VP1, VP2 or VP3 capsid protein of an AAV known in the art. It can be a VP1, VP2 or VP3 capsid protein of a naturally occurring or non-naturally occurring AAV variant.

The non-naturally occurring VP1, VP2, or VP3 capsid protein includes a capsid protein generated by biological or chemical alteration or in silico design, or variation of a naturally occurring AAV capsid protein. Accordingly, the reference AAV capsid protein includes, but is not limited to, a capsid protein of various AAV serotypes (e.g., AAV1, AAV2, AAV3B, AAV5, AAV6, AAV8, and AAV9) or a variant thereof. A non-naturally occurring VP1, VP2, or VP3 capsid protein further includes an artificial capsid protein created by in silico design or synthesis. An artificial capsid protein includes, but is not limited to, AAV capsid proteins disclosed in PCT/US2014/060163, U.S. Pat. No. 9,695,220, PCT/US2016/044819, PCT/US2018/032166, PCT/US2019/031851, and PCT/US2019/047546, which are incorporated herein by reference in their entireties.

In some embodiments, the reference AAV capsid protein is the capsid protein of AAV9 (Genbank Ace. No: AAS99264.1), AAV1 (Genbank Ace. No: AAD27757.1), AAV2 (Genbank Ace. No: AAC03780.1), AAV3 (Genbank Ace. No: AAC55049.1), AAV3b (Genbank Ace. No: AF028705.1), AAV4 (Genbank Ace. No: AAC58045.1), AAV5 (Genbank Ace. No: AAD13756.1). AAV6 (Genbank Ace. No: AF028704.1), AAV7 (Genbank Ace. No: AAN03855.1), AAV 8 (Genbank Ace. No: AAN03857.1), AAV10 (Genbank Ace. No: AAT46337.1), AAVrh10 (Genbank Ace. No: AY243015.1), AAV11 (Genbank Ace. No: AAT46339.1), AAV12 (Genbank Ace. No: AB116639.1), or AAV13 (Genbank Ace. No: ABZ10812.1), AAVpol (Genbank Ace. No: FJ688147.1). In certain embodiments, the AAV capsid protein is the capsid protein of AAV9 (Genbank Ace. No: AA599264.1).

The reference AAV capsid protein can be VP1 capsid protein having a sequence selected from: SEQ ID NO: 54 (AAV1 (AAD27757)), SEQ ID NO: 55 (AAV2 (AAC03780)), SEQ ID NO: 56 (AAV3 (AAC55049)), SEQ ID NO: 57 (AAV5 (AAD13756)), SEQ ID NO: 58 (AAV6 (AAB95450)), SEQ ID NO: 59 (AAV7 (AF513851_2)), SEQ ID NO: 60 (AAV8 (AF513852_2)), SEQ ID NO: 61 (AAV9 (AAS99264)), SEQ ID NO: 62 (AAV10 (AAT46337)), SEQ ID NO: 63 (AAV hu.68), SEQ ID NO: 64 (AAV LK03), SEQ ID NO: 65 (AAV hu.1 (AAS99260)), SEQ ID NO: 66 (AAV hu.2 (AA599270)), SEQ ID NO: 67 (AAV hu.3 (AAS99280)), SEQ ID NO: 68 (AAV hu.4 (AAS99287)), SEQ ID NO: 69 (AAV hu.6 (AA599306)), SEQ ID NO: 70 (AAV hu.7 (AAS99313)), SEQ ID NO: 71 (AAV hu.9 (AAS99314)), SEQ ID NO: 72 (AAV hu.10 (AAS99261)), SEQ ID NO: 73 (AAV hu.11 (AAS99262)), SEQ ID NO: 74 (AAV hu.15 (AA S99265)), SEQ ID NO: 75 (AAV hu.16 (AA S99266)), SEQ ID NO: 76 (AAV hu.17 (AAS99267)), SEQ ID NO: 77 (AAV hu.18 (AAS99268)), SEQ ID NO: 78 (AAV hu.20 (AAS99271)), SEQ ID NO: 79 (AAV hu.21 (AAS99272)), SEQ ID NO: 80 (AAV hu.22 (AAS99273)), SEQ ID NO: 81 (AAV hu.23 (AAS99274)), SEQ ID NO: 82 (AAV hu.25 (AAS99276)), SEQ ID NO: 83 (AAV hu.27 (AAS99277)), SEQ ID NO: 84 (AAV hu.28 (AAS99278)), SEQ ID NO: 85 (AAV hu.29 (AAS99279)), SEQ ID NO: 86 (AAV hu.31 (AAS99281)), SEQ ID NO: 87 (AAV hu.32 (AA599282)), SEQ ID NO: 88 (AAV hu.34 (AAS99283)), SEQ ID NO: 89 (AAV hu.37 (AA S99285)), SEQ ID NO: 90 (AAV hu.39 (AAS99286)), SEQ ID NO: 91 (AAV hu.41 (AAS99289)), SEQ ID NO: 92 (AAV hu.42 (AAS99290)), SEQ ID NO: 93 (AAV hu.43 (AA S99291)), SEQ ID NO: 94 (AAV hu.44 (AAS99292)), SEQ ID NO: 95 (AAV hu.45 (AAS99293)), SEQ ID NO: 96 (AAV hu.46 (AAS99294)), SEQ ID NO: 97 (AAV hu.47 (AAS99295)), SEQ ID NO: 98 (AAV hu.48 (AAS99296)), SEQ ID NO: 99 (AAV hu.51 (AAS99298)), SEQ ID NO: 100 (AAV hu.52 (AAS99299)), SEQ ID NO: 101 (AAV hu.53 (AAS99300)), SEQ ID NO: 102 (AAV hu.54 (AAS99301)), SEQ ID NO: 103 (AAV hu.55 (AAS99302)), SEQ ID NO: 104 (AAV hu.56 (AAS99303)), SEQ ID NO: 105 (AAV hu.57 (AAS99304)), SEQ ID NO: 106 (AAV hu.60 (AAS99307)), SEQ ID NO: 107 (AAV hu.61 (AAS99308)), SEQ ID NO: 108 (AAV hu.63 (AAS99309)), SEQ ID NO: 109 (AAV hu.66 (AAS99311)), SEQ ID NO: 110 (AAV hu.67 (AAS99312)), SEQ ID NO: 111 (AAV rh.10 (AA088201)), SEQ ID NO: 112 (AAV rh.13 (AA088199)), SEQ ID NO: 113 (AAV rh.19 (AA088194)), SEQ ID NO: 114 (AAV rh.22 (AA088192)), SEQ ID NO: 115 (AAV rh.23 (AA088191)), SEQ ID NO: 116 (AAV rh.24 (AA088190)), SEQ ID NO: 117 (AAV rh.35 (AA088186)), SEQ ID NO: 118 (AAV rh.43 (AAS99245)), SEQ ID NO: 119 (AAV rh.48 (AAS99246)), SEQ ID NO: 120 (AAV rh.49 (AAS99247)), SEQ ID NO: 121 (AAV rh.50 (AAS99248)), SEQ ID NO: 122 (AAV rh.51 (AAS99249)), SEQ ID NO: 123 (AAV rh.52 (AAS99250)), SEQ ID NO: 124 (AAV rh.53 (AAS99251)), SEQ ID NO: 125 (AAV rh.54 (AAS99252)), SEQ ID NO: 126 (AAV rh.55 (AAS99253)), SEQ ID NO: 127 (AAV rh.57 (AAS99254)), SEQ ID NO: 128 (AAV rh.58 (AAS99255)), SEQ ID NO: 129 (AAV rh.62 (AAS99258)), SEQ ID NO: 130 (AAV rh.64 (AAS99259)), SEQ ID NO: 131 (AAV rh.56 (JA400164)), SEQ ID NO: 143 (Anc80L1), SEQ ID NO: 144 (Anc80L27), SEQ ID NO: 145 (Anc80L33), SEQ ID NO: 146 (Anc80L36), SEQ ID NO: 147 (Anc80L44), SEQ ID NO: 148 (Anc80L59), SEQ ID NO: 149 (Anc80L60), SEQ ID NO: 150 (Anc80L62), SEQ ID NO: 151 (Anc82DI), and SEQ ID NO: 152 (AAV rh.74). The reference AAV capsid protein can be a VP2 or VP3 protein having a part of one of the sequences. For example, VP2 protein can have a sequence corresponding to amino acids 138 to 736 of AAV9 VP1 and VP3 protein can have a sequence corresponding to amino acids 138 to 736 of AAV9 VP1 protein.

The reference AAV capsid protein can be VP1 capsid protein having any member sequence of the ancestral AAV library selected from SEQ ID NO: 132 (Anc80), SEQ ID NO: 133 (Anc81 (AKU89596)), SEQ ID NO: 134 (Anc82 (AKLT89597)), SEQ ID NO: 135 (Anc83 (AKU89598)), SEQ ID NO: 136 (Anc84 (AKU89599)), SEQ ID NO: 137 (Anc94) SEQ ID NO: 138 (Anc110 (AKU89600)), SEQ ID NO: 139 (Anc113 (AKU89601)), SEQ ID NO: 140 (Anc126 (AKU89602)), SEQ ID NO: 141 Anc127 (AKU89603), and SEQ ID NO: 142 (Anc80L65 (AKU89595)). The reference AAV capsid protein can be a VP2 or VP3 protein having a part of one of the sequences. For example, VP2 protein can have a sequence corresponding to amino acids 138 to 736 of AAV9 VP1 and VP3 protein can have a sequence corresponding to amino acids 138 to 736 of AAV9 VP1 protein. When a SEQ ID NO for a library sequence is used in this disclosure, it refers to a sequence of any one member of the library.

In some embodiments, the reference AAV capsid protein is a liver-toggle mutant described in WO2019/217911, which is incorporated by reference in its entirety herein.

In some embodiments, the reference AAV capsid protein is a capsid protein (VP1, VP2 or VP3) of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; rh74; hu.67-E; hu.17-E; hu.6-E; hu.66-E; rh.38-E; hu.32-F; AAV9/hu; hu.31-F; Anc80L27; Anc80L59; Anc80L60; Anc80L62; Anc80L65; Anc80L33; Anc80L36; Anc80L44; Anc80L1; Anc110; and Anc80DI. In some embodiments, the reference AAV capsid protein is a capsid protein of any member protein of an ancestral AAV library selected from: Anc80; Anc8l; Anc82; Anc83; Anc84; Anc94; Anc113; Anc126; and Anc127.

In some embodiments, the reference AAV capsid protein is a protein having a sequence selected from SEQ ID Nos: 54-131 and 143-152. In some embodiments, the reference AAV capsid protein is a protein having a VP2 (corresponding to amino acids 138 to 736 of AAV9 VP1) or VP3 portion (corresponding to amino acids 138 to 736 of AAV9 VP1) of the protein having a sequence selected from SEQ ID NOs: 54-131 and 143-152.

In some embodiments, the reference AAV capsid protein is a capsid protein of the AAV variant modified to include one or more liver-toggle mutations described in WO2019/217911. In some embodiments, the reference AAV capsid protein comprises (1) an alanine (A) or glycine (G) amino acid residue at an amino acid position corresponding to position 266 in Anc80 VP1 and/or (2) a lysine (K) or arginine (R) amino acid residue at an amino acid position corresponding to position 168 in Anc80 VP1. In some embodiments, the reference AAV capsid protein comprises (i) an alanine (A) amino acid residue at an amino acid position corresponding to position 266 in Anc80 VP1 and/or b) a lysine (K) amino acid residue at an amino acid position corresponding to position 168 in Anc80 VP1. In some embodiments, the reference AAV capsid protein comprises (ii) an alanine (A) amino acid residue at an amino acid position corresponding to position 267 in AAV9 VP1 protein and/or a threonine (T) amino acid residue at an amino acid position corresponding to position 269 in AAV9 VP1.

6.2.2. Liver-Toggle Mutant

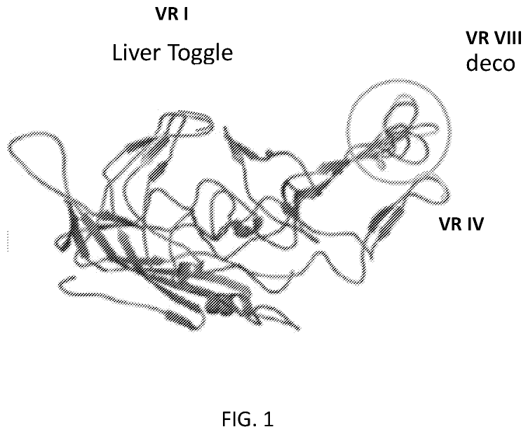

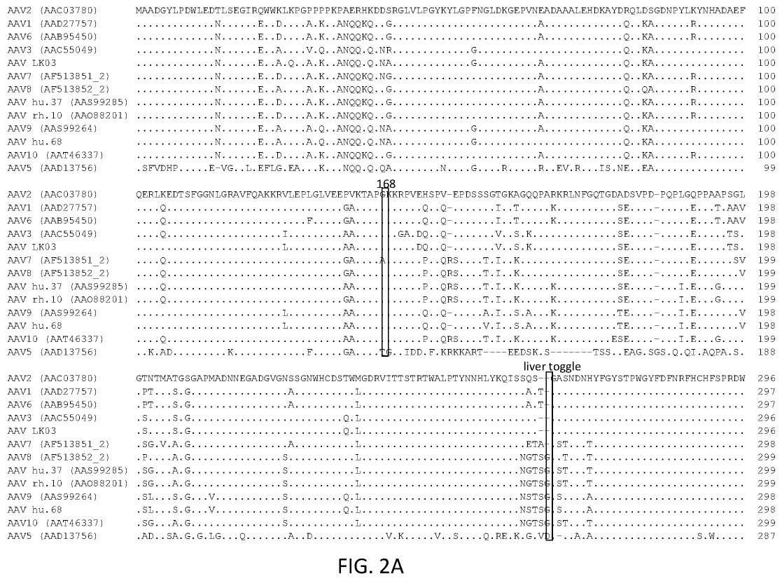

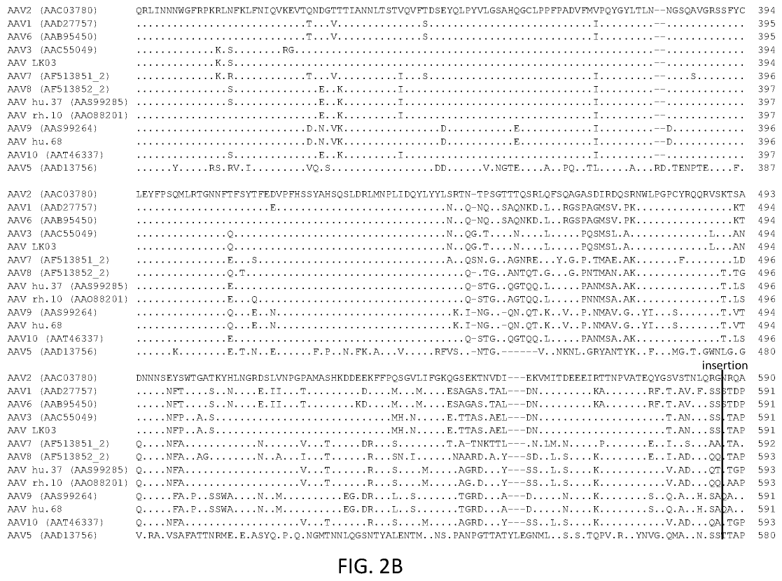

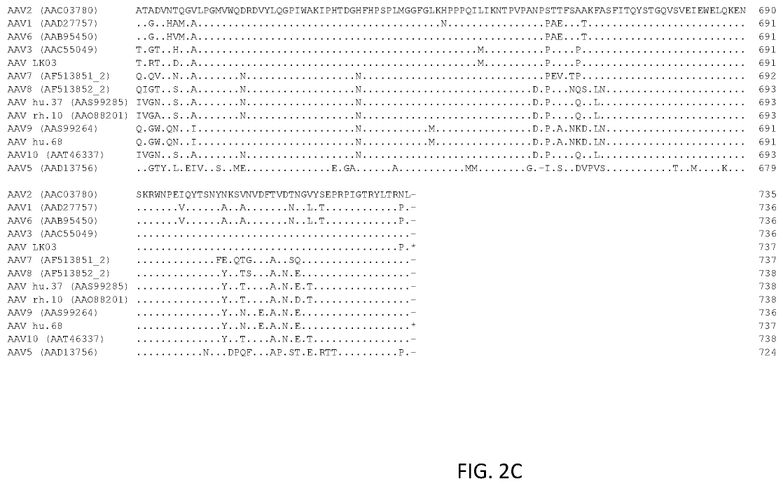

In some embodiments, the modified AAV capsid protein comprises a liver-toggle mutant of a reference AAV capsid protein. In some embodiments, the liver-toggle mutant is different from the reference AAV capsid protein by having one or more amino acid substitutions at a variable region of the reference AAV capsid protein. In some embodiments, the one or more amino acid substitutions is at a variable region, VR I, of the reference AAV capsid protein (FIG. 1).

The liver-toggle mutant can be a natural protein or a protein genetically engineered, or biologically or chemically produced. The liver toggle mutant can have tropism, specificity or localization different from the reference AAV capsid protein, particularly in liver, when administered to a mammalian subject. The mammalian subject can be a human, non-human primate (NHP), mice, rats, birds, rabbits, guinea pigs, hamsters, farm animals (including pigs and sheep), dogs, or cats.

In some embodiments, the liver-toggle mutant comprises a sequence different from a reference AAV capsid protein by having an amino acid substitution at an amino acid position corresponding to position 266 in Anc80 VP1 and/or at an amino acid position corresponding to position 168 in Anc80 VP1.

In some embodiments, the liver-toggle mutant comprises a sequence different from a reference AAV capsid protein by having (1) an alanine (A) amino acid residue at an amino acid position corresponding to position 266 in Anc80 VP1 or (2) a lysine (K) amino acid residue at an amino acid position corresponding to position 168 in Anc80 VP1. In some embodiments, the liver-toggle mutant comprises a sequence different from a reference AAV capsid protein by having (1) an alanine (A) amino acid residue at an amino acid position corresponding to position 266 in Anc80 VP1 and (2) a lysine (K) amino acid residue at an amino acid position corresponding to position 168 in Anc80 VP1.

In some embodiments, the liver-toggle mutant comprises a sequence different from a reference AAV capsid protein by having a glycine (G) amino acid residue at an amino acid position corresponding to position 266 in Anc80; or an arginine (R) amino acid residue at an amino acid position corresponding to position 168 in Anc80. In some embodiments, the liver-toggle mutant comprises a sequence different from a reference AAV capsid protein by having a glycine (G) amino acid residue at an amino acid position corresponding to position 266 in Anc80; and an arginine (R) amino acid residue at an amino acid position corresponding to position 168 in Anc80.

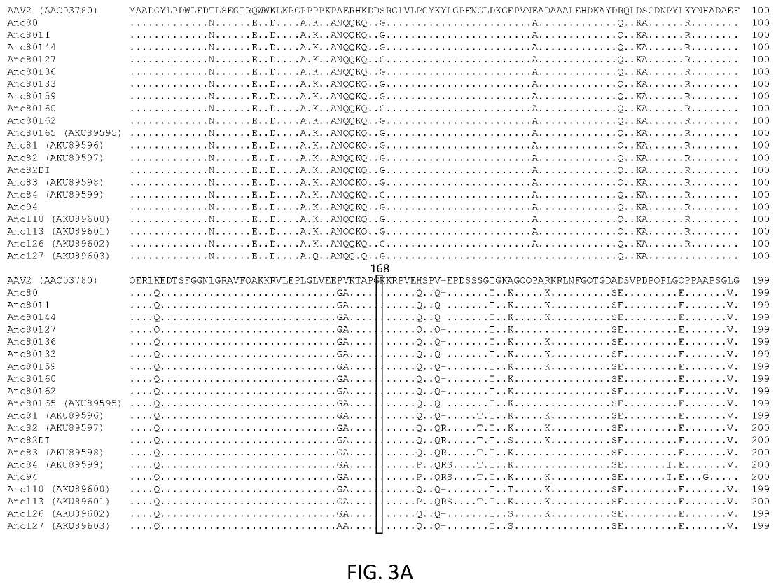

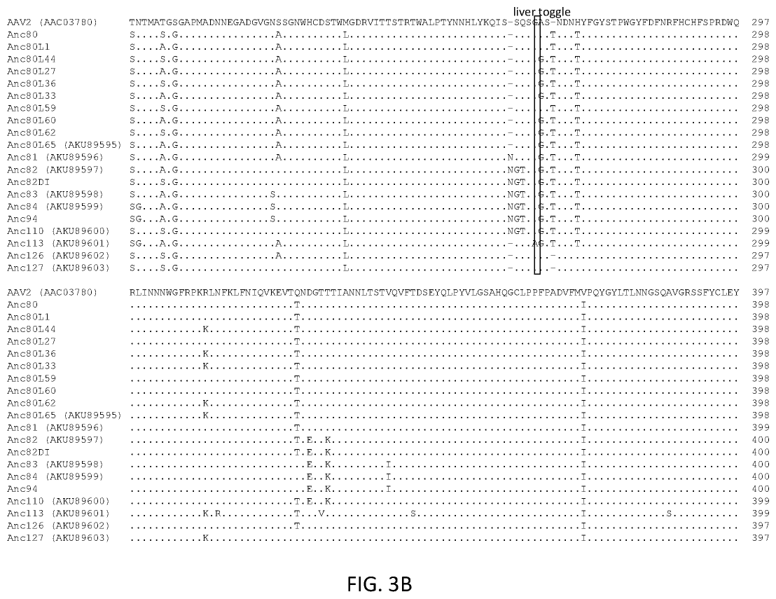

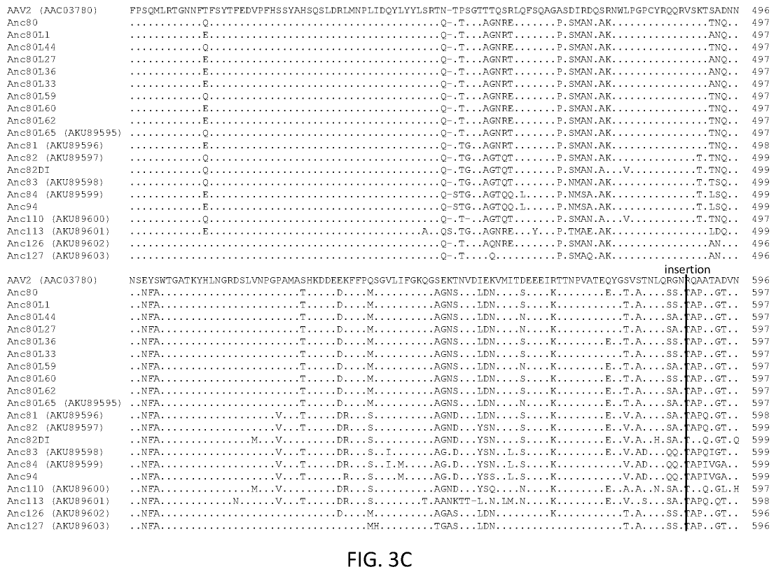

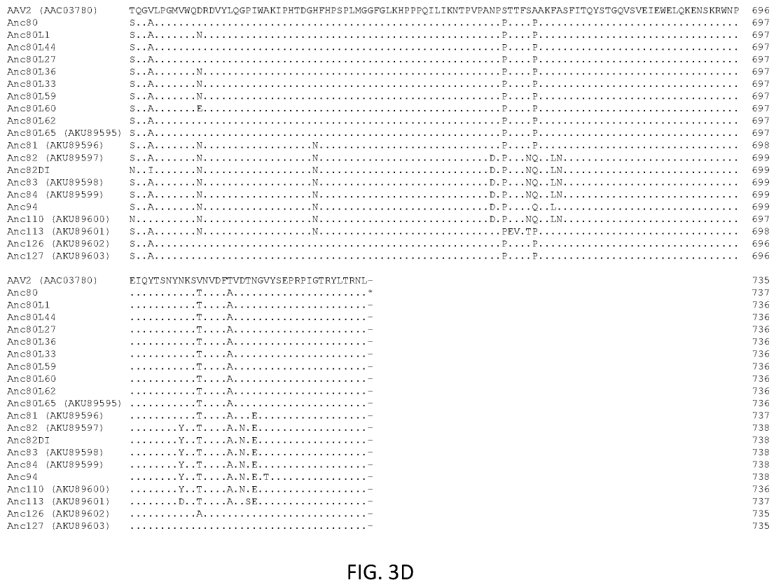

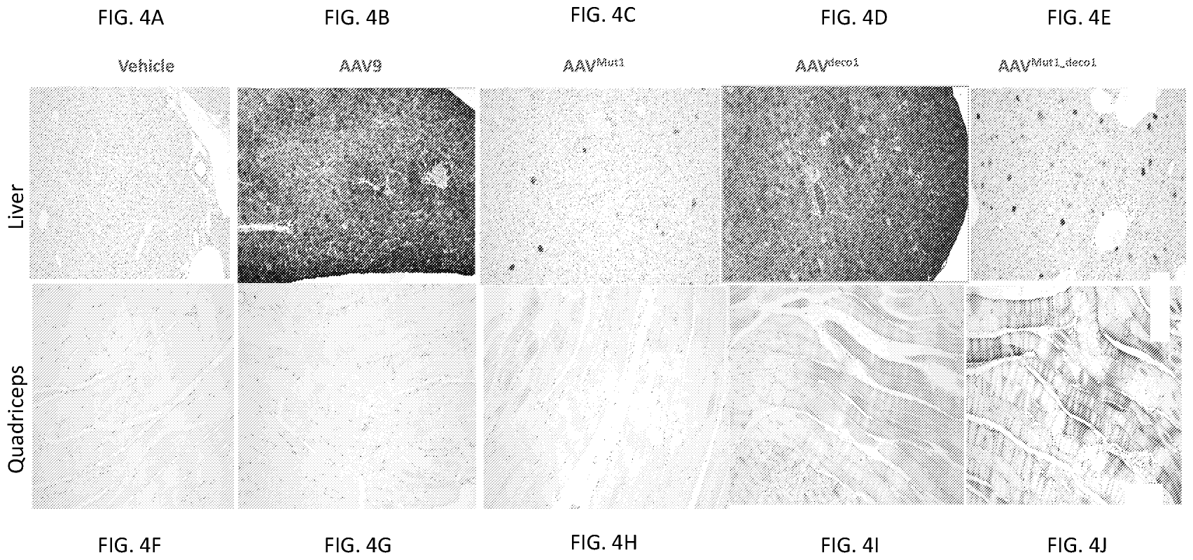

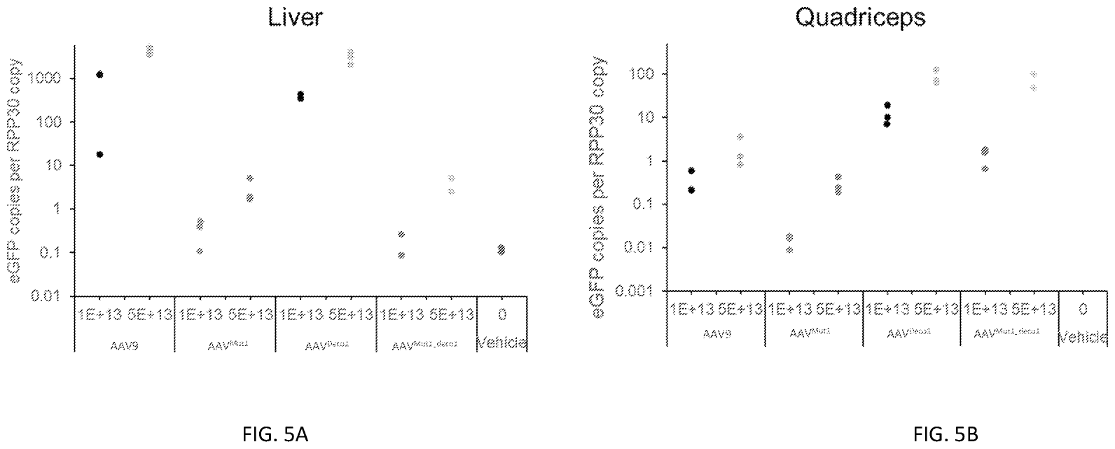

An amino acid position corresponding to position 266 in Anc80 VP1 and an amino acid position corresponding to position 168 in Anc80 VP1 in various VP1 protein sequences are indicated with boxes in FIGS. 3A-C and FIGS. 4A-D.

In some embodiments, a liver-toggle mutant is different from a reference AAV capsid protein only at an amino acid position corresponding to position 266 in Anc80 VP1 or an amino acid position corresponding to position 168 in Anc80 VP1. In some embodiments, a liver-toggle mutant is different from a reference AAV capsid protein only at two amino acid positions—an amino acid position corresponding to position 266 in Anc80 VP1 and an amino acid position corresponding to position 168 in Anc80 VP1. In some embodiments, a liver-toggle mutant is different from a reference AAV capsid protein by more than the two amino acid substitutions.

In some embodiments, the liver-toggle mutant comprises a sequence different from a reference AAV capsid protein by having an alanine (A) amino acid residue at an amino acid position corresponding to position 267 in AAV9 VP1 protein or a threonine (T) amino acid residue at an amino acid position corresponding to position 269 in AAV9 VP1. In some embodiments, the liver-toggle mutant comprises a sequence different from a reference AAV capsid protein by having an alanine (A) amino acid residue at an amino acid position corresponding to position 267 in AAV9 VP1 protein and a threonine (T) amino acid residue at an amino acid position corresponding to position 269 in AAV9 VP1. In some embodiments, a liver-toggle mutant is different from a reference AAV capsid protein only at an amino acid position corresponding to position 267 in AAV9 VP1 protein or an amino acid position corresponding to position 269 in AAV9 VP1. In some embodiments, a liver-toggle mutant is different from a reference AAV capsid protein only at two amino acid positions—an amino acid position corresponding to position 267 in AAV9 VP1 protein and an amino acid position corresponding to position 269 in AAV9 VP1.

In some embodiments, a liver-toggle mutant is an AAV capsid protein disclosed in WO2019/217911, which is incorporated by reference in its entirety herein.

In particular, an AAV capsid protein that is described therein to generate a “liver off” (“liver de-targeting”) AAV can be used in embodiments herein. In other embodiments, an AAV capsid protein that is described therein to generate a “liver on” (“liver targeting”) AAV can be used herein.

In some embodiments, two amino acid positions corresponding to position 266 and position 168 of Anc80 VP1 protein are used as liver-toggle positions. In some embodiments, AAV with a capsid protein having an alanine (A) amino acid residue at an amino acid position corresponding to position 266 in Anc80 VP1 or b) a lysine (K) amino acid residue at an amino acid position corresponding to position 168 in Anc80 VP1 exhibits liver-off phenotypes. In some embodiments, AAV with a capsid protein having a glycine (G) amino acid residue at an amino acid position corresponding to position 266 in Anc80 VP1 or b) an arginine (R) amino acid residue at an amino acid position corresponding to position 168 in Anc80 VP1 exhibits liver-on phenotypes.

In some embodiments, more than one toggle region residues are introduced to enhance the liver-off or the liver-on phenotypes. In some embodiments, a double mutant AAV9 G267A S269T is used.

In some embodiments, a liver-toggle mutant is Anc80L65 capsid protein with a G266A mutation. In some embodiments, a liver-toggle mutant is AAV9 capsid protein with a G267A mutation. In some embodiments, a liver-toggle mutant is AAV9 capsid protein with G267A and S269T mutations.

In some embodiments, the liver-toggle mutant comprises (1) an alanine (A) amino acid residue at an amino acid position corresponding to position 504 in AAV9; and (2) an alanine (A) amino acid residue at an amino acid position corresponding to position 505 in AAV9.

6.2.3. Targeting Peptide

In some embodiments, a modified AAV capsid protein of the present disclosure comprises a targeting peptide.

The target peptide can vary in length. For example, the targeting peptide can be, or be at least, three, four, five, six, seven, eight, nine, ten, eleven, twelve, fifteen, eighteen, twenty, twenty-five, thirty, or a range between any two of these values, amino acids long. In some embodiments, the targeting peptide is, or is about, seven amino acids long. In some embodiments, the targeting peptide is, or is about, eleven amino acids long. In some embodiments, the targeting peptide is, or is about, seven to eleven amino acids long.

In typical embodiments, the targeting peptide is capable of changing tropism and/or specificity of an AAV when the AAV is formed with a capsid protein containing the targeting peptide. In some embodiments, the targeting peptide increases targeting of the AAV to a target cell, tissue or organ. In some embodiments, the targeting peptide decreases targeting of the AAV to an off-target cell, tissue or organ. In some embodiments, the targeting peptide increases targeting of the AAV to a target cell, tissue or organ after systemic administration (e.g., after intravenous administration). In some embodiments, the targeting peptide decreases targeting of the AAV to an off-target cell, tissue or organ after systemic administration (e.g., after intravenous administration). In some embodiments, the targeting peptide increases targeting of the AAV to a target cell, tissue or organ after local administration. In some embodiments, the targeting peptide decreases targeting of the AAV to an off-target cell, tissue or organ after local administration.

The targeting peptide can vary in length. For example, the targeting peptide can be, or be at least, three, four, five, six, seven, eight, nine, ten, eleven, twelve, fifteen, eighteen, twenty, twenty-five, thirty, or a range between any two of these values, amino acids long.

In some embodiments, the modified AAV capsid protein comprises a single copy of the targeting peptide. In some embodiments, the modified AAV capsid protein comprises more than one copy of the targeting peptide.

In some embodiments, the targeting peptide can enhance targeting of an AAV to a brain, muscle, spinal cord, eye, liver, muscle, or other organ. In some embodiments, the targeting peptide can decrease targeting of an AAV to a brain, muscle, spinal cord, eye, liver, muscle, or other organ.

Sequences of exemplary targeting peptides that can be used various embodiments of the present disclosure are provided in SEQ ID Nos: 1-53, 153-157, and 160-162.

6.2.3.1 7-Mer Peptide

In some embodiments, the targeting peptide is a 7-mer peptide. In some embodiments, the 7-mer peptide has the sequence RGDX¹X²X³X⁴ (SEQ ID NO: 52), wherein X¹ to X⁴ are independently selected amino acid residues. As used herein, the term “amino acid” comprises naturally occurring L- and D-amino acids and artificial, i.e. non-naturally occurring, α-amino acids. Preferably, the amino acid is a naturally occurring amino acid. In preferred embodiments, the amino acid is a naturally occurring L-α-amino acid.

In some embodiments, in the targeting peptide according to SEQ ID NO: 52, X¹, X², and X³ are independently selected from L, G, V, and A; and X⁴ is S, V, A, G, or L.

In some embodiments, X¹ is selected from L, Q, D, H, M, P, and K. In some embodiments, X¹ is L. In some embodiments, X² is selected from G, V, S, D, M, and N. In some embodiments, X² is G. In some embodiments, X³ is selected from V, M, P, S, and D. In some embodiments, X³ is V. In some embodiments, X⁴ is selected from S, N, L, H, and M. In some embodiments, X⁴ is S.

In some embodiments, the targeting peptide is according to SEQ ID NO: 52, wherein X¹ is L. In further embodiments, the targeting peptide is according to SEQ ID NO: 52, wherein X² is G. In further embodiments, the targeting peptide is according to SEQ ID NO: 52, wherein X³ is L. In further embodiments, the targeting peptide is according to SEQ ID NO: 52, wherein X⁴ is S.

In some embodiments, the targeting peptide is according to SEQ ID NO: 52, wherein X¹ is A. In further embodiments, the targeting peptide is according to SEQ ID NO: 52, wherein X² is V. In further embodiments, the targeting peptide is according to SEQ ID NO: 52, wherein X³ is G. In further embodiments, the targeting peptide is according to SEQ ID NO: 52, wherein X⁴ is V.

In some embodiments, the targeting peptide is according to SEQ ID NO: 52, wherein X¹ is L. In further embodiments, the targeting peptide is according to SEQ ID NO: 52, wherein X² is L. In further embodiments, the targeting peptide is according to SEQ ID NO: 52, wherein X³ is L. In further embodiments, the targeting peptide is according to SEQ ID NO: 52, wherein X⁴ is S.

In some embodiments, X¹ is L; X² is selected from G, L and V; X³ is selected from L and G; and/or X⁴ is selected from S, V and L. In a further embodiment, in the targeting peptide according to SEQ ID NO: 52, X¹ is L, X² is G or L, and/or X⁴ is S. In certain embodiments, in the targeting peptide according to SEQ ID NO: 52, at least one of X² and X³ is G or L.

In certain embodiments, in the targeting peptide according to SEQ ID NO: 52, X¹, X², and X³ are independently selected from L, V, and A; at least two of X¹, X², and X¹ are independently L. In some embodiments, X¹, X², and X³ are L.

In certain embodiments, in the targeting peptide according to SEQ ID NO: 52, X² is L.

In certain embodiments, the targeting peptide comprises, alternatively consists of, the amino acid sequence selected from RGDLRVS (SEQ ID NO: 153), RGDAVGV (SEQ ID NO: 154), RGDFTPTS (SEQ ID NO: 155), RGDLGLS (SEQ ID NO: 156), and RGDMSRE (SEQ ID NO: 157), and/or a sequence comprising at most two, preferably at most one, amino acid substitution compared to one of the aforesaid specific sequences. In certain embodiments, the targeting peptide does not comprise an amino acid sequence selected from RGDLRVS (SEQ ID NO: 153), RGDAVGV (SEQ ID NO: 154), RGDFTPTS (SEQ ID NO: 155), RGDLGLS (SEQ ID NO: 156), and RGDMSRE (SEQ ID NO: 157).

In some embodiments, the targeting peptide has a sequence of RGDLLLS (SEQ ID NO: 1).

6.2.3.2 BBB Peptide

In some embodiments, the targeting peptide is the targeting peptide disclosed in US2017/0166926, incorporated by reference in its entirety herein.

The targeting peptide can have any of the sequences selected from SEQ ID NOs: 2-51 and 53 provided herein. In some embodiments, the targeting peptide is the 7-mer peptide TLAVPFK (SEQ ID NO: 53).

6.2.4. Insertion Sites

A modified AAV capsid protein of the present disclosure comprises a targeting peptide inserted into an insertion site of a reference AAV capsid protein or a liver-toggle mutant of a reference AAV capsid protein.

Preferably, the targeting peptide is inserted at a site exposed to the exterior of the capsid, preferably based on structure predictions and/or experimental data. More preferably, the insertion site of the targeting peptide is at a site exposed to the exterior of the AAV capsid in a manner that does not interfere with the activity of said protein in capsid assembly.

In some embodiments, the insertion site is located in one of the variable regions, VR I, VR VIII, or VR IV, of the capsid protein (FIG. 1). In some embodiments, the insertion site is in the variable region, VR VIII (deco).

An insertion site in an AAV capsid protein that “corresponds to” an insertion site in the AAV9 capsid protein can be established by the skilled person by known methods, preferably by aligning the amino acids of the capsid proteins. In some embodiments, the insertion site of the targeting peptide corresponds to amino acid position 588 of the AAV9 VP1 capsid protein. In some embodiments, the insertion site of the targeting peptide corresponds to amino acid position 589 of the AAV9 VP1 capsid protein.

The insertion site can be any one of those described in WO2019/207132, incorporated by reference in its entirety herein. Some of the insertion sites are provided below in Table 1 and highlighted in FIGS. 3A-3C and FIGS. 4A-4D. In Table 1, the preferred insertion sites are indicated by a “-” relative to wild type VP1 capsid polypeptide.

TABLE 1
Exemplary insertion sites

| | Insertion sites 1 | | Insertion sites 2 | |
|---|---|---|---|---|
| AAV1 | D590 P591 | STD-PAT | S588 T589 | SSS-TDP |
| AAV2 | R588 Q589 | GNR-QAA | N587 R588 | RGN-RQA |
| AAV3b | S586 S587 | LQS-SNT | N588 T589 | SSN-TAP |
| AAV4 | S584 N585 | DQS-NSN | S586 N587 | SNS-NLP |
| AAV5 | S575 S576 | NQS-STT | T577 T578 | SST-TAP |
| AAV6 | D590 P591 | STD-PAT | S588 T589 | SSS-TDP |
| AAV7 | N589 T590 | AAN-TAA | | |
| AAV8 | N590 T591 | QQN-TAP | | |
| AAV9 | Q588 A589 | SAQ-AQA | | |
| AAVrh10 | N590 A591 | QQN-AAP | | |
| AAVpo.1 | N567 S568 | NQN-SNT | N569 T570 | NSN-THP |
| AAV12 | N592 A593 | NQN-ATT | T594 T595 | NAT-TAP |

6.2.5. Various Embodiments of Modified AAV Capsid Proteins

The present disclosure provides various embodiments of modified AAV capsid proteins. In one aspect, the modified AAV capsid protein comprises (i) a reference AAV capsid protein, and (ii) a 7-mer peptide having the sequence RGDLLLS (SEQ ID NO: 1) inserted into a site within VR VIII of the reference AAV capsid protein.

In some embodiments, the modified AAV capsid protein is an AAV9 capsid protein containing a targeting peptide, RGDLLLS (SEQ ID NO: 1), inserted into the VR VIII. In one embodiment, the modified AAV capsid protein has a sequence of SEQ ID NO: 158. In some embodiments, the modified AAV capsid protein has the amino acids 138 to 736 of SEQ ID NO: 158. In some embodiments, the modified AAV capsid protein has the amino acids 203 to 736 of SEQ ID NO: 158.

In some embodiments, the modified AAV capsid protein has a sequence having at least 90%, 91%, 92%, 93%, 94%, 95%, 96%, 97%, 98%, or 99% identity to SEQ ID NO: 158.

In another aspect, the present disclosure provides a modified AAV capsid protein comprising (i) a liver-toggle mutant of a reference AAV capsid protein, comprising a) an alanine (A) amino acid residue at an amino acid position corresponding to position 266 in Anc80; or b) a lysine (K) amino acid residue at an amino acid position corresponding to position 168 in Anc80; and (ii) a targeting peptide inserted into a site within VR VIII of the liver-toggle mutant.

In one embodiment, the modified AAV capsid protein is an AAV9 capsid protein containing a targeting peptide, RGDLLLS (SEQ ID NO: 1), inserted into the VR VIII and a liver-toggle mutation. In one embodiment, the modified AAV capsid protein has a sequence of SEQ ID NO: 159. In some embodiments, the modified AAV capsid protein has the amino acids 138 to 736 of SEQ ID NO: 159. In some embodiments, the modified AAV capsid protein has the amino acids 203 to 736 of SEQ ID NO: 159.

In some embodiments, the modified AAV capsid protein has a sequence having at least 90%, 91%, 92%, 93%, 94%, 95%, 96%, 97%, 98%, or 99% identity to SEQ ID NO: 159.

A modified AAV capsid protein of the present disclosure can change the tropism, specificity and/or bio-distribution of an AAV comprising the modified AAV capsid protein. In preferred embodiments, an AAV comprising the modified AAV capsid protein has increased targeting to a target cell, tissue or organ when administered to a subject. In some embodiments, an AAV comprising the modified AAV capsid protein has decreased distribution outside of a target cell, tissue or organ when administered to a subject.

### 6.3. Polynucleotides Encoding Modified AAV Capsid Proteins; Vectors; Host Cells

In another aspect, the present disclosure provides a polynucleotide encoding a modified AAV capsid protein described herein. In some embodiments, the polynucleotide is codon optimized for expression in a bacterial or mammalian cell.

In some embodiments, the polynucleotide is inserted into an expression vector. In some embodiments, the polynucleotide is operably linked to a promoter or a sequence inducing expression of a protein from the polynucleotide. The present disclosure provides a vector including the polynucleotide encoding a modified AAV capsid protein. The vector can be used for generation of the modified AAV capsid protein. In some embodiments, the vector is used to generate an AAV virion comprising the modified AAV capsid protein. In some embodiments, the vector further comprises an AAV rep protein or a fragment thereof. In some embodiments, the reference capsid protein for the modified AAV capsid protein and the rep protein are originated from an AAV of the same clade. In some embodiments, the reference capsid protein for the modified AAV capsid protein and the rep protein are originated from an AAV of different clades.

In some embodiments, the polynucleotide is transfected to a host cell. The present disclosure provides a host cell comprising the polynucleotide encoding a modified AAV capsid protein. The host cell can be a prokaryotic cell or eukaryotic cell. In some embodiments, the host cell is a mammalian cell or a yeast cell.

In some embodiments, the host cell further comprises another polynucleotide encoding an AAV protein. In some embodiments, the host cell comprises a functional rep gene; a recombinant nucleic acid vector comprising AAV inverted terminal repeats (ITRs) and an expressible polynucleotide; and sufficient helper functions to permit packaging of the recombinant nucleic acid vector into the modified AAV capsid protein.

In some embodiments, the components required for the host cell to package a recombinant nucleic acid vector in a modified AAV capsid protein are provided to the host cell in trans. In some embodiments, any one or more of the required components (e.g., a recombinant nucleic acid vector, rep sequences, cap sequences, and/or helper functions) are provided by a stable host cell which has been engineered to contain one or more of the required components using methods known to those of skill in the art. In some embodiments, such a stable host cell contains the required component(s) under the control of an inducible promoter. In some embodiments, the required component(s) is under the control of a constitutive promoter.

### 6.4. Recombinant Nucleic Acid Vector Containing an Expressible Polynucleotide

In one aspect, the present disclosure provides a recombinant nucleic acid vector containing an expressible polynucleotide. In some embodiments, the recombinant nucleic acid vector is encapsulated in the modified AAV capsid proteins disclosed herein. In some embodiments, the recombinant nucleic acid vector is encapsulated in the reference AAV capsid protein. In preferred embodiments, the expressible polynucleotide comprises a transgene (in cis or trans configuration with other viral sequences).

The transgene can be, for example, a reporter gene (e.g., beta-lactamase, beta-galactosidase (LacZ), alkaline phosphatase, thymidine kinase, green fluorescent polypeptide (GFP), chloramphenicol acetyltransferase (CAT), or luciferase, or fusion polypeptides that include an antigen tag domain such as hemagglutinin or Myc), or a therapeutic gene (e.g., genes encoding hormones or receptors thereof, growth factors or receptors thereof, differentiation factors or receptors thereof, immune system regulators (e.g., cytokines and interleukins) or receptors thereof, enzymes, RNAs (e.g., inhibitory RNAs or catalytic RNAs), or target antigens (e.g., oncogenic antigens, autoimmune antigens). In some embodiments, the modified rAAV comprises an expressible polynucleotide encoding a therapeutic tRNA, miRNA, gene editing guide RNA, or RNA-editing guide RNA.

The transgene can be selected depending, at least in part, on the particular disease or deficiency being treated. Simply by way of example, gene transfer or gene therapy can be applied to the treatment of hemophilia, retinitis pigmentosa, cystic fibrosis, leber congenital amaurosis, lysosomal storage disorders, inborn errors of metabolism (e.g., inborn errors of amino acid metabolism including phenylketonuria, inborn errors of organic acid metabolism including propionic acidemia, inborn errors of fatty acid metabolism including medium-chain acyl-CoA dehydrogenase deficiency (MCAD)), cancer, achromatopsia, cone-rod dystrophies, macular degenerations (e.g., age-related macular degeneration), lipopolypeptide lipase deficiency, familial hypercholesterolemia, spinal muscular atrophy, Duchenne's muscular dystrophy, Alzheimer's disease, Parkinson's disease, obesity, inflammatory bowel disorder, diabetes, congestive heart failure, hypercholesterolemia, hearing loss, coronary heart disease, familial renal amyloidosis, Marfan's syndrome, fatal familial insomnia, Creutzfeldt-Jakob disease, sickle-cell disease, Huntington's disease, fronto-temporal lobar degeneration, Usher syndrome, lactose intolerance, lipid storage disorders (e.g., Niemann-Pick disease, type C), Batten disease, choroideremia, glycogen storage disease type II (Pompe disease), ataxia telangiectasia (Louis-Bar syndrome), congenital hypothyroidism, severe combined immunodeficiency (SCID), and/or amyotrophic lateral sclerosis (ALS). A transgene also can be, for example, an immunogen that is useful for immunizing a subject (e.g., a human, an animal (e.g., a companion animal, a farm animal, an endangered animal). For example, immunogens can be obtained from an organism (e.g., a pathogenic organism) or an immunogenic portion or component thereof (e.g., a toxin polypeptide or a by-product thereof). By way of example, pathogenic organisms from which immunogenic polypeptides can be obtained include viruses (e.g., picornavirus, enteroviruses, orthomyxovirus, reovirus, retrovirus), prokaryotes (e.g., Pneumococci, Staphylococci, Listeria, Pseudomonas), and eukaryotes (e.g., amebiasis, malaria, leishmaniasis, nematodes). It would be understood that the methods described herein and compositions produced by such methods are not to be limited by any particular transgene.

### 6.4.1. MTM1 Transgene

In certain embodiment, the transgene is the MTM1 transgene for treatment of subjects (preferably human subjects) suffering from XLMTM and/or carrying mutations in the MTM1 gene. “Treatment” of MTM encompasses a complete reversal or cure of the disease, or any range of improvement in conditions and/or adverse effects attributable to MTM. Merely to illustrate, “treatment” of MTM includes an improvement in any of the following effects associated with MTM or combination thereof: short life expectancy, respiratory insufficiency (partially or completely), poor muscle tone, drooping eyelids, poor strength in proximal muscles, poor strength in distal muscles, facial weakness with or without eye muscle weakness, abnormal curvature of the spine, joint deformities, and weakness in the muscles that control eye movement (ophthalmoplegia). Improvements in any of these conditions can be readily assessed according to standard methods and techniques known in the art.

A modified rAAV of the present disclosure can be administered to a subject in a suitable pharmaceutical carrier, e.g., as described herein.

The rAAV of the disclosure are typically administered in sufficient amounts to transduce or infect the desired cells and to provide sufficient levels of gene transfer and expression to provide a therapeutic benefit to subjects suffering from XLMTM or carrying a mutation in the MTM1 gene, without undue adverse effects.

Transduction and/or expression of the MTM1 transgene can be monitored at various time points following administration by DNA, RNA, or protein assays.

The MTM1 transgene can encode an MTM1 polypeptide, i.e., a polypeptide comprising the amino acid sequence of MTM1 or a functional fragment or a functional variant thereof.

The structure and various motifs of the MTM1 polypeptide have been well characterized in the art (see, e.g., Laporte et al, 2003, Human Molecular Genetics, 12(2):R285-R292; Laporte et al., 2002, Journal of Cell Science 15:3105-3117; Lorenzo et al., 2006, Journal of Cell Science 119:2953-2959). As such, in certain embodiments, various functional fragments or variants of the MTM1 polypeptides can be designed and identified by screening polypeptides made, for example, recombinantly from the corresponding fragment of the nucleic acid encoding an MTM1 polypeptide. For example, several domains of MTM1 have been shown to be important for its phosphatase activity or localization. To illustrate, these domains include: Glucosyltransferase, Rab-like GTPase Activator and Myotubularins (GRAM; amino acid positions 29-97 or up to 160 of SEQ ID NO:165), Rac-Induced recruitment Domain (RID; amino acid positions 161-272 of SEQ ID NO: 165), PTP/DSP homology (amino acid positions 273-471 of SEQ ID NO: 165; catalytic cysteine is amino acid 375 of SEQ ID NO: 165), and SET-interacting domain (SID; amino acid positions 435-486 of SEQ ID NO: 165). Accordingly, any combination of such domains may be constructed to identify fragments or variants of MTM1 that exhibit a biologically activity of native MTM1.

Exemplary functional fragments of an MTM1 polypeptide include fragments comprising amino acids 29-486 of SEQ ID NO:165 (i.e., the amino acid sequence of SEQ ID NO: 164). Thus, in certain embodiments, the MTM1 polypeptides comprise amino acid residues 29-486 of SEQ ID NO:165 or the amino acid sequence of SEQ ID NO:164.

In some embodiments, the MTM1 polypeptide comprises an amino acid sequence having at least 90%, at least 95%, at least 98%, at least 99% or 100% identity to a functional fragment of human MTM1 having the amino acid sequence of SEQ ID NO:164. In some embodiments, the MTM1 polypeptide is a full length MTM1 polypeptide (e.g., a polypeptide of SEQ ID NO:165).

In other embodiments, the MTM1 polypeptide is a fusion polypeptide comprising an amino acid sequence having at least at least 90%, at least 95%, at least 98%, at least 99% or 100% identity to SEQ ID NO:164 fused to another polypeptide portion, e.g., one or more polypeptide portions that enhance one or more of in vivo stability, in vivo half-life, uptake/administration, and/or purification. In some embodiments, the polypeptide portion is an internalizing moiety.

In some embodiments, the MTM1 coding sequence comprises a nucleotide sequence having at least 80% sequence identity to SEQ ID NO:166, which is of the native MTM1 coding sequence, or a portion thereof encoding a functional fragment of wild type MTM1, e.g., the functional fragment corresponding to amino acids 29-486 of MTM1 (SEQ ID NO:164). In certain embodiments, the MTM1 coding sequence comprises a nucleotide sequence at least 80%, at least 85%, at least 90%, at least 95%, at least 97%, at least 98%, at least 99% or 100% identical to SEQ ID NO: 166 or a portion thereof encoding a functional fragment of wild type MTM1, e.g., the functional fragment corresponding to amino acids 29-486 of MTM1 (SEQ ID NO:164).

In other embodiments, the MTM1 coding sequence comprises a nucleotide sequence having at least 80% sequence identity to any of SEQ ID NOs:167, 168 and 169, which are codon-optimized for expression in human cells, or to a portion of any of SEQ ID NOs:167, 168 and 169 encoding a functional fragment of wild type MTM1, e.g., the functional fragment corresponding to amino acids 29-486 of MTM1 (SEQ ID NO:164). In certain embodiments, the MTM1 coding sequence comprises a nucleotide sequence at least 80%, at least 85%, at least 90%, at least 95%, at least 97%, at least 98%, at least 99% or 100% identical to any one of SEQ ID NOs:167, 168 and 169, or to or to a portion of any of SEQ ID NOs:167, 168 and 169 encoding a functional fragment of wild type MTM1, e.g., the functional fragment corresponding to amino acids 29-486 of MTM1 (SEQ ID NO:164).

In some embodiments, the MTM1 coding sequence may further comprise a nucleotide sequence that encodes a linker and/or an internalizing moiety. In some embodiments, the internalizing moiety is an antibody or an antigen-binding fragment thereof.

### 6.4.2. Regulatory Sequences

The recombinant nucleic acid vector of the disclosure typically comprise regulatory sequences operably linked to expressible polynucleotide (e.g., the MTM1 coding sequence). The regulatory sequence will generally be appropriate for a cell to be transduced with the expressible polynucleotide (e.g., MTM1 coding sequence), such as skeletal muscle cells. Numerous types of regulatory sequence and are known the art and may include, but are not limited to, promoter sequences, leader or signal sequences, ribosomal binding sites, transcriptional start and termination sequences, translational start and termination sequences, and enhancer or activator sequences.

In some embodiments, the regulatory sequence includes expression regulatory elements (EREs), e.g., EREs comprising a promoter and optionally an enhancer. The promoter is a major DNA regulatory element in the rAAV genome that determines the level of the expressible polynucleotide (e.g., MTM1 coding sequence) expression and in which cells it will be expressed. The choice of promoter is therefore a key aspect of the design of AAV vectors. Furthermore, the size of the promoter is also relevant as AAVs have a maximum packaging capacity of ˜4700 nucleotides.

In some embodiments, the promoter is a constitutive promoter. In other embodiments, the promoter is a tissue-specific (e.g., muscle-specific) promoter. In yet other embodiments, the promoter is an inducible promoter.

Other suitable features of the rAAV include ITR sequences (e.g., wild type ITRs or a combination of wild type ITR sequences and an ITR sequence lacking a functional terminal resolution site, for example as set forth in SEQ ID NO: 178 and SEQ ID NO: 179), a intron (e.g., a chimeric intron comprising human herpesvirus beta and human globin 3 intronic sequences, for example as set forth in SEQ ID NO: 176), a splice acceptor sequence 5′ of the MTM1 coding sequence (e.g., a human globin 3 splice acceptor sequence, for example as set forth in SEQ ID NO:180), a polyadenylation sequence (e.g., a rabbit globin polyadenylation sequence, for example as set forth in SEQ ID NO: 177).

### 6.4.2.1 CAG Promoters

Certain embodiments of the present disclosure are based in part on the discovery that an ERE comprising a CAG promoter can drive far greater expression levels of the expressible polynucleotide (e.g., MTM1 coding sequence) than the desmin promoter in clinical development. Without being bound by theory, it is believed the rAAV with the MTM1 coding sequence under the control of the CAG promoter can be therapeutically effective at lower doses than corresponding vectors in which the MTM1 coding sequence is under the control of the desmin promoter, and thus such vectors are believed to have improved therapeutic indexes as compared to corresponding vectors in which the MTM1 coding sequence is under the control of the desmin promoter.

Accordingly, the present disclosure provides rAAV comprising an expressible polynucleotide operably linked to an ERE comprising a CAG promoter (referred to as a “CAG ERE” for convenience). In some embodiments, the expressible polynucleotide is an MTM1 coding sequence.

In some embodiments, the CMV enhancer component of the CAG promoter or ERE comprises a sequence having at least 90%, at least 95%, at least 98%, at least 99% or 100% sequence identity to SEQ ID NO:171.

In some embodiments, the chicken beta actin promoter component of the CAG promoter or ERE comprises a nucleotide sequence having at least 90%, at least 95%, at least 98%, at least 99% or 100% sequence identity to SEQ ID NO:172.

In some embodiments, the CAG promoter or ERE comprises a nucleotide sequence having at least 90%, at least 95%, at least 98%, at least 99% or 100% sequence identity to SEQ ID NO:173.

An exemplary CAG ERE is used in the rAVE expression cassette (GeneDetect.com).

In some embodiments, the CAG ERE further comprises a chimeric intron, for example a chimeric intron formed from introns from the human betaherpes virus and rabbit beta globin. In some embodiments, the chimeric intron comprises a nucleotide sequence having at least 90%, at least 95%, at least 98%, at least 99% or 100% sequence identity to SEQ ID NO:174.

Further modifications of the CAG promoter can be used in the rAAV of the disclosure. For example, the intron in the 5′ untranslated region (UTR) of the CAG promoter can be truncated to accommodate larger inserts (Richardson et al., 2009, PLoS One, 4(4), e5308. doi: 10.1371/joumal.pone.0005308). Deletions in intron A of the hCMV promoter can also result in enhanced expression (Quilici et al., 2013, Biotechnol Lett. 35(1), 21-27. doi: 10.1007/s10529-012-1043-z). Thus, a person skilled in the art could modify the CAG ERE or promoter sequences without compromising the high MTM1 expression levels observed with the constructs disclosed in Example 7.

6.4.2.2 Other Promoters

The rAAV of the disclosure may comprise, in lieu of a CAG ERE, an ERE comprising another constitutive promoter or a tissue specific or inducible promoter. Promoters that drive lower expression levels than a CAG promoter may be combined with other features that increase transgene expression (e.g., using codon optimized coding sequences) and/or reduce off target tropism of the virus (e.g., using muscle targeting and/or liver toggle capsid proteins).

In various embodiments the promoter is a constitutive, tissue-specific (e.g., muscle-specific) or inducible promoter. The promoters may be either naturally occurring promoters, or hybrid promoters that combine elements of more than one promoter.

Examples of constitutive promoters include, without limitation, a retroviral Rous sarcoma virus (RSV) LTR promoter (optionally with an RSV enhancer), a cytomegalovirus (CMV) promoter (optionally with a CMV enhancer), a SV40 promoter, a dihydrofolate reductase promoter, a β-actin promoter, a phosphoglycerol kinase (PGK) promoter, and a EF1α promoter.

Examples of tissue-specific promoters include, without limitation a synapsin-1 (Syn) promoter, a creatine kinase (MCK) promoter, a mammalian desmin (DES) promoter, an α-myosin heavy chain (a-MHC) promoter, or a cardiac Troponin T (cTnT) promoter.

Examples of inducible promoters include a zinc-inducible metallothionine (MT) promoter, a dexamethasone (Dex)-inducible mouse mammary tumor virus (IMMTV) promoter, a tetracycline-inducible promoter, or a rapamycin-inducible promoter.

### 6.5. Modified AAV Virions

The present disclosure further provides a modified recombinant AAV (rAAV) virion comprising a modified AAV capsid protein described herein. In some embodiments, the modified rAAV comprises a modified AAV capsid protein and a recombinant nucleic acid vector.

In some embodiments, the modified rAAV comprising a modified AAV capsid protein achieves higher infection of a target following administration to a mammalian subject as compared to an rAAV comprising a corresponding reference AAV capsid protein. In some embodiments, the modified rAAV achieves higher expression in a target of an expressible polynucleotide within the recombinant nucleic acid vector following administration to a mammalian subject when compared to expression of the expressible polynucleotide administered in an rAAV comprising a corresponding reference AAV capsid protein.

In some embodiments, the modified rAAV comprising a modified AAV capsid protein achieves lower infection of an off-target following administration to a mammalian subject as compared to an rAAV comprising a corresponding reference AAV capsid protein. In some embodiments, the modified rAAV achieves lower expression in an off-target of an expressible polynucleotide within the recombinant nucleic acid vector following administration to a mammalian subject as compared to expression of the expressible polynucleotide administered in an rAAV comprising a corresponding reference AAV capsid protein. In typical embodiments, the corresponding reference AAV capsid protein is a capsid protein identical to the modified AAV capsid protein except that it does not include a targeting peptide and/or a liver-toggle mutation described above.

In some embodiments, the target is brain, muscle, spinal cord, eye, liver, muscle, or other organ. In some embodiments, the off-target tissue is brain, muscle, spinal cord, eye, liver, muscle, or other organ. In one embodiment, the target is muscle.

In some embodiments, the modified rAAV has less liver toxicity than an rAAV comprising a corresponding reference AAV capsid protein administered by the same route of administration and in the same dose. In some embodiments, the less liver toxicity is because of de-targeting of the modified rAAV to a liver.

### 6.6. Methods of Producing rAAV

The rAAV of the disclosure comprise a recombinant nucleic acid vector containing an expressible polynucleotide. In some embodiments, the expressible polynucleotide is operably linked to an ERE. The expressible polynucleotide and ERE optionally replace the AAV genomic coding region (e.g., replace the AAV rep and cap genes). The expressible polynucleotide and ERE are generally flanked on either side by AAV inverted terminal repeat (ITR) regions, although a single ITR may be sufficient to carry out the functions normally associated with configurations comprising two ITRs (see, for example, WO 94/13788), and vector constructs with only one ITR can thus be employed in conjunction with the rAAV of the present disclosure.

In some embodiments, the rAAV of the disclosure comprise an MTM1 coding sequence operably linked to an ERE. The MTM1 coding sequence and ERE optionally replace the AAV genomic coding region (e.g., replace the AAV rep and cap genes).

In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV JTRs and in one embodiment do not share any substantial homology with the rAAV genome.

The rAAV vector construct, and the complementary packaging gene constructs can be implemented in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., WO 95/13392; WO 98/23018; and U.S. Pat. No. 5,656,785). The AAV cap gene, providing the encapsidation proteins as described above, can be provided together with an AAV rep gene or separately (see, e.g., the above-referenced patent documents as well as WO 98/27204.

Thus, the rAAV of the disclosure can be assembled by, for example, expression of its components in a packaging host cell. The components of a virus particle (e.g., rep sequences, cap sequences, inverted terminal repeat (ITR) sequences) can be introduced into a packaging host cell using one or more viral vectors.

Once assembled, rAAV particles can be purified, if desired, using routine methods. As used herein, “purified” virus particles refer to virus particles that are removed from components in the mixture in which they were made such as, but not limited to, viral components (e.g., rep sequences, cap sequences), packaging host cells, and partially- or incompletely-assembled virus particles.

### 6.7. Pharmaceutical Composition Comprising Modified rAAV

In one aspect, the present disclosure provides a pharmaceutical composition comprising a modified AAV capsid protein or a modified rAAV of the present disclosure and a pharmaceutically acceptable carrier. The modified rAAV can comprise a modified AAV capsid protein as described herein and a recombinant nucleic acid vector containing an expressible polynucleotide.

In particular embodiments, the present disclosure provides a pharmaceutical composition comprising an rAAV whose genome comprising an MTM1 coding sequence operably linked to an expression regulatory element (ERE); and one, two or all three of the following features: (a) the ERE is a hybrid expression regulatory element (ERE) comprising a CMV enhancer and a chicken beta actin promoter operably linked to the MTM1 coding sequence; and/or (b) the rAAV comprises a modified AAV capsid protein comprising at least one liver-toggle mutation and/or one muscle-targeting element; and/or (c) the MTM1 coding sequence is codon optimized for expression in human cells, optionally wherein the coding sequence has at least 90%, at least 95%, at least 98% or at least 99% sequence identity to any one of SEQ ID NOS:167 to 170.

The pharmaceutical composition can be used to deliver the recombinant nucleic acid vector to a target within a mammalian subject. When the pharmaceutical composition is administered, the modified rAAV can achieve a higher infection of target cells following administration to a mammalian subject as compared to an rAAV comprising a corresponding reference AAV capsid protein administered by the same route of administration and in the same dose. In some embodiments, the modified rAAV achieves higher expression in target cells of an expressible polynucleotide within the recombinant nucleic acid genome following administration to a mammalian subject as compared to the expressible polynucleotide administered in an rAAV comprising a corresponding reference AAV capsid protein administered by the same route of administration and in the same dose.

The pharmaceutical composition can be formulated using one or more carriers, excipients, stabilizers and adjuvants to, for example: (1) increase stability; (2) increase cell transfection or transduction; (3) permit the sustained or delayed release; (4) alter the biodistribution (e.g., target the rAAV particle to specific tissues or cell types); (5) increase the translation of encoded protein in vivo; and/or (6) alter the release profile of encoded protein in vivo.

Formulations of the pharmaceutical compositions provided herein can include, without limitation, saline, which may be formulated with a variety of buffering solutions (e.g., phosphate buffered saline), lactose, sucrose, calcium phosphate, gelatin, dextran, agar, pectin, water, lipidoids, liposomes, lipid nanoparticles, polymers, lipoplexes, core-shell nanoparticles, peptides, proteins, nanoparticle mimics and combinations thereof.

Formulations of the pharmaceutical compositions described herein can be prepared by any method known or hereafter developed in the art of pharmacology. In general, such preparatory methods include the step of associating the active ingredient with a carrier and/or one or more other accessory ingredients (e.g., excipients, stabilizers and adjuvants).

A pharmaceutical composition in accordance with the present disclosure can be prepared, packaged, and/or sold in bulk, as a single unit dose, and/or as a plurality of single unit doses. As used herein, a unit dose refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g., rAAV), the pharmaceutically acceptable carrier, and/or any additional ingredients in a pharmaceutical composition in accordance with the present disclosure can vary, depending upon the identity, size, and/or condition of the subject being treated and further depending upon the route by which the composition is to be administered.

Various carriers, excipients, stabilizers and adjuvants for formulating pharmaceutical compositions and techniques for preparing the composition are known in the art (see Remington: The Science and Practice of Pharmacy, 22nd Revised Ed., Pharmaceutical Press, 2012; incorporated herein by reference in its entirety). The use of suitable conventional carriers, excipients, stabilizers and adjuvants is contemplated within the scope of the present disclosure.

In some embodiments, the pharmaceutical composition is in the form of a solution containing concentrations of from about 1×101 to about 1×1016 genome copies (GCs)/ml of rAAV (e.g., a solution containing concentrations of from about 1×103 to about 1×1014 GCs/ml).

6.7.1. Routes of Administration

A modified rAAV of the present disclosure can be administered to a subject (e.g., a human or non-human mammal) in a suitable carrier. Suitable carriers include saline, which may be formulated with a variety of buffering solutions (e.g., phosphate buffered saline), lactose, sucrose, calcium phosphate, gelatin, dextran, agar, pectin, and water. A modified rAAV typically is administered in sufficient amounts to transduce or infect the desired cells and to provide sufficient levels of gene transfer and expression to provide a therapeutic benefit without undue adverse effects. Conventional and pharmaceutically acceptable routes of administration include, but are not limited to, direct delivery to an organ such as, for example, the muscle, liver or lung, orally, intranasally, intratracheally, intrathecally, intravenously, intramuscularly, intraocularly, subcutaneously, intradermally, or by other routes of administration. Routes of administration can be combined, if desired.

6.7.2. Dosages

The dose of a viral vector administered to a subject will depend primarily on factors such as the condition being treated, and the age, weight, and health of the subject. For example, a therapeutically effective dosage of a viral vector to be administered to a human subject generally is in the range of from about 0.1 ml to about 10 ml of a solution containing concentrations of from about 1×10¹ to about 1×10¹⁶ genome copies (GCs)/ml of viruses (e.g., a solution containing concentrations of from about 1×10³ to about 1×10¹⁴ GCs/ml). In some embodiments, the total dose of the rAAV administered to a subject is less than 3×10¹⁴ GCs, e.g., 1×10¹⁴ GCs or less, 5×10¹³ GCs or less, 1×10¹³ GCs or less, 5×10¹² GCs or less, or 1×10¹² GCs or less.

In another embodiment, a therapeutically effective dosage of a viral vector to be administered to a human subject generally is in the range of from about 0.1 ml to about 10 ml of a solution containing concentrations of from about 1×10¹ to 1×10¹² genome copies (GCs) of viruses (e.g., about 1×10³ to 1×10⁹ GCs). Transduction and/or expression of a transgene can be monitored at various time points following administration by DNA, RNA, or protein assays. In some instances, the levels of expression of the transgene can be monitored to determine the frequency and/or amount of dosage. Dosage regimens similar to those described for therapeutic purposes also may be utilized for immunization.

6.7.3. Targeting

Targeting of modified rAAVs can be tested in an experimental animal by measuring rAAV infection or expression of an expressible polynucleotide. In some embodiments, targeting is measured in a non-human primate (NHP), mice, rats, birds, rabbits, guinea pigs, hamsters, farm animals (including pigs and sheep), dogs, or cats.

Targeting of modified rAAVs can be measured after systemic or local administration of rAAVs. In some embodiments, targeting of modified rAAVs is measured after intravenous infusion of rAAVs.

6.7.3.1 RNA Data—Muscle:Liver Infection Ratio

In some embodiments, targeting of modified rAAVs is measured by measuring the ratio between the copy numbers of the transgene transcripts and housekeeping gene (e.g., RPP30) transcripts. In a particular embodiment, the transcripts are measured by RT-ddPCR. In some embodiments, the ratio is measured after a first administration into a mammal, e.g., a mouse, or a non-human primate such as a marmoset or rhesus macaque.

In some embodiments, a muscle:liver infection ratio (RNA) is measured by comparing the ratios between the copy numbers of the transgene transcripts and housekeeping gene (e.g., RPP30) transcripts in the two different organs (e.g., muscle v. liver).

$$muscle : liver infection ratio ( RNA ) = \frac{( \frac{transgene transcripts}{housekeeping transcrits} ) in muscle}{( \frac{transgene transcripts}{housekeeping transcrits} ) in liver}$$

In some embodiments, modified rAAV of the present disclosure provides a (transgene transcripts/housekeeping transcripts) ratio in liver of less than 1000, less than 900, less than 800, less than 700, less than 600, less than 500, less than 400, less than 300, less than 200, less than 100, less than 90, less than 80, less than 70, less than 60, less than 50, less than 40, less than 30, less than 20, or less than 10.

In certain embodiments, when the (transgene transcripts/housekeeping transcripts) in the liver is zero or below detection limits, the muscle:liver infection ratio is reported as >10,000 by convention.

In some embodiments, the modified rAAV of the present disclosure provides a muscle:liver infection ratio (RNA) of at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 500, at least 1000. In some embodiments, the muscle is triceps surae, biceps, heart or quadricep.

In some embodiments, modified rAAV of the present disclosure provides a muscle:liver infection ratio (RNA) of 1 to 10, 1 to 100, 10 to 20, 10 to 50, 10 to 80, 10 to 100, 20 to 100, 100 to 500, 100 to 1000, or 500 to 1000. In some embodiments, the muscle is triceps surae, bicep, heart or quadricep.

6.7.3.2 DNA Data—Muscle:Liver Infection Ratio

In some embodiments, targeting of modified rAAVs is measured by measuring the ratio between the copy numbers of the transgene DNA genomes to copy numbers of host genes or genetic loci (e.g., RPP30). In a particular embodiment, the genomes are measured by RT-ddPCR. In some embodiments, the ratio is measured after a first administration into a mammal, e.g., a mouse, or a non-human primate such as a marmoset or rhesus macaque.

In some embodiments, a muscle:liver infection ratio (DNA) is measured by comparing the ratios between the copy numbers of the transgene DNA genomes and housekeeping gene (e.g., RPP30) genomes in the two different organs (e.g., muscle v. liver).

$$muscle : liver infection ratio ( DNA ) = \frac{( \frac{transgene DNA genomes}{housekeeping genomes} ) in muscle}{( \frac{transgene DNA genomes}{housekeeping genomes} ) in liver}$$

In some embodiments, modified rAAV of the present disclosure provides a (transgene genomes/housekeeping genomes) ratio in liver of less than 1, or in a range from 1 to 10, 1 to 5, 1 to 2, 0.1 to 1, 0 to 1, 0.01 to 0.1, 0.01 to 0.5, or 0.01 to 0.05.

In certain embodiments, when the (transgene genomes/housekeeping genomes) in the liver is zero or below detection limits, the muscle:liver infection ratio is reported as >10,000 by convention.

In some embodiments, the modified rAAV of the present disclosure provides a muscle:liver infection ratio (DNA) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1,000 or at least 10,000. In some embodiments, the muscle is triceps surae, biceps, heart or quadricep.

In some embodiments, modified rAAV of the present disclosure provides a muscle:liver infection ratio (DNA) in the range of 0.5 to 1, 0.5 to 5, 0.5 to 10, 1 to 10, 1 to 100, 2 to 8, 5 to 10, 10 to 20, 20 to 80, 10 to 50, 10 to 100, 50 to 80, 100 to 500, 100 to 1000, or 500 to 1000. In some embodiments, the muscle is triceps surae, biceps, heart, or quadricep. In some embodiments, the modified rAAV achieves a muscle:liver infection ratio (DNA) of at least 2, at least 5, at least 10, at least 20, at least 30, at least 40, at least 50, at least 60, at least 70, at least 80, at least 90, at least 100, at least 150, at least 200, at least 500, at least 1000. In some embodiments, the modified rAAV achieves a muscle:liver infection ratio of 0.1 to 1, 1 to 5, 1 to 10, 1 to 20, 1 to 50, 1 to 100, 1 to 200, 1 to 300, 100 to 500, 250 to 750, or 500 to 1000.

6.7.3.3 IHC data Muscle:Liver Infection Ratio

In some embodiments, targeting of modified rAAVs is calculated using the % of cells that have been successfully transduced and express a transgene in a tissue (e.g., eGFP). In a particular embodiment, the transgene expression is measured by immunohistochemistry. In some embodiments, the ratio is measured after a first administration into a mammal, e.g., a mouse, or a non-human primate such as a marmoset or rhesus macaque.

In some embodiments, a muscle:liver infection ratio (IHC) is measured by comparing the ratios between the transgene % GFP+cells and housekeeping gene (e.g., RPP30) % GFP+cells in the two different organs (e.g., muscle v. liver).

$$muscle : liver infection ratio ( IHC ) = \frac{( \frac{transgene \% GFP + cells}{housekeeping \% GFP + cells} ) in muscle}{( \frac{transgene \% GFP + cells}{housekeeping \% GFP + cells} ) in liver}$$

In some embodiments, modified rAAV of the present disclosure provides a (transgene % GFP/housekeeping % GFP) ratio in liver of less than 1, less than 5, less than 10, or in a range from 1 to 10, 1 to 5, 1 to 2, 0.1 to 1, 0 to 1, 0.01 to 0.1, 0.01 to 0.5, or 0.01 to 0.05.

In certain embodiments, when the (transgene % GFP/housekeeping % GFP) in the liver is zero or below detection limits, the muscle:liver infection ratio is reported as >10,000 by convention.

In some embodiments, the modified rAAV of the present disclosure provides a muscle:liver infection ratio (IHC) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1000. In some embodiments, the muscle is triceps surae, biceps, heart or quadricep.

In some embodiments, modified rAAV of the present disclosure provides a muscle:liver infection ratio (IHC) of 1 to 5, 1 to 10, 1 to 100, 2 to 8, 10 to 20, 20 to 30, 10 to 50, 10 to 100, 20 to 80, 50 to 80, 100 to 500, 100 to 1000, or 500 to 1000. In some embodiments, the muscle is triceps surae, bicep, heart or quadricep.

### 6.8. Method of Use

A modified rAAV as described herein can be used in research and/or therapeutic applications. In some embodiments, a modified rAAV is for genetically modifying a cell in vitro or in vivo. In some embodiments, a modified rAAV is used for gene therapy or for vaccination in a human or animal. More specifically, a modified rAAV can be used for gene addition, gene augmentation, genetic delivery of a polypeptide therapeutic, genetic vaccination, gene silencing, genome editing, gene therapy, RNAi delivery, cDNA delivery, mRNA delivery, miRNA delivery, miRNA sponging, genetic immunization, optogenetic gene therapy, transgenesis, DNA vaccination, or DNA immunization of liver cells or non-liver cells.

In some embodiments, a modified rAAV of the present disclosure is used for treatment of a muscle disease. In some embodiments, the disease is a muscular disease and/or the condition is muscle degeneration. In some embodiments, said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome. In some embodiments, the modified rAAV is used to deliver the rAAV to a striated muscle, preferably heart or a skeletal muscle or diaphragm.

In some embodiments, the rAAVs or pharmaceutical compositions described are useful in the treatment of subjects (preferably human subjects) suffering from XLMTM and/or carrying mutations in the MTM1 gene. “Treatment” of MTM encompasses a complete reversal or cure of the disease, or any range of improvement in conditions and/or adverse effects attributable to MTM. Merely to illustrate, “treatment” of MTM includes an improvement in any of the following effects associated with MTM or combination thereof: short life expectancy, respiratory insufficiency (partially or completely), poor muscle tone, drooping eyelids, poor strength in proximal muscles, poor strength in distal muscles, facial weakness with or without eye muscle weakness, abnormal curvature of the spine, joint deformities, and weakness in the muscles that control eye movement (ophthalmoplegia). Improvements in any of these conditions can be readily assessed according to standard methods and techniques known in the art.

A modified rAAV of the present disclosure can be administered to a subject in a suitable pharmaceutical carrier, e.g., as described in Section 6.7.

The rAAV of the disclosure are typically administered in sufficient amounts to transduce or infect the desired cells and to provide sufficient levels of gene transfer and expression to provide a therapeutic benefit to subjects suffering from a disease. In particular embodiments, the rAAV is administered in sufficient amounts to provide a therapeutic benefit to subjects suffering from XLMTM or carrying a mutation in the MTM1 gene, without undue adverse effects.

Conventional and pharmaceutically acceptable routes of administration include, but are not limited to, direct delivery to an organ such as, for example, the muscle, liver or lung, orally, intranasally, intratracheally, intrathecally, intravenously, intramuscularly, intraocularly, subcutaneously, intradermally, or by other routes of administration. Routes of administration can be combined, if desired.

The dose of a viral vector administered to a subject will depend primarily on factors such as the age, weight, and health (e.g., disease progression) of the subject. For example, a therapeutically effective dosage of a viral vector to be administered to a human subject generally is in the range of from about 0.1 ml to about 10 ml of a solution containing concentrations of from about 1×10¹ to about 1×10¹⁶ genome copies (GCs)/ml of viruses (e.g., a solution containing concentrations of from about 1×10³ to about 1×10¹⁴ GCs/ml). In some embodiments, the total dose of the rAAV administered to a subject is less than 3×10¹⁴ GCs, e.g., 1×10¹⁴ GCs or less, 5×10¹³ GCs or less, 1×10¹³ GCs or less, 5×10¹² GCs or less, or 1×10¹² GCs or less.

Transduction and/or expression of the transgene can be monitored at various time points following administration by DNA, RNA, or protein assays.

Accordingly, the present disclosure provides a method of treating and/or preventing a muscular disease and/or muscle degeneration by administering a modified rAAV described herein.

### 7. EXAMPLES

Experiment AFT-MR0001 was designed to test the hypothesis that the peptide RGDLLLS (SEQ ID NO:1), when inserted into VR VIII to create a modified adeno-associated virus capsid protein, enhances gene delivery to skeletal muscle versus the unmodified protein. Further, the experiment was designed to test the hypothesis that the liver toggle mutation provides a structure that can determine efficiency of liver gene delivery and that the peptide insertion into VR VIII can act independently and/or synergistically. Two doses were also used, a low dose of 1×10¹³ gc/kg and a high dose of 5×10¹³ gc/kg, seeking to demonstrate possible equivalence of eGFP expression afforded by the modified AAV vector at a low dose with the eGFP expression observed with the unmodified vector at high dose. This experiment proved both hypotheses: the targeting peptide insertion into VR VIII of an adeno-associated virus VP1 protein enhances gene delivery to the muscle, and that combination with the liver-detargeting phenotype produces a vector with robust gene delivery to the muscle with the expected reduction in liver. Further, at a 5× lower dose, the level of expression between high dose unenhanced variants and matching low dose enhanced variants is not statistically significant.

### 7.1. Example 1: AAV Variants

The polynucleotide encoding the wild-type AAV9 VP1 capsid protein (SEQ ID NO: 61) or AAV^{mut1} capsid protein (SEQ ID NO: 163) was modified by inserting the 7-mer peptide RGDLLLS (SEQ ID NO: 1) between amino acid residues 588 and 589. The produced modified polynucleotides encode modified VP1 proteins referred to as: AAV^{deco1} capsid protein (SEQ ID NO: 158) or AAV^{mut1_deco1} capsid protein (SEQ ID NO: 159).

Corresponding AAV vectors were manufactured with the modified capsid proteins in the Affinia Therapeutics Vector Core via standard triple transfection into HEK293 cells. The AAV vectors produced by the method include AAV9 CAG.GFP (CAG.GFP construct encapsulated in AAV9 capsid), AAV^{mut1} CAG.GFP (CAG.GFP construct encapsulated in a capsid comprising the AAV^{mut1} capsid protein), AAV9^{deco1} CAG.GFP (CAG.GFP construct encapsulated in a capsid comprising the AAV^{deco1} capsid protein), and AAV^{mut1_deco1} CAG.GFP (CAG.GFP construct encapsulated in a capsid comprising the AAV^{mut1_deco1} capsid protein). Successful gene transfer by these vectors was detected by GFP expression in target cells. Please note that the vectors comprising a particular modified capsid protein is referred to in the Figures related to the following examples by the abbreviated term for the capsid protein itself, as will be clear from the context of the experiment.

### 7.2. Example 2: Confirmed Enhanced Muscle Tropism with Limited Liver Tropism for AAV^{mut1_deco1} in C57BL/6 Mice at Both Low and High Dosage Regimes

Gene transfer efficacy of each AAV vector prepared according to Example 1 was tested with three C57BL/6 mice, injected with one of the vectors at one of the two different doses by intravenous tail vein injection. Total twenty-four mice were injected in total as summarized in the below table. The low dose was 1×10¹³ gc/kg (total 2×10¹¹ gc), and the high dose was 5×10¹³ gc/kg (total 1×10¹² gc). Additionally, a control mouse was injected with vehicle (1×PBS, 35 mM NaCl, 0.001% pluronic) alone. Thus, a total 25 mice comprised the study.

| | low dose (1 × | high dose (5 × |
|---|---|---|
| Vector | 10¹³ gc/kg) | 10¹³ gc/kg) |
| AAV9 CAG.GFP “AAV9” | 3 mice | 3 mice |
| AAVmut1 CAG.GFP “AAV^{mut1}” | 3 mice | 3 mice |
| AAV9deco1 CAG.GFP “AAV^{deco1}” | 3 mice | 3 mice |
| AAVmut1_deco1 CAG.GFP | 3 mice | 2 mice |
| “AAV^{mut1}^{_{—}}^{deco1}” | | |

| | |
|---|---|
| Control (vehicle) | 2 mice |

The mice were sacrificed 28 days after the injection. Individual tissues, notably the liver, major skeletal muscles of the bind limb, heart, and diaphragm, were collected at the time of necropsy. Tissue were immediately placed into the preservative RNAlater, after which the RNAlater was removed and the tissue flash frozen. The same tissues were fixed and embedded for sectioning and anti-GFP staining by immunohistochemistry (IHC).

GFP expression was assessed by anti-GFP IHC, and ddRT-PCR for the eGFP vector genome copies per DPG (DNA) and transcript (mRNA). The eGFR transcript level was compared against the transcript of a housekeeping standard RPP30. IHC was performed at Histoserv Inc. (Germantown, MD). ddRT-PCR was performed at Affinia Therapeutics (Waltham, MA).

Images of exemplary liver and skeletal muscle tissue cross-sections obtained from the anti-GFP IHC are provided in FIGS. 4A-4J. The tissue cross-sections were stained with an anti-GFP primary antibody followed by an HRP-linked secondary staining and substrate addition. Brown staining of cells above the counterstain for intact cells and nuclei indicates eGFP expression. The vehicle control tissues from liver or skeletal muscle show the structure and organization expected from healthy tissues. AAV9 at 5×10¹³ gc/kg robustly transduces the liver and muscle cells (brown individual cells). GFP expression within the liver is reduced in mice injected with AAV-mut1-deco1, such that isolated individual cells are stained. On the other hand, GFP expression within muscle tissue was significantly increased in the mice injected with AAV-mut1-deco1.

Transgene transfer and expression capabilities of administered vectors were also evaluated with ddPCR, by measuring amounts of DNA and mRNA of the transgene (eGFP) in the various tissue samples 28 days after injection. DNA genome copies and mRNA transcript copies of the transgene (eGFP) were quantified in comparison to the amounts of DNA genome copies or mRNA transcript copies of a house keeping gene (RPP30), respectively. Specifically, DNA genome copies are reported as vector genomes copies per diploid genome (VGC/DG). The formula for calculating the output is VGC/DG=(eGFP cp/μL÷RPP30 cp/μL)×2. RNA transcript copies are reported as % eGFP expression, which is calculated according to the formula, % eGFP expression=(eGFP cp/μL÷RPP30 cp/μL)×100.

Tissues were homogenized in a Qiagen Tissuelyser It (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 uL of buffer. Prior to analysis, DNA and RNA concentration and quality were determined using a NanoDrop One, using the nucleic acid (DNA or RNA) program. DNA samples were analyzed for biodistribution of vector genomes using a duplexed ddPCR method targeting the transgene (eGFP) and a reference gene (RPP30). RNA samples were analyzed for expression of the eGFP transgene using a duplexed, one-step RT-ddPCR method and a reference gene (RPP30).

mRNA was extracted from 30 mg sections of liver, and quadriceps. The results of the ddPCR assays are shown in FIG. 5A and FIG. 5B which show that AAV^{Mut1} reduces liver tropism but does not enhance muscle tropism, AAV^{Deco1} has high liver tropism and comparatively high muscle tropism, and that AAV^{Mut1_deco1} has decreased liver tropism and increased muscle tropism compared to AAV9 (WT). Other muscle tissues examined, discussed below, and showed a similar trend and the DNA and RNA results generally agree.

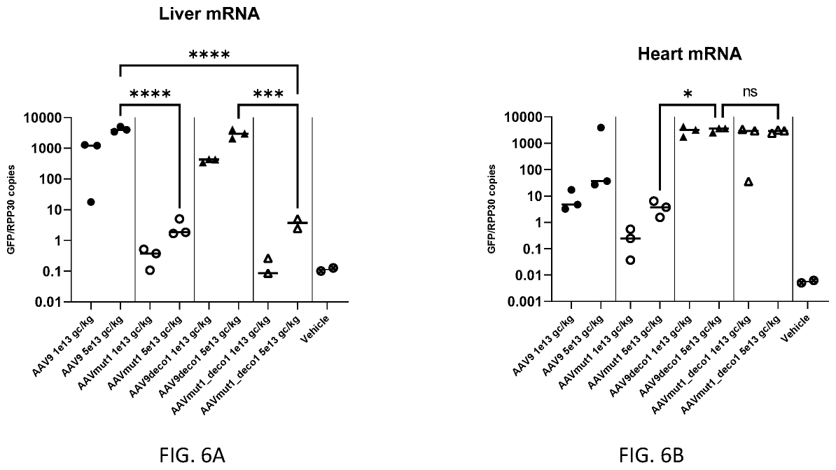

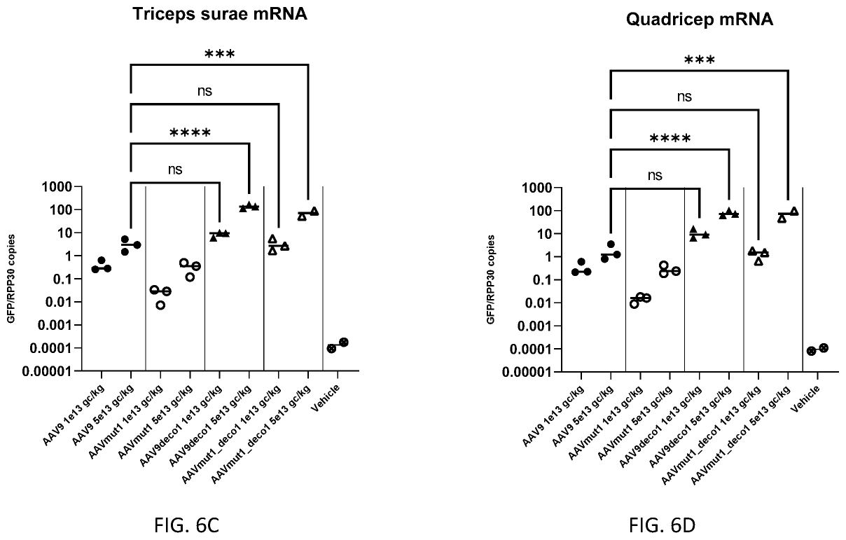

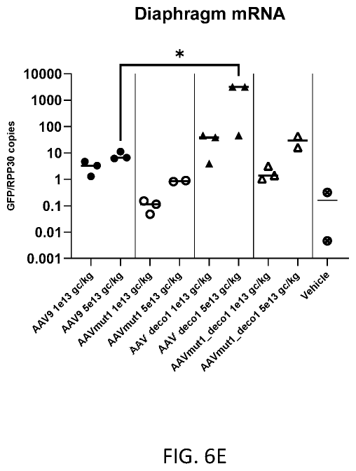

eGFP mRNA expression in various tissues was measured by RT-ddPCR and presented as the ratio of eGFP transcripts over RPP30 transcripts, a rough indicator of eGFP mRNA copies per cell. The results are provided in FIG. 6A (liver), FIG. 6B (heart), FIG. 6C (tricep surae), FIG. 6D (quadricep), and FIG. 6E (diaphragm). For each tissue, results from three biological replicates are provided for each AAV variant at each dose (high or low dose). Statistically significant differences were determined by an ANOVA 1-way test with P-values and indicated with asterisks. * P<0.1, * P<0.01, *** P<0.001, **** P<0.0001, ns=not significant.

FIG. 6A provides the ratio of eGFP to RPP30 transcripts in the liver. At 5×10¹³ gc/kg dose, both AAV^{mut1} and AAV^{mut1_deco1} had a greater than 3-log lower levels of eGFF expression in the liver compared to AAV9. AAV^{deco1} had almost 3-logs higher expression in the liver than AAV^{mut1_deco1}.

FIG. 6B shows the ratio of eGFP to RPP30 transcripts in the heart. Both AAV^{deco1} and AAV^{mut1_deco1} had higher expression in the heart, although the difference and significance are reduced by a single outlier within the AAV9 at 5×10³ gc/kg dose group, and possible signal saturation within the AAV^{deco} and AAV^{mut1_deco1} high dose group. The level of expression is significantly higher in AAV^{deco1} compared to AAVmut1 at 5×10¹³ gc/kg, and there was no significant difference between high dose AAV^{deco1} and AAV^{mut1_deco1}, notwithstanding the possible signal saturation.

FIG. 6C shows the ratio of eGFP to RPP30 transcripts in the triceps surae. Within the 5×10¹³ gc/kg groups, there was more than 1-log increase in the eGFP per RPP30 mRNA ratio in AAV9^{deco1} and AAV^{mut1_deco1} compared to AAV9 in the calf muscle tissue of the study subjects. Importantly, there was no significant difference between the high dose AAV9 group and the 1×10¹³ gc/kg low dose groups of AAV^{deco1} and AAV^{mut1_deco1}.

FIG. 6D shows the ratio of eGFP to RPP30 transcripts in the quadricep. Results were similar to the triceps surae, the other skeletal muscle tested in this study. Within the 5×10¹³ gc/kg groups, there is more than 1 log increase in eGFP per RPP30 mRNA ratio in AAV^{deco1} and AAV^{mut1_deco1} compared to AAV9 in quadricep tissue of the study subjects. Importantly, there is no significant difference between the high dose AAV9 group and the 1×10¹³ gc/kg low dose groups of AAV^{deco1} and AAV^{mut1_deco1}.

FIG. 6E shows the ratio of eGFP to RPP30 transcripts in the diaphragm. Increase of gene delivery efficacy in deco-containing vectors was also observed in the diaphragm, but in this study all but one comparison exceeded the threshold of significance: high dose AAV9 versus high dose AAV^{deco1}.

### 7.3. Example 3: Enhanced Muscle Tropism with Limited Liver Tropism for AAV^{mut1-deco1} Confirmed at the Earlier d14 Time Point in C57BL/6 Mice

Gene transfer efficacy of AAV9 vector and AAV^{mut1_deco1} vector was tested with groups of three C57BL/6 mice, injected with one of the vectors by intravenous tail vein injection. Total thirteen mice were injected in total as summarized in the below table. The dose was 1×10¹³ gc/kg (total 2×10¹¹ gc). Additionally, a control mouse was injected with vehicle (1×PBS, 35 mM NaCl, 0.001% pluronic) alone.

| | | Dose | No. of | Study | |
|---|---|---|---|---|---|
| Treatment | Route | (vgs) | Animal | Duration | Necroscopy |
| Vehicle/control | IV | 0 | 1 | 14 days | Organ |
| AAV^{mut1} | IV | 1.00E+13 | 3 | | collection |
| AAV^{mut1}^{_{—}}^{deco1} | IV | 1.00E+13 | 3 | | |
| AAV^{mut1} | IV | 1.00E+13 | 3 | 28 days | |
| AAV^{mut1}^{_{—}}^{deco1} | IV | 1.00E+13 | 3 | | |

The mice were sacrificed 14 or 28 days after the injection. Individual tissues, notably the liver and major skeletal muscles of the hind limb (quad), were collected at the time of necropsy. Tissues were immediately placed into the preservative RNAlater, after which the RNAlater was removed and the tissue flash frozen. The same tissues were fixed and embedded for sectioning and anti-GFP staining by immunohistochemistry (IHC).

eGFP expression was assessed by anti-GFP IHC. IHC was performed at Histoserv Inc. (Germantown, MD). ddRT-PCR was performed at Affinia Therapeutics (Waltham, MA) as described above.

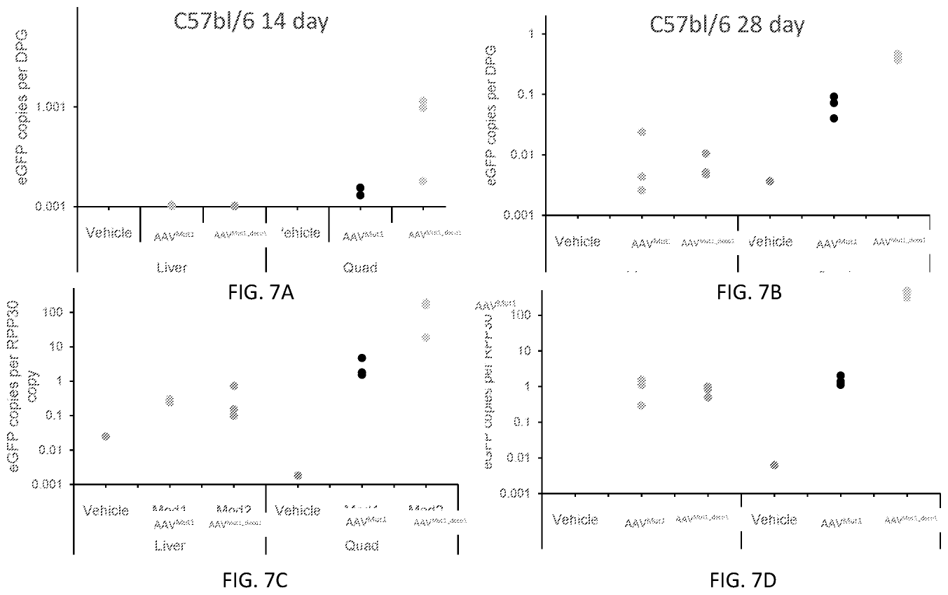

DNA and RNA were extracted from 30 mg sections. DNA and RNA samples were assayed for eGFP vector genome or mRNA transcript by ddRT-PCR and normalized to murine RPP30 genomic copies or RPP30 mRNA copies, respectively. Triplicate technical replicates were performed. The results are shown in FIGS. 7A-7D.

FIGS. 7A-7B show eGFP vector genome (DNA) in liver and quad tissues of C57BL/6 mice 14 days (FIG. 7A) or 28 days (FIG. 7B) after treatment with vehicle, AAVMut1 and AAVMut1-deco1 AAV vectors. FIGS. 7C-7D show eGFP mRNA expression in liver and quad tissues of C57BL/6 mice 14 days (FIG. 7C) or 28 days (FIG. 7D) after treatment with vehicle, AAV^{Mut1} and AAV^{Mut1_deco1} AAV vectors.

As can be seen from these data, AAV^{Mut1_deco1} enhancement of muscle tropism is observable at d14; AAV^{Mut1} and AAV^{Mut1_deco1} vector genome copies (VGs) are stable from d14 to d28; AAV^{Mut1_deco1} enhancement leads to greater accumulation of eGFP signal; and liver tropism is consistently low through all samples.

### 7.4. Example 4: Confirmed Enhanced Muscle Tropism with Limited Tropism to Liver and Other Organs for AAV^{-mut1_deco1} in BALB/c Mice

Gene transfer efficacy of AAV^{mut1} and AAV^{mut1_deco1} were tested with three or six BALB/c mice, injected with one of the vectors at 5×10¹³ gc/kg (total 1×10¹² gc) by intravenous tail vein injection. Additionally, control mice were injected with vehicle (1×PBS, 35 mM NaCl, 0.001% pluronic) alone. Total twelve mice were injected in total as summarized in the below table.

| Vector | 5 × 10¹³ gc/kg (total 1 × 10¹² gc) |
|---|---|
| Control (vehicle) | 3 mice |
| AAVmut1 CAG.GFP | 6 mice |
| AAVmut1_deco1 CAG.GFP | 3 mice |

The mice were sacrificed 28 days after the injection. Individual tissues, notably the liver, major skeletal muscles of the hind limb, heart, diaphragm, brain, spinal cord, and spleen were collected at the time of necropsy.

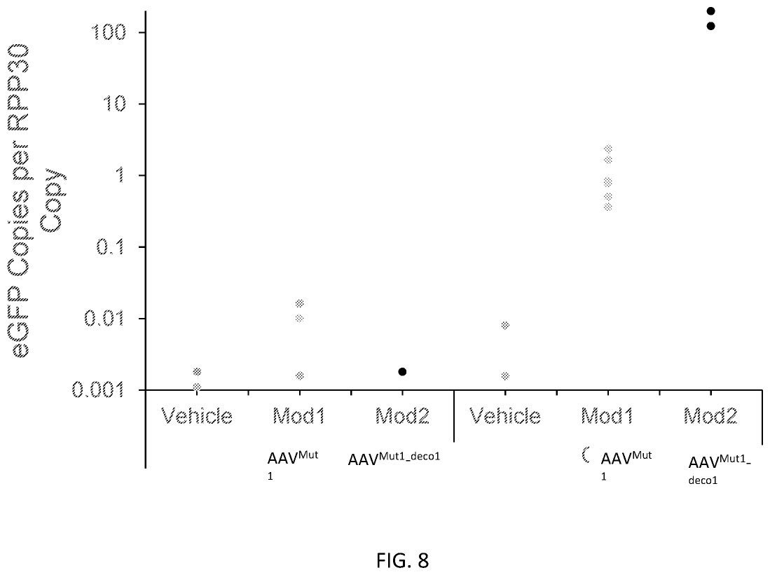

DNA and RNA were extracted from 30 mg sections of liver and quadricep. DNA and RNA samples were assayed for eGFP vector genome or mRNA by ddRT-PCR and normalized to murine RPP30 genomic copies or RPP30 mRNA copies. Triplicate technical replicates were performed. The results are provided in FIG. 8.

The results show no increase in liver tropism with AAV^{mut1 deco1} but increase of tropism in the quadriceps compared to AAV^{mut1}. Further the data showed a similar AAV^{mut1 deco1} enhancement in the heart, triceps surae, and diaphragm compared to AAV^{mut1}. There was no significant difference found in the spleen, spinal cord, or liver.

### 7.5. Example 5: Summary of Mouse Data

The below Table exemplifies the Muscle:Liver infection ratios calculated for the DNA biodistribution data, the RNA expression data and the IHC expression data obtained for administration of AAV^{mut1_deco1} vector compared to AAV9 vector in Mice.

| | | | DNA | | RNA | | IHC |
|---|---|---|---|---|---|---|---|
| | | | Muscle:Liver | | Muscle:Liver | | Muscle:Liver |
| | | DNA | infection | RNA | infection | IHC | infection |
| Tissue | Treatment | Biodistribution† | ratio | % Expression† | ratio | Expression† | ratio |
| Liver | AAV9 | 151.4 | | 418083 | | 99 | |
| Liver | AAV^{mut1}^{_{—}}^{deco1} | 0* | | 373.9 | | 4.3 | |
| Quadriceps | AAV9 | 0.9 | 0.01 | 184.0 | 0.00 | 0.7 | 0.01 |
| Quadriceps | AAV^{mut1}^{_{—}}^{deco1} | 0.3 | >10,000* | 7277.0 | 19.46 | 8.7 | 2.02 |
| Triceps | AAV9 | 0.3 | 0.00 | 322.2 | 0.00 | 0.0 | 0.00 |
| Surae | | | | | | | |
| Triceps | AAV^{mut1}^{_{—}}^{deco1} | 0.5 | >10,000* | 7092.3 | 18.97 | 15.0 | 3.49 |
| Surae | | | | | | | |
| Heart | AAV9 | 0.5 | 0.00 | 4975.2 | 0.01 | 10.7 | 0.11 |
| Heart | AAV^{mut1}^{_{—}}^{deco1} | 3.8 | >10,000* | 322.2 | 0.86 | 68.3 | 15.88 |

*when liver value is zero, then the ratio is >10,000 by convention.

### 7.6. Example 6: Confirmed Enhanced AAV^{-mut1-deco1} Muscle Tropism with Limited Liver Tropism Confirmed in NHP

The objective of this study is to confirm liver retargeting and muscle transduction superiority of AAV^{mut1_deco1} vector compared to AAV9 vector in non-human primates (NHP) as was observed in mice. The results confirm enhanced muscle transduction superiority and liver de-targeting of AAV^{Mut1_Deco1} vector compared to AAV9.

Two AAV constructs were used in the experiment: (i) AAV^{-mut1.deco1}-CAG-GFP, and (ii) AAV9-CAG-GFP, each including an AAV genome construct containing a coding sequence of GFP. GFP was used to detect distribution of AAVs and expression of the transgene. Marmoset monkeys were used as the subject animals.

Total of 7 animals were divided into 3 groups as summarized in the below TABLE #. Immunosuppression of the animals began 7 days prior to vector administration. Group 1 is a control animal administered with vehicle. Animals in Group 2 and 3 were administered with 1×10¹⁴ vg (viral genome or GC) of AAV9 vector or AAV^{mut1_deco1} vector by IV to the right saphenous vein. Animals were sacrificed on day 28 after the vehicle or AAV vector administration and their organ samples were collected for analysis.

| | Route of | Dose | No. of | Volume | Study |
|---|---|---|---|---|---|
| Treatment | Administration | (vgs) | Animal | (ml) | Duration |
| Vehicle/control | IV | 0 | 1 | 0.625 | 28 days |
| AAV9 (WT) vector | IV | 1.00E+14 | 3 | 0.625 | |
| AAV^{mut1}^{_{—}}^{deco1}vector | IV | 1.00E+14 | 3 | 0.625 | |

IHC for GFP expression were scored (blinded) by a pathologist. A second pathologist peer reviewed the data. Initial assessment for GFP expression by IHC was conducted on one section per tissue-referred to as Run 1 tissues and included liver, heart and skeletal muscle (right and left sides-tibialis, biceps, quadriceps, gastrocnemius. Two additional sections per muscle group were run to assess consistency of expression within each muscle group-referred to as Run 2.

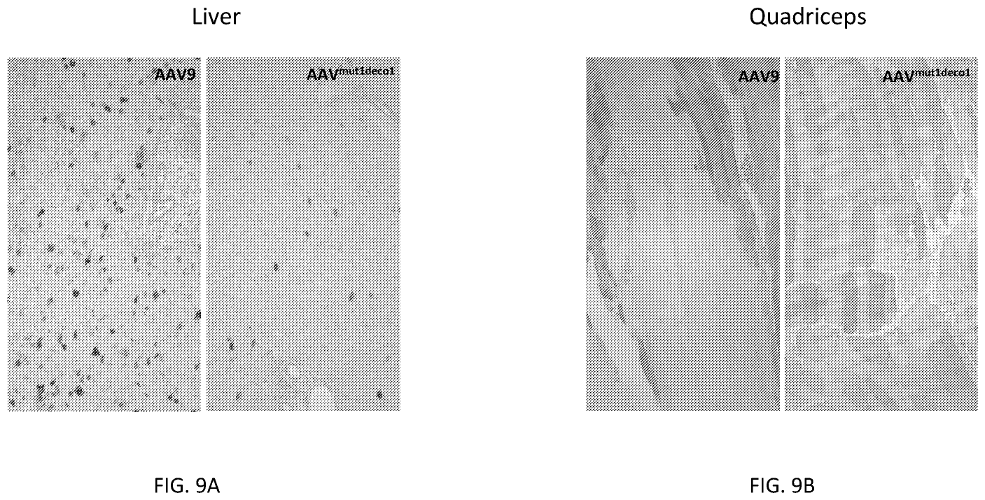

Analysis of Run 1 tissue samples is shown in FIGS. 9A and 9B. Exemplary IHC liver tissue is shown in FIG. 9A, obtained from AAV9 treated animal on the left, and AAV^{mut1_deco1} treated animal illustrated on the right side of the chart. Exemplary IHC quadriceps tissue is shown in FIG. 9B, obtained from AAV9 treated animal on the left, and AAV^{mut1_deco1} treated animal on the right.

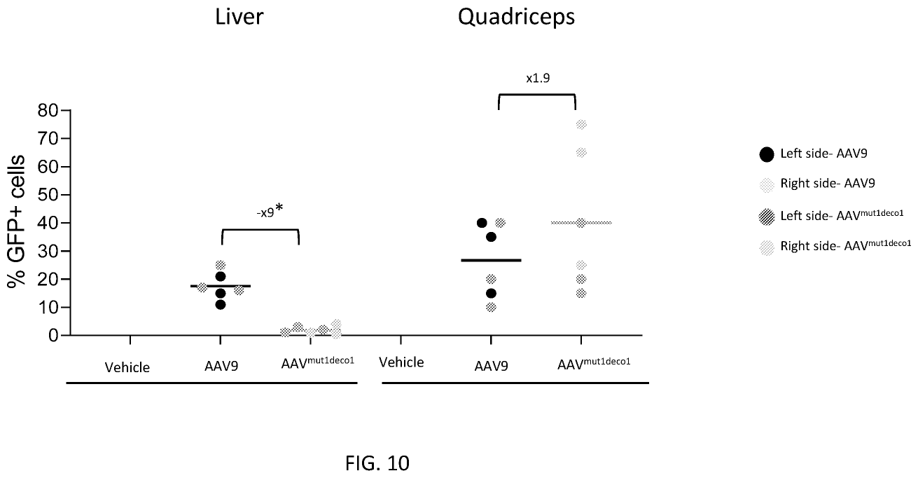

FIG. 10 shows the % GFP positive cells in the liver tissue (right and left side of the organ) and quadriceps tissue (right and left leg) in slides obtained from Run 1 for each animal administered vehicle or vector (AAV9 or AAV^{mut1deco1}).

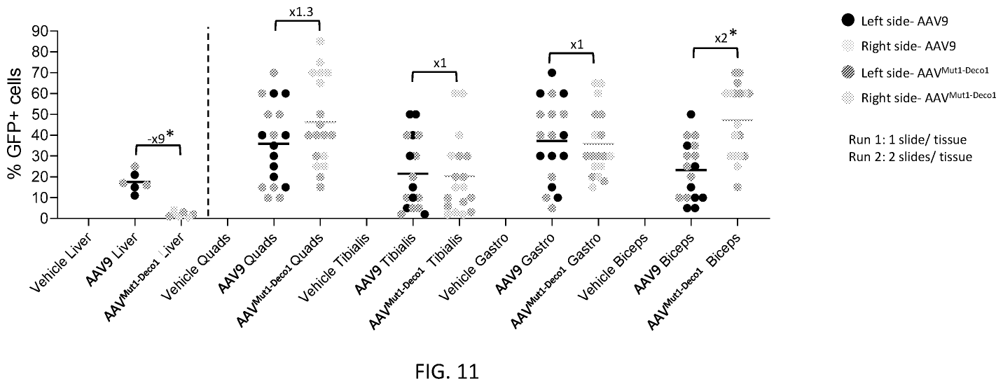

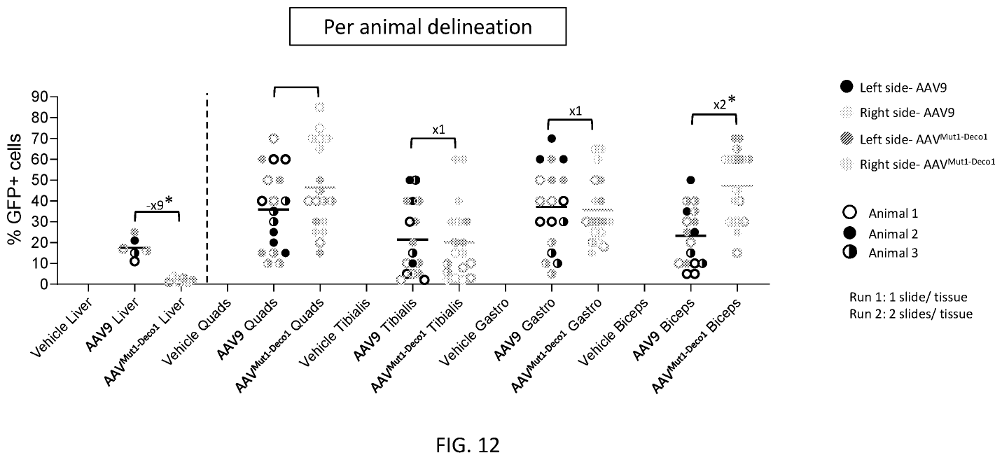

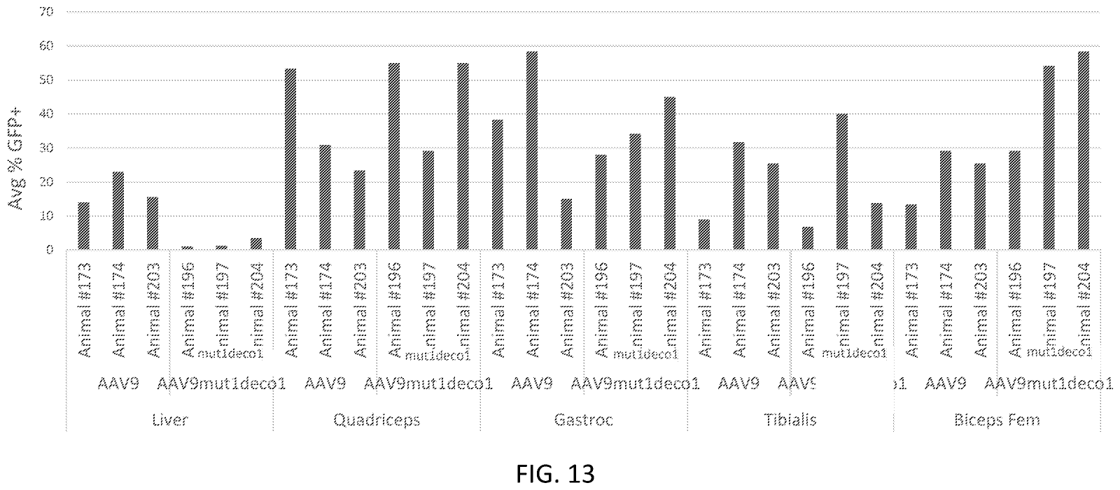

FIG. 11 shows the % GFP positive cells in various skeletal muscle and liver tissue (average from Runs 1 and 2) for each animal administered vehicle and vector (AAV9 or AAV^{mut1deco1}). FIG. 12 shows the % GFP positive cells per animal in various skeletal muscle and liver tissue (average from Runs 1 and 2) for each animal administered vehicle and vector (AAV9 or AAV^{mut1deco1}). FIG. 13 shows the average combined quantification of % GFP positive cells per animal in various skeletal muscle and liver tissue (average from Runs 1 and 2) for each animal administered vehicle and vector (AAV9 or AAV^{mut1deco1}).

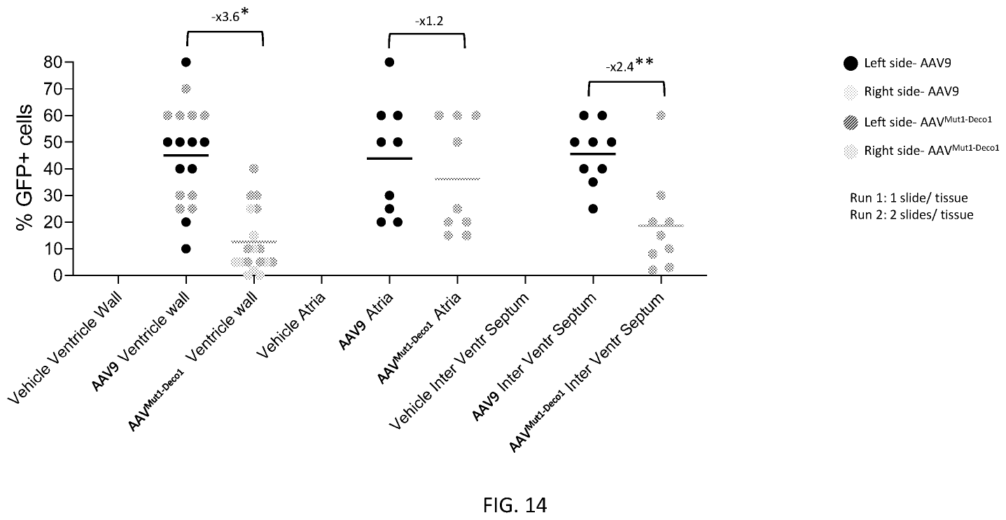

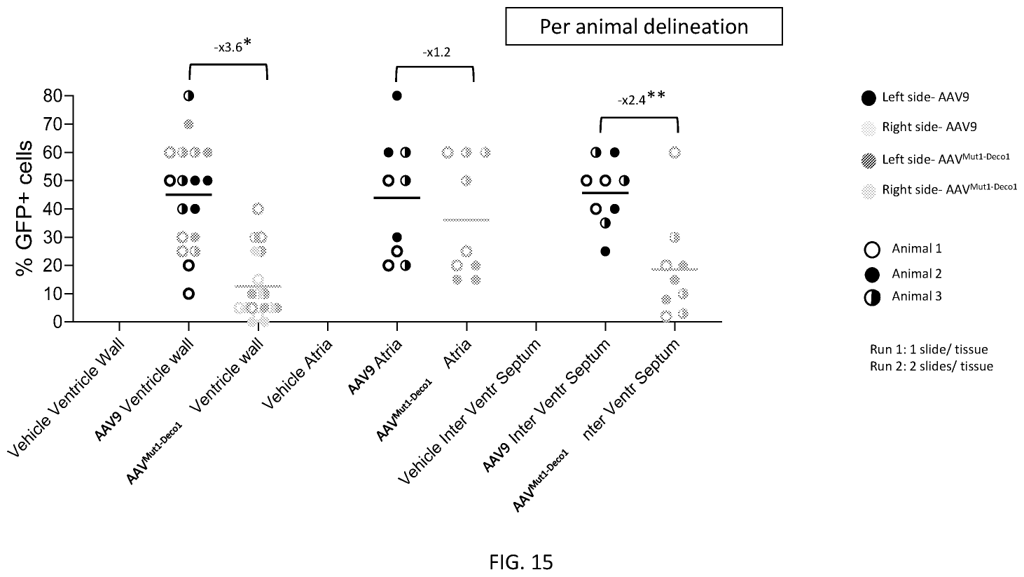

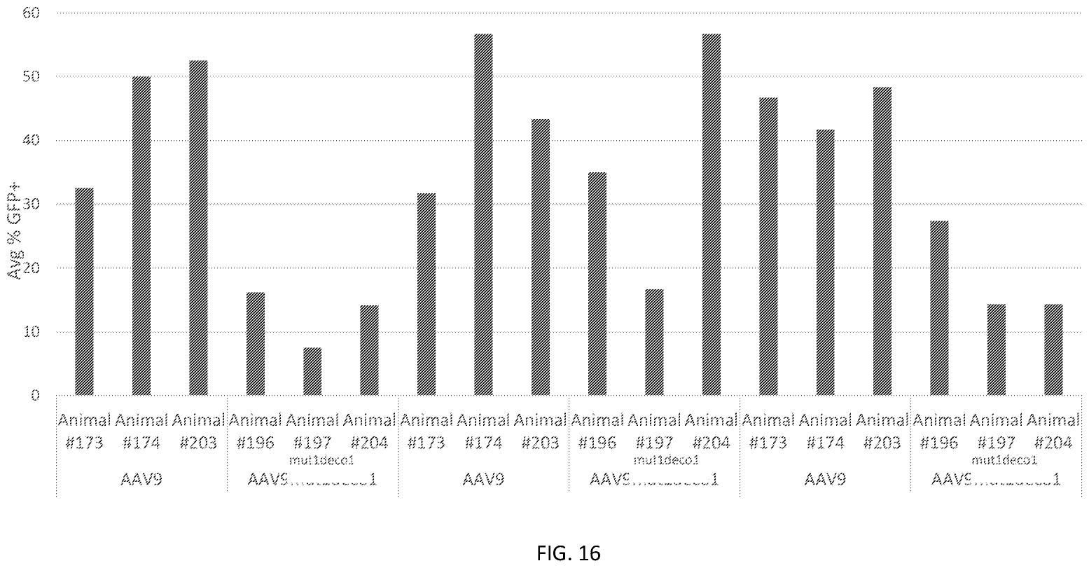

FIG. 14 shows the % GFP positive cells in various cardiac tissue (average from Runs 1 and 2) for each animal administered vehicle and vector (AAV9 or AAV^{mut1deco1}) FIG. 15 shows the % GFP positive cells per animal in various cardiac muscle (average from Runs 1 and 2) for each animal administered vehicle and vector (AAV9 or AAV^{mut1deco1}). FIG. 16 shows the average % GFP positive cells per animal in various cardiac muscle (average from Runs 1 and 2) for each animal administered vehicle and vector (AAV9 or AAV^{mut1deco1}).

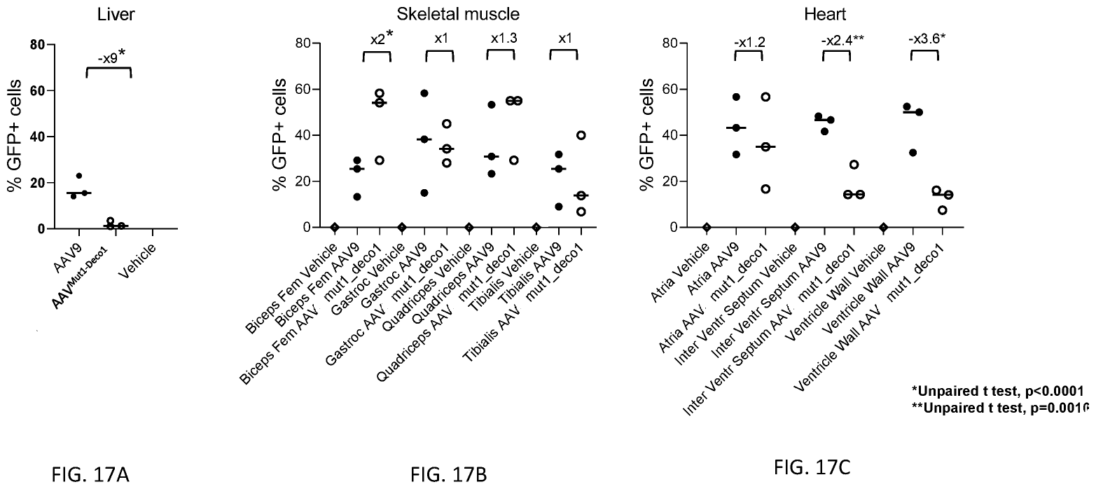

FIGS. 17A-17C show the average % GFP positive cells per animal in various tissues (average from Runs 1 and 2) for vehicle, AAV9 and AAV^{mut1_deco1} vectors. FIG. 17A shows average % GFP positive cells per animal in liver tissue. FIG. 17B shows average % GFP positive cells per animal in various skeletal muscle tissue. FIG. 17C shows average % GFP positive cells per animal in various cardiac tissue.

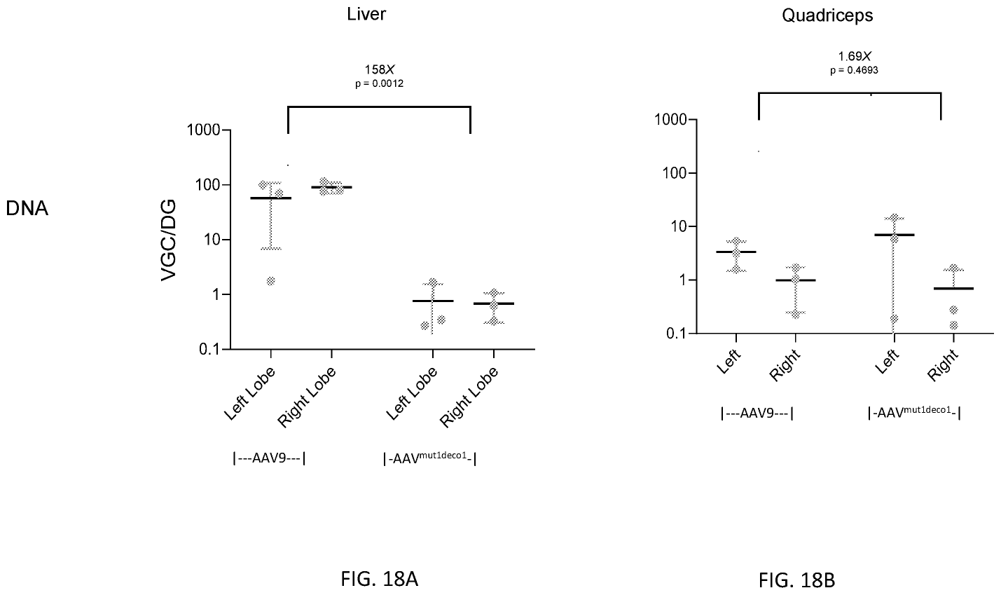

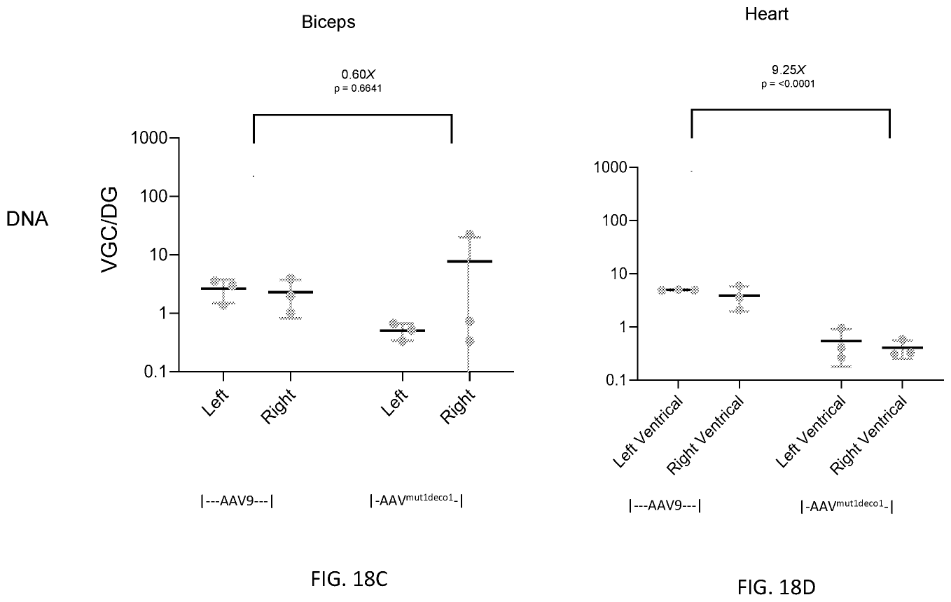

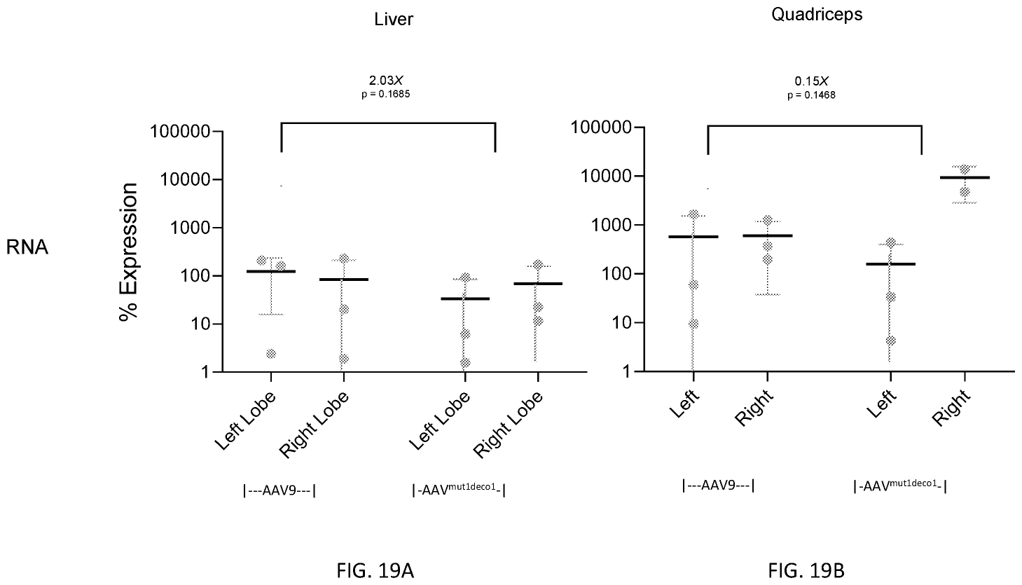

DNA samples were analyzed for biodistribution of vector genomes in the liver and quadriceps tissue using a duplexed ddPCR method targeting the transgene (eGFP) and a reference gene (RPP30). The results are shown in FIGS. 18A (liver), 18B (quadriceps), 18C (biceps), 18D (heart) where the x-axis represents AAV vectors (wild type AAV9 on the left and AAV^{mut1deco1} on the right) and whether the sample was taken from the left or right side of the organ/animal.

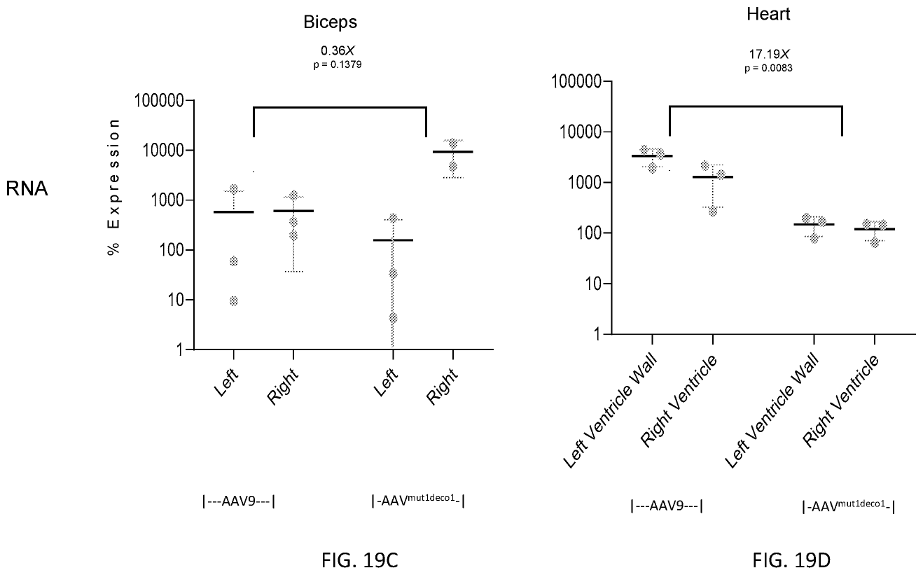

mRNA transcript amounts measured by eGFP copies of eGFP over RPP30 mRNA are shown in FIGS. 19A (liver), 19B (quadriceps), 19C (biceps), 19D (heart). The x-axis represents AAV vectors (wild type AAV9 on the left and AAV^{mut1deco1} on the right) and whether the sample was taken from the left or right side of the organ/animal.

The DNA, RNA and IHC expression data obtained from NHP experiments are quantified and summarized in the below Table where each IHC stain is a technical replicate, data from all tissues combined including left and right sides; averages of the data obtained for all three animals is shown. Notably, heart data includes data from ventricles and atria but does not include septum.

| | | DNA | | | | IHC | |
|---|---|---|---|---|---|---|---|
| | | Biodistribution^{†} | | RNA | | Expression^{†} | |
| Tissue | Treatment | VGC/DG | | % Expression^{†} | | % GFP | |
| Liver | AAV9 | 73.7 | (32.7) | 104.1 | (107.6) | 17.5 | (4.9) |
| Liver | AAV^{mut1}^{_{—}}^{deco1} | 0.7 | (0.6) | 51.1 | (68.3) | 1.9 | (1.4) |
| Quadriceps | AAV9 | 2.2 | (1.3) | 589.5 | (693.1) | 35.8 | (17.7) |
| Quadriceps | AAV^{mut1}^{_{—}}^{deco1} | 3.8 | (3.6) | 3814.4 | (5954.5) | 46.4 | (21.0) |
| Biceps | AAV9 | 2.5 | (1.0) | 1327.3 | (3005.6) | 22.6 | (11.6) |
| Biceps | AAV^{mut1}^{_{—}}^{deco1} | 4.4 | (6.0) | 3677.6 | (3962.5) | 47.2 | (14.9) |
| Heart | AAV9 | 3.6 | (2.2) | 2303.6 | (1503.9) | 44.6 | (10.1) |
| Heart | AAV^{mut1}^{_{—}}^{deco1} | 0.5 | (1.5) | 134.3 | (52.1) | 20.44 | (15.8) |

^{†}Mean (St. Dev.)

The below Table exemplifies the Muscle:Liver infection ratios calculated for the DNA biodistribution data, the RNA expression data and the IHC expression data obtained for administration of AAV^{mut1_deco1} vector compared to AAV9 vector in non-human primates (NHP) as shown in the table above.

| | | | DNA | | RNA | | IHC |
|---|---|---|---|---|---|---|---|
| | | | Muscle:Liver | | Muscle:Liver | | Muscle:Liver |
| | | DNA | infection | RNA | infection | IHC | infection |
| Tissue | Treatment | Biodistribution† | ratio | % Expression† | ratio | Expression† | ratio |
| Liver | AAV9 | 73.7 | | 104.1 | | 17.5 | |
| Liver | AAV^{mut1}^{_{—}}^{deco1} | 0.7 | | 51.1 | | 1.9 | |
| Quadriceps | AAV9 | 2.2 | 0.03 | 589.5 | 5.66 | 35.8 | 2.05 |
| Quadriceps | AAV^{mut1}^{_{—}}^{deco1} | 3.8 | 5.43 | 3814.4 | 74.65 | 46.4 | 24.42 |
| Biceps | AAV9 | 2.5 | 0.03 | 1327.3 | 12.75 | 22.6 | 1.29 |
| Biceps | AAV^{mut1}^{_{—}}^{deco1} | 4.4 | 6.29 | 3677.6 | 71.97 | 47.2 | 24.84 |
| Heart | AAV9 | 3.6 | 0.05 | 2303.6 | 22.13 | 44.6 | 2.55 |
| Heart | AAV^{mut1}^{_{—}}^{deco1} | 0.5 | 0.71 | 134.3 | 2.63 | 20.44 | 10.76 |

### 7.7. Example 7: Development of rAAV Constructs with High MTM1 Expression

Myotubular myopathy (XLMTM, OMIM 310400) is a severe congenital muscular disease due to mutations in the myotubularin gene (MTM1) and characterized by the presence of small myofibers with frequent occurrence of central nuclei. Myotubularin is a ubiquitously expressed phosphoinositide phosphatase with a muscle-specific role in man and mouse that is poorly understood.

The objective of the current study was to identify a promoter that provides a broad biodistribution of expression within skeletal muscle. We have constructed nine human MTM1 expressing AAV gene expression constructs with various promoters and transgenes to transduce skeletal muscle and express adequate amounts of MTM1 protein for the treatment of XLMTM.

7.7.1. Materials & Methods

7.7.1.1 Cloning of MTM1 Expression Constructs

A nucleotide sequence was synthesized to include the untranslated first exon and a portion of the intron from the human Cytomegalovirus (hCMV) IE gene, a portion of the intron of the second intron of the human beta globin gene, a portion of the 3^{rd} exon of the human beta globin gene, a NotI restriction site, a predicted optimal Kozak sequence, a codon optimized human MTM1 CDS with a modified stop codon using a sequence provided by Genscript, a Pac restriction site which overlaps with the modified stop codon, the Rabbit beta-globin PolyA signal sequence, an AvrII site, and the first 10 bp of the AAV2 ITR. This fragment and SA024, an AAV2 ITR plasmid containing a Desmin promoter, a chimeric intron and exon of the CMV IE gene and human beta globin, a gene of interest, and a Rabbit beta-globin PolyA signal, were digested with BstBI, which has a site in CMV IE exon 1, and XhoI, which has a site in the Rabbit beta-globin PolyA signal. Portions of SA024 containing the first ITR and expression regulatory sequences 5′ to the gene of interest are provided as SEQ ID NO:204 and the portions of SA024 from the 3′ of the open reading frame of the gene of interest through the second ITR are provided as SEQ ID NO:205. The 2443 bp long fragment containing MTM1 and the 6693 bp long fragment containing the plasmid elements were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the codon optimized MTM1 sequence and the additional features. The portions of the vector within the ITRs (and including the ITRs) are provided as SEQ ID NO:181.

Two additional codon optimizations for the MTM1 CDS using sequences derived from algorithms provided by GeneArt and Eurofins as well as the native MTMT CDS (SEQ ID NO:202) were synthesized (used in SEQ ID NOS:182, 183 and 184 described below, respectively). These three sequences with the addition of a NotI site and a Kozak site at the 5′ end and a modified stop codon and PacI site at the 3′ end of the sequence were synthesized at GeneArt, Eurofins, and Genscript, respectively, An additional sequence was synthesized by Genscript using an algorithm to both optimize the codons for expression and reduce the number of CpG sequences within the synthetic product (used in SEQ ID NO:185). These fragments or plasmids containing these fragments were digested with NotI and PacI along with the vector containing SEQ ID NO:181. The 1823 bp fragment containing the MTM1 CDSs and the 7350 bp fragment containing the plasmid and ITR sequence and other SEQ ID NO:181 features were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the codon optimized or native MTM1 sequence and the additional features (the portions of these vectors within (and including) the ITRs are provided as SEQ ID NOS:182-185).

Constructs containing a promoter which is a hybrid of the CMV immediate early enhancer, and the chicken beta-actin promoter were also made. The hybrid is referred to as the CAG promoter. The CAG promoter was amplified from construct 7701591057 (the portion of which within (and including) the ITRs is provided as SEQ ID NO:201), which had been synthesized previously, using a 5′ primer with the sequence SEQ ID NO: 209 (ttttGGTACCgacattgattattgactagttatt) which contains a KpnI restriction site and a Poly T tag to aid in restriction digestion and a region matching the start of the CMV immediate early promoter in a linear amplification reaction. The amplification product was isolated from the amplification mixture with NEB Monarch DNA Gel isolation kit. A second amplification step was performed with a primer with the sequence SEQ ID NO: 210 (aaaaaa gatatc cgcccgccgcgc) which contains a region matching the reverse complement of the Chicken beta actin promoter, an EcoRV restriction site, and a poly A sequence to aid in fragment digestion. The 675 base pair fragment (SEQ ID NO:200) was isolated by agarose gel electrophoresis and eluted from the agarose using NEB Monarch DNA Gel isolation kit. The 675 bp fragment and SEQ ID NO:181 vector were digested with KpnI and EcoRV. The 663 bp digested fragment of the amplification and the 8581 bp fragment of the SEQ ID NO:181 vector were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the CAG promoter sequence into the vector containing SEQ ID NO:181 resulting in a vector containing SEQ ID NO:186, which includes an MTM1 CDS with codon optimizations provided by Genscript.

To insert the GeneArt codon optimized MTM1 sequence and the Eurofins codon optimized MTM1 sequence into the SEQ ID NO:186 containing vector, vectors with SEQ ID NO:182, SEQ ID NO:183, and SEQ ID NO: 186 were digested with KpnI and EcoRV. The 663 base pair fragment from the SEQ ID NO: 186 containing vector and the 8581 bp fragment from the digests of the vector with SEQ ID NO:182 and SEQ ID NO:183 were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the CAG promoter sequence into the SEQ ID NO:182 and SEQ ID NO:183 vectors, resulting in vectors containing SEQ ID NO:187 (GeneArt) and SEQ HD NO:188 (Eurofins).

To insert the native MTI sequence into the vector containing SEQ ID NO: 186, the native MTM1 sequence was amplified from the vector containing SEQ ID NO:184 using a 5′ primer with the sequence TTTGAGCGGCCGCCA which corresponds to the Kozak and start sequence of MTM1 and contains a NotI restriction site and a 3′ primer with the sequence GATCTTAATTAAAAGTGAGTTTGCACATGGG which contains the reverse complement to the 3′ end of MTM1, an altered stop codon, and a Pac restriction site. The 1837 base pair PCR product (SEQ ID NO:19) was isolated by agarose gel electrophoresis and eluted from the agarose using NEB Monarch DNA Gel isolation kit. The purified amplicon and the vector containing SEQ ID NO: 186 were digested with NotI and PacI. The 1823 bp fragment containing the MTM1 CDS and the 7350 bp fragment containing the plasmid and ITR sequence and other SEQ ID NO:186 features were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the native MTM1 sequence and the additional features SEQ ID NO:186 resulting in a vector with SEQ ID NO: 189.

7.7.1.2 Cloning of self-complementary MTM1 Expression Constructs

The vectors described above are single stranded vectors. To overcome potential limitations on expression from these vectors, self-complementary vectors were constructed. Genscript synthesized a vector which contained the sequence of the miniTK promoter, an alternate gene of interest, the Rabbit beta-globin PolyA signal, and an AAV2 ITR which contains a deletion in the D region of the ITR. A miniTK portion of the vector is provided as SEQ ID NO:190 and the portion of the vector containing the rabbit globin poly A and ITR is provided as SEQ ID NO: 191. This synthetic sequence was flanked by SalI site at the 5′ end and AscI at the 3′ end. This fragment was introduced into the vector comprising SEQ ID NO:201 via restriction enzyme digestion, agarose gel fragment isolation, and T4 DNA ligase ligation. Successful ligation products were isolated from E. coli transformants and confirmed by restriction digest and Sanger sequencing to confirm the insertion of self-complementary vector sequence into the vector comprising SEQ ID NO:201 resulting in a vector containing SEQ ID NOS:190 and 191. The first ITR and miniTK portions of the vector are provided as SEQ ID NO:206 and the rabbit poly and second ITR portions of the vector are provided as SEQ ID NO:207.

To create self-complementary vectors of the appropriate size for successful packaging into AAV capsids, the miniTK promoter was synthesized with KpnI restriction site at the 5′ end and a NotI site at the 3′ end. Additionally, bases were added to the synthesize product to enhance the efficiency of restriction digestion SEQ ID NO: 192. The fragment containing SEQ ID NO:192 was digested with KpnI and NotI and inserted into a vector containing SEQ ID NO:184 via the same restriction sites following agarose gel electrophoresis, gel extraction, T4 ligation. This vector (the portion of which within (and including) the ITRs is provided as SEQ ID NO:11B) after sequencing, was determined to have an undesired deletion in the 5′ ITR. However, the insert of SEQ ID NO:193 was identical to the desired sequence. The miniTK-native MTM1 sequence was PCR amplified using the 5′ primer SEQ ID NO: 211 (tttttGtcGACTTCGCATATTAAGGTGACGCGT) which contains a polyT sequence to aid in restriction digestion, the KpnI site, and the 5′ end of the miniTK promoter and the 3′ primer SEQ ID NO: 212 (tttttt cctagg gagTGAGAGACACAAAAAATTCCAACACAC), which contains a polyT sequence to aid in restriction digestion, an AvrII site, and the reverse complement of the 3′ end of the Rabbit beta-globin PolyA signal creating SEQ ID NO: 194. SEQ ID NO:194 and the vector containing SEQ ID NOS:190 and 191 were digested with KpnI and SalI. The 2024 bp fragment with SEQ ID NO: 194 and the 6603 bp fragment comprising SEQ ID NO:190 and SEQ ID NO:191 were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the promoter and native MTM1 CDS into the vector with appropriate ITRs for creating a self-complementary AAV vector. The vector created this way contains a full ITR, the miniTK promoter, the native MTM1 CDS, the Rabbit beta-globin Poly A signal and an ITR with an appropriate deletion to create a self-complementary AAV vector. The portion of this vector within (and including) the ITRs is provided as SEQ ID NO:208.

To create a similar vector with a miniaturized version of the Desmin promoter, the mini Desmin promoter was synthesized with KpnI site at the 5′ end and NotI site at the 3′ end (SEQ ID NO: 195). Additional bases were added to the synthesized product to enhance the efficiency of restriction digestion (SEQ ID NO:196). The fragment containing SEQ ID NO:196 was digested with KpnI and NotI and inserted into a vector containing SEQ ID NO:184 via the same restriction sites following agarose gel electrophoresis, gel extraction, T4 ligation. This vector (the portion of which within (and including) the ITRs is provided as SEQ ID NO:197), after sequencing, was determined to have a undesired deletion in the 5′ ITR. However, the insert of SEQ ID NO:197 was identical to the desired sequence. The miniDes-native MTM1 sequence was PCR amplified using the 5′ primer SEQ ID NO: 213 (tttttGtcGACCCTCTATAAATACCCGCTCTGG) which contains a polyT sequence to aid in restriction digestion, the KpnI site, and the 5′ end of the miniDesmin promoter and the 3′ primer SEQ ID NO: 214 (tttttt cctagg gagTGAGAGACACAAAAAATTCCAACACAC) which contains a polyT sequence to aid in restriction digestion, an AvrII site, and the reverse complement of the 3′ end of the Rabbit beta-globin PolyA signal creating SEQ ID NO:198. SEQ ID NO:198 and the vector comprising SEQ ID NO:190 and SEQ ID NO:191 were digested with KpnI and SalI. The 2185 bp fragment containing SEQ ID NO:198 and the 6603 bp fragment containing comprising SEQ ID NO:190 and SEQ ID NO:191 were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the promoter and native MTM1 CDS into the vector with appropriate ITRs for creating a self-complementary AAV vector. The vector created this way contains a full ITR, the miniDesmin promoter, the native MTM1 CDS, the Rabbit beta-globin Poly A signal and an ITR with an appropriate deletion to create a self-complementary AAV vector. The portion of this vector within (and including) the ITRs is provided as SEQ ID NO:199.

7.7.1.3 Expression Studies by Cell Transfection

The RD cell line (ATCC CCL-136) was used for our in vitro expression studies. RD cells are derived from patients with Rhabdomyosarcoma, a rare form of pediatric cancer that develops from skeletal muscles. RD cells were maintained in 10% FBS DMEM inside a humidified 37 degrees C. incubator with 5% CO₂ air with serial passage every three to four days following TrypLE non-enzymatic lifting and replating at ¼th density.

24 h prior to transfection, RD cells were lifted with TrypLE, pelleted (7′, room temperature, 1400×g), and resuspended in media. Viability was determined by Trypan Blue exclusion using two chambers of a Countess automated cell counter (Thermo Fisher). Average cell density was adjusted to 3.2E5 live cells per mL and 1.6E5 viable cells were plated in 500 uL media. In a 24 well plate.

On the day of transfection, all reagents were warmed to room temperature before use. Plasmid DNA was diluted to 250 ng/uL in TE buffer. Enough reagent was used to transfect 4 wells per plasmid. 100 uL OptiMEM (gibco) plus 6 uL Lipofectamine 3000 (Thermofisher, lot 2170726) was prepared per plasmid. Separately, 100 uL of OptiMEM plus 6 ug of DNA (24 uL of 250 ng/uL diluted plasmid) plus 12 uL 3000 Reagent (Thermo Fisher) were combined. The diluted DNA was then mixed with the diluted Lipofectamine 3000, spun down briefly, and incubated at room temperature for 16 minutes. 57 uL of the mixture was added to each of 4 wells per plasmid. Some wells of cells were left untransfected to serve as a negative control.

24 hours post-transfection, RD cells were imaged on a BioTek Lionheart for eGFP expression to confirm successful transfection and to estimate % transfection efficiency. A 1 second exposure using the LED intensity setting of 10 was used. After imaging, the cells were washed with 500 uL DPBS. The DPBS was removed and 125 uL TrypLE was added and incubated for 5 minutes at 37 degrees C. in a humidified incubator with 5% CO2. The cells were triturated to resuspend and pelleted at 140×g at 4 degrees C. for 7 minutes. 5 mL of 2× lysis was buffer was prepared in sterile filtered dH2O with 10×RIPA buffer (Cell Signaling) with 1 tablet of Roche EDTA-free Mini Complete Protease Inhibitor. 12 uL of the 2× lysis buffer was added to the cell pellet. The lysate was vortexed briefly and spun down. Samples, lysed or unlysed, were stored at minus 80 degrees C.

Plasmids used: In addition to the MTM1 expression constructs, certain reference plasmids were used. Plasmid 7701591057, a fully-synthesized plasmid vector, which contains AAV2 ITRs and eGFP under the control of the CAG promoter and the Rabbit beta-globin PolyA signal was used as a transfection control for fluorescently visualizing eGFP and percent of cells successfully transfected as well as a negative control for antibody-mediated MTM1 detection. pCDNA3.1+C/(K)DYK with human native MTM1 under the control of the CMV promoter, an in-frame DYK epitope tag, and a bovine Growth Hormone PolyA signal. This was obtained from Genscript.

7.7.1.4 Automated Western Analysis

In vitro expression of MTM1 was analyzed using the ProteinSimple Jess automated western blot. Jess was utilized to fully automate capillary loading, protein separation, incubation, and detection. Protein lysates from transfected and untransfected RD cells were diluted 1:4 in 0.1× Sample Buffer+1× Fluorescent Master Mix (ProteinSimple, PS-STOIEZ-8) and 3 uL was loaded onto a separation module (ProteinSimple, SM-W004). Capillaries were incubated with an MTM1 polyclonal antibody at a 1:15 dilution (Proteintech, 13924-1-AP) and a Secondary Mouse Antibody conjugated with HRP (ProteinSimple, DM-001). In addition to an immunoassay, the Total Protein Detection module (ProteinSimple, DM-TPO1-1) was included to allow for normalization of MTM1 expression by total protein load. Total protein and MTM1 were detected by the chemiluminescence channel.

7.7.2. Results

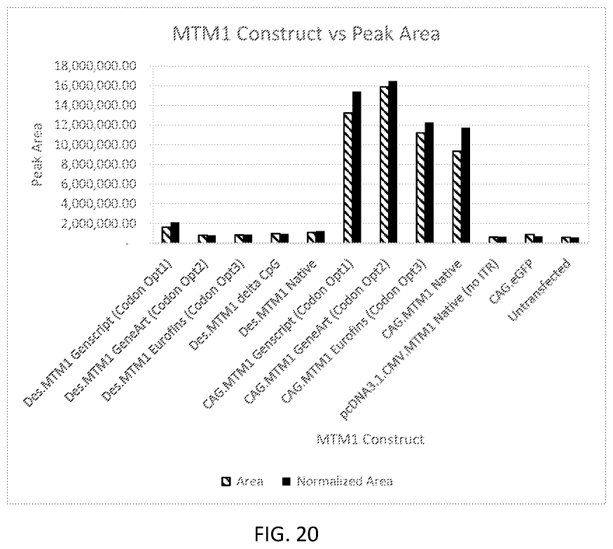

Results are shown in FIG. 20. Total human myotubularin protein levels were quantified in RD muscle cells following transfection with 9 different MTM1 containing expression plasmids. All Mtm1 expression plasmids expressed levels of MTM protein significantly greater than controls (untransfected and GFP transfected controls). The CAG promoter expressed higher MTM1 protein levels in RD cells compared to Desmin promoter containing plasmids. Codon optimization of the MTM1 transgene using GeneArt, Genscript, and Eurofins algorithms had minimal impact on expression of MTM1 protein in RD cells. These findings indicate that the CAG promoter drove higher levels of MTM1 protein expression in human RD muscle cells in vitro compared to plasmids containing the Desmin promoter.

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### 9. SEQUENCES

Many of the nucleotide sequences provided below are obtained from double stranded vectors. Thus, one of skill in the art would appreciate that, unless the references throughout the specification and claims to nucleotide sequences provided herein also include references to the complementary sequences unless the context dictates otherwise

| | SEQUENCE (X or X″ can be any of the standard amino acids; |
|---|---|
| | for Anc library sequences (Anc80; Anc81; Anc82; Anc83; Anc84; Anc94; Anc110; Anc113; |
| SEQ ID | Anc126; and Anc127), X can be any one of the amino acids listed below for each toggle |
| NO | site) |
| SEQ ID | RGDLLLS |
| NO: 1 | |
| SEQ ID | AQTLAWPFKAQ |
| NO: 2 | |
| SEQ ID | AQSWSKPFLAQ |
| NO: 3 | |
| SEQ ID | DGTLAVPFKAO |
| NO: 4 | |
| SEQ ID | ESTLAVPFKAO |
| NO: 5 | |
| SEQ ID | ESTLAVPFKAO |
| NO: 6 | |
| SEQ ID | GGTLAVPFKAQ |
| NO: 7 | |
| SEQ ID | AQTLATPFKAQ |
| NO: 8 | |
| SEQ ID | ATTLATPFKAO |
| NO: 9 | |
| SEQ ID | DGTLATPFKAO |
| NO: 10 | |
| SEQ ID | GGTLATPFKAQ |
| NO: 11 | |
| SEQ ID | SGSLAWPFKAQ |
| NO: 12 | |
| SEQ ID | AQTLAQPFKAQ |
| NO: 13 | |
| SEQ ID | AQTLQQPFKAQ |
| NO: 14 | |
| SEQ ID | AQTLSNPFKAQ |
| NO: 15 | |
| SEQ ID | AOTLAVPFSNP |
| NO: 16 | |
| SEQ ID | QGTLAVPFKAQ |
| NO: 17 | |
| SEQ ID | NQTLAVPFKAQ |
| NO: 18 | |
| SEQ ID | EGSLAVPFKAQ |
| NO: 19 | |
| SEQ ID | SGNLAVPFKAQ |
| NO: 20 | |
| SEQ ID | EGTLAVPFKAQ |
| NO: 21 | |
| SEQ ID | DSTLAVPFKAQ |
| NO: 22 | |
| SEQ ID | AVTLAVPFKAQ |
| NO: 23 | |
| SEQ ID | AQTLSTPFKAQ |
| NO: 24 | |
| SEQ ID | AQTLPQPFKAQ |
| NO: 25 | |
| SEQ ID | AQTLSQPFKAQ |
| NO: 26 | |
| SEQ ID | AQTLQLPFKAQ |
| NO: 27 | |
| SEQ ID | AQTLTMPFKAQ |
| NO: 28 | |
| SEQ ID | AQTLTTPFKAQ |
| NO: 29 | |
| SEQ ID | AQYTLSQGWAQ |
| NO: 30 | |
| SEQ ID | AQMNATKNVAQ |
| NO: 31 | |
| SEQ ID | AQVSGGHHSAQ |
| NO: 32 | |
| SEQ ID | AQTLPQPFKAQ |
| NO: 33 | |
| SEQ ID | AQTLATPFKAQ |
| NO: 34 | |
| SEQ ID | AQTLTMPFKAQ |
| NO: 35 | |
| SEQ ID | AQTLTAPFKAQ |
| NO: 36 | |
| SEQ ID | AQTLSKPFKAQ |
| NO: 37 | |
| SEQ ID | QAVRTSL |
| NO: 38 | |
| SEQ ID | YTLSQGW |
| NO: 39 | |
| SEQ ID | LAKERLS |
| NO: 40 | |
| SEQ ID | LAKERLS |
| NO: 41 | |
| SEQ ID | SVSKPFL |
| NO: 42 | |
| SEQ ID | FTLTTPK |
| NO: 43 | |
| SEQ ID | MNSTKNV |
| NO: 44 | |
| SEQ ID | VSGGHHS |
| NO: 45 | |
| SEQ ID | SAQTLAVPFKAQAQ |
| NO: 46 | |
| SEQ ID | SXXXLAVPFKAQAQ |
| NO: 47 | |
| SEQ ID | SAQXXXVPFKAQAQ |
| NO: 48 | |
| SEQ ID | SAQTLXXXFKAQAQ |
| NO: 49 | |
| SEQ ID | SAQTLAVXXXAQAQ |
| NO: 50 | |
| SEQ ID | SAQTLAVPFXXXAQ |
| NO: 51 | |
| SEQ ID | RGDX¹X²X³X⁴ |
| NO: 52 | |
| SEQ ID | TLAVPFK |
| NO: 53 | |
| SEQ ID | MAADGYLPDWLEDNLSEGIREWWDLKPGAPKPKANQQKODDGRGLVLPGYKYLGPFNGLDKGE |
| NO: 54 | PVNAADAAALEHDKAYDQQLKAGDNPYLRYNHADAEFQERLQEDTSFGGNLGRAVFQAKKRVLE |
| (AAV1 | PLGLVEEGAKTAPGKKRPVEQSPQEPDSSSGIGKTGQQPAKKRLNFGQTGDSESVPDPQPLGEPPA |
| (AAD27757)) | TPAAVGPTTMASGGGAPMADNNEGADGVGNASGNWHCDSTWLGDRVITTSTRTWALPTYNN |
| | HLYKQISSASTGASNDNHYFGYSTPWGYFDFNRFHCHFSPRDWQRLINNNWGFRPKRLNFKLFNI |
| | QVKEVTTNDGVTTIANNLTSTVQVFSDSEYQLPYVLGSAHQGCLPPFPADVFMIPQYGYLTLNNGS |
| | QAVGRSSFYCLEYFPSQMLRTGNNFTFSYTFEEVPFHSSYAHSQSLDRLMNPLIDQYLYYLNRTQN |
| | QSGSAQNKDLLFSRGSPAGMSVQPKNWLPGPCYRQQRVSKTKTDNNNSNFTWTGASKYNLNGR |
| | ESIINPGTAMASHKDDEDKFFPMSGVMIFGKESAGASNTALDNVMITDEEEIKATNPVATERFGTV |
| | AVNFQSSSTDPATGDVHAMGALPGMVWQDRDVYLQGPIWAKIPHTDGHFHPSPLMGGFGLKN |
| | PPPQILIKNTPVPANPPAEFSATKFASFITQYSTGQVSVEIEWELQKENSKRWNPEVQYTSNYAKSA |
| | NVDFTVDNNGLYTEPRPIGTRYLTRPL |
| SEQ ID | MAADGYLPDWLEDTLSEGIRQWWKLKPGPPPPKPAERHKDDSRGLVLPGYKYLGPFNGLDKGEP |
| NO: 55 | VNEADAAALEHDKAYDRQLDSGDNPYLKYNHADAEFQERLKEDTSFGGNLGRAVFQAKKRVLEPL |
| (AAV2 | GLVEEPVKTAPGKKRPVEHSPVEPDSSSGTGKAGQQPARKRLNFGQTGDADSVPDPQPLGQPPA |
| (AAC03780)) | APSGLGTNTMATGSGAPMADNNEGADGVGNSSGNWHCDSTWMGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFTFSYTFEDVPFHSSYAHSQSLDRLMNPLIDQYLYYLSRTNTP |
| | SGTTTQSRLQFSQAGASDIRDQSRNWLPGPCYRQQRVSKTSADNNNSEYSWTGATKYHLNGRDS |
| | LVNPGPAMASHKDDEEKFFPQSGVLIFGKQGSEKTNVDIEKVMITDEEEIRTTNPVATEQYGSVST |
| | NLQRGNRQAATADVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDNLSEGIREWWALKPGVPQPKANQQHQDNRRGLVLPGYKYLGPGNGLDKG |
| NO: 56 | EPVNEADAAALEHDKAYDQQLKAGDNPYLKYNHADAEFQERLQEDTSFGGNLGRAVFQAKKRILE |
| (AAV3 | PLGLVEEAAKTAPGKKGAVDQSPQEPDSSSGVGKSGKQPARKRLNFGQTGDSESVPDPQPLGEPP |
| (AAC55049)) | AAPTSLGSNTMASGGGAPMADNNEGADGVGNSSGNWHCDSQWLGDRVITTSTRTWALPTYN |
| | NHLYKQISSQSGASNDNHYFGYSTPWGYFDFNRFHCHFSPRDWQRLINNNWGFRPKKLSFKLFNI |
| | QVRGVTQNDGTTTIANNLTSTVQVFTDSEYQLPYVLGSAHQGCLPPFPADVFMVPQYGYLTLNNG |
| | SQAVGRSSFYCLEYFPSQMLRTGNNFQFSYTFEDVPFHSSYAHSQSLDRLMNPLIDQYLYYLNRTQ |
| | GTTSGTTNQSRLLFSQAGPQSMSLQARNWLPGPCYRQQRLSKTANDNNNSNFPWTAASKYHLN |
| | GRDSLVNPGPAMASHKDDEEKFFPMHGNLIFGKEGTTASNAELDNVMITDEEEIRTTNPVATEQY |
| | GTVANNLQSSNTAPTTGTVNHQGALPGMVWQDRDVYLQGPIWAKIPHTDGHFHPSPLMGGFG |
| | LKHPPPQIMIKNTPVPANPPTTFSPAKFASFITQYSTGQVSVEIEWELQKENSKRWNPEIQYTSNYN |
| | KSVNVDFTVDTNGVYSEPRPIGTRYLTRNL |
| SEQ ID | MSFVDHPPDWLEEVGEGLREFLGLEAGPPKPKPNQQHQDQARGLVLPGYNYLGPGNGLDRGEP |
| NO: 57 | VNRADEVAREHDISYNEQLEAGDNPYLKYNHADAEFQEKLADDTSFGGNLGKAVFQAKKRVLEPF |
| (AAV5 | GLVEEGAKTAPTGKRIDDHFPKRKKARTEEDSKPSTSSDAEAGPSGSQQLQIPAQPASSLGADTMS |
| (AAD13756)) | AGGGGPLGDNNQGADGVGNASGDWHCDSTWMGDRVVTKSTRTWVLPSYNNHQYREIKSGSV |
| | DGSNANAYFGYSTPWGYFDFNRFHSHWSPRDWQRLINNYWGFRPRSLRVKIFNIQVKEVTVQDS |
| | TTTIANNLTSTVQVFTDDDYQLPYVVGNGTEGCLPAFPPQVFTLPQYGYATLNRDNTENPTERSSFF |
| | CLEYFPSKMLRTGNNFEFTYNFEEVPFHSSFAPSQNLFKLANPLVDQYLYRFVSTNNTGGVQFNKN |
| | LAGRYANTYKNWFPGPMGRTQGWNLGSGVNRASVSAFATTNRMELEGASYQVPPQPNGMTN |
| | NLQGSNTYALENTMIFNSQPANPGTTATYLEGNMLITSESETQPVNRVAYNVGGQMATNNQSST |
| | TAPATGTYNLQEIVPGSVWMERDVYLQGPIWAKIPETGAHFHPSPAMGGFGLKHPPPMMLIKNT |
| | PVPGNITSFSDVPVSSFITQYSTGQVTVEMEWELKKENSKRWNPEIQYTNNYNDPQFVDFAPDST |
| | GEYRTTRPIGTRYLTRPL |
| SEQ ID | MAADGYLPDWLEDNLSEGIREWWDLKPGAPKPKANQQKQDDGRGLVLPGYKYLGPFNGLDKGE |
| NO: 58 | PVNAADAAALEHDKAYDQQLKAGDNPYLRYNHADAEFQERLQEDTSFGGNLGRAVFQAKKRVLE |
| (AAV6 | PFGLVEEGAKTAPGKKRPVEQSPQEPDSSSGIGKTGQQPAKKRLNFGQTGDSESVPDPQPLGEPP |
| (AAB95450)) | ATPAAVGPTTMASGGGAPMADNNEGADGVGNASGNWHCDSTWLGDRVITTSTRTWALPTYN |
| | NHLYKQISSASTGASNDNHYFGYSTPWGYFDFNRFHCHFSPRDWQRLINNNWGFRPKRLNFKLF |
| | NIQVKEVTTNDGVTTIANNLTSTVQVFSDSEYQLPYVLGSAHQGCLPPFPADVFMIPQYGYLTLNN |
| | GSQAVGRSSFYCLEYFPSQMLRTGNNFTFSYTFEDVPFHSSYAHSQSLDRLMNPLIDQYLYYLNRTQ |
| | NQSGSAQNKDLLFSRGSPAGMSVQPKNWLPGPCYRQQRVSKTKTDNNNSNFTWTGASKYNLN |
| | GRESIINPGTAMASHKDDKDKFFPMSGVMIFGKESAGASNTALDNVMITDEEEIKATNPVATERFG |
| | TVAVNLQSSSTDPATGDVHVMGALPGMVWQDRDVYLQGPIWAKIPHTDGHFHPSPLMGGFGL |
| | KHPPPQILIKNTPVPANPPAEFSATKFASFITQYSTGQVSVEIEWELQKENSKRWNPEVQYTSNYAK |
| | SANVDFTVDNNGLYTEPRPIGTRYLTRPL |
| SEQ ID | MAADGYLPDWLEDNLSEGIREWWDLKPGAPKPKANQQKQDNGRGLVLPGYKYLGPFNGLDKGE |
| NO: 59 | PVNAADAAALEHDKAYDQQLKAGDNPYLRYNHADAEFQERLQEDTSFGGNLGRAVFQAKKRVLE |
| (AAV7 | PLGLVEEGAKTAPAKKRPVEPSPQRSPDSSTGIGKKGQQPARKRLNFGQTGDSESVPDPQPLGEPP |
| (AF513851_ | AAPSSVGSGTVAAGGGAPMADNNEGADGVGNASGNWHCDSTWLGDRVITTSTRTWALPTYNN |
| 2)) | HLYKQISSETAGSTNDNTYFGYSTPWGYFDFNRFHCHFSPRDWQRLINNNWGFRPKKLRFKLFNI |
| | QVKEVTTNDGVTTIANNLTSTIQVFSDSEYQLPYVLGSAHQGCLPPFPADVFMIPQYGYLTLNNGS |
| | QSVGRSSFYCLEYFPSQMLRTGNNFEFSYSFEDVPFHSSYAHSQSLDRLMNPLIDQYLYYLARTQSN |
| | PGGTAGNRELQFYQGGPSTMAEQAKNWLPGPCFRQQRVSKTLDQNNNSNFAWTGATKYHLNG |
| | RNSLVNPGVAMATHKDDEDRFFPSSGVLIFGKTGATNKTTLENVLMTNEEEIRPTNPVATEEYGIVS |
| | SNLQAANTAAQTQVVNNQGALPGMVWQNRDVYLQGPIWAKIPHTDGNFHPSPLMGGFGLKH |
| | PPPQILIKNTPVPANPPEVFTPAKFASFITQYSTGQVSVEIEWELQKENSKRWNPEIQYTSNFEKQTG |
| | VDFAVDSQGVYSEPRPIGTRYLTRNL |
| SEQ ID | MAADGYLPDWLEDNLSEGIREWWALKPGAPKPKANQQKQDDGRGLVLPGYKYLGPFNGLDKGE |
| NO: 60 | PVNAADAAALEHDKAYDQQLQAGDNPYLRYNHADAEFQERLQEDTSFGGNLGRAVFQAKKRVLE |
| (AAV8 | PLGLVEEGAKTAPGKKRPVEPSPQRSPDSSTGIGKKGQQPARKRLNFGQTGDSESVPDPQPLGEPP |
| (AF513852_ | AAPSGVGPNTMAAGGGAPMADNNEGADGVGSSSGNWHCDSTWLGDRVITTSTRTWALPTYN |
| 2)) | NHLYKQISNGTSGGATNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFQFTYTFEDVPFHSSYAHSQSLDRLMNPLIDQYLYYLSRTQT |
| | TGGTANTQTLGFSQGGPNTMANQAKNWLPGPCYRQQRVSTTTGQNNNSNFAWTAGTKYHLN |
| | GRNSLANPGIAMATHKDDEERFFPSNGILIFGKQNAARDNADYSDVMLTSEEEIKTTNPVATEEYGI |
| | VADNLQQONTAPQIGTVNSQGALPGMVWQNRDVYLQGPIWAKIPHTDGNFHPSPLMGGFGLK |
| | HPPPQILIKNTPVPADPPTTFNQSKLNSFITQYSTGQVSVEIEWELQKENSKRWNPEIQYTSNYYKST |
| | SVDFAVNTEGVYSEPRPIGTRYLTRNL |
| SEQ ID | MAADGYLPDWLEDNLSEGIREWWALKPGAPQPKANQQHQDNARGLVLPGYKYLGPGNGLDKG |
| NO: 61 | EPVNAADAAALEHDKAYDQQLKAGDNPYLKYNHADAEFQERLKEDTSFGGNLGRAVFQAKKRLLE |
| (AAV9 | PLGLVEEAAKTAPGKKRPVEQSPQEPDSSAGIGKSGAQPAKKRLNFGQTGDTESVPDPQPIGEPPA |
| (AAS99264)) | APSGVGSLTMASGGGAPVADNNEGADGVGSSSGNWHCDSQWLGDRVITTSTRTWALPTYNNH |
| | LYKQISNSTSGGSSNDNAYFGYSTPWGYFDFNRFHCHFSPRDWQRLINNNWGFRPKRLNFKLFNI |
| | QVKEVTDNNGVKTIANNLTSTVQVFTDSDYQLPYVLGSAHEGCLPPFPADVFMIPQYGYLTLNDGS |
| | QAVGRSSFYCLEYFPSQMLRTGNNFQFSYEFENVPFHSSYAHSQSLDRLMNPLIDQYLYYLSKTING |
| | SGQNQQTLKFSVAGPSNMAVQGRNYIPGPSYRQQRVSTTVTQNNNSEFAWPGASSWALNGRN |
| | SLMNPGPAMASHKEGEDRFFPLSGSLIFGKQGTGRDNVDADKVMITNEEEIKTTNPVATESYGQV |
| | ATNHQSAQAQAQTGWVQNQGILPGMVWQDRDVYLQGPIWAKIPHTDGNFHPSPLMGGFGM |
| | KHPPPQILIKNTPVPADPPTAFNKDKLNSFITQYSTGQVSVEIEWELQKENSKRWNPEIQYTSNYYK |
| | SNNVEFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 62 | PVNAADAAALEHDKAYDQQLKAGDNPYLRYNHADAEFQERLQEDTSFGGNLGRAVFQAKKRVLE |
| (AAV10 | PLGLVEEAAKTAPGKKRPVEPSPQRSPDSSTGIGKKGQQPAKKRLNFGQTGESESVPDPQPIGEPP |
| (AAT46337)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFEFSYTFEDVPFHSSYAHSQSLDRLMNPLIDQYLYYLSRTQS |
| | TGGTQGTQQLLFSQAGPANMSAQAKNWLPGPCYRQQRVSTTLSQNNNSNFAWTGATKYHLNG |
| | RDSLVNPGVAMATHKDDEERFFPSSGVLMFGKQGAGRDNVDYSSVMLTSEEEIKTTNPVATEQY |
| | GVVADNLQQANTGPIVGNVNSQGALPGMVWQNRDVYLQGPIWAKIPHTDGNFHPSPLMGGF |
| | GLKHPPPQILIKNTPVPADPPTTFSQAKLASFITQYSTGQVSVEIEWELQKENSKRWNPEIQYTSNYY |
| | KSTNVDFAVNTEGTYSEPRPIGTRYLTRNL |
| SEQ ID | MAADGYLPDWLEDNLSEGIREWWALKPGAPQPKANQQHQDNARGLVLPGYKYLGPGNGLDKG |
| NO: 63 | EPVNEADAAALEHDKAYDQQLKAGDNPYLKYNHADAEFQERLKEDTSFGGNLGRAVFQAKKRLLE |
| (AAVhu.68) | PLGLVEEAAKTAPGKKRPVEQSPQEPDSSVGIGKSGAQPAKKRLNFGQTGDTESVPDPQPIGEPPA |
| | APSGVGSLTMASGGGAPVADNNEGADGVGSSSGNWHCDSQWLGDRVITTSTRTWALPTYNNH |
| | LYKQISNSTSGGSSNDNAYFGYSTPWGYFDFNRFHCHFSPRDWQRLINNNWGFRPKRLNFKLFNI |
| | QVKEVTDNNGVKTIANNLTSTVQVFTDSDYQLPYVLGSAHEGCLPPFPADVFMIPQYGYLTLNDGS |
| | QAVGRSSFYCLEYFPSQMLRTGNNFQFSYEFENVPFHSSYAHSQSLDRLMNPLIDQYLYYLSKTING |
| | SGQNQQTLKFSVAGPSNMAVQGRNYIPGPSYRQQRVSTTVTQNNNSEFAWPGASSWALNGRN |
| | SLMNPGPAMASHKEGEDRFFPLSGSLIFGKQGTGRDNVDADKVMITNEEEIKTTNPVATESYGQV |
| | ATNHQSAQAQAQTGWVONQGILPGMVWQDRDVYLQGPIWAKIPHTDGNFHPSPLMGGFGM |
| | KHPPPQILIKNTPVPADPPTAFNKDKLNSFITQYSTGQVSVEIEWELQKENSKRWNPEIQYTSNYYK |
| | SNNVEFAVNTEGVYSEPRPIGTRYLTRNL* |
| SEQ ID | MAADGYLPDWLEDNLSEGIREWWALQPGAPKPKANQQHQDNARGLVLPGYKYLGPGNGLDKG |
| NO: 64 | EPVNAADAAALEHDKAYDQQLKAGDNPYLKYNHADAEFQERLKEDTSFGGNLGRAVFQAKKRLLE |
| (AAVLK03) | PLGLVEEAAKTAPGKKRPVDQSPQEPDSSSGVGKSGKQPARKRLNFGQTGDSESVPDPQPLGEPP |
| | AAPTSLGSNTMASGGGAPMADNNEGADGVGNSSGNWHCDSQWLGDRVITTSTRTWALPTYN |
| | NHLYKQISSQSGASNDNHYFGYSTPWGYFDFNRFHCHFSPRDWQRLINNNWGFRPKKLSFKLFNI |
| | QVKEVTQNDGTTTIANNLTSTVQVFTDSEYQLPYVLGSAHQGCLPPFPADVFMVPQYGYLTLNNG |
| | SQAVGRSSFYCLEYFPSQMLRTGNNFQFSYTFEDVPFHSSYAHSQSLDRLMNPLIDQYLYYLNRTQ |
| | GTTSGTTNQSRLLFSQAGPQSMSLQARNWLPGPCYRQQRLSKTANDNNNSNFPWTAASKYHLN |
| | GRDSLVNPGPAMASHKDDEEKFFPMHGNLIFGKEGTTASNAELDNVMITDEEEIRTTNPVATEQY |
| | GTVANNLQSSNTAPTTRTVNDQGALPGMVWQDRDVYLQGPIWAKIPHTDGHFHPSPLMGGFG |
| | LKHPPPQIMIKNTPVPANPPTTFSPAKFASFITQYSTGQVSVEIEWELQKENSKRWNPEIQYTSNYN |
| | KSVNVDFTVDTNGVYSEPRPIGTRYLTRPL* |
| SEQ ID | MAADGYLPDWLEDTLSEGIRQWWKLKPGPPPPKPAERHKDDSRGLVLPGYKYLGPFNGLDKGEP |
| NO: 65 | VNEADAAALEHDKAYDRQLDSGDNPYLKYNHADAEFQERLKEDTSFGGNLGRAVFQAKKRVLEPL |
| (AAVhu.1 | GLVEEPVKTAPGKKRPVEHSPVEPDSSSGTGKAGQQPARKRLNFGQTGDADSVPDPQPLGQPPA |
| (AAS99260)) | ÅPSGLGSTTMATGSGAPMADNNEGADGVGNSSGNWHCDSQWLGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNGGTTTIANNLTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANGNNNSNFPWTAATKYHLNG |
| | RDSLVNPGPAMASHKDDEEKFFPMHGTLIFGKQGTNANDADLENVMITDEEEIRATNPVATEQY |
| | GTVSNNLQNSNTGPTTGTVNHQGALPGMVWQDRDVYLQGPIWAKIPHTDGHFHPSPLTGGFGL |
| | KHPPPQIMIKNTPVPANPPTNFSSAKFASFITQYSTGQVSVEIEWELQKENSKRWNPEIQYTSNYNK |
| | SVNVDFTVDTNGVYSEPRPIGTRYLTRNL |
| SEQ ID | MAADGYPPDWLEDTLSEGIRQWWKLKPGPPPPKPAERHKDDSRGLVLPGYKYLGPFNGLDKGEP |
| NO: 66 | VNEADAAALEHDKAYDRQLDSGDNPYLKYNHADAEFQERLKEDTSFGGNLGRAVFQAKKRVLEPL |
| (AAVhu.2 | GLVEEPVKTAPGKKRPVEHSPVEPDSSSGTGKAGQRPARKRLNFGQTGDADSVPDPQPLGQPPAA |
| (AAS99270)) | PSGLGSTTMATGSGAPMADNNEGADGVGNSSGNWHCDSQWLGDRVITTSTRTWALPTYNNHL |
| | YKQISSQSGASNDNHYFGYSTPWGYFDFNRFHCHFSPRDWQRLINNNWGFRPKRLNFKLFNIQVK |
| | EVTQNDGTTTIANNLTSTVQVFTDSEYQLPYVLGSAHQGCLPPFPADVFMVPQYGYLTLNNGSQA |
| | VGRSSFYCLEYFPSQMLRTGNNFQFSYTFEDVPFHSSYAHSQSLDRLMNPLIDQYLYYLNKTQTNSG |
| | TLQQSRLLFSQAGPTNMSLQAKNWLPGPCYRQQRLSKQANDNNNSNFPWTAATKYHLNGRDSL |
| | VNPGPAMASHKDDEEKFFPMHGTLIFGKQGTNANDADLENVMITDEEEIRATNPVATEQYGTVS |
| | NNLQNSNTGPTTGTVNRQGALPGMVWQDRDVYLQGPIWAKIPHTDGHFHPSPLMGGFGLKHP |
| | PPQIMIKNTPVPANPPTNFSSAKFASFITQYSTGQVSVEIEWELQKENSKRWNPEIQYTSNYNKSVN |
| | VDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 67 | VNEADAAALEHDKAYDRQLDSGDNPYLKYNHADAEFQERLKEDTSFGGNLGRAVFQAKKRVLEPL |
| (AAVhu.3 | RPGLRKPVKTAPGKKRPVEHSPVEPDSSSGTGKAGQQPARKRLNFGQTGDADSVPDPQPLGQPP |
| (AAS99280)) | AAPSGLGSTTMATGSGAPMADNNEGADGVGNSSGNWHCDSQWLDDRVIATSTRTWALPTYN |
| | NHLYKQISSQSGACNDNHYFGYSTPWGYFDFNRFHCHFSPRDWQRLINSNWGFRPKRLNFKLFNI |
| | QVKEVTQNDGTTTIANNLTSTVQVFTDSEYQLPYVPGSAHQGCLPPFPADVFMVPQYGYLTLNNG |
| | SQAVGRSSFYCLEYFPSQMLRTGNNFQFSYTFEDVPFHSSYAHCQSLDRLMNPLIDQYLYYLNKTQ |
| | TNSGTLQQSRLLFSQAGPTNMSLQAKNWLPGPCYRQQRLSKQANDNNNCNFPWTAATKYHLN |
| | GRDSLVNPGPAMASHKDDEEKFFPMHGTLIFGKQGTNANDADLENVMITDEEEIRPTNPVATEQ |
| | YGTVSNNLQNSNTGPTTGTVNHQGALPGMVWQDRDVYLQGPIWAKIPHTDGHFHPSPLMGGF |
| | GLKHPPPQIMIKSTPVPANPPTNFSSAKFASSITQYSTGQVSVEIEWELQKENSKRWNPEIQYTSNY |
| | NKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 68 | VNEADAAALEHDKAYDRQLDSGDNPYLKYNHADAEFQERLKEDTSFGGNLGRAVFQAKKRVLEPL |
| (AAVhu.4 | GLVEEPVKTAPGKKRPVEHSPVEPDSSSGTGKAGQQPARKRLNFGQTGDADSVPDPQPLGQPPA |
| (AAS99287)) | APSGLGSTTMATGSGAPMADNNEGADGVGNSSGNWHCDSQWLGDRVITTSTRTWALPTYNN |
| | HLYKQISSQSGASNDNHYFGYSTPWGYFDFNRFHCHFSPRDWQRLVNNNRGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANDNNNSNFPWTAATKYHLNGR |
| | DSLVNPGPAMASHKDDEEKFFPMHGTLIFGKQGTNANDADLENVMITDEEEIRATNPVATEQYG |
| | TVSNNLQNSNTGPTTGTVNHQGALPGMVWQDRDVYLQGPIWAKIPHTDGHFHPSPLMGGFGL |
| | KHPPPQIMIKNTPVPANPPTNFSSAKFASFITQYSTGQVSVEIEWELQKENSKRWNPEIQYTSNYNK |
| | SVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 69 | PVNAADAAALEHDKAYDQQLKAGDNPYLRYNHADAEFQERLQEDTSFGGNLGRAVFQAKKRVLE |
| (AAVhu.6 | PLGLVEEGAKTAPGKKRPVEPSPQRSPDSSTGIGKTGQQPAKKRLNFGQTGDSESVPDPQPIGEPP |
| (AAS99306)) | AGPSGLGSGTMAAGGGAPMADNNEGADGVGSSSGNWHCDSAWLGDRVITTSTRPWALPTYN |
| | NHLYKQISNGTSGGSTNDNTYFGYSTPWGYFDFNRFHCHFSPRDWQRLINNNWGFRPKRLNFKL |
| | FNIQVKEVTQNEGTKTIANNLTSTIQVFTDSEYQLPYVLGSAHQGCPPPFPADVFMIPQYGYLTLNN |
| | GSQAVGRSSFYCLEYFPSQMRRTGNNFEFSYQFEDVPFHSSYAHSQSLDRLMNPLIDQYLYYLSRT |
| | QSTGGTAGTQQLLFSQAGPNNMSAQAKNWLPGPCYRQQRVSTTLSQNNNSNFAWTGATKYHL |
| | NGRDSLVNPGVAMATHKDDEERFFPSSGVLMFGKQGAGKDNVDYSSVMLTSEEEIKTTNPVATE |
| | QYGVVADNLQQQNAAPIVGAVNSQGALPGMVWQNRDVYLQGPIWAKIPHTDGNFHPSPLMG |
| | GFGLKHPPPQILIKNTPVPADPPTTFSQAKLASFITQYSTGQVSVEIEWELQKENSKRWNPEIQYTSN |
| | YYKSTNVDFAVNTEGTYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 70 | VNEADAAALEHDKAYDRQLDSGDNPYLKYNHADAEFQERLKEDTSFGGNLGRAVFQAKKRVLEPL |
| (AAVhu.7 | GLVEGPVKTAPGKKRPVEHSPAEPDSSSGTGKAGQQPARKRLNFGQTGDADSVPDPQPLGQPPA |
| (AAS99313)) | APSGLGSTTMATGSGAPMADNNEGADGVGNSSGNWHCDSQWLGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRLMNPLIDQYLYYLNKTQS |
| | NSGTLQQSRLLFSQAGPTSMSLQAKNWLPGPCYRQQRLSKQANDNNNSNFPWTAATKYHLNGR |
| | DSLVNPGPAMASHKDDEEKFFPMHGTLIFGKQGTNANDADLDNVMITDEEEIRTTNPVATEQYG |
| | YVSNNLQNSNTGPTTGTVNHQGALPGMVWQDRDVYLQGPIWAKIPHTDGHFHPSPLMGGFGL |
| | KHPPPQIMIKNTPVPANPPTNFSSAKFASFITQYSTGQVSVEIEWELQKENSKRWNPEIQYTSNYNK |
| | SVNVDFTVDTNGVYSEPRPIGTRYLTRNL |
| SEQ ID | MAADGYLPDWLEDTLSEGIRQWWKLKPGPPPPKPAERHQDNSRGLVLPGYKYLGPSNGLDKGEP |
| NO: 71 | VNEADAAALEHDKAYDRQLDSGDNPYLKYNHADAEFQERLKEDTSFGGNLGRAVFQAKKRVLEPL |
| (AAVhu.9 | GLVEEPVKTAPGKKRPVEHSPVEPDSSSGTGKAGHQPARKRLNFGQTGDADSVPDPQPLGQPPA |
| (AAS99314)) | APTSLGSTTMATGSGAPMADNNEGADGVGNSSGNWHCDSQWLGDRVITTSTRTWALPTYNNH |
| | LYKQISSQSGASNDNHYFGCSTPWGYFDFNRFHCHFSPRDWQRLINNNWGFRPKRLNFKLFNIQV |
| | KEVTQNDGTTTIANNLTSTVQVFTDSEYPLPYVLGSAHQGCLPPFPADVFMVPQYGYLTLNNGSQ |
| | AVGRSSFYCLEYFPSQMLRTGNNFTFSYTFEDVPFHSSYAHSQSLDRLMNPLIDQYLYYLNRTQSNS |
| | GTLQQSRLLFSQAGPTSMSLQAKNWLPGPCYRQQRLSKQANDNNNSNFPWTAATKYHLNGRDS |
| | LVNPGPAMASHKDDEEKFFPMHGTLIFGKQGTNANDADLEHVMITDEEEIRTTNPVATEQYGNV |
| | SNNLQNSNTGPTTENVNHQGALPGMVWQDRDVYLQGPIWAKIPHTDGHFHPSPLMGGFGLKH |
| | PPPQIMIKNTPVPANPPTNFSSAKFASFITQYSTGQVSVEIEWELQKENSKRWNPEIQYTSNYNKSV |
| | NVDFTVDTNGVYSEPCPIGTRYLTRNL |
| SEQ ID | MAADGYLPDWLEDTLSEGIRQWWKLKPGPPPPKLAERHQDDSRGLVLPGYKYLGPFNGLDKGEP |
| NO: 72 | VNEADAAALEHDKAYDRQLDSGDNPYLKYNHADAEFQERLKEDTSFGGNLGRAVFQAKKRVLEPL |
| (AAVhu.10 | GLVEEPVKTAPGKKRPVEHSPVEPDSSSGTGKAGHQPARKRLNFGQTGDADSVPDPQPLGQPPA |
| (AAS99261)) | APTSLGSTTMATGSGAPMADNNEGADGVGNSSGNWHCDSQWLGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFTVPQYGYLTLNNGSQA |
| | VGRSSFYCLEYFPSQMLRTGNNLTFSYTFEDVPFHSSYAHSQSLDRLMNPLIDQYLYYLNRTQSNSG |
| | TLQQSRLLFSQAGPTSMSLQAKNWLPGPCYRQQRLSKQANDNNNSNFPWTAATKYHLNGRDSL |
| | VNPGPAMASHKDDEEKFFPMHGTLIFGKQGTNANDADLEHVMITDEEEIRTTNPVATEQYGNVS |
| | NNLQNSNTGPTTENVNHQGALPGMVWQDRDVYLQGPIWAKIPHTDGHFHPSPLMGGFGLKHP |
| | PPQIMIKNTPVPANPPTNYSSAKFASFITQYSTGQVSVEIEWELRKENSKRWNPEIQYTSNYNKSVN |
| | VDFTVDTNGVYSEPRPIGTRYLTRNL |
| SEQ ID | MAADGYLPDWLEDTLSEGIRQWWKLKPGPPPPKPAERHQDDSRGLVLPGYKYLGPFNGLDKGEP |
| NO: 73 | VNEADAAALEHDKAYDRQLDSGDNPYLKYNHADAEFQERLKEDTSFGGNLGRAVFQAKKRVLEPL |
| (AAVhu.11 | GLVEEPVKTAPGKKRPVEHSPVEPDSSSGTGKAGHQPARKRLNFGQTGDADSVPDPQPLGQPPA |
| (AAS99262)) | APTSLGSTTMATGSGAPMADNNEGADGVGNSSGNWHCDSQWLGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFTFSYTFEDVPFHSSYAHSQSLDRLMNPLIDQYLYYLNRTQSNS |
| | GTLQQSRLLFSQAGPTSMSLQAKNWLPGPCYRQQRLSKQANDNNNSNFPWTAATKYRLNGRDS |
| | LVNPGPAMASHKDDEEKFFPMHGTLIFGKQGTNANDADLEHVMITDEEEIRTTNPVATEQYGNV |
| | SNNLQNSNTGPTTENVNHQGALPGMVWQDRDVYLQGPIWAKIPHTDGHFHPSPLMGGFGLKH |
| | PPPQIMIKNTPVPANPPTNFSSAKFASFITQYSTGQVSVEIEWELQKENSKRWNPEIQYTSNYNKSV |
| | NVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLYKGEP |
| NO: 74 | VDEADAAALEHDKAYDRQLDSGDNPYLKYNHADAEFQERLKEDTSFGGNLGRAVFQAKKRVLEPL |
| (AAVhu.15 | GLVGEPVKTAPGKKRPVEHSPVEPDSSSGTGKAGNQPARKRLNFGQTGDADSVPDPQPLGQPPA |
| (AAS99265)) | APSGLGSTTMATGSGAPVADNNEGADGVGNSSGNWHCDSQWLGDRVITTSTRTWALPTYNNH |
| | LYKQISSQSGASNDNHYFGYSTPWGYFDFNRFHCHFSPRDRQRLINNNWGFRPKRLNFKLFNIQV |
| | KEVTQNDGTTTIANNLTSTVQVFTDSGYQLPYVLGLAHQGCLPPFPADVFMVPQYGYLTLNNGSQ |
| | AVGRSSFYCLEYFPSQMLRTGNNFQFSYTFEDVPFHSSYAHSQSLDRLMNPLIDQYLYYLNKTQSNS |
| | GTLQQSRLLFSQAGPTSMSLQAKNWLPGPCYRQQRLSKQANDNNNSNFPWTAATKYHLNGRDS |
| | LVNPGPAMASHKDDEEKFFPMHGTLIFGKQGTNANDADLDNVMITDEEEIRTTNPVATEQYGYV |
| | SNNLQNSNTGPTTGTVNHQGALPGMVWQDRDVYLQGPIWAKIPHTDGHFHPSPLMGGFGLKH |
| | PPPQIMIKNTPVPANPPTNFSSAKFASFITQYSTGQVSVEIEWELQKEDSKRWNPEIQYTSNYNKPV |
| | NVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLYKGEP |
| NO: 75 | VNEADAAALEHDKAYDRQLDSGDNPYLKYNHAGAEFQERLKEDTSFGGNLGRAVFQAKKRVLEPL |
| (AAVhu.16 | GLVEEPVKTAPGKKRPVEHSPVEPDSSSGTGKAGNQPARKRLNFGQTGDADSVPDPQPLGQPPA |
| (AAS99266)) | APSGLGSTTMATGSGAPVADNNEGADGVGNSSGNWHCDSQWLGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFQFSYTFEDVPFHSSYAHSQSLDRLMNPLIDQYLYYLNKTQSNS |
| | GTLQQSRLLFSQAGPTSMSLQAKNWLPGPCYRQQRLSKQANDNNNSNFPWTAATKYHLNGRDS |
| | LVNPGPAMASHKDDEEKFFPMHGTLIFGKQGTNANDADLDNVMITDEEEIRTTNPVATEQYGYV |
| | SNNLQDSNTGPTTGTVNHQGALPGMVWQDRDVYLQGPIWAKIPHTDGHFHPSPLMGGFGLKH |
| | PPPQIMIKNTPVPANPPTNFSSAKFASFITQYSTGQVSVEIEWELQKENSKRWNPEIQYTSNYNKSV |
| | NVDFTVDTNGVYSEPRPIGTRYLTRNL |
| SEQ ID | MAADGYLPDWLEDNLSEGIREWWDLKPGAPKPKANQQKQDDGRGLVLPGCKYLGPFNGLDKGE |
| NO: 76 | PVNAADAAALEHDKAYDQQLKAGDNPYLRYNHADAEFQERLQEDTSFGGNLGRAVFQAKKRVLE |
| (AAVhu.17 | PLGLVEEGAKTAPGKKRPVEPSPQRSPDSSTGIGKTGQQPAKKRLNFGQTGDSESVPDPQPIGEPP |
| (AAS99267)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLNFKL |
| | FNIQVKEVTQNEGTKTIANNLTSTIQVFTDSEYQLPYVLGSAHQGCPPPFPADVFMIPQYGYLTLNN |
| | GSQAVGRSSFYCLEYFPSQMRRTGNNFEFSYQFEDVPFHSSYAHSQSLDRLMNPLIDQYLYYLSRT |
| | QSTGGTAGTQQLLFSQAGPNNMSAQAKNWLPGPCYRQQRVSTTLSQNNNSNFAWTGATKYHL |
| | NGRDSLVNPGVAMATHKDDEERFFPSSGVLMFGKQGAGKDNVDYSSVMLTSEEEIKTTNPVATE |
| | QYGVVADNLQQQNAAPIVGAVNSQGALPGMVWQNRDVYLQGPIWAKIPHTDGNFHPSPLMG |
| | GFGLKHPPPQILIKNTPVPADPPTTFSQAKLASFITQYSTGQVSVEIEWELQKENSKRWNPEIQYTSN |
| | YNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 77 | VNEADAAALEHDKAYDRQLESGDNPYLKYNHADAEFQERLKEDTSFGGNLGRAVFQAKKRVLEPL |
| (AAVhu.18 | GLVEEPVKTAPGKKRPVEHSPVEPDSSSGTGKAGQQPARKRLNFGQTGDADSVPDPQPLGQPPA |
| (AAS99268)) | APSGLGSTTMASGSGAPVADNNEGADGVGNSSGNWHCDSQWLGDRVITTSTRTWALPTYNNH |
| | LYKQISSQSGASNDNHYFGYSTPWGYFDFNRFHCHFSPRDWQRLINNSWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFQFSYTFEDVPFHSSYAHSQSLDRLLNPLIDQYLYYLNKTQSNS |
| | GTLQQSRLLFSQAGPTSMSLQAKNWLPGPCYRQQRLSKQANDNNNSNFPWTAATKYHLNGRDS |
| | LVNPGPAMASHKDDEEKFFPMHGTLIFGKQGTNANDADLDNVMITDEEEIRTTNPVATEQYGYV |
| | SNNLQNSNTGPTTGTVNHQGALPGMVWQDRDVYLQGPIWAKIPHTDGHFHPSPLMGGFGLKH |
| | PPPQIMIKNTPVPANPPTNFSSSKFASFITQYSTGQVSVEIEWELQKENSKRWNPEIQYTSNYNKSV |
| | NVDFTVDTNGVYSEPRPIGTRYPTRNL |
| SEQ ID | MAADGYLPDWLEDTLSEGIRQWWKLKPGPPPPKPAERHKDDSRGLVLPGYRYLGPFNGLDKGEP |
| NO: 78 | VNEADAAALEHDKAYDRQLDSGDNPYLKYNHVDAEFQERLKEDTSFGGNLGRAVFQAKKRILEPL |
| (AAVhu.20 | GLVEEPVKAAPGEKRPVEHSPAEPDSSSGTGKAGQQPARKRLNFGQTGDADSVPDPQPLGQPPA |
| (AAS99271)) | APSGLGTNTMASGSGAPMADNNEGADGVGNSSGNWHCDSTWMGDRVITTSTRTWALPTYNN |
| | HLYKQISSQSGASNDNHYFGYSTPWGHFDFNRFHCHFSPRDWQRLINNNWGFRPKRLSFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFTFSYTFEDVPFHSSYAHSQSLDRLMNPLIDQYLYYLSRTNTP |
| | SGTTTMSRLQFSQAGASDIRDQSRNWLPGPCYRQQRVSKTAADNNNSDYSWTGATKYHLNGRD |
| | SLVNPGPAMASHKDDEEKYFPQSGVLIFGKQDSGKTNVDIEKVMITDEEEIRTTNPVATEQYGSVS |
| | TNLQSGNTQAATSDVNTQGVLPGMVWQDRDVYLQGPIWAKIPHTDGHFHPSPPMGGFGLKHP |
| | PPQILIKNTPVPANPSTTFSAAKFASFITQYSTGQVSVEIEWELQKENSKRWNPEIQYTSNYNKSVNV |
| | DFTVDTNGVYSEPRPIGARYLTRNL |
| SEQ ID | MAADGYLPDWLEDTLSEGIRQWWKLKPGPPPPKPAERHKDDSRGLVLPGYKYLGPFNGLDKGEP |
| NO: 79 | VNEADAAALEHDKAYDRQLDSGDNPYLKYNHADAEFQERLKEDTSFGGNLGRAVFQAKKRILEPL |
| (AAVhu.21 | GLVEEPVKTAPGKKRPVEHSPAEPDSSSGTGKAGQQPARKRLNFGQTGDADSVPDPRPLGQPPAA |
| (AAS99272)) | PSGLGTNTMASGSGAPMADNNEGADGVGNSSGNWHCDSTWMGDRVITTSTRTWALPTYNNH |
| | LYKQISSQSGASNDNHYFGYSTPWGYFDFNRFHCHFSPRDWQRLINNNWGFRPKRLSFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFTFSYTFEDVPFHSSYAHSQSLDRLMNPLIDQYLYYLSRTNTPS |
| | GTTTMSRLQFSQAGASDIRDQSRNWLPGPCYRQQRVSKTAADNNNSDYSWTGATKYHLNGRDS |
| | LVNPGPAMASHKDDEEKYFPQSGVLIFGKQDSGKTNVDIEKVMITDEEEIRTTNPVATEQYGSVST |
| | NLQSGNTQAATSDVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 80 | VNEADAAALEHDKAYDRQLDSGDNPYLKYNHADAEFQERLKGDTSFGGNLGRAVFQAKKRILEPL |
| (AAVhu.22 | GLVEEPVKTAPGKKRPVEHSPAEPDSSSGTGKAGQQPARKRLNFGQTGDADSVPDPQPLGQPPA |
| (AAS99273)) | APSGLGTNTMASGSGAPMADNNEGADGVGNSSGNWHCDSTWMGGRVITTSTRTWALPTYNN |
| | HLYKQISSQSGASNDNHYFGYSTPWGYFDFNRFHCHFSPRDWQRLINNNWGFRPKRLSFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQTLRTGNNFTFSYTFEDVPFHSSYAHSQSLDRLMNPLIDQYLYYLSRTNTPS |
| | GTTTMSRLQFSQAGASDIRDQSRNWLPGPCYRQQRVSKTAADNNNSDYSWTGATKYHLNGRDS |
| | LVNPGPAMASHKDDEEKYFPQSGVLIFGKQDSGKTNVDIEKVMITDEEEIRTTNPVATEQYGSVST |
| | NLQSGNTQAATSDVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 81 | VNEADAAALEHDKAYDRQLDSGDNPYLKYNHADAEFQERLKEDTSFGGNLGRAVFQAKKRILEPL |
| (AAVhu.23 | GLVEEPVKTAPGKKRPVEHSPAEPDSSSGTGKAGQQPARKRLNFGQTGDADSVPDPQPLGQPPA |
| (AAS99274)) | APSGLGTNTMASGSGAPMADNNEGADGVGNSSGNWHCDSTWMGDRVITTSTRTWALPTCNN |
| | HLYKQISSQSGASNDNHYFGYSTPWGYFDFNRFHCHFSPRDWQRLINNNWGFRPKRLSFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFTFSYTFEDVPFHSSYAHSQSLDRLMNPLIDQYLYYLSRTNTP |
| | SGTTTMSRLQFSQAGASDIRDQSRNWLPGPCYRQQRVSKTAADNNNSDYSWTGATKYHLNGRD |
| | SLVNPGPAMASHKDDEEKYFPQSGVLIFGKQDSGKTNVDIEKVMITDEEEIRTTNPVATEQYGSVS |
| | TYLQSGNTQAATSDVNTQGVLPGMVWQDRDVYLRGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDGSRGLVLPGYKYLGPFNGLDKGEP |
| NO: 82 | VNEADAAALEHDKAYDRQLNSGDNPYLKYNHADAEFQERLKEDTSFGGNLGRAVFQAKKRVLEPL |
| (AAVhu.25 | GLVEEPVKTAPGKKRPVEHSPAEPDSSSGTGKAGQQPARKRLNFGQTGDADSVPDPQPLGQPPA |
| (AAS99276)) | APSGLGSTTMATGSGAPMADNNEGADGVGNSSGNWHCDSQWLGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSPFYCLEYFPSQMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANDNNNSNFPWTAATKYHLNGR |
| | DSLVNPGPAMASHKDDEEKFFPMHGTLIFGKQGTNANDADLENVMITDEEEIRTTNPVATEQYGT |
| | VSNNLQNSNTGPTTGTVNHQGALPGMVWQDRDVYLQGPIWAKIPHTDGHFHPSPLMGGFGLK |
| | HPPPQIMIKNTPVPANPPTNFSSAKFASFITQYSTGQVSVEIEWELQKENSKRWNPEIQYTSNYNKS |
| | VNVDFTVDNNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 83 | VNEADAAALEHDKAYDRQLDSGDNPYLKYNHADAEFQERLKEDTSFGGNLGRAVFQAKKRILEPL |
| (AAVhu.27 | GLVEEPVKTAPGKKRPVEHSPAEPDSSSGTGKAGQQPARKRLNFGQTGDADSVPDPQPLGQPPA |
| (AAS99277)) | APSGLGTNTMASGSGAPMADNNEGADGVGNSSGNWHCDSTWMGDRVITTSTRTWALPTYNN |
| | HLYKQISSQSGASNDNHYFGYSTPWGYFDFNRFHCHFSPRDWQRLINNNWGFRPKRLSFKLFNIQ |
| | VKEVTQNDGTTTIANNLTSTVQVFTDSGYQLPYVLGSAHQGCLPPFPADVFMVPQYGYLTLNNGS |
| | QAVGRSSFYCLEYFPSQMLRTGNNFTFSYTFEDVPFHSSYAHGQSLDRLMNPLIDQYLYYLSRTNTP |
| | SGTTTMSRLQFSQAGASDVRDQSRNWLPGPCYRQQRVSKTAADNNNSDYSWTGATKYHLNGR |
| | DSLVNPGPAMASHKDDEEKYFPQSGVLVFGKQDSGKTNVDIEKVMITDEEEIRTTNPAATEQYGS |
| | VSTNLQSGNTQAATSDVNTQGVLPGMVWQDRDVYLQGPIWAKIPHTDGHFHPSPLMGGFGLK |
| | HPPPQILIKNTPVPANPSTTFSAAKFVSFITQYSTGQVSVEIEWELQKENSKRWNPEIQYTSNYNKSV |
| | NVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 84 | VNEADAAALEHDKAYDRQLDSGDNPYLKYNHADAEFQERLKEDTSFGGNLGRAVFQAKKRVLEPL |
| (AAVhu.28 | SLVEEPVKTAPGKKRPVEHSPAEPDSSSGTGKSGNQPARKRLNFGQTGDSDSVPDPQPLGQPPAA |
| (AAS99278)) | PSGLGTNTMATGSGAPMADNNEGADGVGNSSGNWHCDSTWMGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFTFSYTFEDVPFHSSYAHSQSLDRLMNPLIDQYLYYLSRTNTPS |
| | GTTTQSRLQFSQAGASDIQDQSRNWLPGPCYRQQRVSKTSADNNNSEYSWTGATKYHLNGRDSL |
| | VNPGPAMASHKDDEEKFFPQSGVLIFGKQGSEKTNVDIEKVMITDEEEIRTTNPVATEQYGSVSTN |
| | LQSGNTQAATADVNTQGVLPGMVGQDRDVYLQGPTWAKIPHTDGHFHPSPLMGGFGLKHPPP |
| | QILIKNTPVPANPSTTFSAAKFASFITQYSTGQVSVEIEWELQKENSKRWNPEIQYTSNYNKSVNVD |
| | FTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 85 | VNEADAAALEHDKAYDRQLDSGDNPYLKYNHADAEFQERLKEDTSFGGNLGRAVFQAKKRVLEPL |
| (AAVhu.29 | GLVEEPVKTAPGKKRPVEHSPAEPDSSSGTGKSGNQPARKRLNFGQTGDSDSVPDPQPLGQPPAA |
| (AAS99279)) | PSGLGTNTMATGSGAPMADNNEGADGVGNSSGNWHCDSTWMGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLGYFPSQMLRTGNNFTFSYTFEDVPFHSSYAHSQSLDRLMNPLIDQYLYYLSRTNTPS |
| | GTTTQSRLQFSQAGASDIRDQSRNWLPGPCYRQQRVSKTSADNNNSEYSWTGATKYHLNGRDSL |
| | VNPGPAMASHKDDEEKFFPQSGVLIFGKQGPEKTNVDIEKVMITDEEEIRTTNPVATEQYGSVSTN |
| | LQSGNTQAATADVNTQGVLPGMVWQDRDVYLQGPIWAKIPHTDGHFHPSPLMGGFGLKHPPP |
| | QILIKNTPVPANPSTTFSAAKFASFITQYSTGQVSVEIEWELQKENSKRWNPEIQYTSNYNKSVNVD |
| | FTVDTNGVYSEPRPIGTRYLTRNL |
| SEQ ID | MAADGYLPDWLEDTLSEGIRQWWKLKPGPPPPKPAERHKDDSRGLVLPGYKYLGPGNGLDKGEP |
| NO: 86 | VNAADAAALEHDKAYDQQLKAGDNPYLKYNHADAEFQERLKEDTSFGGNLGRAVFQAKKRLLEPL |
| (AAVhu.31 | GLVEEAAKTAPGKKRPVEQSPQEPDSSAGIGKSGSQPAKKKLNFGQTGDTESVPDPQPIGEPPAAP |
| (AAS99281)) | SGVGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTLNDGGQ |
| | AVGRSSFYCLEYFPSQMLRTGNNFQFSYEFENVPFHSSYAHSQSLDRLMNPLIDQYLYYLSKTINGS |
| | GQNQQTLKFSVAGPSNMAVQGRNYIPGPSYRQQRVSTTVTQNNNSEFAWPGASSWALNGRNS |
| | LMNPGPAMASHKEGEDRFFPLSGSLIFGKQGTGRDNVDADKVMITNEEEIKTTNPVATESYGQVA |
| | TNHQSAQAQAQTGWVQNQGILPGMVWQDRDVYLQGPIWAKIPHTDGNFHPSPLMGGFGMK |
| | HPPPQILIKNTPVPADPPTAFNKDKLNSFITQYSTGQVSVEIEWELQKENSKRWNPEIQYTSNYYKS |
| | NNVEFAVSTEGVYSEPRPIGTRYLTRNL |
| SEQ ID | MAADGYLPDWLEDTLSEGIRQWWKLKPGPPPPKPAERHKDDSRGLVLPGYKYLGPGNGLDKGEP |
| NO: 87 | VNAADAAALEHDKAYDQQLKAGDNPYLKYNHADAEFQERLKEDTSFGGNLGRAVFQAKKRLLEPL |
| (AAVhu.32 | GLVEEAAKTAPGKKRPVEQSPQEPDSSAGIGKSGSQPAKKKLNFGQTGDTESVPDPQPIGEPPAAP |
| (AAS99282)) | SGVGSLTMASGGGAPVADNNEGADGVGSSSGNWHCDSQWLGDRVITTSTRTWALPTYNNHLY |
| | KQISNSTSGGSSNDNAYFGYSTPWGYFDFNRFHCHFSPRDWQRLINNNWGFRPKRLNFKLFNIQV |
| | KEVTDNNGVKTIANNLTSTVQVFTDSDYQLPYVLGSAHEGCLPPFPADVFMIPQYGYLTLNDGSQA |
| | VGRSSFYCLEYFPSQMLRTGNNFQFSYEFENVPFHSSYAHSQSLDRLMNPLIDQYLYYLSKTINGSG |
| | QNQQTLKFSVAGPSNMAVQGRNYIPGPSYRQQRVSTTVTQNNNSEFAWPGASSWALNGRNSL |
| | MNPGPAMASHKEGEDRFFPLSGSLIFGKQGTGRDNVDADKVMITNEEEIKTTNPVATESYGQVAT |
| | NHQSAQAQAQTGWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKRWNPEIQYTSNYYKSN |
| | NVEFAVNTEGVYSEPRPIGTRYLTRNL |
| SEQ ID | MAADGYLPDWLEDTLSEGIRQRWKLKPGPPPPEPAERHKDDSRGLVLPGYKYLGPFNGLDKGEPV |
| NO: 88 | NEADAAALEHDKAYDRQLDSGDNPYLKYNHADAEFQERLKEDTSFGGNLGRAVFQAKKRVLEPLG |
| (AAVhu.34 | LVEEPVKTAPGKKRPVEHSPVEPDSSSGTGKAGQQPARKRLNFGQTGDADSVPDPQPLGQPPAAP |
| (AAS99283)) | SGLGTNTMATGSGAPMADNNEGADGVGNSSGNWHCDSTWMGDRVITTSTRTWALPTYNNHL |
| | YKQISSQSGASNDNHYFGYSTPWGYFDFNRFHCHFSPRDWQRLINNNWGFRPKRLNFKLFNIQVK |
| | EVTQNDGTTTIANNLTSTVQVFTDSEYQLPYVLGSAHQGCLPPFPADVFMVPQYGYLTLNNESQA |
| | VGRSSFYCLEYFPSQMLRTGNNFTFSYTFEDVPFHSSYAHSQSLGRLMNPLIDQYLYYLSRTNTPSGT |
| | TTQSRLQFSQAGASDIRDQSRNWLPGPCYRQQRVSKTSADNNNSEYSWTGATKYHLNGRDSLVN |
| | PGPAMASHKDDEEKFFPQSGVLIFGKQGSEKTNVDIEKVMITDEEEIRTTNPVATEQYGSVSTNLQ |
| | RGNRQAATADVNTQGVLPGMVWQDRDVYLQGPIWAKIPHTDGHFHPSPLMGGFGLKHPPPQI |
| | LIKNTPVPANPSTTFSAAKFASFITQYSTGQVSVEIEWELQKENSKRWNPEIQYTSNYNKSVNVDFT |
| | VDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 89 | PVNAADAAALEHDKAYDQQLKAGDNPYLRYNHADAEFQERLQEDTSFGGNLGRAVFQAKKRVLE |
| (AAVhu.37 | PLGLVEEAAKTAPGKKRPVEPSPQRSPDSSTGIGKKGQQPAKKRLNFGQTGDSESVPDPQPIGEPP |
| (AAS99285)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFEFSYTFEDVPFHSSYAHSQSLDRLMNPLIDQYLYYLSRTQS |
| | TGGTQGTQQLLFSQAGPANMSAQAKNWLPGPCYRQQRVSTTLSQNNNSNFAWTGATKYHLNG |
| | RDSLVNPGVAMATHKDDEERFFPSSGVLMFGKQGAGRDNVDYSSVMLTSEEEIKTTNPVATEQY |
| | GVVADNLQQTNTGPIVGNVNSQGALPGMVWQNRDVYLQGPIWAKIPHTDGNFHPSPLMGGFG |
| | LKHPPPQILIKNTPVPADPPTTFSQAKLASFITQYSTGQVSVEIEWELQKENSKRWNPEIQYTSNYYK |
| | STNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 90 | PVNAADAAALEHDKAYDQQLKAGDNPYLRYNHADAEFQERLQEDTSFGGNLGRAVFQAKKRVLE |
| (AAVhu.39 | PLGLVEEGAKTAPGKKRPVEPSPQRSPDSSTGIGKKGQQPARKRLNFGRTGDSESVPDPQPIGEPP |
| (AAS99286)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYLDFNRFHCHFSPRDWQRLINNNWGFRPKRLSFKLFN |
| | IQVKEVTQNEGTKTIANNLASTIQVFTDSEYQPPYVLGSAHQGCLPPFPADVFMIPQYGYLTLNNGS |
| | QAVGRSSFYCLEYFPSQMLRTGNNFSFSYTFEDVPFHSSYAHSQSLDRLMNPLIDQYLYYLSRTQST |
| | GGTAGTQQLLFSRAGPSNMSAQARNWLPGPCYRQQRVSTTLSQNNNSNFAWTGATKYHLNGR |
| | DSLVNPGVAMATNKDDEDRFFPSSGILMFGKQGAGKDNVDYSNVMLTSEEEIKTTNPVATEQYG |
| | VVADNLQQQNTAPTVGAVNSQGALPGMVWQNRDVYLQGPIWAKIPHTDGNFHPSPLMGGFG |
| | LKHPPPQILIKNTPVPADPPTAFNQAKLNSFIAQYSTGQVSVEIEWELQKENSKRWNPEIQYTSNYY |
| | KSTNADFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 91 | PVNAADAAALEHDKAYDQQLKAGDNPYLRYNHADAEFQERLQEDTSFGGNLGRAVFQAKKRVLE |
| (AAVhu.41 | PLGPVEEAAKTAPGKKRPVEPPPQRSPDSSTGIGKKGQQPAKKRLNFGQTGDSESVPDPQPIGEPP |
| (AAS99289)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLSFKLF |
| | NIQVKEVTQNEGTKTVANNLTSTIQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | STGGTQGTQQLLFSQAGPANMSAQAKNWLPGPCYRQQRVSTTLSQNNNSNFAWTGATKYHLN |
| | GRDSLVNPGVAMATHKDDEERFFPSSGVLMFGKQGAGRDNVDYSSVMLTSEEEIKTTNPVATEQ |
| | YGVVADNLQQTNTGPIVGNVNSQGALPGMVWQNRDVYLQGPIWAKIPHTDGNFHPSPLMGGF |
| | GLKHPPPQILIKNTPVPADPPTTFSQAKLASFITQYSTGQVSVEIEWELQKENSKRWNPEIQYTSNYY |
| | KSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 92 | PVNAADAAALEHDKAYDQQLKAGDNPYLRYNHADAEFQERLQEDTSFGGNLGRAVFQAKKRVLE |
| (AAVhu.42 | PLGLVEEAAKTAPGKKRPVEPSPQRSPDSSTGIGKKGQQPAKKRLNFGQTGDSESVPDPQPIGEPP |
| (AAS99290)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFEFSYTFEDVPFHSSYAHSQSLDRLMNPLIDQYLYYLSRTQS |
| | TGGTQGTQQLLFSQAGPANMSAQAKNWLPGPCYRQQRVSTTLSQSNNSNFAWTGATKYHLNG |
| | RDSLVNPGVAMATHKDDEERFFPSSGVLMFGKQGAGRDNVDYSSVMLTSEEEIKTTNPVATEQY |
| | GVVADNLQQTNTGPIVGNVNSQGALPGMVWQNRDVYLQGPIWAKIPHTDGNFHPSPLMGGLG |
| | LKHPPPQILIKNTPVPADPPTTFSQAKLASFITQYSTGQVSVEIEWELQKENSKRWNPEIQYTSNYYK |
| | STNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 93 | PVNAADAAALEHDKAYDQQLKAGDNPYPRYNHADAEFQERLQEDTPFGGNLGRAVFQAKKRVLE |
| (AAVhu.43 | PLGLVEEAAKTAPGKKRPVEPSPQRSPDSSTGIGKKGQQPAKKRLNFGQTGDSESVPDPQPIGEPP |
| (AAS99291)) | AGPSGLGSGTMAAGGGAPMADNNEGADGVGNASGNWHCDSTWLGDRVITTSTRTWALPTYN |
| | NHLYKQISSASTGASNDNHYFGYSTPWGYFDFNRFHCHFSPRDWQRLINNNWGFRPKRLNFKLF |
| | NIQVKEVTTNDGVTTIANNLTSTVQVFSDSEYQLPYVLGSAHQGCLPPFPADVFMIPQYGYLTLNN |
| | GSQAVGRSSFYCLEYFPSQMLRTGNNFTFSYTFEEVPLHSSYAHSQSLDRLMNPLIVQYLYYLNRTQ |
| | NQSGSAQNKDLLFSRGSPAGMSVQPKNWLPGPCYRQQRVSKTKTDNNNSNFTWTGASKYNLN |
| | GRESIINPGTAMASHKDDEDKFFPMSGVMIFGKESAGASNTALDNVMITDEEEIKATNPVATERFG |
| | TVAVNFQSSSTDPATGDVHAMGALPGMVWQDRDVYLQGPIWAKIPHTDGHFHPSPLMGGFGL |
| | KNPPPQILIKNTPVPANPPAEFSATKFASFITQYSTGQVSVEIEWELQKENSKRWNPEVQYTSNYAK |
| | SASVDFTVDNNGLYTEPRPIGTRYLTRPL |
| SEQ ID | MAADGYLPDWLEDTLSEGIRQWWKLRPGPPPPKPAERHKDDSRGLVLPGYKYLGPFNGLDKGEP |
| NO: 94 | VNEADAAALEHDKAYDRQLDSGDNPYLKYNHADAEFQERLKEDTSFGGNLGRAVFQAKKRVLEPL |
| (AAVhu.44 | GLVEEGAETAPGKKRPVEQSPQGPDSSSGIGKTGQQPAKKRLNFGQTGDSESVPDPQPLGEPPAT |
| (AAS99292)) | PAAVGPTTMASGGGAPMADNNEGADGVGNASGNWHCDSTWLGDRVITTSTRTWALPTYNNH |
| | LYKQISSASTGASNDNHYFGYSTPWGYFDFNRFHCHFSPRDWQRLINNNWGFRPKRLNFKLFNIQ |
| | VKEVTTNDGVTTIANNLTSTVQVFSDSEYQLPYVLGSAHQGCLPPFPADVFMIPQYGYLTLNNGSQ |
| | AVGRSSFYCLEYFPSQMLRTGNNFTFSYTFEEVPFHSSYAHSQSLDRLMNPLIDQYLYYPNRTQNQS |
| | GSAQNKDLLFSRGSPAGMSVQPKNWLPGPCYRQQRVSKTKTDNNNSNFTWTGASKYNLNGRES |
| | IINPGTAMASHKDDEDKFFPMSGVMIFGKESAGASNTALDNVMITDEEEIKATNPVATERFGTVA |
| | VNFQSSSTDPATGDVHAMGALPGMVWQGRDVYLQGPIWAKIPHTDGHFHPSPLMGGFGLKNP |
| | PPQILIKNTPVPANPPAEFSATKFASFITQYSTGQVSVEIEWELQKENSKRWNPEVQYTSNYAKSAN |
| | VDFTVDNNGLYTEPRPIGTRYLTRPL |
| SEQ ID | MAADGYLPDWLEDTLSEGIRQWWKLKPGPPPPKPAERHRDDSRGLVLPGYKYLGPFNGLDKGEP |
| NO: 95 | VNEADAAALEHDKAYDRQLDSGDNPYLKYNHADAEFQERLKEDTSFGGNLGRAVFQAKKRVLEPL |
| (AAVhu.45 | GLVEEPVKTAPGKKRPVEHSPVEPDSSSGTGKAGQQPARKRLNFGQTGDADSVPDPQPLGQPPA |
| (AAS99293)) | APSGLGTNTMATGSGAPMADNNEGADGVGNSSGNWHCDSTWMGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSGYQLPYVLGSAHQGCLPPFPADVFMVPQYGYPTLNNGS |
| | QAVGRSSFYCLEYFPSQMLRTGNNFTFSYTFEDVPFHSSYAHSQSLDRLMNPLIDQYLYYLSTTNTP |
| | SGTTTQSRLQFSQAGASDIRDQSRNWLPGPCYRQQRVSKTSADNNNSEYSWTGATKYHLNGRDS |
| | LVNPGPAVASHKDDEEKFFPQSGVLIFGKQGSEKTNVDIEKVMITDEEEIRTTNPVATEQYGSVSTN |
| | LQRGNRQAATADVNTQGVLPGMVWQDRDVYLQGPIWAKIPHTDGHFHPSPLMGGFGLKHPPP |
| | QILIKNTPVPANPSTTFSAAKFASFITQYSTGQVSVEIEWELQKENSKRWNPEIQYTSNYNKSVNVD |
| | FTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 96 | VNEADAAALEHDKAYDRQLDSGDNPYLKYNHADAEFQERLKEDTSFGGNLGRAVFQAKKRVLEPL |
| (AAVhu.46 | GLVEEGAKTAPGKKRPVEQSPQEPDSPSGIGKTGQQPAKKRLNFGQTGDSESVPDPQPLGEPPAT |
| (AAS99294)) | PAAVGPTTMASGGGAPMADNNEGADGVGNASGNWHCDSTWLGDRVITTSTRTWALPTYNNH |
| | LYKQISSASTGASNDNHYFGYSTPWGYFDFNRFHCHFSPRDWQRLINNNWGFRPKRLNFKLFNIQ |
| | VKEVTTNDGVTTIANNLTSTVQVFSDSEYQLPYVLGSAHQGRLPPFPADVFMIPQYGYLTLNNGSQ |
| | AVGRSSSYCLEYFPSQMLRTGNNFTFSYTFEEVPLHSSCAHSQSLDRLMNPLIDQYLYYLNRTQNQS |
| | GSAQNRDLLFSRGSPAGMSVQPKNWLPGPCYRQQRVSKTKTDNNNSNFTWTGASKYNLNGRES |
| | IINPGTAMASHKDDEDKFFPMSGVMIFGKESAGASNTALDNVMITDEEEIKATNPVATERFGTVÅ |
| | VNFQSSSTDPATGDVHAMGALPGMVWQDRDVYLQGPIWAKIPHTDGHFHPSPLMGGFGLKNP |
| | PPQILIKNTPVPANPPAEFSATKFASFITQYSAGQVSVEIEWELQKENSKRWNPEVQYTSNYAKSAN |
| | VDFTVDNNGLYTEPRPIGTRYLTRPL |
| SEQ ID | MAADGYLPDWLEDTLSEGIRQWWKLKPGPPPPKPAERHRDDSRGLVLPGYKYLGPFNGLDKGEP |
| NO: 97 | VNEADAAALEHDKAYDRQLDSGDNPYLKYNHADAEFQERLKEDTSFGGNLGRAVFQAKKRVLEPL |
| (AAVhu.47 | GLVGEPVKTAPGKKRPVEHSPVEPDSSSGTGKAGQQPARKRLNFGQTGDADSVPDPQPLGQPPA |
| (AAS99295)) | APSGLGTNTMATGSGAPMADNNEGADGVGNSSGNWHCDSTWMGDRVITTSTRTWALPTYNN |
| | HLYKQISSQSGASNDSHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFTFSYTFEDVPFHSSYAHSQSLDRLMNPLIDQYLYYLSTTNTP |
| | SGTTTQSRLQFSQAGASDIRDQSRNWLPGPCYRQQRVSKTSADNNNSEYSWTGATKYHLNGRDS |
| | LVNPGPAMASHKDNEEKFFPQSGVLIFGKQGSEKTNVDIEKVMITDEEEIRTTNPVATEQYGSVST |
| | NLQRGNRQAATADVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 98 | PVNAADAAALEHDKAYDQQLKAGDNPYLRYNHADAEFQERLQEDTSFGGNLGRAVFQAKKRVLE |
| (AAVhu.48 | PLGLVEEGAKTAPGKKRPVEQSPQEPDSSSGIGKTGQQPAKKRLNFGQTGDSESVPDPQPLGEPPA |
| (AAS99296)) | TPAAVGPTTMASGGGAPMADNNEGADGVGNASGNWHCDSTWLGDRVITTSTRTWALPTYNN |
| | HLYKQISSTSTGASNDNHYFGYGTPWGYFDFNRFHCHFSPRDWQRLINNNWGFRPKRLNFKLFNI |
| | QVEEVTTNDGVTTIANNLTSTVQVFSDSEYQLPYVLGSAHQGCLPPFPADVFMIPQYGYLTLNNGS |
| | QAVGRSSFYCLEYFPSQMLRTGNNFTFSYTFEEVPFHSSYAHSQSLDRLMNPLIDQYLYYLNRTQN |
| | QSGSAQNKDLLFSRGSPAGMSVQPKNWLPGPCYRQQRVSKTKTDNNNSNFTWTGASKYNLNGR |
| | ESIINPGTAVASHKDDEDKFFPMSGVMIFGKESAGASSTALDNVMITDEEEIKATNPVATERFGTVA |
| | VNFQSSSTDPATGDVHAMGALPGMVWQDRDVYLQGPIWAKIPHTDGHFHPSPLMGGFGLKNP |
| | PPQILIKNTPVPANPPAEFSATKFASFITQYSTGQVSVEIEWELQKENSKRWNPEVQYTSNYAKSAN |
| | VDFTVDNNGLYTEPRPIGTRYLTRPL |
| SEQ ID | MAADGYLPDWLEDTLSEGIRQWWKLKPGPPPPKPAERHKDDSRGLVLPGYKYLGPFNGLDKGEP |
| NO: 99 | VNEADAAALEHDKAYDRQLDSGDNPYLKYNHADAEFQERLKEDTSFGGNLGRAVFQAKKRVLEPL |
| (AAVhu.51 | GLVGEPVKTAPGKKRPVEHSPVEPDSSSGTGKAGQQPARKRLNFGQTGDADSVPDPQPLGQPPA |
| (AAS99298)) | ÅPSGLGTNTMATGSGAPMADNNEGADGVGNSSGNWHCDSTWMGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFTFSYTFEDVPFHSGYAHSQSLDRLMNPLIDQYLYYLSTTNTP |
| | SGTTTQSRLQFSQAGASDIRDQSRNWLPGPCYRQQRVSKTSADNNNSEYSWTGATKYHLNGRDS |
| | LVNPGPAMASHKDNEEKFFPQSGVLIFGKQGSEKTNVDIEKVMITDEEEIRTTNPVATEQYGSVST |
| | NLQRGNRQAATADVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 100 | VNEADAAALEHDKAYDRQLDSGDNPYLKYNHADAEFQERLKEDTSFGGNLGRAVFQAKKRVLEPL |
| (AAVhu.52 | GLVGEPVKTAPGKKRPVEHSPVEPDSSSGTGKAGQQPARKRLNFGQTGDADSVPDPQPLGQPPA |
| (AAS99299)) | APSGLGTNTMATGSGAPMADNNEGADGVGNSSGNRHCDSTWMGDRVITTSTRTWALPTYNN |
| | HLYRQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLSNGS |
| | QAVGRSSFYCPEYFPSQMLRTGNNFTFSYTFEDVPFHSSYAHSQSLDRLMNPLIDQYLYYLSTTNTP |
| | SGTTTQSRLQFSQAGASDIRDQSRNWLPGPCYRQQRVSKTSADNNNSEYSWTGATKYHLNGRDS |
| | LVNPGPAMASHKDNEEKFFPQSGVLIFGKQGSEKTNVDIEKVMITDEEEIRTTNPVATEQYGSVST |
| | NLQRGNRQAATADVNTQGVLPGMVWQDRDVYLQGPIWAKIPHTDGHFHPSPLMGGFGPKHP |
| | PPQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 101 | VNEADAAALEHDKAYDRQLDSGDNPYLKYNHADAEFQERLKEDTSFGGNLGRAVFQAKKRVLEPL |
| (AAVhu.53 | GLVEEPVKTAPGKKRPVEHSPAEPDSSSGTGKAGQQPARKRLNFGQTGDADSVPDPQPLRQPPA |
| (AAS99300)) | APTSLGSTTMATGSGAPMADNNEGADGVGNSSGNWHCDSQWLGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFQFSYTFEDVPFHSSYAHSQSLDRLMNPLIDQYLYYLNRTQTAS |
| | GTQQSRLLFSQAGPTSMSLQAKNWLPGPCYRQQRLSKQANDNNNSNFPWTGATKYYLNGRDSL |
| | VNPGPAMASHKDDEEKFFPMHGTLIFGKEGTNATNAELENVMITDEEEIRTTNPVATEQYGYVSN |
| | NLQNSNTAASTETVNHQGALPGMVWQDRDVYLQGPIWAKIPHTDGHFHPSPLMGGFGLKHPP |
| | PQIMIKNTPVPANPPTNFSSAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 102 | VNEADAAALEHDKAYDRQLDSGDNPYLKYNHADAEFQERLKEDTSFGGNLGRAVFQAKKRVLEPL |
| (AAVhu.54 | GLVEEPVKTAPGKKRPVEHSPAEPDSSSGTGKAGQQPARKRLNFGQTGDADSVPDPQPLGQPPÅ |
| (AAS99301)) | APTSLGSTTMATGSGAPMADNNEGADGVGNSSGNWHCDSQWLGDRVITTSTRTWALPTYNNH |
| | LYKQISSQSGASNDNHYFGYSTPWGYFDFNRFHCRFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLEYFPSQMLRTGNNFQFSYTFEDVPFHSSYAHSQGLDRLMNPLIDQYLYYLNRTQTA |
| | SGTQQSRLLFSQAGPTSMSLQAKNWLPGPCYRQQRLSKQANDNNNSNFPWTGATKYHLNGGDS |
| | LVNPGPAMASHKDDEEKFFPMHGTLIFGKEGTNATNAELENVMITDEEEIRTTNPVATEQYGYVS |
| | NNLQNSNTAASTETVNHQGALPGMVWQDRDVYLRGPIWAKIPHADGHFHPSPLMGGFGLKHP |
| | PPQIMIKNTPVPANPPTNFSSAKFASFITQYSTGQVSVEIEWELQKENSKRWNPEIQYTSNYNKSVN |
| | VDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 103 | VNEADAAALEHDKAYDRQLDSGDNPYLKYNHADAEFQERLKEDTSFGGNLGRAVFQAKKRVLEPL |
| (AAVhu.55 | GLVEEPVKTAPGKKRPVEHSPAEPDSSSGTGKAGQQPARKRLNFGQTGDADSVPDPQPLGQPPA |
| (AAS99302)) | APTSLGSTTMATGSGAPMADNNEGADGVGNSSGNWHCDSQWLGDRVITTSTRTWALPTYNNH |
| | LYKQISSQSGASNDNHYFGYSTPWGYFDFNRFHCHFSPRDWQRLINNNWGFRPKRLNFKLFNIQV |
| | KEVTQNDGTTTIANNLTSTVQVFTDSEYQLPYVLGSAHQGCLPPFPADVFMVPQYGYLTLNNGSQ |
| | AVGRSSFYCLECFPSQMLRTGNNFQFSYTFEDVPFHSSYAHSQSLDRLMNPLIDQYLYYLNRTQTAS |
| | GTQQSRLLFSQAGPTSMSLQAKNWLPGPCYRQQRLSKQANDNNNSNFPWTGATKYHLNGRDSL |
| | VNPGPAMASHKDDEEKFFPMHGTLIFGKEGTNATNAELENVMITDEEEIRTTNPVATEQYGYVSN |
| | NLQNSNTAASTETVNHQGALPGMVWQDRDVYLQGPIWAKIPHTDGHFHPSPLMGGFGLKHPP |
| | PQIMIKNTPVPANPPTNFSSAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 104 | VNEADAAALEHDKAYDRQLDSGDNPYLKYNHADAEFQERLKEDTSFGGNLGRAVFQAKKRVLEPL |
| (AAVhu.56 | GLVEEPVKTAPGKKRPVEHSPVEPDSSSGTGKAGNQPARKRLNFGQTGDADSVPDPQPLGQPPAS |
| (AAS99303)) | PSGLGTNTMATGSGAPMADNNEGADGVGNSSGNWHCDSTWMGDRVVTTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDLEYQLPYVLGSAHQGCLPPFPADVFMVPQYGYLTLNNGS |
| | QAVGRSSFYCLEYFPSQMLRTGNNFTFSYTFEDVPFHSSYAHSQSLDRLMNPLIDQYLYYLSRTNTP |
| | SGTTTQSRLQFSQAGASDIRDQSRNWLPGPCYRQQRVSKTAADNNNSEYSWTGATKYHLNGRDS |
| | LVNPGPAMASHKDDEEKFFPQSGVLIFGKQGSEKTNVDIEKVMITDEEEIRTTNPVATEQYGSVST |
| | NLQSGNTQAATSDVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDTLSEGIRQWWKLKPGPPPKPAERHKDDSRGLVLPGYKYLGPFNGLDKGEPV |
| NO: 105 | NEADAAALEHDKAYDRQLDSGDNPYLKYNHADAEFQERLKEDTSFGGNLGRAVFQAKKRVLEPLG |
| (AAVhu.57 | LVEEPVKTAPGKKRPVEHSPVEPDSSSGTGKAGNQPARKRLNFGQTGDADSVPDPQPLGQPPAAP |
| (AAS99304)) | SGLGTNTMATGSGAPMADNNEGADGVGNSSGDWHCDSTWMGDRVITTSTRTWALPTYNNHL |
| | YKQISSQSGASNDNHYFGYSTPWGYFDFNRFHCHFSPRDWQRLINNNWGFRPKRLNLKLFNIQVK |
| | EVTQNDGTTTIANNLTSTVQVFTDLEYQLPYVLGSAHQGCLPPFPADVFMVPQYGYLTLNNGSQA |
| | VGRSSFYCLEYFPSQMLRTGNNFTFSYTFEDVPFHSSYAHSQSLDRLMNPLIDQYLYYLSRTNTPSGT |
| | TTQSRLQFSQAGASDIRDQSRNWLPGPCYRQQRVSKTAADNNNGEYSWTGATKYHLNGRDSLV |
| | NPGPAMASHKDDEEKFFPQSGVLIFGKQGSEKTNVDIEKVMITDEEEIRTTNPVATEQYGSVSTNL |
| | QSGNTRAATSDVNTQGVLPGMVWQDRDVYLQGPIWAKIPHTDGHFHPSPLMGGFGLKHPPPQI |
| | LIKNTPVPANPSTTFSAAKFASFITQYSTGQVSVEIEWELQKENSKRWNPEIQYTSNYNKSVNVDFT |
| | VDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 106 | VNEADAAALEHDKAYDRQLDSGDNPYLKYNHADAEFQERLKEDTSFGGNLGRAVFQAKKRVLEPL |
| (AAVhu.60 | GLVEEPVKTAPGKKRPVEHSPVEPDSSSGTGKAGQQPARKRLNFGQTGDADSVPDPQPLGQPPA |
| (AAS99307)) | APSGLGSTTMATGSGAPMADNNEGADGVGNSSGNWHCDSQWLGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRLMNPLVDQYLYYLNKTQT |
| | NSGTLQQSRLLFSQAGPTNMSLQAKNWLPGPCYRQQRLSKQANDNNNSNFPWTAATKYHLNGR |
| | DSLVNPGPAMASHKDDEEKFFPMHGTLIFGKQGTNANDADLENVMITDEEEIRTTNPVATEQYGT |
| | VSNNLQNSNTGPTTGTVNHQGALPGMVWQDRDVYLQGPIWAKIPHTDGHFHPSPLMGGFGLK |
| | HPPPQIMIKNTPVPANPPTNFSSAKFASFITQYSTGQVSVEIEWELQKENSKRWNPEIQYTSNYNKS |
| | VNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 107 | VNEADAAALEHDKAYDRQLDSGDNPYLKYNHADAEFQERLKEDTSFGGNLGRAVFQAKKRVLEPL |
| (AAVhu.61 | GLVEEPVKTAPGKKRPVEHPPVEPDSSSGTGKAGQQPARKRLNFGQTGDADSVPDPQPLGQPPA |
| (AAS99308)) | APSGLGSTTMATGSGAPMADNNEGADGVGNSSGNWHCDSQWLGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRLMNPLIDQYLYYLNKTQT |
| | NSGTLQQSRLLFSQAGPTNMSLQAKNRLPGPCYRQQRLSKOANDNNNSNFPWTAATKYHLNGR |
| | DSLVNPGPAMASHKDDEEKFFPMHGTLIFGKQGTNANDADLENVMITDEEEIRTTNPVATEQYGT |
| | VSNNLQNSNTGPTTGTVNHQGALPGMVWQDRDVYLQGPIWAKIPHTDGHFHPSPLVGGFGLK |
| | HPPPQIMIKNTPVPANPPTNFSSAKFASFITQYSTGQVSVEIEWELQKENSKRWNPEIQYTSNYNKS |
| | VNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 108 | VNEADAAALEHDKAYDRQLDSGDNPYPKYNHADAEFQERLKEDTSFGGNLGRAVFQAKKRVLEPL |
| (AAVhu.63 | GLVEEPVKTAPGKKRPVEHSPAEPDSSSGTGKAGQQPARKRLNFGQTGDADSVPDPQPLGQPPA |
| (AAS99309)) | APSGLGTNTMATGSGAPMADNNEGADGVGNSSGNWHCDSTWMGDRVITTSTRTWALPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFTFSYTFEDVPFHSSYAHSQSLDRLMNPLIDQYLYYLSRTNTP |
| | SGTTTQSRLQFSQAGASDIRDQSRNWLPGPCYRQQRVSKTSADNNNSEYSWTGATKYHLNGRDS |
| | LVNPGPAMASHKDDEEKFFPQSGVLIFGKQDSGKTNVDIEKVMITDEEEIRTTNPVATEQYGSVST |
| | NLQSGNTQAATSDVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNYNKSVNV |
| | DFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 109 | PVNAADAAALEHDKAYDQQLKAGDNPYLRYNHADAEFQERLQEDTSFGGNLGRAVFQAKKRVLE |
| (AAVhu.66 | PLGLVEEAAKTAPGKKRPVEPSPQRSPDSSAGIGKKGQQPAKKRLNFGQTGDSESVPDPQPIGEPP |
| (AAS99311)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLSFKLF |
| | NIQVKEVTQNEGTETIANNLTSTIQVFTDSEYQLPYVLGSAHQGCLPPFPADVFMIPQYGYLTLNNG |
| | SQAVGRSSFYCLEYFPSQMLRTGNNFEFSYTFEDVPFHSSCAHSQSSDRLMNPLIDQYLYYLSRTRS |
| | TGGTQGTQQLLFSQAGPANMSAQAKNWLPGPCYRQQRVSTTLSQNNNSNFAWTGATKYHLNG |
| | RDSLVNPGVAMATHKDDEERFFPSSGVLMFGKQGAGRDNVDYSSVMLTSEEEIKTTNPVATEQY |
| | GVVADNLQQTNTGPIVGNVNSQGALPGMVWQNRDVYLQGPIWAKIPHTDGNFHPSPLMGGFG |
| | LKHPPPQILIKNTPVPADPPTTFSQAKLASFITQYSTGQVSVEIEWELQKENSKRWNPEIQYTSNYYK |
| | STNVDFAVNTEGTYSEPRPIGTRYLTRNL |
| SEQ ID | MAADGYLPDWLEDNLSEGIREWWDLKPGAPKPKANQQKQDDGRGLVLLGYKYLGPFNGLDKGE |
| NO: 110 | PVNAADAAALEHDKAYDQQLKAGDNPYLRYNHADAEFQERLQEDTSFGGNLGRAVFQAKKRVLE |
| (AAVhu.67 | PLGLVEEAAKTAPGKKRPVEPSPQRSPDSSTGIGKKGQQPAKKRLNFGQTGDSESVPDPQPIGEPP |
| (AAS99312)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFEFSYTFEDVPFHSGYAHSQSLDRLMNPLIDQYLYYLSRTQS |
| | TGGTQGTQQLLFSQAGPANMSAQAKNWLPGPCYRQQRVSTTLSQNNNSNFAWTGATKYHLNG |
| | RDSLVNPGVAMATHKDDEERFFPSSGVLMFGKQGAGRDNVDYSSVMLTSEEEIKTTNPVATEQY |
| | GVVADNLQQTNTGPIVGNVNSQGALPGMVWQNRDVYLQGPIWAKIPHTDGNFHPSPLMGGFG |
| | LKHPPPQILIKNTPVPADPPTTFSQAKLASFITQYSTGQVSVEIEWELQKENSKRWNPEIQYTSNYYK |
| | STNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 111 | PVNAADAAALEHDKAYDQQLKAGDNPYLRYNHADAEFQERLQEDTSFGGNLGRAVFQAKKRVLE |
| (AAVrh.10 | PLGLVEEGAKTAPGKKRPVEPSPQRSPDSSTGIGKKGQQPAKKRLNFGQTGDSESVPDPQPIGEPP |
| (AAO88201)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLNFKL |
| | FNIQVKEVTQNEGTKTIANNLTSTIQVFTDSEYQLPYVLGSAHQGCLPPFPADVFMIPQYGYLTLNN |
| | GSQAVGRSSFYCLEYFPSQMLRTGNNFEFSYQFEDVPFHSSYAHSQSLDRLMNPLIDQYLYYLSRTQ |
| | STGGTAGTQQLLFSQAGPNNMSAQAKNWLPGPCYRQQRVSTTLSQNNNSNFAWTGATKYHLN |
| | GRDSLVNPGVAMATHKDDEERFFPSSGVLMFGKQGAGKDNVDYSSVMLTSEEEIKTTNPVATEQ |
| | YGVVADNLQQQNAAPIVGAVNSQGALPGMVWQNRDVYLQGPIWAKIPHTDGNFHPSPLMGGF |
| | GLKHPPPQILIKNTPVPADPPTTFSQAKLASFITQYSTGQVSVEIEWELQKENSKRWNPEIQYTSNYY |
| | KSTNVDFAVNTDGTYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 112 | PVNEADAAALEHDKAYDKQLEQGDNPYLKYNHADAEFQERLQEDTSFGGNLGRAVFQAKKRVLE |
| (AAVrh.13 | PLGLVEEGAKTAPGKKRPIESPDSSTGIGKKGQQPAKKKLNFGQTGDSESVPDPQPLGEPPAAPSGL |
| (AAO88199)) | GSGTMAAGGGAPMADNNEGADGVGNASGNWHCDSTWLGDRVITTSTRTWALPTYNNHLYKQ |
| | ISSQSGATNDNHFFGYSTPWGYFDFNRFHCHFSPRDWQRLINNNWGFRPRKLRFKLFNIQVKEVT |
| | TNDGVTTIANNLTSTIQVFSDSEYQLPYVLGSAHQGCLPPFPADVFMIPQYGYLTLNNGSQSVGRSS |
| | FYCLEYFPSQMLRTGNNFEFSYTFEEVPFHSSYAHSQSLDRLMNPLIDQYLYYLARTQSTTGSTRELQ |
| | FHQAGPNTMAEQSKNWLPGPCYRQQRLSKNIDSNNNSNFAWTGATKYHLNGRNSLTNPGVAM |
| | ATNKDDEDQFFPINGVLVFGETGAANKTTLENVLMTSEEEIKTTNPVATEEYGVVSSNLQSSTAGP |
| | QTQTVNSQGALPGMVWQNRDVYLQGPIWAKIPHTDGNFHPSPLMGGFGLKHPPPQILIKNTPV |
| | PANPPEVFTPAKFASFITQYSTGQVSVEIEWELQKENSKRWNPEIQYTSNYAKSNNVEFAVNNEGV |
| | YTEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 113 | PVNEADAAALEHDKAYDKQLEQGDNPYLKYNHADAEFQERLQEDTSFGGNLGRAVFQAKKRVLE |
| (AAVrh.19 | PLGLVEEGAKTAPGKKRPVEPSPQRSPDSSTGIGKTGQQPAKKRLNFGQTGDSESVPDPQPIGEPP |
| (AAO88194)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPRKLRFKLF |
| | NIQVKEVTTDDGVTTIANNLTSTIQVFSDSEYQLPYVLGSAHQGCLPPFPADVFMIPQYGYLTLNNG |
| | SQSVGRSSFYCLEYFPSQMLRTGNNFEFSYTFEEVPFHSSYAHSQSLDRLMNPLIDQYLYYLARTQST |
| | TGSTRELQFHQAGPNTMAEQSKNWLPGPCYRQQRLSKNIDSNNNSNFAWTGATKYHLNGRNSL |
| | TNPGVAMATNKDDEDQFFPINGVLVFGKTGAANKTTLENVLMTSEEEIKTTNPVATEEYGVVSSNL |
| | QSSTAGPQTQTVNSQGALPGMVWQNRDVYLQGPIWAKIPHTDGNFHPSPLMDGFGLKHPPPQI |
| | LIKNTPVPANPPEVFTPAKFASFITQYSTGQVSVEIEWELQKENSKRWNPEIQYTSNYAKSNNVEFA |
| | VNNEGVYTEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 114 | PVNAADAAALEHDKAYDQQLKAGDNPYLRYNHADAEFQERLQEDTSFGGNLGRAVFQAKKRVLE |
| (AAVrh.22 | PLGLVEEGAKTAPGKKRPIESPDSSTGIGKKGQQPAKKKLNFGQTGDSESVPDPQPIGEPPAGPSGL |
| (AAO88192)) | GSGTMAAGGGAPMADNNEGADGVGNASGNWHCDSTWLGDRVITTSTRTWALPTYNNHLYKQ |
| | ISSQSGATNDNHFFGYSTPWGYFDFNRFHCHFSPRDWQRLINNNWGFRPRKLRFKLFNIQVKEVT |
| | TNDGVTTIANNLTSTIQVFSDSEYQLPYVLGSAHQGCLPPFPADVFMIPQYGYLTLNNGSQSVGRSS |
| | FYCLEYFPSQMLRTGNNFEFSYTFEEVPFHSSYAHSQSLDRLMNPLIDQYLYYLARTQSTTGSTRELQ |
| | FHQAGPNTMAEQSKNWLPGPCYRRQRLSKDIDSNNNSNFAWTGATKYHLNGRNSLTNPGVAM |
| | ATNKDDEDQFFPINGVLVFGKTGAANKTTLENVLMTSEEEIKTTNPVATEEYGVVSSNLQSSTAGP |
| | QTQTVNSQGALPGMVWQNRDVYLQGPIWAKIPHTDGNFHPSPLMGGFGLKHPPPQILIKNTPV |
| | PANPPEVFTPAKFASFITQYSTGQVSVEIEWELQKENSKRWNPEIQYTSNYAKSNNVEFAVNNEGV |
| | YTEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 115 | PVNEADAAALEHDKAYDKOLEQGDNPYLKYNHADAEFQERLQEDTSFGGNLGRAVFQAKKRVLE |
| (AAVrh.23 | PLGLVEEGAKTAPGKKRPVEPSPQRSPDSSTGIGKTGQQPAKKRLNFGQTGDSESVPDPQPIGEPP |
| (AAO88191)) | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLNFKL |
| | FNIQVKEVTQNEGTKTIANNLTSTIQVFTDSEYQLPYVLGSAHQGCLPPFPADVFMIPQYGYLTLNN |
| | GSQAVGRSSFYCLEYFPSQMLRTGNNFEFSYQFEDVPFHSSYAHSQSLDRLTNPLIDQYLYYLARTQ |
| | STTGSTRGLQFHQAGPNTMAEQSKNWLPGPCYRQQRLSKNIDSNNNSNFAWTGATKYHLNGRN |
| | SLTNPGVAMATNKDDEDQFFPINGVLVFGKTGAANKTTLENVLMTSEEEIKTTNPVATEEYGVVSS |
| | NLQSSTAGPQTQTVNSQGALPGMVWQNRDVYLQGPIWAKIPHTDGNFHPSPLMGGFGLKHPP |
| | PQILIKYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 116 | PVNAADAAALEHDKAYDQQLKAGDNPYLRYNHADAEFQERLQEDTSFGGNLGRAVFQAKKRVLE |
| (AAVrh.24 | PLGLVEEGAKTAPGKKRPIESPDSSTGIGKKGQQPAKKKLNFGQTGDSESVPDPQPIGEPPAGPSGL |
| (AAO88190)) | GSGTMAAGGGAPMADNNEGADGVGSSSGNWHCDSTWLGDRVITTSTRTWALPTYNNHLYKQI |
| | SNGTSGGSTNDNTYFGYSTPWGYFDFNRFHCHFSPRDWQRLINNNWGFRPKRLNFKLFNIQVKE |
| | VTQNEGTKTIANNLTSTIQVFTDSEYQLPYVLGSAHQGCLPPFPADVFMIPQYGYLTLNNGSQAVG |
| | RSSFYCLEYFPSQMLRTGNNFEFSYQFEDVPFHSSYAHSQSLDRLMNPLIDQYLYYLSRTQSTGGTA |
| | GTQQLLFSQAGPNNMSAQAKNWLPGPCYRQQRVSTTVSQNNNSNFAWTGATKYHLNGRDSLV |
| | NPGVAMATHKGDEERFFPSSGVLMFGKQGAGKDNVDYSSVMLTSEEEIKTTNPVATEQYGVVAD |
| | NLQQQNAAPIVGAVNSQGALPGMVWQNRDVYLQGPIWAKIPHTDGNFHPSPLMGGFGLKHPP |
| | PQILIKNTPVPADPPTTFSQAKLASFITQYSTGQVSVEIEWELQKENSKRWNPEIQYTSNYYKSTNVD |
| | FAVNTEGTYSEPRPIGTRYLTRSL |
| SEQ ID | MAADGYLPDWLEDNLSEGIREWWDLKPGAPKPKANQQKQDDGRGLVLPGYKYLGPFNGLDKGE |
| NO: 117 | PVNAADAAALEHDKAYDQQLKAGDNPYLRYNHADAEFQERLQEDTSFGGNLGRAVFQAKKRVLE |
| (AAVrh.35 | PLGLVEEGAKTAPGKKRPIDSPDSSTGIGKKGQQPAKKKLNFGQTGDSESVPDPQPLGEPPAAPSS |
| (AAO88186)) | VGSGTMAAGGGAPMADNNEGADGVGNASGNWHCDSTWLGDRVITTSTRTWALPTYNNHLYK |
| | QISSSSSGATNDNHYFGYSTPWGYFDFNRFHCHFSPRDWQRLINNNWGFRPKKLRFKLFNIQVKE |
| | VTTNDGVTTIANNLTSTVQVFSDSEYQLPYVLGSAHQGCLPPFPADVFMIPQYGYLTLNNGSQSVG |
| | RSSFYCLEYFPSQMLRTGNNFEFSYSFEDVPFHSSYAHSQSLDRLMNPLIDQYLYYLARTQSTTGSTR |
| | ELQFHQAGPNTMAEQSKNWLPGPCYRQQGLSKNLDFNNNSNFAWTAATKYHLNGRNSLTNPGI |
| | PMATNKDDEDQFFPINGVLVFGKTGAANKTTLENVLMTSEEEIKTTNPVATEEYGVVSSNLQPSTA |
| | GPQSQTINSQGALPGMVWQNRDVYLQGPIWAKIPHTDGNFHPSPLMGGFGLKHPPPQILIKNTP |
| | VPANPPEVFTPAKFASFITQYSTGQVSVEIEWELQKENSKRWNPEIQYTSNYAKSNNVEFAVNPDG |
| | VYTEPRPIGTRYLPRNL |
| SEQ ID | MAADGYLPDWLEDNLSEGIREWWDLKPGAPKPKANQQKQDDGRGLVLPGYKYLGPFNGLDKGE |
| NO: 118 | PVNAADAAALEHDKAYDQQLEAGDNPYLRYNHADAEFQERLQEDTSFGGNLGRAVFQAKKRVLE |
| (AAVrh.43 | PLGLVEEGAKTAPGKKRPVEQSPQEPDSSSGIGKKGQQPARKRLNFGQTGDSESVPDPQPLGEPP |
| (AAS99245)) | AAPSGVGPNTMAAGGGAPMADNNEGADGVGSSSGNWHCDSTWLGDRVITTSTRTWALPTYN |
| | NHLYKQISNGTSGGATNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFQFTYTFEDVPFHSSYAHSQSLDRLMNPLIDQYLYYLSRTQT |
| | TGGTANTQTLGFSQGGPNTMANQAKNWLPGPCYRQQRVSTTTGQNNNSNFAWTAGTKYHLN |
| | GRNSLANPGIAMATHKDDEERFFPVTGSCFWQQNAARDNADYSDVMLTSEEEIKTTNPVATEEY |
| | GIVADNLQQQNTAPQIGTVNSQGALPGMVWQNRDVYLQGPIWAKIPHTDGNFHPSPLMGGFG |
| | LKHPPPQILIKNTPVPADPPTTFNQSKLNSFITQYSTGQVSVEIEWELQKENSKRWNPEIQYTSNYYK |
| | STSVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 119 | PVNAADAAALEHDKAYDQQLKAGDNPYLRYNHADAEFQERLQEDTSFGGNLGRAVFQAKKRVLE |
| (AAVrh.48 | PLGLVEEAAKTAPGKKRPVEPSPQRSPDSSTGIGKKGQQPARKRLNFGQTGDSESVPDPQPIGEPP |
| (AAS99246)) | AGPSGLGSGTMAAGGGAPMADNNKGADGVGNASGNWHCDSTWLGDRVITTSTRTWALPTYN |
| | NHLYKQISSQSAGSTNDNVYFGYSTPWGYFDFNRFHCHFSPRDWQRLINSNWGFRPKKLNFKLFN |
| | IQVKEVTTNDGVTTIANNLTSTVQVFSDSEYQLPYVLGSAHQGCLPPFPADVFMIPQYGYLTLNNG |
| | SQSVGRSSFYCLEYFPSQMLRTGNNFTFSYTFEDVPFHSSYAHSQSLDRLMNPLIDQYLYYLARTQS |
| | NAGGTAGNRELQFYQGGPTTMAEQAKNWLPGPCFRQQRVSKTLDQNNNSNFAWTGATKYHLN |
| | GRNSLVNPGVAMATHKDDEERFFPSSGVLIFGKTGAANKTTLENVLMTNEEEIRPTNPVATEEYGT |
| | VSSNLQAANTAAQTQVVNNQGALPGMVWQNRDVYLQGPIWAKIPHTDGNFHPSPLMGGFGLK |
| | HPPPQILIKNTPVPANPPEVFTPAKFASFITQYSTGQVSVEIEWELQKENSKRWNPEIQYTSNFDKQ |
| | TGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 120 | PVNAADAAALEHDKAYDQQLKAGDNPHLRYNHADAEFQERLQEDTSFGGNLGRAVFQAKKRVLE |
| (AAVrh.49 | PLGLVEEGAKTAPGKKRPVEPSPQRSPDSSTGIGKKGQQPARKRLNFGQTGDSESVPDPQLIGEPP |
| (AAS99247)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANNLTSTIQVFTDSEYQLPYVLGSAHQGCLPPFPADVFMIPQYGNLTLNNGS |
| | QAVGRSSFYCLEYFPSQMLRTGNNFSFSYTFEDVPFHSSYAHSQSLDRLMNPLIDQYLYYLSRTQST |
| | GGTAGTQQLLFSQAGPSNMSAQARNWLPGPCYRQQRVSTTLSQNNNSNFAWTGATKYHLNGR |
| | DSLVNPGVAMATNKDDEDRFFPSSGILMFGKQGAGKDNMGYSNVMLTSEEEIKTTNPVATEQYG |
| | VVADNLQQQNTAPIVGAVNSQGALPGMVWQNRDVYLQGPIWAKIPHTDGNFHPSPLMGGFGL |
| | KHPPPQILIKNTPVPADPPTAFNQAKLNSFITQYGTGQVSVEIEWELQKENSKRWNPEIQYTSNYYK |
| | STNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 121 | PVNAADAAALEHDKAYDQQLKAGDNPYLRYNHADAEFQERLQEDTSFGGNLGRAVFQAKKRVLE |
| (AAVrh.50 | PLGLVEEGAKTAPGKKRPVEPSPQRSPDSSTGIGKKGQQPAGKRLNFGQTGDSESVPDPQPIGEPP |
| (AAS99248)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANNLTSTIQVFTDSEYQLPYVLGSAHQGCLPPFPADVFMIPQYGYLTLNNGS |
| | QAVGRSSFYCLEYFPSQMLRTGNNFSFSYTFEDVPFHSSYAHSQSLDRLMNPLVDQYLYYLSRTQST |
| | GGTAGTQQLLFSQAGPSNMSAQARNWLPGPCYRQQRVSTTLSQNNNSNFAWTGATKYHLNGR |
| | DSLVNPGVAMATNKDDEDRFFPSSGILMFGKQGAGKDNVDYSNVMLTSEEEIKTTNPVATEQYG |
| | VVADNLQQQNTAPIVGAVNSQGALPGMVWQNRDVYLQGPIWAKIPHTDGNFHPSPLMGGFGL |
| | KHPPPQILIKNTPVPADPPTAFNQAKLNSFITQYSTGQVSVEIEWELQKENSKRWSPEIQYTSNYYKS |
| | TNVDFAVNTEGVYSEPRPIGTRYLTRNL |
| SEQ ID | MVADGYLPDWLEDNLSEGIREWWDLKPGAPKPKANQQKQGDGRGLVLPGYKYLGPFNGLDKGE |
| NO: 122 | PVNAADAAALEHDKAYDQQLKAGDNPYLRYNHADAELQERLQEDTSFGGNLGRAVFQAKKRVLE |
| (AAVrh.51 | PLGLVEEGAKTAPGKKRPVEPSPQRSPDSSTGIGKKGQQPARKRLNFGQTGDSESVPDPQPIGEPP |
| (AAS99249)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANNLTSTIQVFTDSEYQLPYVLGSAHQGCQPPFPADVFMIPQYGYLTLNNGS |
| | QAVGRSSFYCLEYFPSQMLRTGNNFSFSYTFEDVPFHSSYAHSQSLDRLMNPLIDQYLYYLSRTQST |
| | GGTAGTQQLLFSQAGPSNMSAQARNWLPGPCYRQQRVSTTLSQNNNSNFAWTGATKYHLNGR |
| | DSLVNPGVAMATNKDDEDRFFPSSGILMFGKQGAGKDNVDYSNVMLTSEEEIKTTNPVATEQYG |
| | VVADNLQQONTAPIVGAVNSQGALPGMVWQNRDVYLQGPIWAKIPHTDGNFHPSPLMGGFGL |
| | KHPPPQILIKNTPVPADPPTAFNOAKLNSFITQYSTGQVSVEIEWEPQKENSKRWNPEIQYTSNYYK |
| | STNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 123 | PVNAADAAALEHDKAYDQQLKAGDNPYLRYNHADAEFQERLQEDTSFGGNLGRAVFQAKKRVLE |
| (AAVrh.52 | PLGLVEEGAKTAPGKKRPVEPSPQRSPDSSTGIGKKGQQPARKRLNFGQTGDSESVPDPQPIGEPP |
| (AAS99250)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANSLTSTIQVFTDSEYQLPYVLGSAHQGCLPPFPADVFMIPQYGYLTPNNGS |
| | QAVGRSSFYCLEYFPSQMLRTGNNFSFSYTFEDVPFHSSYAHSQSLDRLMNPLIDQYLYYLSRTQST |
| | GGTAGTQQLLSSQAGPSNMSAQARNWLPGPCYRQQRVSTTLSQNNNSNFAWTGATKYHLNGR |
| | DSLVNPGVAMATNKDDEDRFFPSSGILMFGKQGAGKDNVDYSNVMLTSEEEIKTTNPVATEQYG |
| | VVADNLQQQNTAPIVGAVNSQGALPGMVWQNRDVYLQGPIWAKIPHTDGNFHPSPLMGGFGL |
| | KHPPPQILIKNTPVPADPPTAFNQAKLNSFITQYSTGQVSVEIEWELQKENSKRWNPEIQYTSNYYK |
| | STNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 124 | PVNAADAAALEHDKAYDQQLKAGDNPYLRYNHADAEFQERLQEDTSFGGNLGRAVFQAKKRVLE |
| (AAVrh.53 | PLGLVEEGAKTAPGKKRPVEPSPQRSPDSSTGIGKKGQQPARKRLNFGQTGDSESVPDPQPIGEPP |
| (AAS99251)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANNLTSTIQVFTDSEYQLPYVLGSAHQGCLPPFPADVFMIPQYGYLTLNNGS |
| | QAVGRSSFYCLEYFPSQMLRTGNNFSFSYTFEDVPFHSSYVHSQSLDRLMNPLIDQYLYYLSRTQST |
| | GGTAGTQQLLFSQAGPSNMSAQARNWLPGPCYRQQRVSTTLSQNNNSNFAWTGATKYHLNGR |
| | DSLVNSGVAMATNKDDEDRFFPSSGILMFGKQGAGKDNVDYSNVMLTSEEEIKTTNPVATEQYG |
| | VVADNLQQQNTAPIVGAVNSQGALPGMVWQNRDVYLQGPIWAKIPHTDGNFHPSPLMGGFGL |
| | KHPPPQILIKNTPVPADPPTAFNQAKLNSFITQYSTGQVSVEIEWELQKENSKRWNPEIQYTSNYYK |
| | STNVDFAVNTEGVYSEPRPIGTRYPTRNL |
| SEQ ID | MAADGYLPDWLEDNLSEGIREWWDLKPGAPKPKANQQKQDDGRGLVLPGYKYLGPFNGLDKGE |
| NO: 125 | PVNAADAAALEHDKAYDQQLKAGDNPYLRYNHADAEFQERLQEDTSFGGNLGRAVFQAKKRVLE |
| (AAVrh.54 | PLGLVEEAAKTAPGKKRPVEPSPQRSPDSSTGIGKKGQQPARKRLNFGQTGDSESVPDPQPLGEPP |
| (AAS99252)) | AGPSGLGSGTMAAGGGAPMADNNEGADGVGNASGNWHCDSTWLGDRVITTSTRTWALPTYN |
| | NHLYKQISSQSAGSTNDNVYFGYSTPWGYFDFNRFHCHFSPRDWQRLINNNWGFRPKKLNFKLF |
| | NIQVKEVTTNDGVTTIANNLTSTVQVFSDSEYQLPYVLGSAHQGCLPPFPADVFMIPQYGYLTLNN |
| | GSQSVGRSSFYCLEYFPSQVLRTGNNFTFSYTFEDVPFHSSYAHSQSLDRLMNPLIDQYLYYLARTQS |
| | NPGGTSGNRELQFYQGGPSTMAEQAKNWLPGPCFRQQRVSKTLDQNNNSNFAWTGATKYHLN |
| | GRNSLVNPGVAMATHKDDEDRFFPSSGVLIFGKTGATNKTTLENVLMTNEEEIRPTNPVATEEYGI |
| | VSSNLQAANTAAQTQVVNNQGALPGMVWQNRDVYLQGPIWAKIPHTDGNFHPSPLMGGFGLK |
| | HPPPQILIKNTPVPANPPEVFTPAKFASFITQYSTGQVSVEIEWELQKENSKRWNPEIQYTSNFDKQ |
| | TGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 126 | PVNAADAAALEHDKAYDQQLKAGDNPYLRYNHADAEFQERLQEDTSFGGNLGRAVFQAKKRVLE |
| (AAVrh.55 | PLGLVEEGAKTAPGKKRPVEPSPQRSPDSSTGIGKKGQQPARKRLNFGQTGDSESVPDPQPIGEPP |
| (AAS99253)) | AAPSSVGSGTMAAGGGAPMADNNEGADGVGSSSGNWHCDSTRLGDRVITTSTRTWALPTYNN |
| | HLYKQISSQSAGSTNDNVYFGYSTPWGYFDFNRFHCHFSPRDWQRLINNNWGFRPKKLNFKLFNI |
| | QVKEVTTNDGVTTIANNLTSTVQVFSDSEYQLPYVLGSAHQGCLPPFPADVFMIPQYGYLTLNNGS |
| | QSVGRSSFYCLEYFPSQMLRTGNNFTFSYTFEDVPFHSSYAHSQSLDRLMNPLIDQYLYYLARTQSN |
| | AGGTAGNRELQFYQGGPTTMAEQAKNWLPGPCFRQRRVSKTLDQNNNSNFAWTGATKYHLNG |
| | RNSLVNPGVAMATHKDDEERFFPSSGVLIFGKTGAANKTTLENVLMTNEEEIRPTNPVATEEYGTV |
| | SSNLQAANTAAQTQVVNNQGALPGMVWQNRDVYLQGPIWAKIPHTDGNFHPSPLMGGFGLKH |
| | PPPQILIKNTPVPANPPEVFTPAKFASFITQYSTGQVSVEIEWELQKENSKRWNPEIQYTSNFDKQT |
| | GVDFAVDSQGVYSEPRPIGTRYLTRNL |
| SEQ ID | MAADGYLPDWLEDNLSEGIREWWALKPGAPKPKANQQKQDDGRGLVLPGYKYLGPFNGLDKGE |
| NO: 127 | PVNAADAAALEHDKAYDQQLQAGDNPYLRYNHADAEFQERLQEDTSFGGNLGRAVFQAKKRVLE |
| (AAVrh.57 | PLGLVEEGAKTAPGKKRPVEPSPQRSPDSSTGIGKKGQQPARKRLNFGQTGDSESVPDPQPIGEPP |
| (AAS99254)) | AAPSSVGSGTMAAGGGAPMADNNEGADGVGSSSGNWHCDSTWLGDRVITTSTRTWALPTYNN |
| | HLYKQTSNGTSGGSTNDNTYFGYSTPWGYFDFNRFHCHFSPRDWQRLINNNWGFRPKRLSFKLF |
| | NIQVKEVTQNEGTKTIANNLTSTIQVFTDSEYQLPYVLGSAHQGCLPPFPADVFMIPQYGYLTLNNG |
| | SQAVGRSSFYCLEYFPSQMLRTGNNFSFSYTFEDVPFHSSYAHSQSLDRLMNPLIDQYLYYLSRTQS |
| | TGGTAGTQQLLFSQAGPSNMSAQARNWLPGPCYRQQRVSTTLSQNNNSNFÅWTGATKYHLNG |
| | RDSLVNPGVAMATNKDDEDRFFPSSGILMFGKQGAGKDNVDYSNVMLTSEEEIKTTNPVATEQY |
| | GVVADNLQQQNTAPIVGAVNSQGALPGMVWQNRDVYLQGPIWAKIPHTDGNFHPSPLMGGF |
| | GLKHPPPQILIKNTPVPADPPTAFNQAKLNSFITQYSTGQVSAEIEWELQKENSKRWNPEIQYTSNY |
| | YKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 128 | PVNAADAAALEHDKAYDQQLKAGDNPYLRYNHADAEFQERLQEDTSFGGNLGRAVFQAKKRVLE |
| (AAVrh.58 | PLGLVEEAAKTAPGKKRPVEPSPQRSPDSSTGIGKKGQQPARKRLNFGQTGDSESVPDPQPIGEPP |
| (AAS99255)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANNLTSTIQVFTDSEYQLPYVLGSAHQGCLPPFPADVFMIPQYGYLTLNNGS |
| | QAVGRSSFYCLEYFPSQMLRTGNNFSFSYTFEDVPFHSSYAHSQSLDRLMNPLIDQYLYYLSRTQST |
| | GGTAGTQQLLFSQAGPSNMSAQARNWLPGPCYRQQRVSTTLSQNNNSNFAWTGATKYHLNGR |
| | DSLVNPGVAMATNKDDEDRFFPSSGILMFGKQGAGKDNVDYSNVMLTSEEEIKTTNPVATEQYG |
| | VVADNLQQQNTAPIVGAVNSQGALPGMVWQNRDVYLQGPIWAKIPHTDGNFHPSPLMGGFGL |
| | KHPPPQILIKSTPVPADPPTAFNOAKLNSFITQYSTGQVSVEIEWELQKENSKCWNPEIQYTSNYYKS |
| | TNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 129 | PVNAADAAALEHDKAYDQQLKAGDNPYLRYNHADAEFQERLQEDTSFGGNLGRAVFQAKKRVLE |
| (AAVrh.62 | PLGLAEEAAKTAPGKKRPVEPSPQRSPDSSTGIGKKGQQPARKRLNFGQTGDSESVPDPQPIGEPP |
| (AAS99258)) | AGPSGLGSGTMAAGGGAPMADNNKGADGVGNASGNWHCDSTWLGDRVITTSTRTWALPTYN |
| | NHLYKQISSQSAGSTNDNVYFGYSTPWGYFDFNRFHCHFSPRDWQRLINNNWGFRPKKLNFKLF |
| | NIQVKEVTTGDGVTTIANNLTSTVQVFSDSEYQLPYVLGSAHQGCLPPFPADVFMIPQYGYLTLNN |
| | DSQSVGRSSFYCLEYFPSQMLRTGNNFTFSYTFEDVPFHSSYAHSQSLDRLMNPLIDQYLYYLARTQ |
| | SNAGGTAGNRELQFYQGGPTTMAEQAKNWLPGPCFRQQRVSKTLDQNNNSNFAWTGATKYHL |
| | NGRNSLVNPGVAMATHKDDEERFFPSSGVLIFGKTGAANKTTLENVLMTNEEEIRPTNPVATEEYG |
| | TVSSNLQAANTAAQTQVVNNQGALPGMVWQNRDVYLQGPIWAKIPHTDGNFHPSPLMGGFGL |
| | KHPPPQILIKNTPVPANPPEVFTPAKFASFITQYSTGQVSVEIEWELQKENSKRWNPEIQYTSNFDK |
| | QTGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 130 | PVNAADAAALEHDKAYDQQLKAGDNPYLRYNHADAEFQERLQEDTSFGGNLGRAVFQAKKRVLE |
| (AAVrh.64 | PLGLVEEGAKTAPGKKRPVEPSPQRSPDSSTGIGKKGQQPARKRLNFGQTGDSESVPDPQPIGEPP |
| (AAS99259)) | AAPSSVGSGTMAAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNNWGFRPKRLSFKLFN |
| | IQVKEVTQNEGTKTIANNLTSTIQVFTDSEYQLPYVLGSAHQGCLPPFPADVFMIPQYGYLTLNNGS |
| | QAVGRSSFYCLEYFPSQMLRTGNNFSFSYTFEDVPFHSSYAHSQSLDRLMNPLIDQYLYYLSRTQST |
| | GGTAGTQQLLFSQAGPSNMSAQARNWLPGPCYRQQRVSTTLSQNNNSNFAWTGATKYHLNGR |
| | DSLVNPGVAMATNKDDEDRFFPSSGILMFGKQGAGKDNVDYSNVMLTSEEEIKTTNPVATEQYG |
| | VVADNLQQQNTAPIVGAVNSQGALPGMVWQNRDVYLQGPIWAKIPHTDGNFHPSPLMGGFGL |
| | KHPPPQILIKNTPVPADPPTAFNQAKLNSFITQYSTGQVSVEIVWELQKENSKRRNPEIQYTSNYYKS |
| | TNVDFAVNTEGVYSEPRPIGTRYLTRNL |
| SEQ ID | MAADGYLPDWLEDTLSEGIRQWWKLKPGPPPPKPAERHKDDSRGLVLPGYKYLGPFNGLDKGEP |
| NO: 131 | VNEADAAALEHDKAYDRQLDSGDNPYLKYNHADAEFQERLKEDTSFGGNLGRAVFQAKKRVLEPL |
| (AAVrh.56 | GLVEEPVKTAPGKKRPVEHSPAEPDSSSGTGKAGQQPARKRLNFGQTGDADSVPDPQPLGQPPA |
| (JA400164)) | APSGLGSTTMATGSGAPMADNNEGADGVGNSSGNWHCDSQWLGDRVITTSTRTWAQPTYNN |
| | HLYKQISSQSGASNDNHYFGYSTPWGYFDFNRFHCHFSPRDWQRLINNNWGFRPKRLNFKLFNIQ |
| | VKEVTQNDGTTTIANNLTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRLMNPLIDQYLYYLNKTQS |
| | NSGALQQSRLLFSQAGPTSMSLQAKNWLPGPCYRQQRLSKQANDNNNSNFPWTAATKYHLNGR |
| | DSLVNPGPAMASHKDDEEKFFPMHGTLIFGKQGTNANDADLDNVMITDEEEIRTTNPVATEQYG |
| | YVSNNLQNSNTGPTTGTVNHRGALPGMVWQDRDVYLQGPIWAKIPHTDGHFHPSPLMGGFGL |
| | KHPPPQIMIKNTPVPANPPTNFSSAKFASFITQYSTGQVSVEIEWELQKENSKRWNPEIQYTSNYNK |
| | SVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLD |
| NO: 132 | KGEPVNAADAAALEHDKAYDQQLKAGDNPYLRYNHADAEFQERLQEDTSFGGNLGRAVFQ |
| (Anc80) | AKKRVLEPLGLVEEGAKTAPGKKRPVEQSPQEPDSSSGIGKKGQQPAXKRLNFGQTGDSE |
| | SVPDPQPLGEPPAAPSGVGSNTMAXGGGAPMADNNEGADGVGNASGNWHCDSTWLGDRVI |
| | TTSTRTWALPTYNNHLYKQISSQSGXSTNDNTYFGYSTPWGYFDFNRFHCHFSPRDWQRL |
| | INNNWGFRPKXLNFKLFNIQVKEVTTNDGTTTIANNLTSTVQVFTDSEYQLPYVLGSAHQ |
| | GCLPPFPADVFMIPQYGYLTLNNGSQAVGRSSFYCLEYFPSQMLRTGNNFXFSYTFEDVP |
| | FHSSYAHSQSLDRLMNPLIDQYLYYLSRTQTTSGTAGNRXLQFSQAGPSSMANQAKNWLP |
| | GPCYRQQRVSKTXNQNNNSNFAWTGATKYHLNGRDSLVNPGPAMATHKDDEDKFFPMSGV |
| | LIFGKQGAGNSNVDLDNVMITXEEEIKTTNPVATEXYGTVATNLQSXNTAPATGTVNSQG |
| | ALPGMVWQXRDVYLQGPIWAKIPHTDGHFHPSPLMGGFGLKHPPPQILIKNTPVPANPPT |
| | TFSPAKFASFITQYSTGQVSVEIEWELQKENSKRWNPEIQYTSNYNKSTNVDFAVDTNGV |
| | YSEPRPIGTRYLTRNL |
| | (168) . . . (168) Lys or Arg |
| | (205) . . . (205) Ala or Ser |
| | (266) . . . (266) Ala or Gly |
| | (311) . . . (311) Arg or Lys |
| | (411) . . . (411) Glu or Gln |
| | (460) . . . (460) Thr or Glu |
| | (493) . . . (493) Ala or Thr |
| | (562) . . . (562) Ser or Asn |
| | (576) . . . (576) Gln or Glu |
| | (587) . . . (587) Ser or Ala |
| | (609) . . . (609) Asn or Asp |
| SEQ ID | MAADGYLPDWLEDNLSEGIREWWDLKPGAPKPKANQQKQDDGRGLVLPGYKYLGPFNGLD |
| NO: 133 | KGEPVNAADAAALEHDKAYDQQLKAGDNPYLRYNHADAEFQERLQEDTSFGGNLGRAVFQ |
| (Anc81 | AKKRVLEPLGLVEEGAKTAPGKKRPVEQSPQEPDSSXGIGKKGQQPAXKRLNFGQTGDSE |
| (AKU89596)) | SVPDPQPLGEPPAAPSGVGSNTMAAGGGAPMADNNEGADGVGNASGNWHCDSTWLGDRVI |
| | TTSTRTWALPTYNNHLYKQISXXQSGGSTNDNTYFGYSTPWGYFDFNRFHCHFSPRDWQR |
| | LINNNWGFRPKXLNFKLFNIQVKEVTTNDGTTTIANNLTSTVQVFTDSEYQLPYVLGSAH |
| | QGCLPPFPADVFMIPQYGYLTLNNGSQAVGRSSFYCLEYFPSQMLRTGNNFXFSYTFEDV |
| | PFHSSYAHSQSLDRLMNPLIDQYLYYLSRTQTTGGTAGNXXLQFSQAGPSSMANQAKNWL |
| | PGPCYRQQRVSKTTNQNNNSNFAWTGATKYHLNGRDSLVNPGVAMATHKDDEDRFFPSSG |
| | VLIFGKQGAGNXNVDXXNVMITXEEEIKTTNPVATEEYGXVATNLQSXNTAPQTGTVNSQ |
| | GALPGMVWQNRDVYLQGPIWAKIPHTDGNFHPSPLMGGFGLKHPPPQILIKNTPVPANPP |
| | TTFXPAKFASFITQYSTGQVSVEIEWELQKENSKRWNPEIQYTSNYNKSTNVDFAVDTEG |
| | VYSEPRPIGTRYLTRNL |
| | (157) . . . (157) Thr or Ser |
| | (168) . . . (168) Lys or Arg |
| | (262) . . . (262) Asn or Ser |
| | (263) . . . (263) Ser or His |
| | (312) . . . (312) Arg or Lys |
| | (412) . . . (412) Glu or Gln |
| | (460) . . . (460) Arg or Gln |
| | (461) . . . (461) Thr or Glu |
| | (552) . . . (552) Asp or Ser |
| | (556) . . . (556) Leu or Tyr |
| | (557) . . . (557) Asp or Ser |
| | (563) . . . (563) Ser or Asn |
| | (580) . . . (580) Val or Ile |
| | (588) . . . (588) Asn or Ser |
| | (664) . . . (664) Ser or Thr |
| SEQ ID | MAADGYLPDWLEDNLSEGIREWWDLKPGAPKPKANQQKODDGRGLVLPGYKYLGPFNGLD |
| NO: 134 | KGEPVNAADAAALEHDKAYDQQLKAGDNPYLRYNHADAEFQERLQEDTSFGGNLGRAVFQ |
| (Anc82 | AKKRVLEPLGLVEEGAKTAPGKKRPVEQSPQREPDSSXGIGKKGQQPAXKRINFGQTGDS |
| (AKU89597)) | ESVPDPQPLGEPPAAPSGVGSNTMAAGGGAPMADNNEGADGVGNSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLNFKLFNIQVKEVTTNEGTKTIANNLTSTVQVFTDSEYQLPYVLGSA |
| | HQGCLPPFPADVFMIPQYGYLTLNNGSQAVGRSSFYCLEYFPSQMLRTGNNFQFSYTFED |
| | VPFHSSYAHSQSLDRLMNPLIDQYLYYLSRTQTTGGTAGTQTLQFSQAGPSSMANQAKNW |
| | LPGPCYRQQRVSTTTNONNNSNFAWTGATKYHLNGRDSLVNPGVAMATHKDDEDRFFPSS |
| | GVLIFGKQGAGNDNVDYSNVMITXEEEIKTTNPVATEEYGVVATNLQSANTAPQTGTVNS |
| | QGALPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (158) . . . (158) Thr or Ser |
| | (169) . . . (169) Lys or Arg |
| | (564) . . . (564) Ser or Asn |
| SEQ ID | MAADGYLPDWLEDNLSEGIREWWDLKPGAPKPKANQQKQDDGRGLVLPGYKYLGPFNGLD |
| NO: 135 | KGEPVNAADAAALEHDKAYDQQLKAGDNPYLRYNHADAEFQERLQEDTSFGGNLGRAVFQ |
| (Anc83 | AKKRVLEPLGLVEEGAKTAPGKKRPVEQSPQREPDSSXGIGKKGQQPAXKRLNFGQTGDS |
| (AKU89598)) | ESVPDPQPLGEPPAAPSGVGSNTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLXFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFXFSYTFED |
| | VPFHSSYAHSQSLDRLMNPLIDQYLYYLSRTQTTGGTAGTQTLQFSQAGPSXMANQAKNW |
| | LPGPCYRQQRVSTTTSQNNNSNFAWTGATKYHLNGRDSLVNPGVAMATHKDDEXRFFPSS |
| | GXLIFGKQGAGKDNVDYSNVMLTSEEEIKTTNPVATEEYGVVADNLQQQNTAPQXGTVNS |
| | QGALPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (158) . . . (158) Thr or Ser |
| | (169) . . . (169) Arg or Lys |
| | (315) . . . (315) Asn or Ser |
| | (413) . . . (413) Gln or Glu |
| | (472) . . . (472) Asn, Thr or Ser |
| | (534) . . . (534) Asp or Glu |
| | (542) . . . (542) Ile or Val |
| | (595) . . . (595) Ile or Val |
| SEQ ID | MAADGYLPDWLEDNLSEGIREWWDLKPGAPKPKANQQKODDGRGLVLPGYKYLGPFNGLD |
| NO: 136 | KGEPVNAADAAALEHDKAYDQQLKAGDNPYLRYNHADAEFQERLQEDTSFGGNLGRAVFQ |
| (Anc84 | AKKRVLEPLGLVEEGAKTAPGKKRPVEPSPQRSPDSSTGIGKKGQQPAXKRLNFGQTGDS |
| (AKU89599)) | ESVPDPQPIGEPPAAPSGVGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLXFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTAGTQQLLFSQAGPSNMSAQAKNW |
| | LPGPCYRQQRVSTTLSQNNNSNFAWTGATKYHLNGRDSLVNPGVAMATHKDDEXRFFPSS |
| | GXLMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (169) . . . (169) Arg or Lys |
| | (315) . . . (315) Asn or Ser |
| | (534) . . . (534) Asp or Glu |
| | (542) . . . (542) Ile or Val |
| SEQ ID | MAADGYLPDWLEDNLSEGIREWWDLKPGAPKPKANQQKQDDGRGLVLPGYKYLGPFNGLD |
| NO: 137 | KGEPVNAADAAALEHDKAYDQQLKAGDNPYLRYNHADAEFQERLQEDTSFGGNLGRAVFQ |
| (Anc94) | AKKRVLEPLGLVEEGAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTAGTQQLLFSQAGPXNMSAQAKNW |
| | LPGPCYRQQRVSTTLSQNNNSNFAWTGATKYHLNGRDSLVNPGVAMATHKDDEERFFPSS |
| | GVLMFGKQGAGKDNVDYSSVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| | (471) . . . (471) Ser or Asn |
| SEQ ID | MAADGYLPDWLEDNLSEGIREWWDLKPGAPKPKANQQKQDDGRGLVLPGYKYLGPFNGLD |
| NO: 138 | KGEPVNAADAAALEHDKAYDQQLKAGDNPYLRYNHADAEFQERLQEDTSFGGNLGRAVFQ |
| (Anc110 | AKKRVLEPLGLVEEGAKTAPGKKRPVEQSPQEPDSSXLGIGKTGQQPAXKRLNFGQTGDS |
| (AKU89600)) | ESVPDPQPLGEPPAAPSGVGSNTMASGGGAPMADNNEGADGVGNSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLNFKLFNIQVKEVTTNEGTKTIANNLTSTVQVFTDSEYQLPYVLGSA |
| | HQGCLPPFPADVFMIPQYGYLTLNNGSQAVGRSSFYCLEYFPSQMLRTGNNFQFSYTFED |
| | VPFHSSYAHSQSLDRLMNPLIDQYLYYLSRTQTTGTXGTQTLXFSQAGPSSMANQARNWV |
| | PGPCYRQQRVSTTTNONNNSNFAWTGAXKXXLNGRDSLMNPGVAMASHKDDEDRFFPSSG |
| | VLIFGKQGAGNDNVDYSXVMITNEEEIKTTNPVATEEYGAVATNXQXLANTQAQTGLVHN |
| | QGVLPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (157) . . . (157) Ser or Thr |
| | (169) . . . (169) Lys or Arg |
| | (457) . . . (457) Ala or Gly |
| | (463) . . . (463) Gln or Ala |
| | (508) . . . (508) Thr or Ala |
| | (510) . . . (510) Tyr or Phe |
| | (511) . . . (511) His or Lys |
| | (558) . . . (558) Gln or Asn |
| | (585) . . . (585) Asn or His |
| | (587) . . . (587) Ser or Ala |
| SEQ ID | MAADGYLPDWLEDNLSEGIREWWDLKPGAPKPKANQQKQDDGRGLVLPGYKYLGPFNGLD |
| NO: 139 | KGEPVNAADAAALEHDKAYDQQLKAGDNPYLRYNHADAEFQERLQEDTSFGGNLGRAVFQ |
| (Anc113 | AKKRVLEPLGLVEEGAKTAPGKKRPVEXSPQRSPDSSTGIGKKGQQPAXKRLNFGQTGDS |
| (AKU89601)) | ESVPDPQPLGEPPAAPSGVGSGTMAAGGGAPMADNNEGADGVGNASGNWHCDSTWLGDRV |
| | ITTSTRTWALPTYNNHLYKQISSQSAGSTNDNTYFGYSTPWGYFDFNRFHCHFSPRDWQR |
| | LINNNWGFRPKKLXFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFEFSYTFEDV |
| | PFHSSYAHSQSLDRLMNPLIDQYLYYLARTQSTTGGTAGNRELQFXQAGPSTMAEQAKNW |
| | LPGPCYRQQRVSKTLDQNNNSNFAWTGATKYHLNGRNSLVNPGVAMATHKDDEDRFFPSS |
| | GVLIFGKTGAANKTTLENVLMTXEEEIKTTNPVATEEYGXVSSNLQSXNTAPQTQTVNSQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNYDKSTNVDFAVDSEG |
| | VYSEPRPIGTRYLTRNL |
| | (148) . . . (148) Pro or Gln |
| | (169) . . . (169) Lys or Arg |
| | (314) . . . (314) Arg or Asn |
| | (466) . . . (466) Tyr or His |
| | (563) . . . (563) Asn or Ser |
| | (580) . . . (580) Val or lle |
| | (588) . . . (588) Ala or Ser |
| SEQ ID | MAADGYLPDWLEDNLSEGIREWWDLKPGAPKPKANQQKQDDGRGLVLPGYKYLGPFNGLD |
| NO: 140 | KGEPVNAADAAALEHDKAYDQQLKAGDNPYLRYNHADAEFQERLQEDTSFGGNLGRAVFQ |
| (Anc126 | AKKRVLEPLGLVEEGAKTAPGKKRPVEQSPQEPDSSSGIGKXGQQPAXKRLNFGQTGDSE |
| (AKU89602)) | SVPDPQPLGEPPAAPSGVGSNTMASGGGAPMADNNEGADGVGNXSGNWHCDSTWLGDRVI |
| | TTSTRTWALPTYNNHLYKQISSQSGASNDNHYFGYSTPWGYFDFNRFHCHFSPRDWQRLI |
| | NNNWGFRPKXLNFKLFNIQVKEVTTNDGTTTIANNLTSTVQVFTDSEYQLPYVLGSAHQG |
| | CLPPFPADVFMIPQYGYLTLNNGSQAVGRSSFYCLEYFPSQMLRTGNNFXFSYTFEDVPF |
| | HSSYAHSQSLDRLMNPLIDQYLYYLXRTQTTSGTAQNRELXFSQAGPSSMXNQAKNWLPG |
| | PCYRQQRVSKTANDNNNSNFAWTGATKYHLNGRDSLVNPGPAMASHKDDEDKFFPMSGVL |
| | IFGKQGAGASNVDLDNVMITDEEEIKTTNPVATEQYGTVATNLQSSNTAPATGTVNSQGA |
| | LPGMVWQDRDVYLQGPIWAKIPHTDGHFHPSPLMGGFGLKHPPPQILIKNTPVPANPPTT |
| | FSPAKFASFITQYSTGQVSVEIEWELQKENSKRWNPEIQYTSNYNKSXNVDFTVDTNGVY |
| | SEPRPIGTRYLTRNL |
| | (162) . . . (162) Ser or Thr |
| | (168) . . . (168) Lys or Arg |
| | (224) . . . (224) Ala or Ser |
| | (310) . . . (310) Arg or Lys |
| | (410) . . . (410) Thr or Gln |
| | (446) . . . (446) Ser or Asn |
| | (461) . . . (461) Gln or Leu |
| | (471) . . . (471) Ala or Ser |
| | (708) . . . (708) Ala or Thr |
| SEQ ID | MAADGYLPDWLEDNLSEGIREWWDLKPGAPQPKANQQHQDDXRGLVLPGYKYLGPFNGLD |
| NO: 141 | KGEPVNEADAAALEHDKAYDQQLKAGDNPYLKYNHADAEFQERLQEDTSFGGNLGRAVFQ |
| Anc127 | AKKRVLEPLGLVEEAAKTAPGKKRPVEQSPQEPDSSSGIGKSGQQPAXKRLNFGQTGDSE |
| (AKU89603) | SVPDPQPLGEPPÅAPSGVGSNTMASGGGAPMADNNEGADGVGNSSGNWHCDSTWLGDRVI |
| | TTSTRTWALPTYNNHLYKQISSQSGASNDNHYFGYSTPWGYFDFNRFHCHFSPRDWQRLI |
| | NNNWGFRPKXLNFKLFNIQVKEVTQNDGTTTIANNLTSTVQVFTDSEYQLPYVLGSAHQG |
| | CLPPFPADVFMIPQYGYLTLNNGSQAVGRSSFYCLEYFPSQMLRTGNNFXFSYTFEDVPF |
| | HSSYAHSQSLDRLMNPLIDQYLYYLXRTQTTSGTTQQSRLXFSQAGPSSMXQQAXNWLPG |
| | PCYRQQRVSKTANDNNNSNFAWTXATKYHLNGRDSLVNPGPAMASHKDDEEKFFPMHGXL |
| | IFGKQGTGASNVDLDNVMITDEEEIRTTNPVATEQYGTVATNLQSSNTAPATGTVNSQGA |
| | LPGMVWQDRDVYLQGPIWAKIPHTDGHFHPSPLMGGFGLKHPPPQILIKNTPVPANPPTT |
| | FSPAKFASFITQYSTGQVSVEIEWELQKENSKRWNPEIQYTSNYNKSVNVDFTVDTNGVY |
| | SEPRPIGTRYLTRNL |
| | (42) . . . (42) Gly or Ser |
| | (168) . . . (168) Arg or Lys |
| | (310) . . . (310) Lys or Arg |
| | (410) . . . (410) Thr or Gln |
| | (446) . . . (446) Ser or Arg |
| | (461) . . . (461) Gln or Leu |
| | (471) . . . (471) Ala or Ser |
| | (475) . . . (475) Lys or Arg |
| | (504) . . . (504) Gly or Ala |
| | (539) . . . (539) Val or Asn |
| SEQ ID | MAADGYLPDWLEDNLSEGIREWWDLKPGAPKPKANQQKODDGRGLVLPGYKYLGPFNGLDKGE |
| NO: 142 | PVNAADAAALEHDKAYDQQLKAGDNPYLRYNHADAEFQERLQEDTSFGGNLGRAVFQAKKRVLE |
| (Anc80L65 | PLGLVEEGAKTAPGKKRPVEQSPQEPDSSSGIGKKGQQPARKRLNFGQTGDSESVPDPQPLGEPP |
| (AKU89595)) | AAPSGVGSNTMAAGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKKLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSNFAWTGATKYHLN |
| | GRDSLVNPGPAMATHKDDEDKFFPMSGVLIFGKQGAGNSNVDLDNVMITNEEEIKTTNPVATEEY |
| | GTVATNLQSANTAPATGTVNSQGALPGMVWQDRDVYLQGPIWAKIPHTDGHFHPSPLMGGFG |
| | LKHPPPQILIKNTPVPANPPTTFSPAKFASFITQYSTGQVSVEIEWELQKENSKRWNPEIQYTSNYNK |
| | STNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 143 | PVNAADAAALEHDKAYDQQLKAGDNPYLRYNHADAEFQERLQEDTSFGGNLGRAVFQAKKRVLE |
| (Anc80L1) | PLGLVEEGAKTAPGKKRPVEQSPQEPDSSSGIGKKGQQPAKKRLNFGQTGDSESVPDPQPLGEPP |
| | AAPSGVGSNTMAAGGGAPMADNNEGADGVGNASGNWHCDSTWLGDRVITTSTRTWALPTYN |
| | NHLYKQISSQSGASTNDNTYFGYSTPWGYFDFNRFHCHFSPRDWQRLINNNWGFRPKRLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSNFAWTGATKYHLN |
| | GRDSLVNPGPAMATHKDDEDKFFPMSGVLIFGKQGAGNSNVDLDNVMITSEEEIKTTNPVATEQY |
| | GTVATNLQSSNTAPATGTVNSQGALPGMVWQNRDVYLQGPIWAKIPHTDGHFHPSPLMGGFGL |
| | KHPPPQILIKNTPVPANPPTTFSPAKFASFITQYSTGQVSVEIEWELQKENSKRWNPEIQYTSNYNKS |
| | TNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 144 | PVNAADAAALEHDKAYDQQLKAGDNPYLRYNHADAEFQERLQEDTSFGGNLGRAVFQAKKRVLE |
| (Anc80L27) | PLGLVEEGAKTAPGKKRPVEQSPQEPDSSSGIGKKGQQPARKRLNFGQTGDSESVPDPQPLGEPP |
| | AAPSGVGSNTMAAGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKRLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSNFAWTGATKYHLN |
| | GRDSLVNPGPAMATHKDDEDKFFPMSGVLIFGKQGAGNSNVDLDNVMITNEEEIKTTNPVATEQ |
| | YGTVATNLQSANTAPATGTVNSQGALPGMVWQDRDVYLQGPIWAKIPHTDGHFHPSPLMGGF |
| | GLKHPPPQILIKNTPVPANPPTTFSPAKFASFITQYSTGQVSVEIEWELQKENSKRWNPEIQYTSNYN |
| | KSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 145 | PVNAADAAALEHDKAYDQQLKAGDNPYLRYNHADAEFQERLQEDTSFGGNLGRAVFQAKKRVLE |
| (Anc80L33) | PLGLVEEGAKTAPGKKRPVEQSPQEPDSSSGIGKKGQQPAKKRLNFGQTGDSESVPDPQPLGEPP |
| | AAPSGVGSNTMAAGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKKLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSNFAWTGATKYHLN |
| | GRDSLVNPGPAMATHKDDEDKFFPMSGVLIFGKQGAGNSNVDLDNVMITSEEEIKTTNPVATEQY |
| | GTVATNLQSSNTAPATGTVNSQGALPGMVWQNRDVYLQGPIWAKIPHTDGHFHPSPLMGGFGL |
| | KHPPPQILIKNTPVPANPPTTFSPAKFASFITQYSTGQVSVEIEWELQKENSKRWNPEIQYTSNYNKS |
| | TNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 146 | PVNAADAAALEHDKAYDQQLKAGDNPYLRYNHADAEFQERLQEDTSFGGNLGRAVFQAKKRVLE |
| (Anc80L36) | PLGLVEEGAKTAPGKKRPVEQSPQEPDSSSGIGKKGQQPAKKRLNFGQTGDSESVPDPQPLGEPP |
| | AAPSGVGSNTMASGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKKLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSNFAWTGATKYHLN |
| | GRDSLVNPGPAMATHKDDEDKFFPMSGVLIFGKQGAGNSNVDLDNVMITSEEEIKTTNPVATEEY |
| | GTVATNLQSSNTAPATGTVNSQGALPGMVWQNRDVYLQGPIWAKIPHTDGHFHPSPLMGGFGL |
| | KHPPPQILIKNTPVPANPPTTFSPAKFASFITQYSTGQVSVEIEWELQKENSKRWNPEIQYTSNYNKS |
| | TNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 147 | PVNAADAAALEHDKAYDQQLKAGDNPYLRYNHADAEFQERLQEDTSFGGNLGRAVFQAKKRVLE |
| (Anc80L44) | PLGLVEEGAKTAPGKKRPVEQSPQEPDSSSGIGKKGQQPAKKRLNFGQTGDSESVPDPQPLGEPP |
| | AAPSGVGSNTMASGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKKLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSNFAWTGATKYHLN |
| | GRDSLVNPGPAMATHKDDEDKFFPMSGVLIFGKQGAGNSNVDLDNVMITNEEEIKTTNPVATEQ |
| | YGTVATNLQSANTAPATGTVNSQGALPGMVWQDRDVYLQGPIWAKIPHTDGHFHPSPLMGGF |
| | GLKHPPPQILIKNTPVPANPPTTFSPAKFASFITQYSTGQVSVEIEWELQKENSKRWNPEIQYTSNYN |
| | KSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 148 | PVNAADAAALEHDKAYDQQLKAGDNPYLRYNHADAEFQERLQEDTSFGGNLGRAVFQAKKRVLE |
| (Anc80L59) | PLGLVEEGAKTAPGKKRPVEQSPQEPDSSSGIGKKGQQPAKKRLNFGQTGDSESVPDPQPLGEPP |
| | AAPSGVGSNTMASGGGAPMADNNEGADGVGNASGNWHCDSTWLGDRVITTSTRTWALPTYN |
| | NHLYKQISSQSGASTNDNTYFGYSTPWGYFDFNRFHCHFSPRDWQRLINNNWGFRPKRLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSNFAWTGATKYHLN |
| | GRDSLVNPGPAMATHKDDEDKFFPMSGVLIFGKQGAGNSNVDLDNVMITNEEEIKTTNPVATEEY |
| | GTVATNLQSANTAPATGTVNSQGALPGMVWQNRDVYLQGPIWAKIPHTDGHFHPSPLMGGFG |
| | LKHPPPQILIKNTPVPANPPTTFSPAKFASFITQYSTGQVSVEIEWELQKENSKRWNPEIQYTSNYNK |
| | STNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 149 | PVNAADAAALEHDKAYDQQLKAGDNPYLRYNHADAEFQERLQEDTSFGGNLGRAVFQAKKRVLE |
| (Anc80L60) | PLGLVEEGAKTAPGKKRPVEQSPQEPDSSSGIGKKGQQPARKRLNFGQTGDSESVPDPQPLGEPP |
| | AAPSGVGSNTMAAGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKRLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | TTSGTAGNRELQFSQAGPSSMANQAKNWLPGPCYRQQRVSKTTNONNNSNFAWTGATKYHLN |
| | GRDSLVNPGPAMATHKDDEDKFFPMSGVLIFGKQGAGNSNVDLDNVMITSEEEIKTTNPVATEEY |
| | GTVATNLQSSNTAPATGTVNSQGALPGMVWQERDVYLQGPIWAKIPHTDGHFHPSPLMGGFGL |
| | KHPPPQILIKNTPVPANPPTTFSPAKFASFITQYSTGQVSVEIEWELQKENSKRWNPEIQYTSNYNKS |
| | TNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 150 | PVNAADAAALEHDKAYDQQLKAGDNPYLRYNHADAEFQERLQEDTSFGGNLGRAVFQAKKRVLE |
| (Anc80L62) | PLGLVEEGAKTAPGKKRPVEQSPQEPDSSSGIGKKGQQPARKRLNFGQTGDSESVPDPQPLGEPP |
| | AAPSGVGSNTMASGGGAPMADNNEGADGVGNASGNWHCDSTWLGDRVITTSTRTWALPTYN |
| | NHLYKQISSQSGGSTNDNTYFGYSTPWGYFDFNRFHCHFSPRDWQRLINNNWGFRPKKLNFKLF |
| | NIQVKEVTTNDGTTTIANNLTSTVQVFTDSEYQLPYVLGSAHQGCLPPFPADVFMIPQYGYLTLNN |
| | GSQAVGRSSFYCLEYFPSQMLRTGNNFEFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSNFAWTGATKYHLN |
| | GRDSLVNPGPAMATHKDDEDKFFPMSGVLIFGKQGAGNSNVDLDNVMITSEEEIKTTNPVATEEY |
| | GTVATNLQSANTAPATGTVNSQGALPGMVWQDRDVYLQGPIWAKIPHTDGHFHPSPLMGGFG |
| | LKHPPPQILIKNTPVPANPPTTFSPAKFASFITQYSTGQVSVEIEWELQKENSKRWNPEIQYTSNYNK |
| | STNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDKGE |
| NO: 151 | PVNAADAAALEHDKAYDQQLKAGDNPYLRYNHADAEFQERLQEDTSFGGNLGRAVFQAKKRVLE |
| (Anc82DI) | PLGLVEEGAKTAPGKKRPVEQSPQREPDSSTGIGKSGQQPAKKRLNFGQTGDSESVPDPQPLGEPP |
| | AAPSGVGSNTMASGGGAPMADNNEGADGVGNSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLNFKL |
| | FNIQVKEVTTNEGTKTIANNLTSTVQVFTDSEYQLPYVLGSAHQGCLPPFPADVFMIPQYGYLTLNN |
| | GSQAVGRSSFYCLEYFPSQMLRTGNNFQFSYTFEDVPFHSSYAHSQSLDRLMNPLIDQYLYYLSRTQ |
| | TTGGTAGTQTLQFSQAGPSSMANQARNWVPGPCYRQQRVSTTTNQNNNSNFAWTGATKYHLN |
| | GRDSLMNPGVAMASHKDDEDRFFPSSGVLIFGKQGAGNDNVDYSNVMITSEEEIKTTNPVATEEY |
| | GVVATNHQSANTQAQTGTVQNQGILPGMVWQNRDVYLQGPIWAKIPHTDGNFHPSPLMGGF |
| | GLKHPPPQILIKNTPVPADPPTTFNQAKLNSFITQYSTGQVSVEIEWELQKENSKRWNPEIQYTSNY |
| | YKSTNVDFAVNTEGVYSEPRPIGTRYLTRNL |
| SEQ ID | MAADGYLPDWLEDNLSEGIREWWDLKPGAPKPKANQQKQDNGRGLVLPGYKYLGPFNGLDKGE |
| NO: 152 | PVNAADAAALEHDKAYDQQLQAGDNPYLRYNHADAEFQERLQEDTSFGGNLGRAVFQAKKRVLE |
| (AAVrh.74) | PLGLVESPVKTAPGKKRPVEPSPQRSPDSSTGIGKKGQQPAKKRLNFGQTGDSESVPDPQPIGEPP |
| | AGPSGLGSGTMAAGGGAPMADNNEGADGVGSSSGNWHCDSTWLGDRVITTSTRTWALPTYN |
| | NHLYKQISNGTSGGSTNDNTYFGYSTPWGYFDFNRFHCHFSPRDWQRLINNNWGFRPKRLNFKL |
| | FNIQVKEVTQNEGTKTIANNLTSTIQVFTDSEYQLPYVLGSAHQGCLPPFPADVFMIPQYGYLTLNN |
| | GSQAVGRSSFYCLEYFPSQMLRTGNNFEFSYNFEDVPFHSSYAHSQSLDRLMNPLIDQYLYYLSRTQ |
| | STGGTAGTQQLLFSQAGPNNMSAQAKNWLPGPCYRQQRVSTTLSQNNNSNFAWTGATKYHLN |
| | GRDSLVNPGVAMATHKDDEERFFPSSGVLMFGKQGAGKDNVDYSSVMLTSEEEIKTTNPVATEQ |
| | YGVVADNLQQQNAAPIVGAVNSQGALPGMVWQNRDVYLQGPIWAKIPHTDGNFHPSPLMGGF |
| | GLKHPPPQILIKNTPVPADPPTTFNOAKLASFITQYSTGQVSVEIEWELQKENSKRWNPEIQYTSNY |
| | YKSTNVDFAVNTEGTYSEPRPIGTRYLTRNL |
| SEQ ID | RGDLRVS |
| NO: 153 | |
| SEQ ID | RGDAVGV |
| NO: 154 | |
| SEQ ID | RGDFTPTS |
| NO: 155 | |
| SEQ ID | RGDLGLS |
| NO: 156 | |
| SEQ ID | RGDMSRE |
| NO: 157 | |
| SEQ ID | MAADGYLPDWLEDNLSEGIREWWALKPGAPQPKANQQHQDNARGLVLPGYKYLGPGNGLDKG |
| NO: 158 | EPVNAADAAALEHDKAYDQQLKAGDNPYLKYNHADAEFQERLKEDTSFGGNLGRAVFQAKKRLLE |
| (AAV9- | PLGLVEEAAKTAPGKKRPVEQSPQEPDSSAGIGKSGAQPAKKRLNFGQTGDTESVPDPQPIGEPPA |
| deco1) | APSGVGSLTMASGGGAPVADNNEGADGVGSSSGNWHCDSQWLGDRVITTSTRTWALPTYNNH |
| | LYKQISNSTSGGSSNDNAYFGYSTPWGYFDFNRFHCHFSPRDWQRLINNNWGFRPKRLNFKLFNI |
| | QVKEVTDNNGVKTIANNLTSTVQVFTDSDYQLPYVLGSÅHEGCLPPFPADVFMIPQYGYLTLNDGS |
| | QAVGRSSFYCLEYFPSQMLRTGNNFQFSYEFENVPFHSSYAHSQSLDRLMNPLIDQYLYYLSKTING |
| | SGQNQQTLKFSVAGPSNMAVQGRNYIPGPSYRQQRVSTTVTQNNNSEFAWPGASSWALNGRN |
| | SLMNPGPAMASHKEGEDRFFPLSGSLIFGKQGTGRDNVDADKVMITNEEEIKTTNPVATESYGQV |
| | ATNHQSAQRGDLLLSAQAQTGWVQNQGILPGMVWQDRDVYLQGPIWAKIPHTDGNFHPSPLM |
| | GGFGMKHPPPQILIKNTPVPADPPTAFNKDKLNSFITQYSTGQVSVEIEWELQKENSKRWNPEIQY |
| | TSNYYKSNNVEFAVNTEGVYSEPRPIGTRYLTRNL |
| SEQ ID | MAADGYLPDWLEDNLSEGIREWWALKPGAPQPKANQQHQDNARGLVLPGYKYLGPGNGLDKG |
| NO: 159 | EPVNAADAAALEHDKAYDQQLKAGDNPYLKYNHADAEFQERLKEDTSFGGNLGRAVFQAKKRLLE |
| (AAV9- | PLGLVEEAAKTAPGKKRPVEQSPQEPDSSAGIGKSGAQPAKKRLNFGQTGDTESVPDPQPIGEPPA |
| deco1- | APSGVGSLTMASGGGAPVADNNEGADGVGSSSGNWHCDSQWLGDRVITTSTRTWALPTYNNH |
| mut1) | LYKQISNSTSGASTNDNAYFGYSTPWGYFDFNRFHCHFSPRDWQRLINNNWGFRPKRLNFKLFNI |
| | QVKEVTDNNGVKTIANNLTSTVQVFTDSDYQLPYVLGSAHEGCLPPFPADVFMIPQYGYLTLNDGS |
| | QAVGRSSFYCLEYFPSQMLRTGNNFQFSYEFENVPFHSSYAHSQSLDRLMNPLIDQYLYYLSKTING |
| | SGQNQQTLKFSVAGPSNMAVQGRNYIPGPSYRQQRVSTTVTQNNNSEFAWPGASSWALNGRN |
| | SLMNPGPAMASHKEGEDRFFPLSGSLIFGKQGTGRDNVDADKVMITNEEEIKTTNPVATESYGQV |
| | ATNHQSAQRGDLLLSAQAQTGWVQNQGILPGMVWQDRDVYLQGPIWAKIPHTDGNFHPSPLM |
| | GGFGMKHPPPQILIKNTPVPADPPTAFNKDKLNSFITQYSTGQVSVEIEWELQKENSKRWNPEIQY |
| | TSNYYKSNNVEFAVNTEGVYSEPRPIGTRYLTRNL |
| SEQ ID | SEQ ID NO: |
| NO: 160 | |
| SEQ ID | NSTSGGST |
| NO: 161 | |
| SEQ ID | NSTSGAST |
| NO: 162 | |
| SEQ ID | MAADGYLPDWLEDNLSEGIREWWALKPGAPQPKANQQHQDNARGLVLPGYKYLGPGNGLDKG |
| NO: 163 | EPVNAADAAALEHDKAYDQQLKAGDNPYLKYNHADAEFQERLKEDTSFGGNLGRAVFQAKKRLLE |
| (AAV9- | PLGLVEEAAKTAPGKKRPVEQSPQEPDSSAGIGKSGAQPAKKRLNFGQTGDTESVPDPQPIGEPPA |
| mut1) | APSGVGSLTMASGGGAPVADNNEGADGVGSSSGNWHCDSQWLGDRVITTSTRTWALPTYNNH |
| | LYKQISNSTSGASTNDNAYFGYSTPWGYFDFNRFHCHFSPRDWQRLINNNWGFRPKRLNFKLFNI |
| | QVKEVTDNNGVKTIANNLTSTVQVFTDSDYQLPYVLGSAHEGCLPPFPADVFMIPQYGYLTLNDGS |
| | QAVGRSSFYCLEYFPSQMLRTGNNFQFSYEFENVPFHSSYAHSQSLDRLMNPLIDQYLYYLSKTING |
| | SGQNQQTLKFSVAGPSNMAVQGRNYIPGPSYRQQRVSTTVTQNNNSEFAWPGASSWALNGRN |
| | SLMNPGPAMASHKEGEDRFFPLSGSLIFGKQGTGRDNVDADKVMITNEEEIKTTNPVATESYGQV |
| | ATNHQSAQAQAQTGWVQNQGILPGMVWQDRDVYLQGPIWAKIPHTDGNFHPSPLMGGFGM |
| | KHPPPQILIKNTPVPADPPTAFNKDKLNSFITQYSTGQVSVEIEWELQKENSKRWNPEIQYTSNYYK |
| | SNNVEFAVNTEGVYSEPRPIGTRYLTRNL |
| SEQ ID | RDLTEAVPRLPGETLITDKEVIYICPFNGPIKGRVYITNYRLYLRSLETDSSLILDVPLGVISRIEKMGGA |
| NO: 164 | TSRGENSYGLDITCKDMRNLRFALKQEGHSRRDMFEILTRYAFPLAHSLPLFAFLNEEKFNVDGWT |
| (A1) | VYNPVEEYRRQGLPNHHWRITFINKCYELCDTYPALLVVPYRASDDDLRRVATFRSRNRIPVLSWIH |
| | PENKTVIVRCSQPLVGMSGKRNKDDEKYLDVIRETNKQISKLTIYDARPSVNAVANKATGGGYESD |
| | DAYHNAELFFLDIHNIHVMRESLKKVKDIVYPNVEESHWLSSLESTHWLEHIKLVLTGAIQVADKVSS |
| | GKSSVLVHCSDGWDRTAQLTSLAMLMLDSFYRSIEGFEILVQKEWISFGHKFASRIGHGDKNHTDA |
| | DRSPIFLQFIDCVWQMSKQFPTAFEFNEQFLIIILDHLYSCRFGTFLFNCESAR |
| SEQ ID | MASASTSKYNSHSLENESIKRTSRDGVNRDLTEAVPRLPGETLITDKEVIYICPFNGPIKGRVYITNYRL |
| NO: 165 | YLRSLETDSSLILDVPLGVISRIEKMGGATSRGENSYGLDITCKDMRNLRFALKQEGHSRRDMFEILT |
| (MTM1 | RYAFPLAHSLPLFAFLNEEKFNVDGWTVYNPVEEYRRQGLPNHHWRITFINKCYELCDTYPALLVVP |
| amino | YRASDDDLRRVATFRSRNRIPVLSWIHPENKTVIVRCSQPLVGMSGKRNKDDEKYLDVIRETNKQIS |
| acid | KLTIYDARPSVNAVANKATGGGYESDDAYHNAELFFLDIHNIHVMRESLKKVKDIVYPNVEESHWL |
| sequence: | SSLESTHWLEHIKLVLTGAIQVADKVSSGKSSVLVHCSDGWDRTAQLTSLAMLMLDSFYRSIEGFEIL |
| A2) | VQKEWISFGHKFASRIGHGDKNHTDADRSPIFLQFIDCVWQMSKQFPTAFEFNEQFLIIILDHLYSC |
| | RFGTFLFNCESARERQKVTERTVSLWSLINSNKEKFKNPFYTKEINRVLYPVASMRHLELWVNYYIR |
| | WNPRIKQQQPNPVEQRYMELLALRDEYIKRLEELQLANSAKLSDPPTSPSSPSQMMPHVQTHF |
| SEQ ID | atggcttctgcatcaacttctaaatataattcacactccttggagaatgagtctattaagaggacgtctcgagatggagtcaat |
| NO: 166 | cgagatctcactgaggctgttcctcgacttccaggagaaacactaatcactgacaaagaagttatttacatatgtcctttcaat |
| (A3: MTM1 | ggccccattaagggaagagtttacatcacaaattatcgtctttatttaagaagtttggaaacggattcttctctaatacttgatg |
| coding | ttcctctgggtgtgatctcgagaattgaaaaaatgggaggcgcgacaagtagaggagaaaattcctatggtctagatattac |
| seq) | ttgtaaagacatgagaaacctgaggttcgctttgaaacaggaaggccacagcagaagagatatgtttgagatcctcacgag |
| | atacgcgtttcccctggctcacagtctgccattatttgcatttttaaatgaagaaaagtttaacgtggatggatggacagtttac |
| | aatccagtggaagaatacaggaggcagggcttgcccaatcaccattggagaataacttttattaataagtgctatgagctct |
| | gtgacacttaccctgctcttttggtggttccgtatcgtgcctcagatgatgacctccggagagttgcaacttttaggtcccgaaa |
| | tcgaattccagtgctgtcatggattcatccagaaaataagacggtcattgtgcgttgcagtcagcctcttgtcggtatgagtgg |
| | gaaacgaaataaagatgatgagaaatatctcgatgttatcagggagactaataaacaaatttctaaactcaccatttatgat |
| | gcaagacccagcgtaaatgcagtggccaacaaggcaacaggaggaggatatgaaagtgatgatgcatatcataacgccga |
| | acttttcttcttagacattcataatattcatgttatgcgggaatctttaaaaaaagtgaaggacattgtttatoctaatgtagaa |
| | gaatctcattggttgtccagtttggagtctactcattggttagaacatatcaagctcgttttgacaggagccattcaagtagca |
| | gacaaagtttcttcagggaagagttcagtgcttgtgcattgcagtgacggatgggacaggactgctcagctgacatccttggc |
| | catgctgatgttggatagcttctataggagcattgaagggttcgaaatactggtacaaaaagaatggataagttttggacata |
| | aatttgcatctcgaataggtcatggtgataaaaaccacaccgatgctgaccgttctcctatttttctccagtttattgattgtgtg |
| | tggcaaatgtcaaaacagttccctacagcttttgaattcaatgaacaatttttgattataattttggatcatctgtatagttgcc |
| | gatttggtactttcttattcaactgtgaatctgctcgagaaagacagaaggttacagaaaggactgtttctttatggtcactgat |
| | aaacagtaataaagaaaaattcaaaaaccccttctatactaaagaaatcaatcgagttttatatccagttgccagtatgcgtc |
| | acttggaactctgggtgaattactacattagatggaaccccaggatcaagcaacaacagccgaatccagtggagcagcgtt |
| | acatggagctcttagccttacgcgacgaatacataaagcggcttgaggaactgcagctcgccaactctgccaagctttctgat |
| | cccccaacttcaccttccagtccttcgcaaatgatgccccatgtgcaaactcacttttaa |
| SEQ ID | TTAAAAGTGGGTCTGGACGTGGGGCATCATCTGTGAGGGGCTGCTTGGTGAAGTAGGTGGGT |
| NO: 167 | CGGACAGCTTGGCGCTATTGGCCAGCTGCAGCTCCTCCAGCCTCTTGATATACTCATCTCTCAG |
| (A4) | GGCCAGCAGCTCCATGTACCGCTGCTCCACAGGGTTGGGCTGCTGCTGCTTGATCCGGGGATT |
| (Genscript | CCAGCGGATATAGTAGTTCACCCACAGCTCCAGGTGTCTCATGGATGCCACAGGATACAGCAC |
| codon | GCGATTGATCTCCTTTGTGTAGAAGGGGTTCTTGAACTTCTCCTTATTGCTGTTGATCAGGCTCC |
| optimized | ACAGAGACACTGTGCGCTCGGTCACCTTCTGCCTCTCTCTGGCAGACTCACAATTAAACAGGAA |
| MTM1) | TGTGCCGAACCTGCAGGAGTACAGGTGGTCCAGGATGATGATCAGAAACTGCTCGTTAAACTC |
| | GAAGGCGGTTGGGAACTGCTTGGACATCTGCCACACGCAGTCGATAAACTGCAGGAAGATAG |
| | GGCTGCGGTCGGCATCGGTGTGATTCTTATCGCCGTGGCCGATCCTGGAGGCAAACTTGTGGC |
| | CGAAGCTGATCCACTCCTTCTGCACCAGGATCTCAAAGCCCTCGATAGATCTATAGAAGGAGTC |
| | CAGCATCAGCATaGCCAGGCTTGTCAGCTGTGCTGTTCTGTCCCACCCATCGGAACAGTGCACC |
| | AGCACGGAGCTCTTGCCAGAGGACACCTTATCGGCCACCTGGATGGCGCCTGTCAGCACCAGC |
| | TTGATGTGCTCCAGCCAGTGGGTAGACTCCAGGCTAGACAGCCAGTGGGACTCCTCCACGTTT |
| | GGGTACACGATGTCCTTCACCTTCTTCAGGGACTCTCTCATCACGTGGATATTGTGGATATCCA |
| | GAAAGAACAGCTCGGCGTTGTGATAGGCGTCATCAGACTCATATCCTCCTCCTGTTGCCTTATT |
| | TGCCACTGCATTCACAGAAGGCCGGGCATCATAGATTGTCAGCTTGCTGATCTGCTTATTTGTC |
| | TCTCTGATCACATCCAGGTACTTCTCGTCATCCTTGTTCCGCTTGCCAGACATTCCCACCAGTGG |
| | CTGGGAGCAGCGCACGATCACTGTCTTATTCTCTGGGTGGATCCAGCTCAGCACAGGGATTCT |
| | GTTCCGGCTCCGGAAGGTGGCCACTCTCCGCAGGTCATCGTCGCTGGCTCTGTAGGGCACCAC |
| | CAGCAGGGCTGGATATGTATCGCACAGCTCGTAGCACTTATTGATAAAGGTGATCCGCCAGTG |
| | GTGGTTAGGCAGGCCCTGGCGCCTATACTCCTCCACGGGGTTGTACACTGTCCAGCCGTCCAC |
| | ATTGAACTTCTCCTCGTTCAGAAAGGCGAACAGAGGCAGAGAGTGGGCCAGGGGAAAGGCAT |
| | ATCTGGTCAGGATCTCGAACATATCTCTCCGGGAGTGGCCCTCCTGCTTCAGGGCAAACCGCA |
| | GATTGCGCATGTCCTTACAGGTGATATCCAGGCCGTAGCTGTTCTCTCCCCTAGAGGTGGCTCC |
| | TCCCATCTTCTCGATTCTGCTGATCACGCCCAGAGGCACGTCCAGGATCAGGGAGCTATCTGTC |
| | TCCAGGCTCCGCAGGTACAGTCTGTAATTGGTGATATACACGCGGCCCTTGATTGGGCCGTTG |
| | AAAGGGCAGATGTAGATCACTTCCTTGTCTGTGATCAGTGTCTCTCCAGGCAGCCTTGGCACTG |
| | CCTCTGTCAGATCTCTATTGACCCCGTCCCTTGAGGTTCTCTTGATGCTCTCGTTTTCCAGGCTAT |
| | GTGAGTTGTATTTGCTTGTGCTTGCTGATGCCAT |
| SEQ ID | ATGGCTTCTGCATCAACTTCTAAATATAATTCACACTCCTTGGAGAATGAGTCTATTAAGAGGA |
| NO: 168 | CGTCTCGAGATGGAGTCAATCGAGATCTCACTGAGGCTGTTCCTCGACTTCCAGGAGAAACAC |
| (A5) | TAATCACTGACAAAGAAGTTATTTACATATGTCCTTTCAATGGCCCCATTAAGGGAAGAGTTTA |
| (Eurofins | CATCACAAATTATCGTCTTTATTTAAGAAGTTTGGAAACGGATTCTTCTCTAATACTTGATGTTC |
| codon | CTCTGGGTGTGATCTCGAGAATTGAAAAAATGGGAGGCGCGACAAGTAGAGGAGAAAATTCC |
| optimized | TATGGTCTAGATATTACTTGTAAAGACATGAGAAACCTGAGGTTCGCTTTGAAACAGGAAGGC |
| MTM1) | CACAGCAGAAGAGATATGTTTGAGATCCTCACGAGATACGCGTTTCCCCTGGCTCACAGTCTGC |
| | CATTATTTGCATTTTTAAATGAAGAAAAGTTTAACGTGGATGGATGGACAGTTTACAATCCAGT |
| | GGAAGAATACAGGAGGCAGGGCTTGCCCAATCACCATTGGAGAATAACTTTTATTAATAAGTG |
| | CTATGAGCTCTGTGACACTTACCCTGCTCTTTTGGTGGTTCCGTATCGTGCCTCAGATGATGACC |
| | TCCGGAGAGTTGCAACTTTTAGGTCCCGAAATCGAATTCCAGTGCTGTCATGGATTCATCCAGA |
| | AAATAAGACGGTCATTGTGCGTTGCAGTCAGCCTCTTGTCGGTATGAGTGGGAAACGAAATAA |
| | AGATGATGAGAAATATCTCGATGTTATCAGGGAGACTAATAAACAAATTTCTAAACTCACCATT |
| | TATGATGCAAGACCCAGCGTAAATGCAGTGGCCAACAAGGCAACAGGAGGAGGATATGAAAG |
| | TGATGATGCATATCATAACGCCGAACTTTTCTTCTTAGACATTCATAATATTCATGTTATGCGGG |
| | AATCTTTAAAAAAAGTGAAGGACATTGTTTATCCTAATGTAGAAGAATCTCATTGGTTGTCCAG |
| | TTTGGAGTCTACTCATTGGTTAGAACATATCAAGCTCGTTTTGACAGGAGCCATTCAAGTAGCA |
| | GACAAAGTTTCTTCAGGGAAGAGTTCAGTGCTTGTGCATTGCAGTGACGGATGGGACAGGACT |
| | GCTCAGCTGACATCCTTGGCCATGCTGATGTTGGATAGCTTCTATAGGAGCATTGAAGGGTTC |
| | GAAATACTGGTACAAAAAGAATGGATAAGTTTTGGACATAAATTTGCATCTCGAATAGGTCAT |
| | GGTGATAAAAACCACACCGATGCTGACCGTTCTCCTATTTTTCTCCAGTTTATTGATTGTGTGTG |
| | GCAAATGTCAAAACAGTTCCCTACAGCTTTTGAATTCAATGAACAATTTTTGATTATAATTTTGG |
| | ATCATCTGTATAGTTGCCGATTTGGTACTTTCTTATTCAACTGTGAATCTGCTCGAGAAAGACAG |
| | AAGGTTACAGAAAGGACTGTTTCTTTATGGTCACTGATAAACAGTAATAAAGAAAAATTCAAA |
| | AACCCATTCTATACTAAAGAAATCAATAGAGTTTTATATCCAGTTGCAAGTATGCGTCACTTGG |
| | AACTCTGGGTGAATTACTACATTAGATGGAACCCCAGGATCAAACAACAACAACCAAATCCAG |
| | TGGAACAACGTTACATGGAACTCTTAGCCTTACGAGATGAATACATAAAACGGCTTGAGGAAC |
| | TGCAACTAGCAAACTCTGCAAAACTTTCTGATCCCCCAACTTCACCTTCCAGTCCTTCTCAAATG |
| | ATGCCACATGTGCAAACTCACTTTTAA |
| SEQ ID | ATGGCCTCTGCCAGCACCTCTAAGTACAACAGCCACTCCCTGGAAAATGAATCCATCAAAAGG |
| NO: 169 | ACCAGCAGAGATGGAGTGAACAGAGACCTAACTGAAGCTGTGCCAAGACTGCCTGGAGAGAC |
| (A6) | CCTGATCACAGACAAGGAAGTGATCTACATCTGCCCCTTCAATGGCCCTATCAAGGGAAGGGT |
| (delCpG | GTACATCACCAACTACAGGCTTTACCTGAGATCCCTGGAGACAGACAGCAGCCTGATCCTGGA |
| Genscript | TGTGCCTCTGGGAGTGATCAGCAGAATAGAGAAGATGGGGGGTGCCACCAGCAGAGGAGAG |
| codon | AACAGCTATGGCCTGGACATCACCTGCAAGGACATGAGAAACCTGAGATTTGCCCTGAAGCAG |
| optimized | GAGGGCCACAGCAGAAGAGACATGTTTGAAATCCTGACCAGGTATGCCTTCCCCCTGGCCCAC |
| MTM1) | TCTCTCCCCCTGTTTGCCTTCCTGAATGAGGAAAAGTTCAATGTTGATGGCTGGACAGTGTACA |
| | ACCCAGTGGAGGAGTACAGAAGACAGGGCCTGCCTAACCACCACTGGAGGATCACCTTCATCA |
| | ACAAGTGCTATGAACTGTGTGACACATACCCTGCCCTGCTGGTGGTGCCTTACAGAGCCTCAGA |
| | TGATGACCTGAGAAGAGTTGCCACCTTCAGAAGCAGGAACAGAATCCCTGTACTGAGCTGGAT |
| | CCACCCTGAGAATAAGACTGTGATTGTGAGGTGCAGCCAGCCCCTGGTGGGCATGAGTGGCA |
| | AGAGAAACAAAGATGATGAAAAGTACCTGGATGTGATCAGAGAGACCAACAAACAGATCAGC |
| | AAGCTCACCATCTATGATGCTAGACCCTCTGTTAATGCTGTGGCCAACAAGGCCACAGGGGGA |
| | GGCTATGAATCTGATGATGCTTACCACAATGCTGAGCTGTTCTTCCTGGACATCCACAACATCC |
| | ATGTGATGAGAGAATCCCTCAAGAAAGTGAAGGACATTGTGTACCCTAATGTGGAAGAAAGTC |
| | ACTGGCTGAGCAGCTTGGAGTCCACCCACTGGCTGGAGCACATCAAGCTGGTCCTGACAGGAG |
| | CCATCCAGGTGGCTGACAAGGTGAGTTCTGGCAAGTCCTCAGTGCTGGTCCACTGCTCTGATG |
| | GCTGGGACAGAACTGCCCAGCTGACCAGTCTGGCCATGCTGATGCTGGACTCCTTCTACAGAA |
| | GCATTGAAGGCTTTGAAATCCTGGTGCAAAAGGAATGGATCTCTTTTGGCCACAAGTTTGCCA |
| | GCAGAATTGGCCATGGTGACAAAAACCACACAGATGCTGACAGAAGCCCTATCTTCCTGCAGT |
| | TCATTGACTGTGTGTGGCAGATGAGCAAGCAGTTCCCCACAGCATTTGAGTTCAATGAGCAGT |
| | TCCTAATAATCATCCTGGACCACCTCTACAGCTGCAGATTTGGCACCTTCCTGTTCAACTGTGAG |
| | TCTGCCAGAGAAAGACAGAAGGTGACAGAGAGGACAGTGAGCCTGTGGAGCCTGATCAACTC |
| | CAACAAGGAGAAGTTCAAGAACCCCTTCTACACCAAGGAAATCAACAGGGTGCTGTACCCTGT |
| | GGCTAGCATGAGGCACCTGGAGCTGTGGGTCAACTACTACATCAGATGGAACCCTAGAATCAA |
| | ACAGCAACAACCCAACCCTGTGGAGCAGAGGTACATGGAGTTACTGGCCCTGAGGGATGAGT |
| | ACATCAAGAGACTGGAGGAGCTGCAGCTGGCCAACTCTGCCAAACTGTCTGACCCTCCTACCTC |
| | CCCCAGCTCCCCCTCTCAGATGATGCCACATGTGCAGACCCACTTTTAA |
| SEQ ID | TTAAAAGTGGGTCTGCACGTGAGGCATCATCTGGCTGGGGCTGCTAGGGCTTGTAGGAGGAT |
| NO 170 | CGCTCAGCTTGGCGCTGTTGGCCAGCTGCAGTTCTTCCAGTCTCTTGATGTACTCGTCCCGCAG |
| (A7) | GGCCAGCAGTTCCATGTACCGCTGTTCCACAGGATTGGGCTGCTGCTGCTTGATTCTGGGGTTC |
| (GeneArt | CACCGGATGTAGTAGTTGACCCACAGTTCCAGATGTCTCATGCTGGCCACGGGGTACAGCACC |
| codon | CGGTTGATTTCTTTGGTGTAGAAGGGGTTCTTGAATTTCTCTTTGTTGCTGTTGATCAGGGACC |
| optimized | ACAGAGACACGGTCCGCTCGGTCACTTTCTGCCGTTCTCTGGCGCTCTCGCAGTTGAACAGGAA |
| MTM1) | GGTGCCGAATCTGCAGCTGTACAGGTGGTCCAGGATGATGATCAGGAACTGCTCGTTGAACTC |
| | GAAGGCGGTAGGGAACTGCTTGGACATCTGCCACACGCAGTCGATGAACTGCAGGAAGATGG |
| | GGCTTCTATCGGCGTCGGTGTGGTTCTTGTCGCCGTGTCCGATTCTGCTGGCGAACTTGTGGCC |
| | GAAGCTGATCCACTCTTTCTGCACCAGGATCTCAAAGCCCTCGATGGATCTGTAGAAGCTGTCC |
| | AGCATCAGCATGGCCAGAGATGTCAGCTGGGCTGTTCTATCCCAGCCGTCGCTACAGTGCACC |
| | AGCACGCTAGACTTGCCAGAGGACACCTTATCGGCCACCTGGATGGCGCCTGTCAGCACCAGC |
| | TTGATGTGTTCCAGCCAGTGTGTGCTTTCCAGACTGCTCAGCCAGTGGCTCTCTTCCACATTGG |
| | GGTACACGATGTCCTTCACTTTCTTCAGGCTTTCCCGCATCACGTGGATGTTGTGGATGTCCAG |
| | AAAGAACAGCTCGGCGTTATGATAGGCGTCGTCGCTCTCGTATCCGCCGCCTGTAGCTTTGTTG |
| | GCCACGGCGTTCACGCTAGGTCTGGCGTCGTAGATGGTCAGCTTGCTGATCTGCTTGTTTGTCT |
| | CGCGGATCACGTCCAGGTACTTCTCGTCGTCCTTGTTTCTCTTGCCAGACATGCCCACGAGGGG |
| | CTGAGAACACCGCACGATCACGGTCTTGTTCTCGGGGTGAATCCAGCTCAGCACAGGGATTCT |
| | GTTCCGGCTCCGAAAGGTGGCCACTCTTCTCAGGTCGTCGTCGGAGGCTCTGTAAGGCACCAC |
| | CAGCAGTGCGGGGTATGTGTCGCACAGCTCGTAGCACTTGTTGATGAAGGTGATCCGCCAGTG |
| | GTGATTAGGCAGGCCCTGTCTCCGATACTCTTCCACGGGGTTGTACACGGTCCAGCCGTCCACG |
| | TTGAACTTCTCTTCGTTCAGGAAGGCGAACAGAGGCAGGGAGTGAGCCAGAGGAAAGGCGTA |
| | TCTGGTCAGGATCTCGAACATGTCCCGTCTGCTGTGGCCCTCTTGCTTCAGGGCGAATCTCAGG |
| | TTCCGCATGTCCTTGCATGTGATGTCCAGGCCGTAGCTATTCTCGCCTCTGGAGGTGGCTCCGC |
| | CCATTTTCTCAATCCGGCTGATCACGCCCAGGGGCACATCCAGGATCAGGCTGCTATCGGTTTC |
| | CAGGGACCGCAGGTACAGCCGGTAGTTGGTGATGTACACGCGGCCCTTGATGGGGCCGTTGA |
| | AGGGGCAGATGTAGATCACTTCTTTGTCGGTGATCAGTGTCTCGCCAGGCAGTCTAGGCACGG |
| | CCTCTGTCAGATCCCTGTTCACGCCATCTCTGCTGGTCCGCTTGATGCTCTCGTTTTCCAGGCTG |
| | TGGCTGTTGTACTTGCTTGTGCTGGCGCTAGCCAT |
| SEQ ID | gacattgattattgactagttattaatagtaatcaattacggggtcattagttcatagcccatatatggagttccgcgttacata |
| NO: 171 | acttacggtaaatggcccgcctggctgaccgcccaacgacccccgcccattgacgtcaataatgacgtatgttcccatagtaa |
| (B1) | cgccaatagggactttccattgacgtcaatgggtggagtatttacggtaaactgcccacttggcagtacatcaagtgtatcat |
| (CMV-IE | atgccaagtacgccccctattgacgtcaatgacggtaaatggcccgcctggcattatgcccagtacatgaccttatgggactt |
| sequence) | tcctacttggcagtacatctacgtattagtcatcgctattaccatggt |
| SEQ ID | Tcgaggtgagccccacgttctgcttcactctccccatctcccccccctccccacccccaattttgtatttatttattttttaattatt |
| NO: 172 | ttgtgcagcgatgggggcggggggggggggggggcgcgcgccaggcggggggggcggggcgaggggggggcggggc |
| (B2) | gaggcggagaggtgcggcggcagccaatcagagcggcgcgctccgaaagtttccttttatggcgaggcggcggcggcggc |
| (chicken | ggccctataaaaagcgaagcgcgcggcgggcg |
| beta | |
| actin | |
| promoter) | |
| SEQ | Gacattgattattgactagttattaatagtaatcaattacggggtcattagttcatagcccatatatggagttccgcgttacat |
| ID NO: | aacttacggtaaatggcccgcctggctgaccgcccaacgacccccgcccattgacgtcaataatgacgtatgttcccatagta |
| 173 | acgccaatagggactttccattgacgtcaatgggtggagtatttacggtaaactgcccacttggcagtacatcaagtgtatca |
| (B3) | tatgccaagtacgccccctattgacgtcaatgacggtaaatggcccgcctggcattatgcccagtacatgaccttatgggact |
| (CMV-IE | ttcctacttggcagtacatctacgtattagtcatcgctattaccatggttcgaggtgagccccacgttctgcttcactctccccat |
| plus | ctcccccccctccccacccccaattttgtatttatttattttttaattattttgtgcagcgatgggggcgggggggggggggggg |
| chicken | cgcgcgccaggcggggggggcggggcgaggggcggggcggggcgaggcggagaggtgcggcggcagccaatcagagc |
| beta | ggcgcgctccgaaagtttccttttatggcgaggcggcggcggcggcggccctataaaaagcgaagcgcgcggcgggcg |
| actin | |
| promoter) | |
| SEQ ID | AAAAAATGCTTTCTTCTTTTAATATACTTTTTTGTTTATCTTATTTCTAATACTTTCCCTAATCTCTT |
| NO: 174 | TCTTTCAGGGCAATAATGATACAATGTATCATGCCTCTTTGCACCATTCTAAAGAATAACAGTG |
| (B4) | ATAATTTCTGGGTTAAGGCAATAGCAATATTTCTGCATATAAATATTTCTGCATATAAATTGTAA |
| (3′ end | CTGATGTAAGAGGTTTCATATTGCTAATAGCAGCTACAATCCAGCTACCATTCTGCTTTTATTTT |
| of | ATGGTTGGGATAAGGCTGGATTATTCTGAGTCCAAGCTAGGCCCTTTTGCTAATCATGTTCATA |
| human | CCTCTTATCTTCCTCCCACAG |
| beta | |
| globin | |
| intron | |
| 2) | |
| SEQ ID | GTGCATTGGAACGCGGATTCCCCGTGCCAAGAGTGACGTAAGTACCGCCTATAGAGTCTATAG |
| NO: 175 | GCCCAC |
| (B5) | |
| (human | |
| betaherpes | |
| virus | |
| intron) | |
| SEQ ID | GTGCATTGGAACGCGGATTCCCCGTGCCAAGAGTGACGTAAGTACCGCCTATAGAGTCTATAG |
| NO: 176 | GCCCACAAAAAATGCTTTCTTCTTTTAATATACTTTTTTGTTTATCTTATTTCTAATACTTTCCCTA |
| (B6) | ATCTCTTTCTTTCAGGGCAATAATGATACAATGTATCATGCCTCTTTGCACCATTCTAAAGAATA |
| (chimeric | ACAGTGATAATTTCTGGGTTAAGGCAATAGCAATATTTCTGCATATAAATATTTCTGCATATAAA |
| intron) | TTGTAACTGATGTAAGAGGTTTCATATTGCTAATAGCAGCTACAATCCAGCTACCATTCTGCTTT |
| | TATTTTATGGTTGGGATAAGGCTGGATTATTCTGAGTCCAAGCTAGGCCCTTTTGCTAATCATG |
| | TTCATACCTCTTATCTTCCTQCCACAG |
| SEQ ID | GATCTTTTTCCCTCTGCCAAAAATTATGGGGACATCATGAAGCCCCTTGAGCATCTGACTTCTG |
| NO: 177 | GCTAATAAAGGAAATTTATTTTCATTGCAATAGTGTGTTGGAATTTTTTGTGTCTCTCA |
| (D1) | |
| SEQ ID | Ttggccactccctctctgcgcgctcgctcgctcactgaggccgcccgggcaaagcccgggcgtcgggcgacctttggtcgccc |
| NO: 178 | ggcctcagtgagcgagcgagcgcgcagagagggagtggccaactccatcactaggggttcct |
| (D2) | |
| SEQ ID | Aggaacccctagtgatggagttggccactccctctctgcgcgctcgctcgctcactgaggccgggcgaccaaaggtcgcccg |
| NO: 179 | acgcccgggctttgcccgggcggcctcagtgagcgagcgagcgcgcagagagggagtggccaa |
| (D3) | |
| SEQ ID | CTCCTGGGCAACGTGCTGGTCTGTGTGCTGGCCCATCACTTTGGCAAAGAATT |
| NO: 180 | |
| (D4) | |
| SEQ ID | ttggccactccctctctgcgcgctcgctcgctcactgaggccgcccgggcaaagcccgggcgtcgggcgacctttggtcgccc |
| NO: 181 | ggcctcagtgagcgagcgagcgcgcagagagggagtggccaactccatcactaggggttcctTGCGCAcaggtaccccc |
| (E1) | ctgcccccacagctcctctcctgtgccttgtttcccagccatgcgttctcctctataaatacccgctctggtatttggggttggca |
| | gctgttgctgccagggagatggttgggttgacatgcggctcctgacaaaacacaaacccctggtgtgtgtgggcgtgggtggt |
| | gtgagtagggggatgaatcagggagggggcgggggacccagggggcaggagccacacaaagtctgtgcggggggggag |
| | cgcacatagcaattggaaactggctgcagacatgcttgctgcctgccctggcgaaggattggtaggcttgccgtcacaggac |
| | ccccgctggctgactcaggggcgcaggctcttgcgggggagctggcctcccgcccccacggccacgggccctttcctggcag |
| | gacagcgggatcttgcagctgtcaggggaggggaggcgggggctgatgtcaggagggatacaaatagtgccgacggctgg |
| | gggccctgtctcccctcgccgcatccactctccggccggccgcctgcccgccgcctcctccgtgcgcccgccagcctcgcccgc |
| | gccgtcaccGATATCtcagatcgcctggagacgccatccacgctgttttgacctccatagaagacaccgggaccgatcca |
| | gcctccgcggattcgaaTCCCGGCCGGGAACGGTGCATTGGAACGCGGATTCCCCGTGCCAAGAGT |
| | GACGTAAGTACCGCCTATAGAGTCTATAGGCCCACAAAAAATGCTTTCTTCTTTTAATATACTTT |
| | TTTGTTTATCTTATTTCTAATACTTTCCCTAATCTCTTTCTTTCAGGGCAATAATGATACAATGTAT |
| | CATGCCTCTTTGCACCATTCTAAAGAATAACAGTGATAATTTCTGGGTTAAGGCAATAGCAATA |
| | TTTCTGCATATAAATATTTCTGCATATAAATTGTAACTGATGTAAGAGGTTTCATATTGCTAATA |
| | GCAGCTACAATCCAGCTACCATTCTGCTTTTATTTTATGGTTGGGATAAGGCTGGATTATTCTGA |
| | GTCCAAGCTAGGCCCTTTTGCTAATCATGTTCATACCTCTTATCTTCCTCCCACAGCTCCTGGGC |
| | AACGTGCTGGTCTGTGTGCTGGCCCATCACTTTGGCAAAGAATTTGAGCGGCCGCCACCATGG |
| | CATCAGCAAGCACAAGCAAATACAACTCACATAGCCTGGAAAACGAGAGCATCAAGAGAACCT |
| | CAAGGGACGGGGTCAATAGAGATCTGACAGAGGCAGTGCCAAGGCTGCCTGGAGAGACACT |
| | GATCACAGACAAGGAAGTGATCTACATCTGCCCTTTCAACGGCCCAATCAAGGGCCGCGTGTA |
| | TATCACCAATTACAGACTGTACCTGCGGAGCCTGGAGACAGATAGCTCCCTGATCCTGGACGT |
| | GCCTCTGGGCGTGATCAGCAGAATCGAGAAGATGGGAGGAGCCACCTCTAGGGGAGAGAAC |
| | AGCTACGGCCTGGATATCACCTGTAAGGACATGCGCAATCTGCGGTTTGCCCTGAAGCAGGAG |
| | GGCCACTCCCGGAGAGATATGTTCGAGATCCTGACCAGATATGCCTTTCCCCTGGCCCACTCTC |
| | TGCCTCTGTTCGCCTTTCTGAACGAGGAGAAGTTCAATGTGGACGGCTGGACAGTGTACAACC |
| | CCGTGGAGGAGTATAGGCGCCAGGGCCTGCCTAACCACCACTGGCGGATCACCTTTATCAATA |
| | AGTGCTACGAGCTGTGCGATACATATCCAGCCCTGCTGGTGGTGCCCTACAGAGCCAGCGACG |
| | ATGACCTGCGGAGAGTGGCCACCTTCCGGAGCCGGAACAGAATCCCTGTGCTGAGCTGGATCC |
| | ACCCAGAGAATAAGACAGTGATCGTGCGCTGCTCCCAGCCACTGGTGGGAATGTCTGGCAAGC |
| | GGAACAAGGATGACGAGAAGTACCTGGATGTGATCAGAGAGACAAATAAGCAGATCAGCAA |
| | GCTGACAATCTATGATGCCCGGCCTTCTGTGAATGCAGTGGCAAATAAGGCAACAGGAGGAG |
| | GATATGAGTCTGATGACGCCTATCACAACGCCGAGCTGTTCTTTCTGGATATCCACAATATCCA |
| | CGTGATGAGAGAGTCCCTGAAGAAGGTGAAGGACATCGTGTACCCAAACGTGGAGGAGTCCC |
| | ACTGGCTGTCTAGCCTGGAGTCTACCCACTGGCTGGAGCACATCAAGCTGGTGCTGACAGGCG |
| | CCATCCAGGTGGCCGATAAGGTGTCCTCTGGCAAGAGCTCCGTGCTGGTGCACTGTTCCGATG |
| | GGTGGGACAGAACAGCACAGCTGACAAGCCTGGCtATGCTGATGCTGGACTCCTTCTATAGAT |
| | CTATCGAGGGCTTTGAGATCCTGGTGCAGAAGGAGTGGATCAGCTTCGGCCACAAGTTTGCCT |
| | CCAGGATCGGCCACGGCGATAAGAATCACACCGATGCCGACCGCAGCCCTATCTTCCTGCAGT |
| | TTATCGACTGCGTGTGGCAGATGTCCAAGCAGTTCCCAACCGCCTTCGAGTTTAACGAGCAGTT |
| | TCTGATCATCATCCTGGACCACCTGTACTCCTGCAGGTTCGGCACATTCCTGTTTAATTGTGAGT |
| | CTGCCAGAGAGAGGCAGAAGGTGACCGAGCGCACAGTGTCTCTGTGGAGCCTGATCAACAGC |
| | AATAAGGAGAAGTTCAAGAACCCCTTCTACACAAAGGAGATCAATCGCGTGCTGTATCCTGTG |
| | GCATCCATGAGACACCTGGAGCTGTGGGTGAACTACTATATCCGCTGGAATCCCCGGATCAAG |
| | CAGCAGCAGCCCAACCCTGTGGAGCAGCGGTACATGGAGCTGCTGGCCCTGAGAGATGAGTA |
| | TATCAAGAGGCTGGAGGAGCTGCAGCTGGCCAATAGCGCCAAGCTGTCCGACCCACCTACTTC |
| | ACCAAGCAGCCCCTCACAGATGATGCCCCACGTCCAGACCCACTTTTAATTAAGATCTTTTTCCC |
| | TCTGCCAAAAATTATGGGGACATCATGAAGCCCCTTGAGCATCTGACTTCTGGCTAATAAAGGA |
| | AATTTATTTTCATTGCAATAGTGTGTTGGAATTTTTTGTGTCTCTCActcgagGCCTaggaacccctagt |
| | gatggagttggccactccctctctgcgcgctcgctcgctcactgaggccgggcgaccaaaggtcgcccgacgcccgggctttg |
| | cccgggcggcctcagtgagcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 182 | ggcctcagtgagcgagcgagcgcgcagagagggagtggccaactccatcactaggggttcctTGCGCAcaggtaccgac |
| (E2) | attgattattgactagttattaatagtaatcaattacggggtcattagttcatagcccatatatggagttccgcgttacataact |
| | tacggtaaatggcccgcctggctgaccgcccaacgacccccgcccattgacgtcaataatgacgtatgttcccatagtaacgc |
| | caatagggactttccattgacgtcaatgggtggagtatttacggtaaactgcccacttggcagtacatcaagtgtatcatatgc |
| | caagtacgccccctattgacgtcaatgacggtaaatggcccgcctggcattatgcccagtacatgaccttatgggactttccta |
| | cttggcagtacatctacgtattagtcatcgctattaccatggtcgaggtgagccccacgttctgcttcactctccccatctcccc |
| | cccctccccacccccaattttgtatttatttattttttaattattttgtgcagcgatgggggcggggggggggggggggcgcgcg |
| | ccaggcggggggggcggggcgaggggcggggggggcgaggcggagaggtgcggcggcagccaatcagagcggcgcg |
| | ctccgaaagtttccttttatggcgaggcggcggcggcggcggccctataaaaagcgaagcgcgcggcgggcgGATATCtc |
| | agatcgcctggagacgccatccacgctgttttgacctccatagaagacaccgggaccgatccagcctccgcggattcgaaTC |
| | CCGGCCGGGAACGGTGCATTGGAACGCGGATTCCCCGTGCCAAGAGTGACGTAAGTACCGCC |
| | TATAGAGTCTATAGGCCCACAAAAAATGCTTTCTTCTTTTAATATACTTTTTTGTTTATCTTATTTC |
| | TAATACTTTCCCTAATCTCTTTCTTTCAGGGCAATAATGATACAATGTATCATGCCTCTTTGCACC |
| | ATTCTAAAGAATAACAGTGATAATTTCTGGGTTAAGGCAATAGCAATATTTCTGCATATAAATA |
| | TTTCTGCATATAAATTGTAACTGATGTAAGAGGTTTCATATTGCTAATAGCAGCTACAATCCAGC |
| | TACCATTCTGCTTTTATTTTATGGTTGGGATAAGGCTGGATTATTCTGAGTCCAAGCTAGGCCCT |
| | TTTGCTAATCATGTTCATACCTCTTATCTTCCTCCCACAGCTCCTGGGCAACGTGCTGGTCTGTG |
| | TGCTGGCCCATCACTTTGGCAAAGAATTTGAGCGGCCGCCACCATGGCTAGCGCCAGCACAAG |
| | CAAGTACAACAGCCACAGCCTGGAAAACGAGAGCATCAAGCGGACCAGCAGAGATGGCGTGA |
| | ACAGGGATCTGACAGAGGCCGTGCCTAGACTGCCTGGCGAGACACTGATCACCGACAAAGAA |
| | GTGATCTACATCTGCCCCTTCAACGGCCCCATCAAGGGCCGCGTGTACATCACCAACTACCGGC |
| | TGTACCTGCGGTCCCTGGAAACCGATAGCAGCCTGATCCTGGATGTGCCCCTGGGCGTGATCA |
| | GCCGGATTGAGAAAATGGGCGGAGCCACCTCCAGAGGCGAGAATAGCTACGGCCTGGACATC |
| | ACATGCAAGGACATGCGGAACCTGAGATTCGCCCTGAAGCAAGAGGGCCACAGCAGACGGGA |
| | CATGTTCGAGATCCTGACCAGATACGCCTTTCCTCTGGCTCACTCCCTGCCTCTGTTCGCCTTCCT |
| | GAACGAAGAGAAGTTCAACGTGGACGGCTGGACCGTGTACAACCCCGTGGAAGAGTATCGGA |
| | GACAGGGCCTGCCTAATCACCACTGGCGGATCACCTTCATCAACAAGTGCTACGAGCTGTGCG |
| | ACACATACCCCGCACTGCTGGTGGTGCCTTACAGAGCCTCCGACGACGACCTGAGAAGAGTGG |
| | CCACCTTTCGGAGCCGGAACAGAATCCCTGTGCTGAGCTGGATTCACCCCGAGAACAAGACCG |
| | TGATCGTGCGGTGTTCTCAGCCCCTCGTGGGCATGTCTGGCAAGAGAAACAAGGACGACGAG |
| | AAGTACCTGGACGTGATCCGCGAGACAAACAAGCAGATCAGCAAGCTGACCATCTACGACGCC |
| | AGACCTAGCGTGAACGCCGTGGCCAACAAAGCTACAGGCGGCGGATACGAGAGCGACGACG |
| | CCTATCATAACGCCGAGCTGTTCTTTCTGGACATCCACAACATCCACGTGATGCGGGAAAGCCT |
| | GAAGAAAGTGAAGGACATCGTGTACCCCAATGTGGAAGAGAGCCACTGGCTGAGCAGTCTGG |
| | AAAGCACACACTGGCTGGAACACATCAAGCTGGTGCTGACAGGCGCCATCCAGGTGGCCGAT |
| | AAGGTGTCCTCTGGCAAGTCTAGCGTGCTGGTGCACTGTAGCGACGGCTGGGATAGAACAGC |
| | CCAGCTGACATCTCTGGCCATGCTGATGCTGGACAGCTTCTACAGATCCATCGAGGGCTTTGAG |
| | ATCCTGGTGCAGAAAGAGTGGATCAGCTTCGGCCACAAGTTCGCCAGCAGAATCGGACACGG |
| | CGACAAGAACCACACCGACGCCGATAGAAGCCCCATCTTCCTGCAGTTCATCGACTGCGTGTG |
| | GCAGATGTCCAAGCAGTTCCCTACCGCCTTCGAGTTCAACGAGCAGTTCCTGATCATCATCCTG |
| | GACCACCTGTACAGCTGCAGATTCGGCACCTTCCTGTTCAACTGCGAGAGCGCCAGAGAACGG |
| | CAGAAAGTGACCGAGCGGACCGTGTCTCTGTGGTCCCTGATCAACAGCAACAAAGAGAAATTC |
| | AAGAACCCCTTCTACACCAAAGAAATCAACCGGGTGCTGTACCCCGTGGCCAGCATGAGACAT |
| | CTGGAACTGTGGGTCAACTACTACATCCGGTGGAACCCCAGAATCAAGCAGCAGCAGCCCAAT |
| | CCTGTGGAACAGCGGTACATGGAACTGCTGGCCCTGCGGGACGAGTACATCAAGAGACTGGA |
| | AGAACTGCAGCTGGCCAACAGCGCCAAGCTGAGCGATCCTCCTACAAGCCCTAGCAGCCCCAG |
| | CCAGATGATGCCTCACGTGCAGACCCACTTTTAATTAAGATCTTTTTCCCTCTGCCAAAAATTAT |
| | GGGGACATCATGAAGCCCCTTGAGCATCTGACTTCTGGCTAATAAAGGAAATTTATTTTCATTG |
| | CAATAGTGTGTTGGAATTTTTTGTGTCTCTCActcgagGCCTaggaacccctagtgatggagttggccactcc |
| | ctctctgcgcgctcgctcgctcactgaggccgggcgaccaaaggtcgcccgacgcccgggctttgcccgggcggcctcagtg |
| | agcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 183 | ggcctcagtgagcgagcgagcgcgcagagagggagtggccaactccatcactaggggttcctTGCGCAcaggtaccccc |
| (E3) | ctgcccccacagctcctctcctgtgccttgtttcccagccatgcgttctcctctataaatacccgctctggtatttggggttggca |
| | gctgttgctgccagggagatggttgggttgacatgcggctcctgacaaaacacaaacccctggtgtgtgtgggcgtgggtggt |
| | gtgagtagggggatgaatcagggagggggcgggggacccagggggcaggagccacacaaagtctgtgcggggggggag |
| | cgcacatagcaattggaaactggctgcagacatgcttgctgcctgccctggcgaaggattggtaggcttgccgtcacaggac |
| | ccccgctggctgactcaggggcgcaggctcttgcgggggagctggcctcccgcccccacggccacgggccctttcctggcag |
| | gacagcgggatcttgcagctgtcaggggaggggaggcgggggctgatgtcaggagggatacaaatagtgccgacggctgg |
| | gggccctgtctcccctcgccgcatccactctccggccggccgcctgcccgccgcctcctccgtgcgcccgccagcctcgcccgc |
| | gccgtcaccGATATCtcagatogcctggagacgccatccacgctgttttgacctccatagaagacaccgggaccgatcca |
| | gcctccgcggattcgaaTCCCGGCCGGGAACGGTGCATTGGAACGCGGATTCCCCGTGCCAAGAGT |
| | GACGTAAGTACCGCCTATAGAGTCTATAGGCCCACAAAAAATGCTTTCTTCTTTTAATATACTTT |
| | TTTGTTTATCTTATTTCTAATACTTTCCCTAATCTCTTTCTTTCAGGGCAATAATGATACAATGTAT |
| | CATGCCTCTTTGCACCATTCTAAAGAATAACAGTGATAATTTCTGGGTTAAGGCAATAGCAATA |
| | TTTCTGCATATAAATATTTCTGCATATAAATTGTAACTGATGTAAGAGGTTTCATATTGCTAATA |
| | GCAGCTACAATCCAGCTACCATTCTGCTTTTATTTTATGGTTGGGATAAGGCTGGATTATTCTGA |
| | GTCCAAGCTAGGCCCTTTTGCTAATCATGTTCATACCTCTTATCTTCCTCCCACAGCTCCTGGGC |
| | AACGTGCTGGTCTGTGTGCTGGCCCATCACTTTGGCAAAGAATTTGAGCGGCCGCCACCATGG |
| | CTTCTGCATCAACTTCTAAATATAATTCACACTCCTTGGAGAATGAGTCTATTAAGAGGACGTCT |
| | CGAGATGGAGTCAATCGAGATCTCACTGAGGCTGTTCCTCGACTTCCAGGAGAAACACTAATC |
| | ACTGACAAAGAAGTTATTTACATATGTCCTTTCAATGGCCCCATTAAGGGAAGAGTTTACATCA |
| | CAAATTATCGTCTTTATTTAAGAAGTTTGGAAACGGATTCTTCTCTAATACTTGATGTTCCTCTG |
| | GGTGTGATCTCGAGAATTGAAAAAATGGGAGGCGCGACAAGTAGAGGAGAAAATTCCTATGG |
| | TCTAGATATTACTTGTAAAGACATGAGAAACCTGAGGTTCGCTTTGAAACAGGAAGGCCACAG |
| | CAGAAGAGATATGTTTGAGATCCTCACGAGATACGCGTTTCCCCTGGCTCACAGTCTGCCATTA |
| | TTTGCATTTTTAAATGAAGAAAAGTTTAACGTGGATGGATGGACAGTTTACAATCCAGTGGAA |
| | GAATACAGGAGGCAGGGCTTGCCCAATCACCATTGGAGAATAACTTTTATTAATAAGTGCTAT |
| | GAGCTCTGTGACACTTACCCTGCTCTTTTGGTGGTTCCGTATCGTGCCTCAGATGATGACCTCCG |
| | GAGAGTTGCAACTTTTAGGTCCCGAAATCGAATTCCAGTGCTGTCATGGATTCATCCAGAAAAT |
| | AAGACGGTCATTGTGCGTTGCAGTCAGCCTCTTGTCGGTATGAGTGGGAAACGAAATAAAGAT |
| | GATGAGAAATATCTCGATGTTATCAGGGAGACTAATAAACAAATTTCTAAACTCACCATTTATG |
| | ATGCAAGACCCAGCGTAAATGCAGTGGCCAACAAGGCAACAGGAGGAGGATATGAAAGTGAT |
| | GATGCATATCATAACGCCGAACTTTTCTTCTTAGACATTCATAATATTCATGTTATGCGGGAATC |
| | TTTAAAAAAAGTGAAGGACATTGTTTATCCTAATGTAGAAGAATCTCATTGGTTGTCCAGTTTG |
| | GAGTCTACTCATTGGTTAGAACATATCAAGCTCGTTTTGACAGGAGCCATTCAAGTAGCAGACA |
| | AAGTTTCTTCAGGGAAGAGTTCAGTGCTTGTGCATTGCAGTGACGGATGGGACAGGACTGCTC |
| | AGCTGACATCCTTGGCCATGCTGATGTTGGATAGCTTCTATAGGAGCATTGAAGGGTTCGAAA |
| | TACTGGTACAAAAAGAATGGATAAGTTTTGGACATAAATTTGCATCTCGAATAGGTCATGGTG |
| | ATAAAAACCACACCGATGCTGACCGTTCTCCTATTTTTCTCCAGTTTATTGATTGTGTGTGGCAA |
| | ATGTCAAAACAGTTCCCTACAGCTTTTGAATTCAATGAACAATTTTTGATTATAATTTTGGATCA |
| | TCTGTATAGTTGCCGATTTGGTACTTTCTTATTCAACTGTGAATCTGCTCGAGAAAGACAGAAG |
| | GTTACAGAAAGGACTGTTTCTTTATGGTCACTGATAAACAGTAATAAAGAAAAATTCAAAAACC |
| | CATTCTATACTAAAGAAATCAATAGAGTTTTATATCCAGTTGCAAGTATGCGTCACTTGGAACTC |
| | TGGGTGAATTACTACATTAGATGGAACCCCAGGATCAAACAACAACAACCAAATCCAGTGGAA |
| | CAACGTTACATGGAACTCTTAGCCTTACGAGATGAATACATAAAACGGCTTGAGGAACTGCAA |
| | CTAGCAAACTCTGCAAAACTTTCTGATCCCCCAACTTCACCTTCCAGTCCTTCTCAAATGATGCC |
| | ACATGTGCAAACTCACTTTTAATTAAGATCTTTTTCCCTCTGCCAAAAATTATGGGGACATCATG |
| | AAGCCCCTTGAGCATCTGACTTCTGGCTAATAAAGGAAATTTATTTTCATTGCAATAGTGTGTT |
| | GGAATTTTTTGTGTCTCTCActcgagGCCTaggaacccctagtgatggagttggccactccctctctgcgcgctcgc |
| | tcgctcactgaggccgggcgaccaaaggtcgcccgacgcccgggctttgcccgggcggcctcagtgagcgagcgagcgcgc |
| | agagagggagtggccaa |
| SEQ ID | ttggccactccctctctgcgcgctcgctcgctcactgaggccgcccgggcaaagcccgggcgtcgggcgacctttggtcgccc |
| NO: 184 | ggcctcagtgagcgagcgagcgcgcagagagggagtggccaactccatcactaggggttcctTGCGCAcaggtaccccc |
| (E4) | ctgcccccacagctcctctcctgtgccttgtttcccagccatgcgttctcctctataaatacccgctctggtatttggggttggca |
| | gctgttgctgccagggagatggttgggttgacatgcggctcctgacaaaacacaaacccctggtgtgtgtgggcgtgggtggt |
| | gtgagtagggggatgaatcagggagggggcgggggacccagggggcaggagccacacaaagtctgtgcggggggggag |
| | cgcacatagcaattggaaactggctgcagacatgcttgctgcctgccctggcgaaggattggtaggcttgccgtcacaggac |
| | ccccgctggctgactcaggggcgcaggctcttgcgggggagctggcctcccgcccccacggccacgggccctttcctggcag |
| | gacagcgggatcttgcagctgtcaggggaggggaggcgggggctgatgtcaggagggatacaaatagtgccgacggctgg |
| | gggccctgtctcccctcgccgcatccactctccggcoggccgcctgcccgccgcctcctccgtgcgcccgccagcctcgcccgc |
| | gccgtcaccGATATCtcagatcgcctggagacgccatccacgctgttttgacctccatagaagacaccgggaccgatcca |
| | gcctocgcggattcgaaTCCCGGCCGGGAACGGTGCATTGGAACGCGGATTCCCCGTGCCAAGAGT |
| | GACGTAAGTACCGCCTATAGAGTCTATAGGCCCACAAAAAATGCTTTCTTCTTTTAATATACTTT |
| | TTTGTTTATCTTATTTCTAATACTTTCCCTAATCTCTTTCTTTCAGGGCAATAATGATACAATGTAT |
| | CATGCCTCTTTGCACCATTCTAAAGAATAACAGTGATAATTTCTGGGTTAAGGCAATAGCAATA |
| | TTTCTGCATATAAATATTTCTGCATATAAATTGTAACTGATGTAAGAGGTTTCATATTGCTAATA |
| | GCAGCTACAATCCAGCTACCATTCTGCTTTTATTTTATGGTTGGGATAAGGCTGGATTATTCTGA |
| | GTCCAAGCTAGGCCCTTTTGCTAATCATGTTCATACCTCTTATCTTCCTCCCACAGCTCCTGGGC |
| | AACGTGCTGGTCTGTGTGCTGGCCCATCACTTTGGCAAAGAATTTGAGCGGCCGCCACCatggct |
| | tctgcatcaacttctaaatataattcacactccttggagaatgagtctattaagaggacgtctcgagatggagtcaatcgaga |
| | tctcactgaggctgttcctcgacttccaggagaaacactaatcactgacaaagaagttatttacatatgtcctttcaatggccc |
| | cattaagggaagagtttacatcacaaattatcgtctttatttaagaagtttggaaacggattcttctctaatacttgatgttoct |
| | ctgggtgtgatctcgagaattgaaaaaatgggaggcgcgacaagtagaggagaaaattcctatggtctagatattacttgta |
| | aagacatgagaaacctgaggttcgctttgaaacaggaaggccacagcagaagagatatgtttgagatcctcacgagatacg |
| | cgtttcccctggctcacagtctgccattatttgcatttttaaatgaagaaaagtttaacgtggatggatggacagtttacaatcc |
| | agtggaagaatacaggaggcagggcttgcccaatcaccattggagaataacttttattaataagtgctatgagctctgtgac |
| | acttaccctgctcttttggtggttccgtatcgtgcctcagatgatgacctccggagagttgcaacttttaggtcccgaaatcgaa |
| | ttccagtgctgtcatggattcatccagaaaataagacggtcattgtgcgttgcagtcagcctcttgtcggtatgagtgggaaac |
| | gaaataaagatgatgagaaatatctcgatgttatcagggagactaataaacaaatttctaaactcaccatttatgatgcaag |
| | acccagcgtaaatgcagtggccaacaaggcaacaggaggaggatatgaaagtgatgatgcatatcataacgccgaactttt |
| | cttcttagacattcataatattcatgttatgcgggaatctttaaaaaaagtgaaggacattgtttatcctaatgtagaagaatct |
| | cattggttgtccagtttggagtctactcattggttagaacatatcaagctcgttttgacaggagccattcaagtagcagacaaa |
| | gtttcttcagggaagagttcagtgcttgtgcattgcagtgacggatgggacaggactgctcagctgacatccttggccatgct |
| | gatgttggatagcttctataggagcattgaagggttcgaaatactggtacaaaaagaatggataagttttggacataaatttg |
| | catctcgaataggtcatggtgataaaaaccacaccgatgctgaccgttctcctatttttctccagtttattgattgtgtgtggca |
| | aatgtcaaaacagttccctacagcttttgaattcaatgaacaatttttgattataattttggatcatctgtatagttgccgatttg |
| | gtactttcttattcaactgtgaatctgctcgagaaagacagaaggttacagaaaggactgtttctttatggtcactgataaaca |
| | gtaataaagaaaaattcaaaaaccccttctatactaaagaaatcaatcgagttttatatccagttgccagtatgcgtcacttg |
| | gaactctgggtgaattactacattagatggaaccccaggatcaagcaacaacagccgaatccagtggagcagcgttacatg |
| | gagctcttagccttacgcgacgaatacataaagcggcttgaggaactgcagctcgccaactctgccaagctttctgatccccc |
| | aacttcaccttccagtccttcgcaaatgatgccccatgtgcaaactcacttttaaTTAAGATCTTTTTCCCTCTGCCA |
| | AAAATTATGGGGACATCATGAAGCCCCTTGAGCATCTGACTTCTGGCTAATAAAGGAAATTTAT |
| | TTTCATTGCAATAGTGTGTTGGAATTTTTTGTGTCTCTCActcgagGCCTaggaacccctagtgatggagt |
| | tggccactccctctctgcgcgctcgctcgctcactgaggccgggcgaccaaaggtcgcccgacgcccgggctttgcccgggc |
| | ggcctcagtgagcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 185 | ggcctcagtgagcgagcgagcgcgcagagagggagtggccaactccatcactaggggttcctTGCGCAcaggtaccccc |
| (E5) | ctgcccccacagctcctctcctgtgccttgtttcccagccatgcgttctcctctataaatacccgctctggtatttggggttggca |
| | gctgttgctgccagggagatggttgggttgacatgcggctcctgacaaaacacaaacccctggtgtgtgtgggcgtgggtggt |
| | gtgagtagggggatgaatcagggagggggcgggggacccagggggcaggagccacacaaagtctgtgcggggggggag |
| | cgcacatagcaattggaaactggctgcagacatgcttgctgcctgccctggcgaaggattggtaggcttgccgtcacaggac |
| | ccccgctggctgactcaggggcgcaggctcttgcgggggagctggcctcccgcccccacggccacgggccctttcctggcag |
| | gacagcgggatcttgcagctgtcaggggaggggaggcgggggctgatgtcaggagggatacaaatagtgccgacggctgg |
| | gggccctgtctcccctcgccgcatccactctccggcoggccgcctgcccgccgcctcctccgtgcgcccgccagcctcgcccgc |
| | gccgtcaccGATATCtcagatcgcctggagacgccatccacgctgttttgacctccatagaagacaccgggaccgatcca |
| | gcctccgcggattcgaaTCCCGGCCGGGAACGGTGCATTGGAACGCGGATTCCCCGTGCCAAGAGT |
| | GACGTAAGTACCGCCTATAGAGTCTATAGGCCCACAAAAAATGCTTTCTTCTTTTAATATACTTT |
| | TTTGTTTATCTTATTTCTAATACTTTCCCTAATCTCTTTCTTTCAGGGCAATAATGATACAATGTAT |
| | CATGCCTCTTTGCACCATTCTAAAGAATAACAGTGATAATTTCTGGGTTAAGGCAATAGCAATA |
| | TTTCTGCATATAAATATTTCTGCATATAAATTGTAACTGATGTAAGAGGTTTCATATTGCTAATA |
| | GCAGCTACAATCCAGCTACCATTCTGCTTTTATTTTATGGTTGGGATAAGGCTGGATTATTCTGA |
| | GTCCAAGCTAGGCCCTTTTGCTAATCATGTTCATACCTCTTATCTTCCTCCCACAGCTCCTGGGC |
| | AACGTGCTGGTCTGTGTGCTGGCCCATCACTTTGGCAAAGAATTTGAGCGGCCGCCACCATGG |
| | CCTCTGCCAGCACCTCTAAGTACAACAGCCACTCCCTGGAAAATGAATCCATCAAAAGGACCAG |
| | CAGAGATGGAGTGAACAGAGACCTAACTGAAGCTGTGCCAAGACTGCCTGGAGAGACCCTGA |
| | TCACAGACAAGGAAGTGATCTACATCTGCCCCTTCAATGGCCCTATCAAGGGAAGGGTGTACA |
| | TCACCAACTACAGGCTTTACCTGAGATCCCTGGAGACAGACAGCAGCCTGATCCTGGATGTGC |
| | CTCTGGGAGTGATCAGCAGAATAGAGAAGATGGGGGGTGCCACCAGCAGAGGAGAGAACAG |
| | CTATGGCCTGGACATCACCTGCAAGGACATGAGAAACCTGAGATTTGCCCTGAAGCAGGAGG |
| | GCCACAGCAGAAGAGACATGTTTGAAATCCTGACCAGGTATGCCTTCCCCCTGGCCCACTCTCT |
| | CCCCCTGTTTGCCTTCCTGAATGAGGAAAAGTTCAATGTTGATGGCTGGACAGTGTACAACCCA |
| | GTGGAGGAGTACAGAAGACAGGGCCTGCCTAACCACCACTGGAGGATCACCTTCATCAACAA |
| | GTGCTATGAACTGTGTGACACATACCCTGCCCTGCTGGTGGTGCCTTACAGAGCCTCAGATGAT |
| | GACCTGAGAAGAGTTGCCACCTTCAGAAGCAGGAACAGAATCCCTGTACTGAGCTGGATCCAC |
| | CCTGAGAATAAGACTGTGATTGTGAGGTGCAGCCAGCCCCTGGTGGGCATGAGTGGCAAGAG |
| | AAACAAAGATGATGAAAAGTACCTGGATGTGATCAGAGAGACCAACAAACAGATCAGCAAGC |
| | TCACCATCTATGATGCTAGACCCTCTGTTAATGCTGTGGCCAACAAGGCCACAGGGGGAGGCT |
| | ATGAATCTGATGATGCTTACCACAATGCTGAGCTGTTCTTCCTGGACATCCACAACATCCATGT |
| | GATGAGAGAATCCCTCAAGAAAGTGAAGGACATTGTGTACCCTAATGTGGAAGAAAGTCACT |
| | GGCTGAGCAGCTTGGAGTCCACCCACTGGCTGGAGCACATCAAGCTGGTCCTGACAGGAGCC |
| | ATCCAGGTGGCTGACAAGGTGAGTTCTGGCAAGTCCTCAGTGCTGGTCCACTGCTCTGATGGC |
| | TGGGACAGAACTGCCCAGCTGACCAGTCTGGCCATGCTGATGCTGGACTCCTTCTACAGAAGC |
| | ATTGAAGGCTTTGAAATCCTGGTGCAAAAGGAATGGATCTCTTTTGGCCACAAGTTTGCCAGCA |
| | GAATTGGCCATGGTGACAAAAACCACACAGATGCTGACAGAAGCCCTATCTTCCTGCAGTTCAT |
| | TGACTGTGTGTGGCAGATGAGCAAGCAGTTCCCCACAGCATTTGAGTTCAATGAGCAGTTCCT |
| | AATAATCATCCTGGACCACCTCTACAGCTGCAGATTTGGCACCTTCCTGTTCAACTGTGAGTCTG |
| | CCAGAGAAAGACAGAAGGTGACAGAGAGGACAGTGAGCCTGTGGAGCCTGATCAACTCCAAC |
| | AAGGAGAAGTTCAAGAACCCCTTCTACACCAAGGAAATCAACAGGGTGCTGTACCCTGTGGCT |
| | AGCATGAGGCACCTGGAGCTGTGGGTCAACTACTACATCAGATGGAACCCTAGAATCAAACAG |
| | CAACAACCCAACCCTGTGGAGCAGAGGTACATGGAGTTACTGGCCCTGAGGGATGAGTACATC |
| | AAGAGACTGGAGGAGCTGCAGCTGGCCAACTCTGCCAAACTGTCTGACCCTCCTACCTCCCCCA |
| | GCTCCCCCTCTCAGATGATGCCACATGTGCAGACCCACTTTTAATTAAGATCTTTTTCCCTCTGC |
| | CAAAAATTATGGGGACATCATGAAGCCCCTTGAGCATCTGACTTCTGGCTAATAAAGGAAATTT |
| | ATTTTCATTGCAATAGTGTGTTGGAATTTTTTGTGTCTCTCActcgagGCCTaggaacccctagtgatgg |
| | agttggccactccctctctgcgcgctcgctcgctcactgaggccgggcgaccaaaggtcgcccgacgcccgggctttgcccgg |
| | gcggcctcagtgagcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 186 | ggcctcagtgagcgagcgagcgcgcagagagggagtggccaactccatcactaggggttcctTGCGCAcaggtaccgac |
| (E6) | attgattattgactagttattaatagtaatcaattacggggtcattagttcatagcccatatatggagttccgcgttacataact |
| | tacggtaaatggcccgcctggctgaccgcccaacgacccccgcccattgacgtcaataatgacgtatgttcccatagtaacgc |
| | caatagggactttccattgacgtcaatgggtggagtatttacggtaaactgcccacttggcagtacatcaagtgtatcatatgc |
| | caagtacgccccctattgacgtcaatgacggtaaatggcccgcctggcattatgcccagtacatgaccttatgggactttccta |
| | cttggcagtacatctacgtattagtcatcgctattaccatggtcgaggtgagccccacgttctgcttcactctccccatctcccc |
| | cccctccccacccccaattttgtatttatttattttttaattattttgtgcagcgatgggggcggggggggggggggggcgcgcg |
| | ccaggcggggcggggggggcgaggggggggcggggcgaggcggagaggtgcggcggcagccaatcagagcggcgcg |
| | ctccgaaagtttccttttatggcgaggcggcggcggcggcggccctataaaaagcgaagcgcgcggcgggcgGATATCtc |
| | agatcgcctggagacgccatccacgctgttttgacctccatagaagacaccgggaccgatccagcctccgoggattcgaaTC |
| | CCGGCCGGGAACGGTGCATTGGAACGCGGATTCCCCGTGCCAAGAGTGACGTAAGTACCGCC |
| | TATAGAGTCTATAGGCCCACAAAAAATGCTTTCTTCTTTTAATATACTTTTTTGTTTATCTTATTTC |
| | TAATACTTTCCCTAATCTCTTTCTTTCAGGGCAATAATGATACAATGTATCATGCCTCTTTGCACC |
| | ATTCTAAAGAATAACAGTGATAATTTCTGGGTTAAGGCAATAGCAATATTTCTGCATATAAATA |
| | TTTCTGCATATAAATTGTAACTGATGTAAGAGGTTTCATATTGCTAATAGCAGCTACAATCCAGC |
| | TACCATTCTGCTTTTATTTTATGGTTGGGATAAGGCTGGATTATTCTGAGTCCAAGCTAGGCCCT |
| | TTTGCTAATCATGTTCATACCTCTTATCTTCCTCCCACAGCTCCTGGGCAACGTGCTGGTCTGTG |
| | TGCTGGCCCATCACTTTGGCAAAGAATTTGAGCGGCCGCCACCATGGCATCAGCAAGCACAAG |
| | CAAATACAACTCACATAGCCTGGAAAACGAGAGCATCAAGAGAACCTCAAGGGACGGGGTCA |
| | ATAGAGATCTGACAGAGGCAGTGCCAAGGCTGCCTGGAGAGACACTGATCACAGACAAGGAA |
| | GTGATCTACATCTGCCCTTTCAACGGCCCAATCAAGGGCCGCGTGTATATCACCAATTACAGAC |
| | TGTACCTGCGGAGCCTGGAGACAGATAGCTCCCTGATCCTGGACGTGCCTCTGGGCGTGATCA |
| | GCAGAATCGAGAAGATGGGAGGAGCCACCTCTAGGGGAGAGAACAGCTACGGCCTGGATAT |
| | CACCTGTAAGGACATGCGCAATCTGCGGTTTGCCCTGAAGCAGGAGGGCCACTCCCGGAGAG |
| | ATATGTTCGAGATCCTGACCAGATATGCCTTTCCCCTGGCCCACTCTCTGCCTCTGTTCGCCTTTC |
| | TGAACGAGGAGAAGTTCAATGTGGACGGCTGGACAGTGTACAACCCCGTGGAGGAGTATAGG |
| | CGCCAGGGCCTGCCTAACCACCACTGGCGGATCACCTTTATCAATAAGTGCTACGAGCTGTGC |
| | GATACATATCCAGCCCTGCTGGTGGTGCCCTACAGAGCCAGCGACGATGACCTGCGGAGAGTG |
| | GCCACCTTCCGGAGCCGGAACAGAATCCCTGTGCTGAGCTGGATCCACCCAGAGAATAAGACA |
| | GTGATCGTGCGCTGCTCCCAGCCACTGGTGGGAATGTCTGGCAAGCGGAACAAGGATGACGA |
| | GAAGTACCTGGATGTGATCAGAGAGACAAATAAGCAGATCAGCAAGCTGACAATCTATGATG |
| | CCCGGCCTTCTGTGAATGCAGTGGCAAATAAGGCAACAGGAGGAGGATATGAGTCTGATGAC |
| | GCCTATCACAACGCCGAGCTGTTCTTTCTGGATATCCACAATATCCACGTGATGAGAGAGTCCC |
| | TGAAGAAGGTGAAGGACATCGTGTACCCAAACGTGGAGGAGTCCCACTGGCTGTCTAGCCTG |
| | GAGTCTACCCACTGGCTGGAGCACATCAAGCTGGTGCTGACAGGCGCCATCCAGGTGGCCGAT |
| | AAGGTGTCCTCTGGCAAGAGCTCCGTGCTGGTGCACTGTTCCGATGGGTGGGACAGAACAGC |
| | ACAGCTGACAAGCCTGGCtATGCTGATGCTGGACTCCTTCTATAGATCTATCGAGGGCTTTGAG |
| | ATCCTGGTGCAGAAGGAGTGGATCAGCTTCGGCCACAAGTTTGCCTCCAGGATCGGCCACGGC |
| | GATAAGAATCACACCGATGCCGACCGCAGCCCTATCTTCCTGCAGTTTATCGACTGCGTGTGGC |
| | AGATGTCCAAGCAGTTCCCAACCGCCTTCGAGTTTAACGAGCAGTTTCTGATCATCATCCTGGA |
| | CCACCTGTACTCCTGCAGGTTCGGCACATTCCTGTTTAATTGTGAGTCTGCCAGAGAGAGGCAG |
| | AAGGTGACCGAGCGCACAGTGTCTCTGTGGAGCCTGATCAACAGCAATAAGGAGAAGTTCAA |
| | GAACCCCTTCTACACAAAGGAGATCAATCGCGTGCTGTATCCTGTGGCATCCATGAGACACCTG |
| | GAGCTGTGGGTGAACTACTATATCCGCTGGAATCCCCGGATCAAGCAGCAGCAGCCCAACCCT |
| | GTGGAGCAGCGGTACATGGAGCTGCTGGCCCTGAGAGATGAGTATATCAAGAGGCTGGAGG |
| | AGCTGCAGCTGGCCAATAGCGCCAAGCTGTCCGACCCACCTACTTCACCAAGCAGCCCCTCACA |
| | GATGATGCCCCACGTCCAGACCCACTTTTAATTAAGATCTTTTTCCCTCTGCCAAAAATTATGGG |
| | GACATCATGAAGCCCCTTGAGCATCTGACTTCTGGCTAATAAAGGAAATTTATTTTCATTGCAAT |
| | AGTGTGTTGGAATTTTTTGTGTCTCTCActcgagGCCTaggaacccctagtgatggagttggccactccctctct |
| | gcgcgctcgctcgctcactgaggccgggcgaccaaaggtcgcccgacgcccgggctttgcccgggcggcctcagtgagcga |
| | gcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 187 | ggcctcagtgagcgagcgagcgcgcagagagggagtggccaactccatcactaggggttcctTGCGCAcaggtaccgac |
| (E7) | attgattattgactagttattaatagtaatcaattacggggtcattagttcatagcccatatatggagttccgcgttacataact |
| | tacggtaaatggcccgcctggctgaccgcccaacgacccccgcccattgacgtcaataatgacgtatgttcccatagtaacgc |
| | caatagggactttccattgacgtcaatgggtggagtatttacggtaaactgcccacttggcagtacatcaagtgtatcatatgc |
| | caagtacgccccctattgacgtcaatgacggtaaatggcccgcctggcattatgcccagtacatgaccttatgggactttccta |
| | cttggcagtacatctacgtattagtcatcgctattaccatggtcgaggtgagccccacgttctgcttcactctccccatctcccc |
| | cccctccccacccccaattttgtatttatttattttttaattattttgtgcagcgatgggggcggggggggggggggggcgcgcg |
| | ccaggcggggcggggcggggcgaggggcggggcggggcgaggcggagaggtgcggcggcagccaatcagagcggcgcg |
| | ctccgaaagtttccttttatggcgaggcggcggcggcggcggccctataaaaagcgaagcgcgcggcgggcgGATATCtc |
| | agatcgcctggagacgccatccacgctgttttgacctccatagaagacaccgggaccgatccagcctccgcggattcgaaTC |
| | CCGGCCGGGAACGGTGCATTGGAACGCGGATTCCCCGTGCCAAGAGTGACGTAAGTACCGCC |
| | TATAGAGTCTATAGGCCCACAAAAAATGCTTTCTTCTTTTAATATACTTTTTTGTTTATCTTATTTC |
| | TAATACTTTCCCTAATCTCTTTCTTTCAGGGCAATAATGATACAATGTATCATGCCTCTTTGCACC |
| | ATTCTAAAGAATAACAGTGATAATTTCTGGGTTAAGGCAATAGCAATATTTCTGCATATAAATA |
| | TTTCTGCATATAAATTGTAACTGATGTAAGAGGTTTCATATTGCTAATAGCAGCTACAATCCAGC |
| | TACCATTCTGCTTTTATTTTATGGTTGGGATAAGGCTGGATTATTCTGAGTCCAAGCTAGGCCCT |
| | TTTGCTAATCATGTTCATACCTCTTATCTTCCTCCCACAGCTCCTGGGCAACGTGCTGGTCTGTG |
| | TGCTGGCCCATCACTTTGGCAAAGAATTTGAGCGGCCGCCACCATGGCTAGCGCCAGCACAAG |
| | CAAGTACAACAGCCACAGCCTGGAAAACGAGAGCATCAAGCGGACCAGCAGAGATGGCGTGA |
| | ACAGGGATCTGACAGAGGCCGTGCCTAGACTGCCTGGCGAGACACTGATCACCGACAAAGAA |
| | GTGATCTACATCTGCCCCTTCAACGGCCCCATCAAGGGCCGCGTGTACATCACCAACTACCGGC |
| | TGTACCTGCGGTCCCTGGAAACCGATAGCAGCCTGATCCTGGATGTGCCCCTGGGCGTGATCA |
| | GCCGGATTGAGAAAATGGGCGGAGCCACCTCCAGAGGCGAGAATAGCTACGGCCTGGACATC |
| | ACATGCAAGGACATGCGGAACCTGAGATTCGCCCTGAAGCAAGAGGGCCACAGCAGACGGGA |
| | CATGTTCGAGATCCTGACCAGATACGCCTTTCCTCTGGCTCACTCCCTGCCTCTGTTCGCCTTCCT |
| | GAACGAAGAGAAGTTCAACGTGGACGGCTGGACCGTGTACAACCCCGTGGAAGAGTATCGGA |
| | GACAGGGCCTGCCTAATCACCACTGGCGGATCACCTTCATCAACAAGTGCTACGAGCTGTGCG |
| | ACACATACCCCGCACTGCTGGTGGTGCCTTACAGAGCCTCCGACGACGACCTGAGAAGAGTGG |
| | CCACCTTTCGGAGCCGGAACAGAATCCCTGTGCTGAGCTGGATTCACCCCGAGAACAAGACCG |
| | TGATCGTGCGGTGTTCTCAGCCCCTCGTGGGCATGTCTGGCAAGAGAAACAAGGACGACGAG |
| | AAGTACCTGGACGTGATCCGCGAGACAAACAAGCAGATCAGCAAGCTGACCATCTACGACGCC |
| | AGACCTAGCGTGAACGCCGTGGCCAACAAAGCTACAGGCGGCGGATACGAGAGCGACGACG |
| | CCTATCATAACGCCGAGCTGTTCTTTCTGGACATCCACAACATCCACGTGATGCGGGAAAGCCT |
| | GAAGAAAGTGAAGGACATCGTGTACCCCAATGTGGAAGAGAGCCACTGGCTGAGCAGTCTGG |
| | AAAGCACACACTGGCTGGAACACATCAAGCTGGTGCTGACAGGCGCCATCCAGGTGGCCGAT |
| | AAGGTGTCCTCTGGCAAGTCTAGCGTGCTGGTGCACTGTAGCGACGGCTGGGATAGAACAGC |
| | CCAGCTGACATCTCTGGCCATGCTGATGCTGGACAGCTTCTACAGATCCATCGAGGGCTTTGAG |
| | ATCCTGGTGCAGAAAGAGTGGATCAGCTTCGGCCACAAGTTCGCCAGCAGAATCGGACACGG |
| | CGACAAGAACCACACCGACGCCGATAGAAGCCCCATCTTCCTGCAGTTCATCGACTGCGTGTG |
| | GCAGATGTCCAAGCAGTTCCCTACCGCCTTCGAGTTCAACGAGCAGTTCCTGATCATCATCCTG |
| | GACCACCTGTACAGCTGCAGATTCGGCACCTTCCTGTTCAACTGCGAGAGCGCCAGAGAACGG |
| | CAGAAAGTGACCGAGCGGACCGTGTCTCTGTGGTCCCTGATCAACAGCAACAAAGAGAAATTC |
| | AAGAACCCCTTCTACACCAAAGAAATCAACCGGGTGCTGTACCCCGTGGCCAGCATGAGACAT |
| | CTGGAACTGTGGGTCAACTACTACATCCGGTGGAACCCCAGAATCAAGCAGCAGCAGCCCAAT |
| | CCTGTGGAACAGCGGTACATGGAACTGCTGGCCCTGCGGGACGAGTACATCAAGAGACTGGA |
| | AGAACTGCAGCTGGCCAACAGCGCCAAGCTGAGCGATCCTCCTACAAGCCCTAGCAGCCCCAG |
| | CCAGATGATGCCTCACGTGCAGACCCACTTTTAATTAAGATCTTTTTCCCTCTGCCAAAAATTAT |
| | GGGGACATCATGAAGCCCCTTGAGCATCTGACTTCTGGCTAATAAAGGAAATTTATTTTCATTG |
| | CAATAGTGTGTTGGAATTTTTTGTGTCTCTCActcgagGCCTaggaacccctagtgatggagttggccactcc |
| | ctctctgcgcgctcgctcgctcactgaggccgggcgaccaaaggtcgcccgacgcccgggctttgcccgggcggcctcagtg |
| | agcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 188 | ggcctcagtgagcgagcgagcgcgcagagagggagtggccaactccatcactaggggttcctTGCGCAcaggtaccgac |
| (E8) | attgattattgactagttattaatagtaatcaattacggggtcattagttcatagcccatatatggagttccgcgttacataact |
| | tacggtaaatggcccgcctggctgaccgcccaacgacccccgcccattgacgtcaataatgacgtatgttcccatagtaacgc |
| | caatagggactttccattgacgtcaatgggtggagtatttacggtaaactgcccacttggcagtacatcaagtgtatcatatgc |
| | caagtacgccccctattgacgtcaatgacggtaaatggcccgcctggcattatgcccagtacatgaccttatgggactttccta |
| | cttggcagtacatctacgtattagtcatcgctattaccatggtcgaggtgagccccacgttctgcttcactctccccatctcccc |
| | cccctccccacccccaattttgtatttatttattttttaattattttgtgcagcgatgggggcggggggggggggggggcgcg |
| | ccaggcggggcggggcggggcgaggggcggggggggcgaggcggagaggtgcggcggcagccaatcagagcggcgcg |
| | ctccgaaagtttccttttatggcgaggcggcggcggcggcggccctataaaaagcgaagcgcgcggcgggcgGATATCtc |
| | agatcgcctggagacgccatccacgctgttttgacctccatagaagacaccgggaccgatccagcctccgcggattcgaaTC |
| | CCGGCCGGGAACGGTGCATTGGAACGCGGATTCCCCGTGCCAAGAGTGACGTAAGTACCGCC |
| | TATAGAGTCTATAGGCCCACAAAAAATGCTTTCTTCTTTTAATATACTTTTTTGTTTATCTTATTTC |
| | TAATACTTTCCCTAATCTCTTTCTTTCAGGGCAATAATGATACAATGTATCATGCCTCTTTGCACC |
| | ATTCTAAAGAATAACAGTGATAATTTCTGGGTTAAGGCAATAGCAATATTTCTGCATATAAATA |
| | TTTCTGCATATAAATTGTAACTGATGTAAGAGGTTTCATATTGCTAATAGCAGCTACAATCCAGC |
| | TACCATTCTGCTTTTATTTTATGGTTGGGATAAGGCTGGATTATTCTGAGTCCAAGCTAGGCCCT |
| | TTTGCTAATCATGTTCATACCTCTTATCTTCCTCCCACAGCTCCTGGGCAACGTGCTGGTCTGTG |
| | TGCTGGCCCATCACTTTGGCAAAGAATTTGAGCGGCCGCCACCATGGCTTCTGCATCAACTTCT |
| | AAATATAATTCACACTCCTTGGAGAATGAGTCTATTAAGAGGACGTCTCGAGATGGAGTCAAT |
| | CGAGATCTCACTGAGGCTGTTCCTCGACTTCCAGGAGAAACACTAATCACTGACAAAGAAGTT |
| | ATTTACATATGTCCTTTCAATGGCCCCATTAAGGGAAGAGTTTACATCACAAATTATCGTCTTTA |
| | TTTAAGAAGTTTGGAAACGGATTCTTCTCTAATACTTGATGTTCCTCTGGGTGTGATCTCGAGA |
| | ATTGAAAAAATGGGAGGCGCGACAAGTAGAGGAGAAAATTCCTATGGTCTAGATATTACTTGT |
| | AAAGACATGAGAAACCTGAGGTTCGCTTTGAAACAGGAAGGCCACAGCAGAAGAGATATGTT |
| | TGAGATCCTCACGAGATACGCGTTTCCCCTGGCTCACAGTCTGCCATTATTTGCATTTTTAAATG |
| | AAGAAAAGTTTAACGTGGATGGATGGACAGTTTACAATCCAGTGGAAGAATACAGGAGGCAG |
| | GGCTTGCCCAATCACCATTGGAGAATAACTTTTATTAATAAGTGCTATGAGCTCTGTGACACTT |
| | ACCCTGCTCTTTTGGTGGTTCCGTATCGTGCCTCAGATGATGACCTCCGGAGAGTTGCAACTTTT |
| | AGGTCCCGAAATCGAATTCCAGTGCTGTCATGGATTCATCCAGAAAATAAGACGGTCATTGTG |
| | CGTTGCAGTCAGCCTCTTGTCGGTATGAGTGGGAAACGAAATAAAGATGATGAGAAATATCTC |
| | GATGTTATCAGGGAGACTAATAAACAAATTTCTAAACTCACCATTTATGATGCAAGACCCAGCG |
| | TAAATGCAGTGGCCAACAAGGCAACAGGAGGAGGATATGAAAGTGATGATGCATATCATAAC |
| | GCCGAACTTTTCTTCTTAGACATTCATAATATTCATGTTATGCGGGAATCTTTAAAAAAAGTGAA |
| | GGACATTGTTTATCCTAATGTAGAAGAATCTCATTGGTTGTCCAGTTTGGAGTCTACTCATTGGT |
| | TAGAACATATCAAGCTCGTTTTGACAGGAGCCATTCAAGTAGCAGACAAAGTTTCTTCAGGGA |
| | AGAGTTCAGTGCTTGTGCATTGCAGTGACGGATGGGACAGGACTGCTCAGCTGACATCCTTGG |
| | CCATGCTGATGTTGGATAGCTTCTATAGGAGCATTGAAGGGTTCGAAATACTGGTACAAAAAG |
| | AATGGATAAGTTTTGGACATAAATTTGCATCTCGAATAGGTCATGGTGATAAAAACCACACCG |
| | ATGCTGACCGTTCTCCTATTTTTCTCCAGTTTATTGATTGTGTGTGGCAAATGTCAAAACAGTTC |
| | CCTACAGCTTTTGAATTCAATGAACAATTTTTGATTATAATTTTGGATCATCTGTATAGTTGCCG |
| | ATTTGGTACTTTCTTATTCAACTGTGAATCTGCTCGAGAAAGACAGAAGGTTACAGAAAGGACT |
| | GTTTCTTTATGGTCACTGATAAACAGTAATAAAGAAAAATTCAAAAACCCATTCTATACTAAAG |
| | AAATCAATAGAGTTTTATATCCAGTTGCAAGTATGCGTCACTTGGAACTCTGGGTGAATTACTA |
| | CATTAGATGGAACCCCAGGATCAAACAACAACAACCAAATCCAGTGGAACAACGTTACATGGA |
| | ACTCTTAGCCTTACGAGATGAATACATAAAACGGCTTGAGGAACTGCAACTAGCAAACTCTGC |
| | AAAACTTTCTGATCCCCCAACTTCACCTTCCAGTCCTTCTCAAATGATGCCACATGTGCAAACTC |
| | ACTTTTAATTAAGATCTTTTTCCCTCTGCCAAAAATTATGGGGACATCATGAAGCCCCTTGAGCA |
| | TCTGACTTCTGGCTAATAAAGGAAATTTATTTTCATTGCAATAGTGTGTTGGAATTTTTTGTGTC |
| | TCTCActcgagGCCTaggaacccctagtgatggagttggccactccctctctgcgcgctcgctcgctcactgaggccgggc |
| | gaccaaaggtcgcccgacgcccgggctttgcccgggcggcctcagtgagcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 189 | ggcctcagtgagcgagcgagcgcgcagagagggagtggccaactccatcactaggggttcctTGCGCAcaggtaccgac |
| (E9) | attgattattgactagttattaatagtaatcaattacggggtcattagttcatagcccatatatggagttccgcgttacataact |
| | tacggtaaatggcccgcctggctgaccgcccaacgacccccgcccattgacgtcaataatgacgtatgttcccatagtaacgc |
| | caatagggactttccattgacgtcaatgggtggagtatttacggtaaactgcccacttggcagtacatcaagtgtatcatatgc |
| | caagtacgccccctattgacgtcaatgacggtaaatggcccgcctggcattatgcccagtacatgaccttatgggactttccta |
| | cttggcagtacatctacgtattagtcatcgctattaccatggtcgaggtgagccccacgttctgcttcactctccccatctcccc |
| | cccctccccacccccaattttgtatttatttattttttaattattttgtgcagcgatgggggcggggggggggggggggcgcgcg |
| | ccaggcggggcggggcggggcgaggggcggggcggggcgaggcggagaggtgcggcggcagccaatcagagcggcgcg |
| | ctccgaaagtttccttttatggcgaggcggcggcggcggcggccctataaaaagcgaagcgcgcggcgggcgGATATCtc |
| | agatcgcctggagacgccatccacgctgttttgacctccatagaagacaccgggaccgatccagcctccgcggattcgaaTC |
| | CCGGCCGGGAACGGTGCATTGGAACGCGGATTCCCCGTGCCAAGAGTGACGTAAGTACCGCC |
| | TATAGAGTCTATAGGCCCACAAAAAATGCTTTCTTCTTTTAATATACTTTTTTGTTTATCTTATTTC |
| | TAATACTTTCCCTAATCTCTTTCTTTCAGGGCAATAATGATACAATGTATCATGCCTCTTTGCACC |
| | ATTCTAAAGAATAACAGTGATAATTTCTGGGTTAAGGCAATAGCAATATTTCTGCATATAAATA |
| | TTTCTGCATATAAATTGTAACTGATGTAAGAGGTTTCATATTGCTAATAGCAGCTACAATCCAGC |
| | TACCATTCTGCTTTTATTTTATGGTTGGGATAAGGCTGGATTATTCTGAGTCCAAGCTAGGCCCT |
| | TTTGCTAATCATGTTCATACCTCTTATCTTCCTCCCACAGCTCCTGGGCAACGTGCTGGTCTGTG |
| | TGCTGGCCCATCACTTTGGCAAAGAATTTGAGCGGCCGCCACCatggcttctgcatcaacttctaaatata |
| | attcacactccttggagaatgagtctattaagaggacgtctcgagatggagtcaatcgagatctcactgaggctgttcctcga |
| | cttccaggagaaacactaatcactgacaaagaagttatttacatatgtcctttcaatggccccattaagggaagagtttacat |
| | cacaaattatcgtctttatttaagaagtttggaaacggattcttctctaatacttgatgttcctctgggtgtgatctcgagaattg |
| | aaaaaatgggaggcgcgacaagtagaggagaaaattcctatggtctagatattacttgtaaagacatgagaaacctgaggt |
| | tcgctttgaaacaggaaggccacagcagaagagatatgtttgagatcctcacgagatacgcgtttcccctggctcacagtctg |
| | ccattatttgcatttttaaatgaagaaaagtttaacgtggatggatggacagtttacaatccagtggaagaatacaggaggc |
| | agggcttgcccaatcaccattggagaataacttttattaataagtgctatgagctctgtgacacttaccctgctcttttggtggt |
| | tccgtatcgtgcctcagatgatgacctccggagagttgcaacttttaggtcccgaaatcgaattccagtgctgtcatggattca |
| | tccagaaaataagacggtcattgtgcgttgcagtcagcctcttgtcggtatgagtgggaaacgaaataaagatgatgagaaa |
| | tatctcgatgttatcagggagactaataaacaaatttctaaactcaccatttatgatgcaagacccagcgtaaatgcagtggc |
| | caacaaggcaacaggaggaggatatgaaagtgatgatgcatatcataacgccgaacttttcttcttagacattcataatattc |
| | atgttatgcgggaatctttaaaaaaagtgaaggacattgtttatcctaatgtagaagaatctcattggttgtccagtttggagt |
| | ctactcattggttagaacatatcaagctcgttttgacaggagccattcaagtagcagacaaagtttcttcagggaagagttca |
| | gtgcttgtgcattgcagtgacggatgggacaggactgctcagctgacatccttggccatgctgatgttggatagcttctatagg |
| | agcattgaagggttcgaaatactggtacaaaaagaatggataagttttggacataaatttgcatctcgaataggtcatggtg |
| | ataaaaaccacaccgatgctgaccgttctcctatttttctccagtttattgattgtgtgtggcaaatgtcaaaacagttccctac |
| | agcttttgaattcaatgaacaatttttgattataattttggatcatctgtatagttgccgatttggtactttcttattcaactgtga |
| | atctgctcgagaaagacagaaggttacagaaaggactgtttctttatggtcactgataaacagtaataaagaaaaattcaaa |
| | aaccccttctatactaaagaaatcaatcgagttttatatccagttgccagtatgcgtcacttggaactctgggtgaattactac |
| | attagatggaaccccaggatcaagcaacaacagccgaatccagtggagcagcgttacatggagctcttagccttacgcgac |
| | gaatacataaagcggcttgaggaactgcagctcgccaactctgccaagctttctgatcccccaacttcaccttccagtccttcg |
| | caaatgatgccccatgtgcaaactcacttttaaTTAAGATCTTTTTCCCTCTGCCAAAAATTATGGGGACAT |
| | CATGAAGCCCCTTGAGCATCTGACTTCTGGCTAATAAAGGAAATTTATTTTCATTGCAATAGTG |
| | TGTTGGAATTTTTTGTGTCTCTCActcgagGCCTaggaacccctagtgatggagttggccactccctctctgcgcg |
| | ctcgctcgctcactgaggccgggcgaccaaaggtcgcccgacgcccgggctttgcccgggcggcctcagtgagcgagcgag |
| | cgcgcagagagggagtggccaa |
| SEQ ID | ttgtcGACTCGGTTCGCATATTAAGGTGACGCGTGTGGCCTCGAACACCGAGCGACCCTGCAGC |
| NO: 190 | GACCCGCTTAA |
| (E10A) | |
| SEQ ID | ccactccctctctgcgcgctcgctcgctcactgaggccgggcgaccaaaggtcgcccgacgcccgggctttgcccgggcggc |
| NO: 191 | ctcagtgagcgagcgagcgcgcagagagggagtggccaa |
| (E10B) | |
| SEQ ID | TTTTTTGGTACCTTCGCATATTAAGGTGACGCGTGTGGCCTCGAACACCGAGCGACCCTGCAGC |
| NO: 192 | GACCCGCTTAAGCGGCCGCCACCATGGCATCAGCAAGCACAAGCAAATACAACTCACATAGCC |
| (E11A) | TGGAAAACG |
| SEQ ID | agtgagcgagcgagcgcgcagagagggagtggccaactccatcactaggggttcctTGCGCAcaGGTACCTTCGCA |
| NO: 193 | TATTAAGGTGACGCGTGTGGCCTCGAACACCGAGCGACCCTGCAGCGACCCGCTTAAGCGGCC |
| (E11B) | GCCACCatggcttctgcatcaacttctaaatataattcacactccttggagaatgagtctattaagaggacgtctcgagatg |
| | gagtcaatcgagatctcactgaggctgttcctcgacttccaggagaaacactaatcactgacaaagaagttatttacatatgt |
| | cctttcaatggccccattaagggaagagtttacatcacaaattatcgtctttatttaagaagtttggaaacggattcttctctaa |
| | tacttgatgttcctctgggtgtgatctcgagaattgaaaaaatgggaggcgcgacaagtagaggagaaaattcctatggtct |
| | agatattacttgtaaagacatgagaaacctgaggttcgctttgaaacaggaaggccacagcagaagagatatgtttgagat |
| | cctcacgagatacgcgtttcccctggctcacagtctgccattatttgcatttttaaatgaagaaaagtttaacgtggatggatg |
| | gacagtttacaatccagtggaagaatacaggaggcagggcttgcccaatcaccattggagaataacttttattaataagtgc |
| | tatgagctctgtgacacttaccctgctcttttggtggttccgtatcgtgcctcagatgatgacctccggagagttgcaacttttag |
| | gtcccgaaatcgaattccagtgctgtcatggattcatccagaaaataagacggtcattgtgcgttgcagtcagcctcttgtcgg |
| | tatgagtgggaaacgaaataaagatgatgagaaatatctcgatgttatcagggagactaataaacaaatttctaaactcacc |
| | atttatgatgcaagacccagcgtaaatgcagtggccaacaaggcaacaggaggaggatatgaaagtgatgatgcatatcat |
| | aacgccgaacttttcttcttagacattcataatattcatgttatgcgggaatctttaaaaaaagtgaaggacattgtttatccta |
| | atgtagaagaatctcattggttgtccagtttggagtctactcattggttagaacatatcaagctcgttttgacaggagccattc |
| | aagtagcagacaaagtttcttcagggaagagttcagtgcttgtgcattgcagtgacggatgggacaggactgctcagctgac |
| | atccttggccatgctgatgttggatagcttctataggagcattgaagggttcgaaatactggtacaaaaagaatggataagtt |
| | ttggacataaatttgcatctcgaataggtcatggtgataaaaaccacaccgatgctgaccgttctcctatttttctccagtttat |
| | tgattgtgtgtggcaaatgtcaaaacagttccctacagcttttgaattcaatgaacaatttttgattataattttggatcatctgt |
| | atagttgccgatttggtactttcttattcaactgtgaatctgctcgagaaagacagaaggttacagaaaggactgtttctttatg |
| | gtcactgataaacagtaataaagaaaaattcaaaaaccccttctatactaaagaaatcaatcgagttttatatccagttgcca |
| | gtatgcgtcacttggaactctgggtgaattactacattagatggaaccccaggatcaagcaacaacagccgaatccagtgga |
| | gcagcgttacatggagctcttagccttacgcgacgaatacataaagcggcttgaggaactgcagctcgccaactctgccaag |
| | ctttctgatcccccaacttcaccttccagtccttcgcaaatgatgccccatgtgcaaactcacttttaaTTAAGATCTTTTT |
| | CCCTCTGCCAAAAATTATGGGGACATCATGAAGCCCCTTGAGCATCTGACTTCTGGCTAATAAA |
| | GGAAATTTATTTTCATTGCAATAGTGTGTTGGAATTTTTTGTGTCTCTCActcgagGCCTaggaaccc |
| | ctagtgatggagttggccactccctctctgcgcgctcgctcgctcactgaggccgggcgaccaaaggtcgcccgacgcccgg |
| | gctttgcccgggcggcctcagtgagcgagcgagcgcgcagagagggagtggccaa |
| SEQ ID | tttttGtcGACTTCGCATATTAAGGTGACGCGTGTGGCCTCGAACACCGAGCGACCCTGCAGCGA |
| NO: 194 | CCCGCTTAAGCGGCCGCCACCatggcttctgcatcaacttctaaatataattcacactccttggagaatgagtctatt |
| (E12) | aagaggacgtctcgagatggagtcaatcgagatctcactgaggctgttcctcgacttccaggagaaacactaatcactgaca |
| | aagaagttatttacatatgtcctttcaatggccccattaagggaagagtttacatcacaaattatcgtctttatttaagaagttt |
| | ggaaacggattcttctctaatacttgatgttcctctgggtgtgatctcgagaattgaaaaaatgggaggcgcgacaagtagag |
| | gagaaaattcctatggtctagatattacttgtaaagacatgagaaacctgaggttcgctttgaaacaggaaggccacagcag |
| | aagagatatgtttgagatcctcacgagatacgcgtttcccctggctcacagtctgccattatttgcatttttaaatgaagaaaa |
| | gtttaacgtggatggatggacagtttacaatccagtggaagaatacaggaggcagggcttgcccaatcaccattggagaat |
| | aacttttattaataagtgctatgagctctgtgacacttaccctgctcttttggtggttccgtatcgtgcctcagatgatgacctcc |
| | ggagagttgcaacttttaggtcccgaaatcgaattccagtgctgtcatggattcatccagaaaataagacggtcattgtgcgt |
| | tgcagtcagcctcttgtcggtatgagtgggaaacgaaataaagatgatgagaaatatctcgatgttatcagggagactaata |
| | aacaaatttctaaactcaccatttatgatgcaagacccagcgtaaatgcagtggccaacaaggcaacaggaggaggatatg |
| | aaagtgatgatgcatatcataacgccgaacttttcttcttagacattcataatattcatgttatgcgggaatctttaaaaaaag |
| | tgaaggacattgtttatcctaatgtagaagaatctcattggttgtccagtttggagtctactcattggttagaacatatcaagct |
| | cgttttgacaggagccattcaagtagcagacaaagtttcttcagggaagagttcagtgcttgtgcattgcagtgacggatggg |
| | acaggactgctcagctgacatccttggccatgctgatgttggatagcttctataggagcattgaagggttcgaaatactggta |
| | caaaaagaatggataagttttggacataaatttgcatctcgaataggtcatggtgataaaaaccacaccgatgctgaccgtt |
| | ctcctatttttctccagtttattgattgtgtgtggcaaatgtcaaaacagttccctacagcttttgaattcaatgaacaatttttga |
| | ttataattttggatcatctgtatagttgccgatttggtactttcttattcaactgtgaatctgctcgagaaagacagaaggttac |
| | agaaaggactgtttctttatggtcactgataaacagtaataaagaaaaattcaaaaaccccttctatactaaagaaatcaat |
| | cgagttttatatccagttgccagtatgcgtcacttggaactctgggtgaattactacattagatggaaccccaggatcaagca |
| | acaacagccgaatccagtggagcagcgttacatggagctcttagccttacgcgacgaatacataaagcggcttgaggaact |
| | gcagctcgccaactctgccaagctttctgatcccccaacttcaccttccagtccttcgcaaatgatgccccatgtgcaaactca |
| | cttttaaTTAAGATCTTTTTCCCTCTGCCAAAAATTATGGGGACATCATGAAGCCCCTTGAGCATCT |
| | GACTTCTGGCTAATAAAGGAAATTTATTTTCATTGCAATAGTGTGTTGGAATTTTTTGTGTCTCT |
| | CActccctaggaaaaaa |
| SEQ ID | tttttGtcGACTTCGCATATTAAGGTGACGCGTGTGGCCTCGAACACCGAGCGACCCTGCAGCGA |
| NO: 195 | CCCGCTTAAGCGGCCGCCACCatggcttctgcatcaacttctaaatataattcacactccttggagaatgagtctatt |
| (E13A) | aagaggacgtctcgagatggagtcaatcgagatctcactgaggctgttcctcgacttccaggagaaacactaatcactgaca |
| | aagaagttatttacatatgtcctttcaatggccccattaagggaagagtttacatcacaaattatcgtctttatttaagaagttt |
| | ggaaacggattcttctctaatacttgatgttcctctgggtgtgatctcgagaattgaaaaaatgggaggcgcgacaagtagag |
| | gagaaaattcctatggtctagatattacttgtaaagacatgagaaacctgaggttcgctttgaaacaggaaggccacagcag |
| | aagagatatgtttgagatcctcacgagatacgcgtttcccctggctcacagtctgccattatttgcatttttaaatgaagaaaa |
| | gtttaacgtggatggatggacagtttacaatccagtggaagaatacaggaggcagggcttgcccaatcaccattggagaat |
| | aacttttattaataagtgctatgagctctgtgacacttaccctgctcttttggtggttccgtatcgtgcctcagatgatgacctcc |
| | ggagagttgcaacttttaggtcccgaaatcgaattccagtgctgtcatggattcatccagaaaataagacggtcattgtgcgt |
| | tgcagtcagcctcttgtcggtatgagtgggaaacgaaataaagatgatgagaaatatctcgatgttatcagggagactaata |
| | aacaaatttctaaactcaccatttatgatgcaagacccagcgtaaatgcagtggccaacaaggcaacaggaggaggatatg |
| | aaagtgatgatgcatatcataacgccgaacttttcttcttagacattcataatattcatgttatgcgggaatctttaaaaaaag |
| | tgaaggacattgtttatcctaatgtagaagaatctcattggttgtccagtttggagtctactcattggttagaacatatcaagct |
| | cgttttgacaggagccattcaagtagcagacaaagtttcttcagggaagagttcagtgcttgtgcattgcagtgacggatggg |
| | acaggactgctcagctgacatccttggccatgctgatgttggatagcttctataggagcattgaagggttcgaaatactggta |
| | caaaaagaatggataagttttggacataaatttgcatctcgaataggtcatggtgataaaaaccacaccgatgctgaccgtt |
| | ctcctatttttctccagtttattgattgtgtgtggcaaatgtcaaaacagttccctacagcttttgaattcaatgaacaatttttga |
| | ttataattttggatcatctgtatagttgccgatttggtactttcttattcaactgtgaatctgctcgagaaagacagaaggttac |
| | agaaaggactgtttctttatggtcactgataaacagtaataaagaaaaattcaaaaaccccttctatactaaagaaatcaat |
| | cgagttttatatccagttgccagtatgcgtcacttggaactctgggtgaattactacattagatggaaccccaggatcaagca |
| | acaacagccgaatccagtggagcagcgttacatggagctcttagccttacgcgacgaatacataaagcggcttgaggaact |
| | gcagctcgccaactctgccaagctttctgatcccccaacttcaccttccagtccttcgcaaatgatgccccatgtgcaaactca |
| | cttttaaTTAAGATCTTTTTCCCTCTGCCAAAAATTATGGGGACATCATGAAGCCCCTTGAGCATCT |
| | GACTTCTGGCTAATAAAGGAAATTTATTTTCATTGCAATAGTGTGTTGGAATTTTTTGTGTCTCT |
| | CActccctaggaaaaaa |
| SEQ ID | tttttGtcGACCCTCTATAAATACCCGCTCTGGGTTGGCAGCTGTTGCTGCGGTGTGTGTGGGCGT |
| NO: 196 | GGGTGGTGTGAGTAGGGGGATGAATCAGGGAGGGGGCGGGGGCAGGGGGCAGGAGCCACA |
| (E13B) | CAAACCTGCCCTGGCGAAGACCCCCGCTGGCTGACTCAGGGATCTTGCAGCTGTCAGGGGGG |
| | AGGGATACAAATAGTGCCGACGGCTGGGGGCCCTGTCTCCCCTCGCCGCGGCCGCCACCatggc |
| | ttctgcatcaacttctaaatataattcacactccttggagaatgagtctattaagaggacgtctcgagatggagtcaatcgag |
| | atctcactgaggctgttcctcgacttccaggagaaacactaatcactgacaaagaagttatttacatatgtcctttcaatggcc |
| | ccattaagggaagagtttacatcacaaattatcgtctttatttaagaagtttggaaacggattcttctctaatacttgatgttcc |
| | tctgggtgtgatctcgagaattgaaaaaatgggaggcgcgacaagtagaggagaaaattcctatggtctagatattacttgt |
| | aaagacatgagaaacctgaggttcgctttgaaacaggaaggccacagcagaagagatatgtttgagatcctcacgagatac |
| | gcgtttcccctggctcacagtctgccattatttgcatttttaaatgaagaaaagtttaacgtggatggatggacagtttacaatc |
| | cagtggaagaatacaggaggcagggcttgcccaatcaccattggagaataacttttattaataagtgctatgagctctgtga |
| | cacttaccctgctcttttggtggttccgtatcgtgcctcagatgatgacctccggagagttgcaacttttaggtcccgaaatcga |
| | attccagtgctgtcatggattcatccagaaaataagacggtcattgtgcgttgcagtcagcctcttgtcggtatgagtgggaa |
| | acgaaataaagatgatgagaaatatctcgatgttatcagggagactaataaacaaatttctaaactcaccatttatgatgca |
| | agacccagcgtaaatgcagtggccaacaaggcaacaggaggaggatatgaaagtgatgatgcatatcataacgccgaact |
| | tttcttcttagacattcataatattcatgttatgcgggaatctttaaaaaaagtgaaggacattgtttatcctaatgtagaagaa |
| | tctcattggttgtccagtttggagtctactcattggttagaacatatcaagctcgttttgacaggagccattcaagtagcagac |
| | aaagtttcttcagggaagagttcagtgcttgtgcattgcagtgacggatgggacaggactgctcagctgacatccttggccat |
| | gctgatgttggatagcttctataggagcattgaagggttcgaaatactggtacaaaaagaatggataagttttggacataaat |
| | ttgcatctcgaataggtcatggtgataaaaaccacaccgatgctgaccgttctcctatttttctccagtttattgattgtgtgtgg |
| | caaatgtcaaaacagttccctacagcttttgaattcaatgaacaatttttgattataattttggatcatctgtatagttgccgatt |
| | tggtactttcttattcaactgtgaatctgctcgagaaagacagaaggttacagaaaggactgtttctttatggtcactgataaa |
| | cagtaataaagaaaaattcaaaaaccccttctatactaaagaaatcaatcgagttttatatccagttgccagtatgcgtcact |
| | tggaactctgggtgaattactacattagatggaaccccaggatcaagcaacaacagccgaatccagtggagcagcgttaca |
| | tggagctcttagccttacgcgacgaatacataaagcggcttgaggaactgcagctcgccaactctgccaagctttctgatccc |
| | ccaacttcaccttccagtccttcgcaaatgatgccccatgtgcaaactcacttttaaTTAAGATCTTTTTCCCTCTGCC |
| | AAAAATTATGGGGACATCATGAAGCCCCTTGAGCATCTGACTTCTGGCTAATAAAGGAAATTTA |
| | TTTTCATTGCAATAGTGTGTTGGAATTTTTTGTGTCTCTCActccctaggaaaaaa |
| SEQ ID | agtgagcgagcgagcgcgcagagagggagtggccaactccatcactaggggttcctTGCGCAcaGGTACCCCTCTA |
| NO: 197 | TAAATACCCGCTCTGGGTTGGCAGCTGTTGCTGCGGTGTGTGTGGGCGTGGGTGGTGTGAGTA |
| (E13C) | GGGGGATGAATCAGGGAGGGGGCGGGGGCAGGGGGCAGGAGCCACACAAACCTGCCCTGG |
| | CGAAGACCCCCGCTGGCTGACTCAGGGATCTTGCAGCTGTCAGGGGGGAGGGATACAAATAG |
| | TGCCGACGGCTGGGGGCCCTGTCTCCCCTCGCCGCGGCCGCCACCatggcttctgcatcaacttctaaat |
| | ataattcacactccttggagaatgagtctattaagaggacgtctcgagatggagtcaatcgagatctcactgaggctgttcct |
| | cgacttccaggagaaacactaatcactgacaaagaagttatttacatatgtcctttcaatggccccattaagggaagagttta |
| | catcacaaattatcgtctttatttaagaagtttggaaacggattcttctctaatacttgatgttcctctgggtgtgatctcgagaa |
| | ttgaaaaaatgggaggcgcgacaagtagaggagaaaattcctatggtctagatattacttgtaaagacatgagaaacctga |
| | ggttcgctttgaaacaggaaggccacagcagaagagatatgtttgagatcctcacgagatacgcgtttcccctggctcacag |
| | tctgccattatttgcatttttaaatgaagaaaagtttaacgtggatggatggacagtttacaatccagtggaagaatacagga |
| | ggcagggcttgcccaatcaccattggagaataacttttattaataagtgctatgagctctgtgacacttaccctgctcttttggt |
| | ggttccgtatcgtgcctcagatgatgacctccggagagttgcaacttttaggtcccgaaatcgaattccagtgctgtcatggat |
| | tcatccagaaaataagacggtcattgtgcgttgcagtcagcctcttgtcggtatgagtgggaaacgaaataaagatgatgag |
| | aaatatctcgatgttatcagggagactaataaacaaatttctaaactcaccatttatgatgcaagacccagcgtaaatgcagt |
| | ggccaacaaggcaacaggaggaggatatgaaagtgatgatgcatatcataacgccgaacttttcttcttagacattcataat |
| | attcatgttatgcgggaatctttaaaaaaagtgaaggacattgtttatcctaatgtagaagaatctcattggttgtccagtttgg |
| | agtctactcattggttagaacatatcaagctcgttttgacaggagccattcaagtagcagacaaagtttcttcagggaagagt |
| | tcagtgcttgtgcattgcagtgacggatgggacaggactgctcagctgacatccttggccatgctgatgttggatagcttctat |
| | aggagcattgaagggttcgaaatactggtacaaaaagaatggataagttttggacataaatttgcatctcgaataggtcatg |
| | gtgataaaaaccacaccgatgctgaccgttctcctatttttctccagtttattgattgtgtgtggcaaatgtcaaaacagttccc |
| | tacagcttttgaattcaatgaacaatttttgattataattttggatcatctgtatagttgccgatttggtactttcttattcaactgt |
| | gaatctgctcgagaaagacagaaggttacagaaaggactgtttctttatggtcactgataaacagtaataaagaaaaattca |
| | aaaaccccttctatactaaagaaatcaatcgagttttatatccagttgccagtatgcgtcacttggaactctgggtgaattact |
| | acattagatggaaccccaggatcaagcaacaacagccgaatccagtggagcagcgttacatggagctcttagccttacgcg |
| | acgaatacataaagcggcttgaggaactgcagctcgccaactctgccaagctttctgatcccccaacttcaccttccagtcctt |
| | cgcaaatgatgccccatgtgcaaactcacttttaaTTAAGATCTTTTTCCCTCTGCCAAAAATTATGGGGACA |
| | TCATGAAGCCCCTTGAGCATCTGACTTCTGGCTAATAAAGGAAATTTATTTTCATTGCAATAGT |
| | GTGTTGGAATTTTTTGTGTCTCTCActcgagGCCTaggaacccctagtgatggagttggccactccctctctgcg |
| | cgctcgctcgctcactgaggccgggcgaccaaaggtcgcccgacgcccgggctttgcccgggcggcctcagtgagcgagcg |
| | agcgcgcagagagggagtggccaa |
| SEQ ID | tttttGtcGACCCTCTATAAATACCCGCTCTGGGTTGGCAGCTGTTGCTGCGGTGTGTGTGGGCGT |
| NO: 198 | GGGTGGTGTGAGTAGGGGGATGAATCAGGGAGGGGGCGGGGGCAGGGGGCAGGAGCCACA |
| (E14) | CAAACCTGCCCTGGCGAAGACCCCCGCTGGCTGACTCAGGGATCTTGCAGCTGTCAGGGGGG |
| | AGGGATACAAATAGTGCCGACGGCTGGGGGCCCTGTCTCCCCTCGCCGCGGCCGCCACCatggc |
| | ttctgcatcaacttctaaatataattcacactccttggagaatgagtctattaagaggacgtctcgagatggagtcaatcgag |
| | atctcactgaggctgttcctcgacttccaggagaaacactaatcactgacaaagaagttatttacatatgtcctttcaatggcc |
| | ccattaagggaagagtttacatcacaaattatcgtctttatttaagaagtttggaaacggattcttctctaatacttgatgttcc |
| | tctgggtgtgatctcgagaattgaaaaaatgggaggcgcgacaagtagaggagaaaattcctatggtctagatattacttgt |
| | aaagacatgagaaacctgaggttcgctttgaaacaggaaggccacagcagaagagatatgtttgagatcctcacgagatac |
| | gcgtttcccctggctcacagtctgccattatttgcatttttaaatgaagaaaagtttaacgtggatggatggacagtttacaatc |
| | cagtggaagaatacaggaggcagggcttgcccaatcaccattggagaataacttttattaataagtgctatgagctctgtga |
| | cacttaccctgctcttttggtggttccgtatcgtgcctcagatgatgacctccggagagttgcaacttttaggtcccgaaatcga |
| | attccagtgctgtcatggattcatccagaaaataagacggtcattgtgcgttgcagtcagcctcttgtcggtatgagtgggaa |
| | acgaaataaagatgatgagaaatatctcgatgttatcagggagactaataaacaaatttctaaactcaccatttatgatgca |
| | agacccagcgtaaatgcagtggccaacaaggcaacaggaggaggatatgaaagtgatgatgcatatcataacgccgaact |
| | tttcttcttagacattcataatattcatgttatgcgggaatctttaaaaaaagtgaaggacattgtttatcctaatgtagaagaa |
| | tctcattggttgtccagtttggagtctactcattggttagaacatatcaagctcgttttgacaggagccattcaagtagcagac |
| | aaagtttcttcagggaagagttcagtgcttgtgcattgcagtgacggatgggacaggactgctcagctgacatccttggccat |
| | gctgatgttggatagcttctataggagcattgaagggttcgaaatactggtacaaaaagaatggataagttttggacataaat |
| | ttgcatctcgaataggtcatggtgataaaaaccacaccgatgctgaccgttctcctatttttctccagtttattgattgtgtgtgg |
| | caaatgtcaaaacagttccctacagcttttgaattcaatgaacaatttttgattataattttggatcatctgtatagttgccgatt |
| | tggtactttcttattcaactgtgaatctgctcgagaaagacagaaggttacagaaaggactgtttctttatggtcactgataaa |
| | cagtaataaagaaaaattcaaaaaccccttctatactaaagaaatcaatcgagttttatatccagttgccagtatgcgtcact |
| | tggaactctgggtgaattactacattagatggaaccccaggatcaagcaacaacagccgaatccagtggagcagcgttaca |
| | tggagctcttagccttacgcgacgaatacataaagcggcttgaggaactgcagctcgccaactctgccaagctttctgatccc |
| | ccaacttcaccttccagtccttcgcaaatgatgccccatgtgcaaactcacttttaaTTAAGATCTTTTTCCCTCTGCC |
| | AAAAATTATGGGGACATCATGAAGCCCCTTGAGCATCTGACTTCTGGCTAATAAAGGAAATTTA |
| | TTTTCATTGCAATAGTGTGTTGGAATTTTTTGTGTCTCTCActccctaggaaaaaa |
| SEQ ID | ttggccactccctctctgcgcgctcgctcgctcactgaggccgcccgggcaaagcccgggcgtcgggcgacctttggtcgccc |
| NO: 199 | ggcctcagtgagcgagcgagcgcgcagagagggagtggccaactccatcactaggggttcctttGtcGACCCTCTATA |
| (E15) | AATACCCGCTCTGGGTTGGCAGCTGTTGCTGCGGTGTGTGTGGGCGTGGGTGGTGTGAGTAG |
| | GGGGATGAATCAGGGAGGGGGCGGGGGCAGGGGGCAGGAGCCACACAAACCTGCCCTGGC |
| | GAAGACCCCCGCTGGCTGACTCAGGGATCTTGCAGCTGTCAGGGGGGAGGGATACAAATAGT |
| | GCCGACGGCTGGGGGCCCTGTCTCCCCTCGCCGCGGCCGCCACCatggcttctgcatcaacttctaaata |
| | taattcacactccttggagaatgagtctattaagaggacgtctcgagatggagtcaatcgagatctcactgaggctgttcctc |
| | gacttccaggagaaacactaatcactgacaaagaagttatttacatatgtcctttcaatggccccattaagggaagagtttac |
| | atcacaaattatcgtctttatttaagaagtttggaaacggattcttctctaatacttgatgttcctctgggtgtgatctcgagaat |
| | tgaaaaaatgggaggcgcgacaagtagaggagaaaattcctatggtctagatattacttgtaaagacatgagaaacctgag |
| | gttcgctttgaaacaggaaggccacagcagaagagatatgtttgagatcctcacgagatacgcgtttcccctggctcacagtc |
| | tgccattatttgcatttttaaatgaagaaaagtttaacgtggatggatggacagtttacaatccagtggaagaatacaggagg |
| | cagggcttgcccaatcaccattggagaataacttttattaataagtgctatgagctctgtgacacttaccctgctcttttggtgg |
| | ttccgtatcgtgcctcagatgatgacctccggagagttgcaacttttaggtcccgaaatcgaattccagtgctgtcatggattc |
| | atccagaaaataagacggtcattgtgcgttgcagtcagcctcttgtcggtatgagtgggaaacgaaataaagatgatgagaa |
| | atatctcgatgttatcagggagactaataaacaaatttctaaactcaccatttatgatgcaagacccagcgtaaatgcagtgg |
| | ccaacaaggcaacaggaggaggatatgaaagtgatgatgcatatcataacgccgaacttttcttcttagacattcataatatt |
| | catgttatgcgggaatctttaaaaaaagtgaaggacattgtttatcctaatgtagaagaatctcattggttgtccagtttggag |
| | tctactcattggttagaacatatcaagctcgttttgacaggagccattcaagtagcagacaaagtttcttcagggaagagttc |
| | agtgcttgtgcattgcagtgacggatgggacaggactgctcagctgacatccttggccatgctgatgttggatagcttctatag |
| | gagcattgaagggttcgaaatactggtacaaaaagaatggataagttttggacataaatttgcatctcgaataggtcatggt |
| | gataaaaaccacaccgatgctgaccgttctcctatttttctccagtttattgattgtgtgtggcaaatgtcaaaacagttcccta |
| | cagcttttgaattcaatgaacaatttttgattataattttggatcatctgtatagttgccgatttggtactttcttattcaactgtg |
| | aatctgctcgagaaagacagaaggttacagaaaggactgtttctttatggtcactgataaacagtaataaagaaaaattcaa |
| | aaaccccttctatactaaagaaatcaatcgagttttatatccagttgccagtatgcgtcacttggaactctgggtgaattacta |
| | cattagatggaaccccaggatcaagcaacaacagccgaatccagtggagcagcgttacatggagctcttagccttacgcga |
| | cgaatacataaagcggcttgaggaactgcagctcgccaactctgccaagctttctgatcccccaacttcaccttccagtccttc |
| | gcaaatgatgccccatgtgcaaactcacttttaaTTAAGATCTTTTTCCCTCTGCCAAAAATTATGGGGACAT |
| | CATGAAGCCCCTTGAGCATCTGACTTCTGGCTAATAAAGGAAATTTATTTTCATTGCAATAGTG |
| | TGTTGGAATTTTTTGTGTCTCTCActcgCctaggccactccctctctgcgcgctcgctcgctcactgaggccgggcg |
| | accaaaggtcgcccgacgcccgggctttgcccgggcggcctcagtgagcgagcgagcgcgcagagagggagtggccaa |
| SEQ ID | ttttGGTACCgacattgattattgactagttattaatagtaatcaattacggggtcattagttcatagcccatatatggagttc |
| NO: 200 | cgcgttacataacttacggtaaatggcccgcctggctgaccgcccaacgacccccgcccattgacgtcaataatgacgtatgt |
| (E16) | tcccatagtaacgccaatagggactttccattgacgtcaatgggtggagtatttacggtaaactgcccacttggcagtacatc |
| | aagtgtatcatatgccaagtacgccccctattgacgtcaatgacggtaaatggcccgcctggcattatgcccagtacatgacc |
| | ttatgggactttcctacttggcagtacatctacgtattagtcatcgctattaccatggtcgaggtgagccccacgttctgcttca |
| | ctctccccatctcccccccctccccacccccaattttgtatttatttattttttaattattttgtgcagcgatgggggcggggggg |
| | gggggggggcgcgcgccaggcggggcggggcggggcgaggggcggggcggggcgaggcggagaggtgcggcggcagc |
| | caatcagagcggcgcgctccgaaagtttccttttatggcgaggcggcggcggcggcggccctataaaaagcgaagcgcgcg |
| | gcgggcggatatctttttt |
| SEQ ID | ttggccactccctctctgcgcgctcgctcgctcactgaggccgcccgggcaaagcccgggcgtcgggcgacctttggtcgccc |
| NO: 201 | ggcctcagtgagcgagcgagcgcgcagagagggagtggccaactccatcactaggggttcctttGTCGACgacattgatt |
| (E17) | attgactagttattaatagtaatcaattacggggtcattagttcatagcccatatatggagttccgcgttacataacttacggta |
| | aatggcccgcctggctgaccgcccaacgacccccgcccattgacgtcaataatgacgtatgttcccatagtaacgccaatag |
| | ggactttccattgacgtcaatgggggagtatttacggtaaactgcccacttggcagtacatcaagtgtatcatatgccaagta |
| | cgccccctattgacgtcaatgacggtaaatggcccgcctggcattatgcccagtacatgaccttatgggactttcctacttggc |
| | agtacatctacgtattagtcatcgctattaccatggtcgaggtgagccccacgttctgcttcactctccccatctcccccccctc |
| | cccacccccaattttgtatttatttattttttaattattttgtgcagcgatgggggcggggggggggggggggcgcgcgccagg |
| | cggggcggggcggggcgaggggcggggggggcgaggcggagaggtgcggcggcagccaatcagagcggcgcgctccg |
| | aaagtttccttttatggcgaggcggcggcggcggcggccctataaaaagcgaagcgcgcggcgggcgggagtGgctgcgc |
| | gctgccttcgccccgtgccccgctccgccgccgcctcgcgccgcccgccccggctctgactgaccgcgttactcccacaggtg |
| | agcgggcgggacggcccttctcctccgggctgtaattagcgcttggtttaatgacggcttgtttcttttctgtggctgcgtgaaa |
| | gccttgaggggctccgggagggccctttgtgcggggggagcggctcggggggtgcgtgcgtgtgtgtgtgcgtggggagcgc |
| | cgcgtgcggctccgcgctgcccggcggctgtgagcgctgcgggcgcggcgcggggctttgtgcgctccgcagtgtgcgcgag |
| | gggagcgcggccgggggcggtgccccgcggtgcggggggggctgcgaggggaacaaaggctgcgtgcggggtgtgtgcgt |
| | gggggggtgagcagggggtgtgggcgcgtcggtcgggctgcaaccccccctgcacccccctccccgagttgctgagcacgg |
| | cccggcttcgggtgcggggctccgtacggggcgtggcgcggggctcgccgtgccgggcggggggggcggcaggtgggggt |
| | gccgggcggggcggggccgcctcgggccggggagggctcgggggaggggcgcggcggcccccggagcgccggcggctgt |
| | cgaggcgcggcgagccgcagccattgccttttatggtaatcgtgcgagagggcgcagggacttcctttgtcccaaatctgtgc |
| | ggagccgaaatctgggaggcgccgccgcaccccctctagcgggcgcggggcgaagcggtgcggcgccggcaggaaggaa |
| | atgggggggagggccttcgtgcgtcgccgcgccgccgtccccttctccctctccagcctcggggctgtccgcggggggacgg |
| | ctgccttcgggggggacggggcagggcggggttcggcttctggcgtgtgaccggcggctctagagcctctgctaaccatgttc |
| | atgccttcttctttttcctacagctcctgggcaacgtgctggttattgtgctgtctcatcattttggcaaaAGATCTgccgccac |
| | catggtgagcaagggcgaggagctgttcaccggggtggtgcccatcctggtcgagctggacggcgacgtaaacggccacaa |
| | gttcagcgtgtccggcgagggcgagggcgatgccacctacggcaagctgaccctgaagttcatctgcaccaccggcaagct |
| | gcccgtgccctggcccaccctcgtgaccaccctgacctacggcgtgcagtgcttcagccgctaccccgaccacatgaagcag |
| | cacgacttcttcaagtccgccatgcccgaaggctacgtccaggagcgcaccatcttcttcaaggacgacggcaactacaaga |
| | cccgcgccgaggtgaagttcgagggcgacaccctggtgaaccgcatcgagctgaagggcatcgacttcaaggaggacggc |
| | aacatcctggggcacaagctggagtacaactacaacagccacaacgtctatatcatggccgacaagcagaagaacggcat |
| | caaggtgaacttcaagatccgccacaacatcgaggacggcagcgtgcagctcgccgaccactaccagcagaacacccccat |
| | cggcgacggccccgtgctgctgcccgacaaccactacctgagcacccagtccgccctgagcaaagaccccaacgagaagc |
| | gcgatcacatggtcctgctggagttcgtgaccgccgccgggatcactctcggcatggacgagctgtacaagTGAGAGCT |
| | Cgatctttttccctctgccaaaaattatggggacatcatgaagccccttgagcatctgacttctggctaataaaggaaatttat |
| | tttcattgcaatagtgtgttggaattttttgtgtctctcactcggaagcctagGaggaacccctagtgatggagttggccactcc |
| | ctctctgcgcgctcgctcgctcactgaggccgggcgaccaaaggtcgcccgacgcccgggctttgcccgggcggcctcagtg |
| | agcgagcgagcgcgcagagagggagtggccaa |
| SEQ ID | CAAAGAATTTGAGCGGCCGCCACCATGGCTTCTGCATCAACTTCTAAATATAATTCACACTCCTT |
| NO: 202 | GGAGAATGAGTCTATTAAGAGGACGTCTCGAGATGGAGTCAATCGAGATCTCACTGAGGCTGT |
| (E18) | TCCTCGACTTCCAGGAGAAACACTAATCACTGACAAAGAAGTTATTTACATATGTCCTTTCAATG |
| | GCCCCATTAAGGGAAGAGTTTACATCACAAATTATCGTCTTTATTTAAGAAGTTTGGAAACGGA |
| | TTCTTCTCTAATACTTGATGTTCCTCTGGGTGTGATCTCGAGAATTGAAAAAATGGGAGGCGCG |
| | ACAAGTAGAGGAGAAAATTCCTATGGTCTAGATATTACTTGTAAAGACATGAGAAACCTGAGG |
| | TTCGCTTTGAAACAGGAAGGCCACAGCAGAAGAGATATGTTTGAGATCCTCACGAGATACGCG |
| | TTTCCCCTGGCTCACAGTCTGCCATTATTTGCATTTTTAAATGAAGAAAAGTTTAACGTGGATGG |
| | ATGGACAGTTTACAATCCAGTGGAAGAATACAGGAGGCAGGGCTTGCCCAATCACCATTGGA |
| | GAATAACTTTTATTAATAAGTGCTATGAGCTCTGTGACACTTACCCTGCTCTTTTGGTGGTTCCG |
| | TATCGTGCCTCAGATGATGACCTCCGGAGAGTTGCAACTTTTAGGTCCCGAAATCGAATTCCAG |
| | TGCTGTCATGGATTCATCCAGAAAATAAGACGGTCATTGTGCGTTGCAGTCAGCCTCTTGTCGG |
| | TATGAGTGGGAAACGAAATAAAGATGATGAGAAATATCTCGATGTTATCAGGGAGACTAATA |
| | AACAAATTTCTAAACTCACCATTTATGATGCAAGACCCAGCGTAAATGCAGTGGCCAACAAGG |
| | CAACAGGAGGAGGATATGAAAGTGATGATGCATATCATAACGCCGAACTTTTCTTCTTAGACA |
| | TTCATAATATTCATGTTATGCGGGAATCTTTAAAAAAAGTGAAGGACATTGTTTATCCTAATGTA |
| | GAAGAATCTCATTGGTTGTCCAGTTTGGAGTCTACTCATTGGTTAGAACATATCAAGCTCGTTTT |
| | GACAGGAGCCATTCAAGTAGCAGACAAAGTTTCTTCAGGGAAGAGTTCAGTGCTTGTGCATTG |
| | CAGTGACGGATGGGACAGGACTGCTCAGCTGACATCCTTGGCCATGCTGATGTTGGATAGCTT |
| | CTATAGGAGCATTGAAGGGTTCGAAATACTGGTACAAAAAGAATGGATAAGTTTTGGACATAA |
| | ATTTGCATCTCGAATAGGTCATGGTGATAAAAACCACACCGATGCTGACCGTTCTCCTATTTTTC |
| | TCCAGTTTATTGATTGTGTGTGGCAAATGTCAAAACAGTTCCCTACAGCTTTTGAATTCAATGAA |
| | CAATTTTTGATTATAATTTTGGATCATCTGTATAGTTGCCGATTTGGTACTTTCTTATTCAACTGT |
| | GAATCTGCTCGAGAAAGACAGAAGGTTACAGAAAGGACTGTTTCTTTATGGTCACTGATAAAC |
| | AGTAATAAAGAAAAATTCAAAAACCCCTTCTATACTAAAGAAATCAATCGAGTTTTATATCCAG |
| | TTGCCAGTATGCGTCACTTGGAACTCTGGGTGAATTACTACATTAGATGGAACCCCAGGATCAA |
| | GCAACAACAGCCGAATCCAGTGGAGCAGCGTTACATGGAGCTCTTAGCCTTACGCGACGAATA |
| | CATAAAGCGGCTTGAGGAACTGCAGCTCGCCAACTCTGCCAAGCTTTCTGATCCCCCAACTTCA |
| | CCTTCCAGTCCTTCGCAAATGATGCCCCATGTGCAAACTCACTTTTAATTAAGATCTTTTT |
| SEQ ID | TTTGAGCGGCCGCCACCatggcttctgcatcaacttctaaatataattcacactccttggagaatgagtctattaagag |
| NO: 203 | gacgtctcgagatggagtcaatcgagatctcactgaggctgttcctcgacttccaggagaaacactaatcactgacaaagaa |
| (E19) | gttatttacatatgtcctttcaatggccccattaagggaagagtttacatcacaaattatcgtctttatttaagaagtttggaaa |
| | cggattcttctctaatacttgatgttcctctgggtgtgatctcgagaattgaaaaaatgggaggcgcgacaagtagaggagaa |
| | aattcctatggtctagatattacttgtaaagacatgagaaacctgaggttcgctttgaaacaggaaggccacagcagaagag |
| | atatgtttgagatcctcacgagatacgcgtttcccctggctcacagtctgccattatttgcatttttaaatgaagaaaagtttaa |
| | cgtggatggatggacagtttacaatccagtggaagaatacaggaggcagggcttgcccaatcaccattggagaataactttt |
| | attaataagtgctatgagctctgtgacacttaccctgctcttttggtggttccgtatcgtgcctcagatgatgacctccggagag |
| | ttgcaacttttaggtcccgaaatcgaattccagtgctgtcatggattcatccagaaaataagacggtcattgtgcgttgcagtc |
| | agcctcttgtcggtatgagtgggaaacgaaataaagatgatgagaaatatctcgatgttatcagggagactaataaacaaat |
| | ttctaaactcaccatttatgatgcaagacccagcgtaaatgcagtggccaacaaggcaacaggaggaggatatgaaagtga |
| | tgatgcatatcataacgccgaacttttcttcttagacattcataatattcatgttatgcgggaatctttaaaaaaagtgaagga |
| | cattgtttatcctaatgtagaagaatctcattggttgtccagtttggagtctactcattggttagaacatatcaagctcgttttga |
| | caggagccattcaagtagcagacaaagtttcttcagggaagagttcagtgcttgtgcattgcagtgacggatgggacaggac |
| | tgctcagctgacatccttggccatgctgatgttggatagcttctataggagcattgaagggttcgaaatactggtacaaaaag |
| | aatggataagttttggacataaatttgcatctcgaataggtcatggtgataaaaaccacaccgatgctgaccgttctcctattt |
| | ttctccagtttattgattgtgtgtggcaaatgtcaaaacagttccctacagcttttgaattcaatgaacaatttttgattataattt |
| | tggatcatctgtatagttgccgatttggtactttcttattcaactgtgaatctgctcgagaaagacagaaggttacagaaagga |
| | ctgtttctttatggtcactgataaacagtaataaagaaaaattcaaaaaccccttctatactaaagaaatcaatcgagttttat |
| | atccagttgccagtatgcgtcacttggaactctgggtgaattactacattagatggaaccccaggatcaagcaacaacagcc |
| | gaatccagtggagcagcgttacatggagctcttagccttacgcgacgaatacataaagcggcttgaggaactgcagctcgcc |
| | aactctgccaagctttctgatcccccaacttcaccttccagtccttcgcaaatgatgcCCCATGTGCAAACTCACTTTT |
| | AATTAAGATC |
| SEQ ID | ttggccactccctctctgcgcgctcgctcgctcactgaggccgcccgggcaaagcccgggcgtcgggcgacctttggtcgccc |
| NO: 204 | ggcctcagtgagcgagcgagcgcgcagagagggagtggccaactccatcactaggggttcctTGCGCAcaggtaccccc |
| (E20A) | ctgcccccacagctcctctcctgtgccttgtttcccagccatgcgttctcctctataaatacccgctctggtatttggggttggca |
| | gctgttgctgccagggagatggttgggttgacatgcggctcctgacaaaacacaaacccctggtgtgtgtgggcgtgggtggt |
| | gtgagtagggggatgaatcaggggggggcgggggacccagggggcaggagccacacaaagtctgtgcggggggggag |
| | cgcacatagcaattggaaactggctgcagacatgcttgctgcctgccctggcgaaggattggtaggcttgccgtcacaggac |
| | ccccgctggctgactcaggggcgcaggctcttgcgggggagctggcctcccgcccccacggccacgggccctttcctggcag |
| | gacagcgggatcttgcagctgtcaggggaggggaggcgggggctgatgtcaggagggatacaaatagtgccgacggctgg |
| | gggccctgtctcccctcgccgcatccactctccggccggccgcctgcccgccgcctcctccgtgcgcccgccagcctcgcccgc |
| | gccgtcaccGATATCtcagatcgcctggagacgccatccacgctgttttgacctccatagaagacaccgggaccgatcca |
| | gcctccgcggattcgaatcccggccgggaacggtgcattggaacgcggattccccgtgccaagagtgacgtaagtaccgcct |
| | atagagtctataggcccacaaaaaatgctttcttcttttaatatacttttttgtttatcttatttctaatactttccctaatctctttc |
| | tttcagggcaataatgatacaatgtatcatgcctctttgcaccattctaaagaataacagtgataatttctgggttaaggcaat |
| | agcaatatttctgcatataaatatttctgcatataaattgtaactgatgtaagaggtttcatattgctaatagcagctacaatcc |
| | agctaccattctgcttttattttatggttgggataaggctggattattctgagtccaagctaggcccttttgctaatcatgttcat |
| | acctcttatcttcctcccacagctcctgggcaacgtgctggtctgtgtgctggcccatcactttggcaaagaattGGATCCgc |
| | cgccacc |
| SEQ ID | taaGTCGACGTAAGTTTTTAAATGTATAAATTGTCTTATttataaATTGGTCTAAAATATATGTAATt |
| NO: 205 | GTCTTAAgatctttttccctctgccaaaaattatggggacatcatgaagccccttgagcatctgacttctggctaataaagg |
| (E20B) | aaatttattttcattgcaatagtgtgttggaattttttgtgtctctcactcgagGCCTaggaacccctagtgatggagttggcc |
| | actccctctctgcgcgctcgctcgctcactgaggccgggcgaccaaaggtcgcccgacgcccgggctttgcccgggcggcctc |
| | agtgagcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 206 | ggcctcagtgagcgagcgagcgcgcagagagggagtggccaactccatcactaggggttcctttgtcGACTCGGTTCG |
| (E21A) | CATATTAAGGTGACGCGTGTGGCCTCGAACACCGAGCGACCCTGCAGCGACCCGCTTAA |
| SEQ ID | AATAAAGGAAATTTATTTTCATTGCAATAGTGTGTTGGAATTTTTTGTGTCTCTCAGAGTCctagg |
| NO: 207 | ccactccctctctgcgcgctcgctcgctcactgaggccgggcgaccaaaggtcgcccgacgcccgggctttgcccgggcggc |
| (E21B) | ctcagtgagcgagcgagcgcgcagagagggagtggccaa |
| SEQ ID | ttggccactccctctctgcgcgctcgctcgctcactgaggccgcccgggcaaagcccgggcgtcgggcgacctttggtcgccc |
| NO: 208 | ggcctcagtgagcgagcgagcgcgcagagagggagtggccaactccatcactaggggttcctttGtcGACTTCGCATA |
| (E22) | TTAAGGTGACGCGTGTGGCCTCGAACACCGAGCGACCCTGCAGCGACCCGCTTAAGCGGCCGC |
| | CACCatggcttctgcatcaacttctaaatataattcacactccttggagaatgagtctattaagaggacgtctcgagatgga |
| | gtcaatcgagatctcactgaggctgttcctcgacttccaggagaaacactaatcactgacaaagaagttatttacatatgtcct |
| | ttcaatggccccattaagggaagagtttacatcacaaattatcgtctttatttaagaagtttggaaacggattcttctctaatac |
| | ttgatgttcctctgggtgtgatctcgagaattgaaaaaatgggaggcgcgacaagtagaggagaaaattcctatggtctaga |
| | tattacttgtaaagacatgagaaacctgaggttcgctttgaaacaggaaggccacagcagaagagatatgtttgagatcctc |
| | acgagatacgcgtttcccctggctcacagtctgccattatttgcatttttaaatgaagaaaagtttaacgtggatggatggaca |
| | gtttacaatccagtggaagaatacaggaggcagggcttgcccaatcaccattggagaataacttttattaataagtgctatga |
| | gctctgtgacacttaccctgctcttttggtggttccgtatcgtgcctcagatgatgacctccggagagttgcaacttttaggtccc |
| | gaaatcgaattccagtgctgtcatggattcatccagaaaataagacggtcattgtgcgttgcagtcagcctcttgtcggtatga |
| | gtgggaaacgaaataaagatgatgagaaatatctcgatgttatcagggagactaataaacaaatttctaaactcaccattta |
| | tgatgcaagacccagcgtaaatgcagtggccaacaaggcaacaggaggaggatatgaaagtgatgatgcatatcataacg |
| | ccgaacttttcttcttagacattcataatattcatgttatgcgggaatctttaaaaaaagtgaaggacattgtttatcctaatgt |
| | agaagaatctcattggttgtccagtttggagtctactcattggttagaacatatcaagctcgttttgacaggagccattcaagt |
| | agcagacaaagtttcttcagggaagagttcagtgcttgtgcattgcagtgacggatgggacaggactgctcagctgacatcc |
| | ttggccatgctgatgttggatagcttctataggagcattgaagggttcgaaatactggtacaaaaagaatggataagttttgg |
| | acataaatttgcatctcgaataggtcatggtgataaaaaccacaccgatgctgaccgttctcctatttttctccagtttattgat |
| | tgtgtgtggcaaatgtcaaaacagttccctacagcttttgaattcaatgaacaatttttgattataattttggatcatctgtatag |
| | ttgccgatttggtactttcttattcaactgtgaatctgctcgagaaagacagaaggttacagaaaggactgtttctttatggtca |
| | ctgataaacagtaataaagaaaaattcaaaaaccccttctatactaaagaaatcaatcgagttttatatccagttgccagtat |
| | gcgtcacttggaactctgggtgaattactacattagatggaaccccaggatcaagcaacaacagccgaatccagtggagca |
| | gcgttacatggagctcttagccttacgcgacgaatacataaagcggcttgaggaactgcagctcgccaactctgccaagcttt |
| | ctgatcccccaacttcaccttccagtccttcgcaaatgatgccccatgtgcaaactcacttttaaTTAAGATCTTTTTCCC |
| | TCTGCCAAAAATTATGGGGACATCATGAAGCCCCTTGAGCATCTGACTTCTGGCTAATAAAGGA |
| | AATTTATTTTCATTGCAATAGTGTGTTGGAATTTTTTGTGTCTCTCActccctaggccactccctctctgcg |
| | cgctcgctcgctcactgaggccgggcgaccaaaggtcgcccgacgcccgggctttgcccgggcggcctcagtgagcgagcg |
| | agcgcgcagagagggagtggccaa |
| SEQ ID | ttttGGTACCgacattgattattgactagttatt |
| NO: 209 | |
| SEQ ID | aaaaaa gatatc cgcccgccgcgc |
| NO: 210 | |
| SEQ ID | TttttGtcGACTTCGCATATTAAGGTGACGCGT |
| NO: 211 | |
| SEQ ID | tttttt cctagg gagTGAGAGACACAAAAAATTCCAACACAC |
| NO: 212 | |
| SEQ ID | tttttGtcGACCCTCTATAAATACCCGCTCTGG |
| NO: 213 | |
| SEQ ID | tttttt cctagg gagTGAGAGACACAAAAAATTCCAACACAC |
| NO: 214 | |

What is claimed is:

1. A modified adeno-associated virus (AAV) capsid protein, comprising:
(i) a reference AAV capsid protein, and
(ii) a 7-mer peptide having the sequence RGDLLLS (SEQ ID NO: 1) inserted into a site within VR VIII of the reference AAV capsid protein.

2. The modified AAV capsid protein of claim 1, wherein the AAV capsid protein is selected from one or more of VP1, VP2 and VP3.

3. The modified AAV capsid protein of claim 1 or claim 2, wherein the reference AAV capsid protein is a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

4. The modified AAV capsid protein of claim 1 or claim 2, wherein the reference AAV capsid protein is a capsid protein having a sequence selected from SEQ ID Nos: 54-152 or a fragment thereof.

5. The modified AAV capsid protein of any one of claims 1-4, wherein the 7-mer peptide is inserted into an amino acid position between 565 and 595 of the reference AAV capsid protein.

6. The modified AAV capsid protein of any one of claims 1-4, wherein:
(i) the reference AAV capsid protein is a capsid protein of AAV1 and the 7-mer peptide is inserted between D590 and P591 or between S588 and T589 of the capsid protein;
(ii) the reference AAV capsid protein is a capsid protein of AAV2 and the 7-mer peptide is inserted between R588 and Q589 or between N587 and R588 of the capsid protein;
(iii) the reference AAV capsid protein is a capsid protein of AAV3b and the 7-mer peptide is inserted between S586 and S587 or between N588 and T589 of the capsid protein;
(iv) the reference AAV capsid protein is a capsid protein of AAV4 and the 7-mer peptide is inserted between S584 and N585 or between S586 and N587 of the capsid protein;
(v) the reference AAV capsid protein is a capsid protein of AAV5 and the 7-mer peptide is inserted between S575 and S576 or between T577 and T578 of the capsid protein;
(vi) the reference AAV capsid protein is a capsid protein of AAV6 and the 7-mer peptide is inserted between D590 and P591 or S588 and T589 of the capsid protein;
(vii) the reference AAV capsid protein is a capsid protein of AAV7 and the 7-mer peptide is inserted between N589 and T590 of the capsid protein;
(viii) the reference AAV capsid protein is a capsid protein of AAV8 and the 7-mer peptide is inserted between N590 and T591 of the capsid protein;
(ix) the reference AAV capsid protein is a capsid protein of AAV9 and the 7-mer peptide is inserted between Q588 and A589 of the capsid protein;
(x) the reference AAV capsid protein is a capsid protein of AAVrh10 and the 7-mer peptide is inserted between N590 and A591 of the capsid protein;
(xi) the reference AAV capsid protein is a capsid protein of AAVpo.1 and the 7-mer peptide is inserted between N567 and S568 or between N569 and T570 of the capsid protein; or
(xii) the reference AAV capsid protein is a capsid protein of AAV12 and the 7-mer peptide is inserted between N592 and A593 or between T594 and T595 of the capsid protein.

7. The modified AAV capsid protein of any one of claims 1-6, having the sequence of SEQ ID NO: 158.

8. The modified AAV capsid protein according to claim 1, wherein the reference AAV capsid protein is a liver-toggle mutant of a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

9. The modified AAV capsid protein according to claim 1, wherein the reference AAV capsid protein is a liver-toggle mutant of a capsid protein having a sequence selected from SEQ ID Nos: 54-152 or a fragment thereof.

10. The modified AAV capsid protein of claim 8 or 9, comprising: an alanine (A) amino acid residue at an amino acid position corresponding to position 266 in Anc80; or a lysine (K) amino acid residue at an amino acid position corresponding to position 168 in Anc80.

11. The modified AAV capsid protein of claim 8 or 9, wherein the reference AAV capsid protein is a liver toggle mutant of a capsid protein of AAV9 comprising an alanine (A) amino acid residue at an amino acid position 267 and a threonine (T) amino acid residue at an amino acid position 269.

12. The modified protein of claim 11, comprising the sequence of SEQ ID NO: 159.

13. A modified adeno-associated virus (AAV) capsid protein, comprising:
(i) a liver-toggle mutant of a reference AAV capsid protein, comprising
a) an alanine (A) or glycine (G) amino acid residue at an amino acid position corresponding to position 266 in Anc80; or
b) a lysine (K) or arginine (R) amino acid residue at an amino acid position corresponding to position 168 in Anc80; and
(ii) a targeting peptide inserted into a site within VR VIII of the liver-toggle mutant.

14. The modified AAV capsid protein of claim 13, wherein the liver-toggle mutant comprises:
a) an alanine (A) amino acid residue at an amino acid position corresponding to position 266 in Anc80; or
b) a lysine (K) amino acid residue at an amino acid position corresponding to position 168 in Anc80.

15. The modified AAV capsid protein of claim 14, wherein the liver-toggle mutant comprises:
a) an alanine (A) amino acid residue at an amino acid position corresponding to position 266 in Anc80; and
b) a lysine (K) amino acid residue at an amino acid position corresponding to position 168 in Anc80.

16. The modified AAV capsid protein of claim 13, wherein the liver-toggle mutant comprises:
a) a glycine (G) amino acid residue at an amino acid position corresponding to position 266 in Anc80; or
b) an arginine (R) amino acid residue at an amino acid position corresponding to position 168 in Anc80.

17. The modified AAV capsid protein of claim 14, wherein the liver-toggle mutant comprises:
a) a glycine (G) amino acid residue at an amino acid position corresponding to position 266 in Anc80; and
b) an arginine (R) amino acid residue at an amino acid position corresponding to position 168 in Anc80.

18. The modified AAV capsid protein of claim 13-17, wherein the targeting peptide is 7-mer peptide having the sequence RGDX¹X²X³X⁴ (SEQ ID NO: 52), wherein X¹ to X⁴ are independently selected amino acid residues.

19. The modified AAV capsid protein of claim 18, wherein X¹, X², and X³ are independently selected from L, G, V, and A; and X⁴ is selected from S, V, A, G, and L.

20. The modified AAV capsid protein of any one of claims 18-19, wherein X¹, X², and X³ are independently selected from L, V, and A; and at least two of X¹, X², and X³ are independently L.

21. The modified AAV capsid protein of any one of claims 18-20, wherein, X² is L.

22. The modified AAV capsid protein of claim 18, wherein 7-mer peptide has a sequence of RGDLLLS (SEQ ID NO: 1).

23. The modified AAV capsid protein of any one of claims 13-15, wherein the targeting peptide is the 7-mer peptide TLAVPFK (SEQ ID NO: 53).

24. The modified AAV capsid protein of any one of claims 13-15, wherein the targeting peptide is a peptide having a sequence selected from SEQ ID Nos: 2-51 and 53.

25. The modified AAV capsid protein of any one of claims 13-24, wherein the reference AAV capsid protein is a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

26. The modified AAV capsid protein of any one of claims 13-24, wherein the reference AAV capsid protein is a capsid protein having a sequence selected from SEQ ID Nos: 54-152 or a fragment thereof.

27. The modified AAV capsid protein of claim 25 or 26, wherein the reference AAV capsid protein is an AAV9 capsid protein.

28. The modified AAV capsid protein of claim 27, wherein the liver-toggle mutant comprises an alanine (A) amino acid residue at position 267.

29. The modified AAV capsid protein of any one of claims 27-28, wherein the liver-toggle mutant comprises a threonine (T) amino acid residue at position 269.

30. The modified AAV capsid protein of claim 27, comprising an alanine (A) amino acid residue at position 267 and a threonine (T) amino acid residue at position 269.

31. The modified AAV capsid protein of any one of claims 13-30, wherein the targeting peptide is inserted into an amino acid position between 565 and 595 of the liver toggle mutant.

32. The modified AAV capsid protein of claim 31, wherein:
(i) the reference AAV capsid protein is a capsid protein of AAV1 and the targeting peptide is inserted between D590 and P591 or between S588 and T589 of the liver-toggle mutant;
(ii) the reference AAV capsid protein is a capsid protein of AAV2 and the targeting peptide is inserted between R588 and Q589 or between N587 and R588 of the liver-toggle mutant;
(iii) the reference AAV capsid protein is a capsid protein of AAV3b and the targeting peptide is inserted between S586 and S587 or between N588 and T589 of the liver-toggle mutant;
(iv) the reference AAV capsid protein is a capsid protein of AAV4 and the targeting peptide is inserted between S584 and N585 or between S586 and N587 of the liver-toggle mutant;
(v) the reference AAV capsid protein is a capsid protein of AAV5 and the targeting peptide is inserted between S575 and S576 or between T577 and T578 of the liver-toggle mutant;
(vi) the reference AAV capsid protein is a capsid protein of AAV6 and the targeting peptide is inserted between D590 and P591 or S588 and T589 of the liver-toggle mutant;
(vii) the reference AAV capsid protein is a capsid protein of AAV7 and the targeting peptide is inserted between N589 and T590 of the liver-toggle mutant;
(viii) the reference AAV capsid protein is a capsid protein of AAV8 and the targeting peptide is inserted between N590 and T591 of the liver-toggle mutant;
(ix) the reference AAV capsid protein is a capsid protein of AAV9 and the targeting peptide is inserted between Q588 and A589 of the liver-toggle mutant;
(x) the reference AAV capsid protein is a capsid protein of AAVrh10 and the targeting peptide is inserted between N590 and A591 of the liver-toggle mutant;
(xi) the reference AAV capsid protein is a capsid protein of AAVpo. 1 and the targeting peptide is inserted between N567 and S568 or between N569 and T570 of the liver-toggle mutant; or
(xii) the reference AAV capsid protein is a capsid protein of AAV12 and the targeting peptide is inserted between N592 and A593 or between T594 and T595 of the liver-toggle mutant.

33. The modified AAV capsid protein of any one of claims 13-32, wherein the liver-toggle mutant comprises a sequence selected from NSTSGASS (SEQ ID NO. 160), NSTSGGST (SEQ ID NO. 161) and NSTSGAST (SEQ ID NO. 162).

34. The modified AAV capsid protein of any one of claims 13-33, wherein the liver-toggle mutant of a reference AAV capsid protein, comprising:
a) an alanine (A) amino acid residue at an amino acid position corresponding to position 266 in Anc80; and
b) a lysine (K) amino acid residue at an amino acid position corresponding to position 168 in Anc80

35. The modified AAV capsid protein of any one of claims 13-33, wherein the liver-toggle mutant of a reference AAV capsid protein, comprising
a) an alanine (A) amino acid residue at an amino acid position corresponding to position 267 in AAV9; and
b) a threonine (T) amino acid residue at an amino acid position corresponding to position 269 in AAV9.

36. The modified AAV capsid protein of any one of claims 13-33, wherein the liver-toggle mutant further comprises
a) an alanine (A) amino acid residue at an amino acid position corresponding to position 504 in AAV9; and
b) an alanine (A) amino acid residue at an amino acid position corresponding to position 505 in AAV9.

37. The modified AAV capsid protein of any one of claims 13-36, having a sequence of SEQ ID NO: 159.

38. A polynucleotide encoding the modified AAV capsid protein of any one of claims 1 to 37.

39. A vector comprising the polynucleotide of claim 38.

40. The vector of claim 39, further comprising a promoter operably linked to the polynucleotide.

41. A host cell comprising the modified AAV capsid protein of any one of claims 1 to 37, the polynucleotide specified in claim 38, or the vector of claim 39 or 40.

42. A recombinant AAV virion (rAAV) comprising the modified AAV capsid protein of any one of claims 1 to 37.

43. The AAV virion of claim 42, further comprising an exogenous polynucleotide.

44. The AAV virion of claim 43, wherein the exogenous polynucleotide comprises a template for homology directed repair.

45. The AAV virion of claim 43, wherein the exogenous polynucleotide comprises an expressible polynucleotide encoding a therapeutic tRNA, miRNA, gene editing guide RNA, or RNA-editing guide RNA.

46. The AAV virion of claim 43, wherein the exogenous polynucleotide comprises an expressible polynucleotide encoding a therapeutic protein.

47. The AAV virion of claim 46, wherein the therapeutic protein is MTM1 or a fragment thereof.

48. The AAV virion of claim 47, wherein the expressible polypeptide comprises the sequence of SEQ ID NO: 165 or a fragment thereof.

49. The AAV virion of claim 47, wherein the expressible polypeptide comprises the sequence having at least 80%, 85%, 90%, 95%, 98%, 99% or 100% sequence identity to any of SEQ ID Nos: 166-170.

50. The AAV virion of any one of claims 43-49, wherein the exogenous polynucleotide further comprises a regulatory sequence.

51. The AAV virion of claim 50, wherein the regulatory sequence comprises expression regulatory elements (EREs).

52. The AAV virion of claim 51, wherein the EREs comprise a CAG promoter.

53. The AAV virion of claim 51, wherein the EREs comprise a sequence having at least 90%, at least 95%, at least 98%, at least 99% or 100% sequence identity to any one of SEQ IDs NO:171-173.

54. A pharmaceutical composition comprising the modified AAV capsid protein of any one of claims 1 to 37 or the AAV virion of any one of claims 42-53.

55. A method for treating or ameliorating or preventing a disease or condition in a subject, comprising administering a therapeutically effective amount of the AAV virion of claim 42 or the pharmaceutical composition of claim 54.

56. The method of treating or ameliorating or preventing a disease according to claim 55, wherein the disease is a muscular disease and/or the condition is muscle degeneration.

57. The method of treating or ameliorating or preventing a disease according to claim 56, wherein said muscle is a striated muscle, preferably heart or a skeletal muscle or diaphragm.

58. The method of treating or ameliorating or preventing a disease according to claim 57, wherein said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

59. The modified adeno-associated virus (AAV) capsid protein of any one of claims 1-37, for use in treating and/or preventing a muscular disease and/or muscle degeneration.

60. An AAV virion comprising the modified AAV capsid protein of any one of claims 1-37 or the AAV virion of any one of claims 42-53 for use in treating and/or preventing a muscular disease and/or in muscle regeneration.

61. A pharmaceutical composition comprising the modified AAV capsid protein of any one of claims 1 to 34, and/or the AAV virion specified in any one of claims 42-53 for use in treating and/or preventing a muscular disease and/or in muscle regeneration.

62. A method of transferring an exogenous polynucleotide into a muscle cell, comprising the step of administering the AAV virion specified in any one of claims 42-53 to a subject.

63. The method of claim 62, wherein the administration results in transfer of the exogenous polynucleotide in the muscle cell, at a muscle:liver infection ratio of greater than 1 when measured by genome copies of the AAV virion.

64. The method of claim 62, wherein the muscle:liver infection ratio ranges from 1 to 100.

65. The method of claim 63, wherein the muscle:liver infection ration ranges from 1 to 10.

66. The method of claim 65, wherein the muscle:liver infection ratio ranges from 2 to 8.

67. The method of any one of claims 62-66, wherein the administration results in expression of the exogenous polynucleotide in the muscle cell, at a muscle:liver expression ratio of greater than 10.

68. The method of claim 67, wherein the muscle:liver expression ratio ranges from 10 to 100.

69. The method of claim 68, wherein the muscle:liver expression ratio ranges from 20 to 80.

70. The method of any one of claims 62-69, wherein the muscle:liver expression ratio ranges from 50 to 80 when measured by mRNA transcript expression.

71. The method of any one of claims 62-70, wherein the muscle:liver expression ratio ranges from 10 to 50 when measured by protein expression.

72. The method of any one of claims 62-71, wherein the muscle cell is selected from triceps surae, biceps, heart and quadricep.

73. Use of the AAV capsid polypeptide of any one of claims 1 to 34, and/or the AAV virion specified in any one of claims 42-53 for transferring an exogenous polynucleotide into a muscle cell.

74. The use according to claim 73, wherein said use is a non-therapeutic use, preferably wherein said use is an in vitro use.

75. The use according to claim 73, wherein the muscle cell is selected from triceps surae, biceps, heart and quadricep.

76. A recombinant adeno-associated virus (rAAV), comprising:
a. a genome comprising an MTM1 coding sequence operably linked to an expression regulatory element (ERE); and
b. one, two or all three of the following features:
i. the ERE is a hybrid expression regulatory element (ERE) comprising a CMV enhancer and a chicken beta actin promoter operably linked to the MTM1 coding sequence; and/or
ii. the rAAV comprises a modified AAV capsid protein comprising at least one liver-toggle mutation and/or one muscle-targeting element; and/or
iii. the MTM1 coding sequence is codon optimized for expression in human cells, optionally wherein the coding sequence has at least 90%, at least 95%, at least 98% or at least 99% sequence identity to any one of SEQ ID NOS:167 to 170.

77. The rAAV of claim 76, wherein the MTM1 sequence encodes a protein comprising an amino acid sequence having at least 95% sequence identity to the amino acid sequence of SEQ ID NO:164.

78. The rAAV of claim 77, wherein the MTM1 protein comprises an amino acid sequence having at least 98% or at least 99% sequence identity to the amino acid sequence of SEQ ID NO:164.

79. The rAAV of claim 78, wherein the MTM1 protein comprises an amino acid sequence having 100% sequence identity to the amino acid sequence of SEQ ID NO:164.

80. The rAAV of claim any one of claims 76 to 79, wherein the MTM1 sequence encodes a protein comprising an amino acid sequence having at least 95% sequence identity to the amino acid sequence of SEQ ID NO:165.

81. The rAAV of claim 80, wherein the MTM1 protein comprises an amino acid sequence having at least 98% sequence identity to the amino acid sequence of SEQ ID NO:165.

82. The rAAV of claim 81, wherein the MTM1 protein comprises an amino acid sequence having at least 99% sequence identity to the amino acid sequence of SEQ ID NO:165.

83. The rAAV of claim 82, wherein the MTM1 protein comprises an amino acid sequence having 100% sequence identity to the amino acid sequence of SEQ ID NO:165.

84. The rAAV of any one of claims 76 to 83, wherein the MTM1 coding sequence comprises a nucleotide sequence having at least 90% sequence identity to SEQ ID NO 166.

85. The rAAV of claim 84, wherein the MTM1 coding sequence comprises a nucleotide sequence having at least 95% sequence identity to SEQ ID NO: 166.

86. The rAAV of claim 85, wherein the MTM1 coding sequence comprises a nucleotide sequence having at least 98% sequence identity to SEQ ID NO:166.

87. The rAAV of claim 86, wherein the MTM1 coding sequence comprises a nucleotide sequence having at least 99% sequence identity to SEQ ID NO: 166.

88. The rAAV of claim 84, wherein the MTM1 coding sequence comprises a nucleotide sequence having 100% sequence identity to SEQ ID NO: 166.

89. The rAAV of any one of claims 76 to 88 wherein the MTM1 coding sequence is codon optimized for expression in human cells.

90. The rAAV of claim 89, wherein the MTM1 coding sequence comprises a nucleotide sequence having at least 90% sequence identity to any one of SEQ ID NOS:167 to 170.

91. The rAAV of claim 90, wherein the MTM1 coding sequence comprises a nucleotide sequence having at least 95% sequence identity to any one of SEQ ID NOS:167 to 170.

92. The rAAV of claim 91, wherein the MTM1 coding sequence comprises a nucleotide sequence having at least 98% sequence identity to any one of SEQ ID NOS:167 to 170.

93. The rAAV of claim 92, wherein the MTM1 coding sequence comprises a nucleotide sequence having at least 99% sequence identity to any one of SEQ ID NOS:167 to 170.

94. The rAAV of claim 93, wherein the MTM1 coding sequence comprises a nucleotide sequence having 100% sequence identity to any one of SEQ ID NOS:167 to 170.

95. The rAAV of any one of claims 90 to 94, wherein the sequence identity is to SEQ ID NO:167.

96. The rAAV of any one of claims 90 to 94, wherein the sequence identity is to SEQ ID NO: 168.

97. The rAAV of any one of claims 90 to 94, wherein the sequence identity is to SEQ ID NO: 169.

98. The rAAV of any one of claims 90 to 94, wherein the sequence identity is to SEQ ID NO:169.

99. The rAAV of any one of claims 76 to 98 which comprises a hybrid expression regulatory element (ERE) comprising a CMV enhancer and a chicken beta actin promoter operably linked to the MTM1 coding sequence.

100. The rAAV of claim 99, wherein the ERE comprises (a) a nucleotide sequence having at least 90% sequence identity to SEQ ID NO:171 and a nucleotide sequence having at least 90% sequence identity to SEQ ID NO: 172 or (b) a nucleotide sequence having at least 90% sequence identity to SEQ ID NO: 173.

101. The rAAV of claim 100, wherein the ERE comprises (a) a nucleotide sequence having at least 95% sequence identity to SEQ ID NO:171 and a nucleotide sequence having at least 95% sequence identity to SEQ ID NO:172 or (b) a nucleotide sequence having at least 95% sequence identity to SEQ ID NO:173.

102. The rAAV of claim 100, wherein the ERE comprises (a) a nucleotide sequence having at least 98% sequence identity to SEQ ID NO:171 and a nucleotide sequence having at least 98% sequence identity to SEQ ID NO:172 or (b) a nucleotide sequence having at least 98% sequence identity to SEQ ID NO:173.

103. The rAAV of claim 100, wherein the ERE comprises (a) a nucleotide sequence having at least 99% sequence identity to SEQ ID NO:171 and a nucleotide sequence having at least 99% sequence identity to SEQ ID NO:172 or (b) a nucleotide sequence having at least 99% sequence identity to SEQ ID NO:173.

104. The rAAV of claim 100, (a) a nucleotide sequence having 100% sequence identity to SEQ ID NO: 171 and a nucleotide sequence having 100% sequence identity to SEQ ID NO: 172 or (b) a nucleotide sequence having 100% sequence identity to SEQ ID NO:173.

105. The rAAV of any one of claims 99 to 104, which further comprises a chimeric intron formed from intron sequences derived from chicken beta actin and/or human beta herpes virus and/or human beta globin and/or operably linked to the MTM1 coding sequence.

106. The rAAV of claim 105, wherein the chimeric intron comprises a nucleotide sequence derived from human beta globin, which optionally comprises a nucleotide sequence having at least 90% sequence identity to SEQ ID NO:174.

107. The rAAV of claim 106, wherein the nucleotide sequence derived from human beta globin comprises SEQ ID NO: 174.

108. The rAAV of any one of claims 105 to 107, wherein the chimeric intron comprises a nucleotide sequence derived from human betaherpes virus, which optionally comprises a nucleotide sequence having at least 90% sequence identity to SEQ ID NO:175.

109. The rAAV of claim 108, wherein the nucleotide sequence derived from human betaherpes virus comprises SEQ ID NO:175.

110. The rAAV of claim 105, wherein the chimeric intron is formed from introns from human betaherpes virus and rabbit beta globin.

111. The rAAV of claim 105, wherein the chimeric intron comprises a nucleotide sequence having at least 90% sequence identity to SEQ ID NO:176.

112. The rAAV of claim 111, wherein the chimeric intron comprises a nucleotide sequence having at least 95% sequence identity to SEQ ID NO:176.

113. The rAAV of claim 112, wherein the chimeric intron comprises a nucleotide sequence having at least 98% sequence identity to SEQ ID NO:176.

114. The rAAV of claim 113, wherein the chimeric intron comprises a nucleotide sequence having at least 99% sequence identity to SEQ ID NO:176.

115. The rAAV of claim 114, wherein the chimeric intron comprises a nucleotide sequence having 100% sequence identity to SEQ ID NO: 176.

116. The rAAV of claim 115, wherein the chimeric intron comprises the nucleotide sequence of SEQ ID NO:176.

117. The rAAV of any one of claims 76 to 116, which comprises an unmodified or modified AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI capsid protein.

118. The rAAV of claim 117, which comprises an unmodified or modified rAAV9 capsid protein.

119. The rAAV of any one of claims 76 to 118 which comprises a VP1, VP2 and/or VP3 capsid protein comprising an amino acid sequence having at least 90% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.61-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

120. The rAAV of any one of claims 76 to 119 which comprises a VP1, VP2 and/or VP3 capsid protein comprising an amino acid sequence having at least 95% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

121. The rAAV of any one of claims 76 to 120 which comprises a VP1, VP2 and/or VP3 capsid protein comprising an amino acid sequence having at least 98% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc13; Anc126; Anc127; Anc80L27; Anc80L59; Anc80L60; Anc80L62; Anc80L65; Anc80L33; Anc80L36; Anc80L44; Anc80L1; And 10; or Anc80DI.

122. The rAAV of any one of claims 76 to 121 which comprises a VP1, VP2 and/or VP3 capsid protein comprising an amino acid sequence having at least 99% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc8l; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

123. The rAAV of any one of claims 76 to 122 which comprises a VP1, VP2 and/or VP3 capsid protein comprising an amino acid sequence having 100% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; or Anc80DI.

124. The rAAV of any one of claims 76 to 123, which comprises a modified AAV capsid protein comprising at least one liver-toggle mutation as compared to a reference capsid protein.

125. The rAAV of claim 124, wherein the reference capsid protein is a VP1, VP2 and/or VP3 protein.

126. The rAAV of claim 124 or claim 125, wherein the reference AAV capsid protein is a capsid protein having any one of SEQ ID NOs: 54-152 or a fragment thereof.

127. The rAAV of any one of claims 124 to 126, wherein the at least one liver-toggle mutation comprises:
a. an alanine (A) or glycine (G) amino acid residue at an amino acid position corresponding to position 266 in Anc80; and/or
b. a lysine (K) or arginine (R) amino acid residue at an amino acid position corresponding to position 168 in Anc80.

128. The rAAV of claim 127, wherein the at least one liver-toggle mutation comprises:
a. an alanine (A) amino acid residue at an amino acid position corresponding to position 266 in Anc80; and/or
b. a lysine (K) amino acid residue at an amino acid position corresponding to position 168 in Anc80.

129. The rAAV of claim 127, wherein the at least one liver-toggle mutation comprises:
a. an alanine (A) amino acid residue at an amino acid position corresponding to position 266 in Anc80; and/or
b. an arginine (R) amino acid residue at an amino acid position corresponding to position 168 in Anc80.

130. The rAAV of claim 127, wherein the at least one liver-toggle mutation comprises:
a. a glycine (G) amino acid residue at an amino acid position corresponding to position 266 in Anc80; and/or
b. a lysine (K) amino acid residue at an amino acid position corresponding to position 168 in Anc80.

131. The rAAV of claim 127, wherein the at least one liver-toggle mutation comprises:
a. a glycine (G) amino acid residue at an amino acid position corresponding to position 266 in Anc80; and/or
b. an arginine (R) amino acid residue at an amino acid position corresponding to position 168 in Anc80.

132. The rAAV of any one of claims 124 to 126, wherein the at least one liver-toggle mutation comprises an alanine (A) at an amino acid position corresponding to position 267 in AAV9.

133. The rAAV of any one of claims 124 to 126, wherein the at least one liver-toggle mutation comprises a threonine (T) at an amino acid position corresponding to position 269 in AAV9.

134. The rAAV of any one of claims 76-133, wherein the capsid protein is a modified AAV9 capsid protein, optionally wherein the capsid protein is a modified AAV9 VP1 capsid protein.

135. The rAAV of any one any one of claims 124 to 126, 132 and 133, wherein the liver-toggle mutation comprises:
a. an alanine (A) amino acid residue at an amino acid position corresponding to position 267 in AAV9; and
b. a threonine (T) amino acid residue at an amino acid position corresponding to position 269 in AAV9.

136. The rAAV of any one any one of claims 124 to 126, 132 and 133, wherein the liver-toggle mutation further comprises
a. an alanine (A) amino acid residue at an amino acid position corresponding to position 504 in AAV9; and/or
b. an Alanine (A) amino acid residue at an amino acid position corresponding to position 505 in AAV9.

137. The rAAV of any one of claims 124 to 136, wherein the liver-toggle mutant comprises the sequence NSTSGASS (SEQ ID NO: 160), NSTSGGST (SEQ ID NO:161) or NSTSGAST (SEQ ID NO: 162).

138. The rAAV of claim 124, wherein the rAAV capsid protein has the sequence of SEQ ID NO 159.

139. The rAAV of claim 124, wherein the rAAV capsid protein has the sequence of SEQ ID NO:163.

140. The rAAV of any one any one of claims 124 to 126, wherein the one or more liver toggle mutations comprise one or more amino acid substitutions at one or more of Q263, S264, G265, A266, S267, N268, H271, N382, G383, S384, Q385, S446, R471, W502, T503, D528, D529, Q589, K706, and V708 as compared to an AAV2 reference capsid protein (SEQ ID NO:1 of WO2021/050614, which is incorporated by reference herein).

141. The rAAV of any one any one of claims 124 to 126 and 140, wherein the one or more liver toggle mutations comprise the amino acid substitution S446R as compared to a reference capsid protein.

142. The rAAV of any one any one of claims 124 to 126, 140 and 141, wherein the one or more liver toggle mutations comprise the amino acid substitution R471A as compared to a reference capsid protein.

143. The rAAV of any one any one of claims 124 to 126 and 140 to 142, wherein the one or more liver toggle mutations comprise the amino acid substitution V708T or V708A as compared to a reference capsid protein.

144. The rAAV of any one of claims 76 to 143, which comprises a modified AAV capsid protein comprising at least one muscle-targeting element as compared to a reference capsid protein.

145. The rAAV of claim 144, wherein the reference capsid protein is a VP1, VP2 and/or VP3 protein.

146. The rAAV of any one of claim 144 or claim 145, wherein the muscle targeting element is 7-mer peptide having the sequence RGDX1X2X3X4 (SEQ ID NO:52), wherein X1 to X4 are independently selected amino acid residues.

147. The rAAV of claim 146, wherein X1, X2, and X3 are independently selected from L, G, V, and A; and X4 is selected from S, V, A, G, and L.

148. The rAAV of any one of claims 146 to 147, wherein X1, X2, and X3 are independently selected from L, V, and A; and at least two of X1, X2, and X3 are independently L.

149. The rAAV of any one of claims 146 to 148, wherein, X2 is L.

150. The rAAV of claim 146, wherein 7-mer peptide has a sequence of RGDLLLS (SEQ ID NO: 1).

151. The rAAV of claim 144 or claim 145, wherein the targeting peptide is the 7-mer peptide TLAVPFK (SEQ ID NO:53).

152. The rAAV of claim 144 or claim 145, wherein the targeting peptide is a peptide having any one of SEQ ID NOs:2-51 and 53.

153. The rAAV of claim 144 or claim 145, wherein the muscle-targeting element consists of a 7-mer peptide having the sequence RGDLLLS (SEQ ID NO:1) inserted into a site within VR VIII of the AAV capsid protein.

154. The rAAV of claim 153, wherein the 7-mer peptide is inserted into an amino acid position between 565 and 595 of the reference AAV capsid protein.

155. The rAAV of any one of claims 144 to 154, wherein:
a. the reference AAV capsid protein is a capsid protein of AAV1 and a 7-mer muscle-targeting peptide is inserted between D590 and P591 or between S588 and T589 of the capsid protein;
b. the reference AAV capsid protein is a capsid protein of AAV2 and the 7-mer muscle-targeting peptide is inserted between R588 and Q589 or between N587 and R588 of the capsid protein;
c. the reference AAV capsid protein is a capsid protein of AAV3b and the 7-mer muscle-targeting peptide is inserted between S586 and S587 or between N588 and T589 of the capsid protein;
d. the reference AAV capsid protein is a capsid protein of AAV4 and the 7-mer muscle-targeting peptide is inserted between S584 and N585 or between S586 and N587 of the capsid protein;
e. the reference AAV capsid protein is a capsid protein of AAV5 and the 7-mer muscle-targeting peptide is inserted between S575 and S576 or between T577 and T578 of the capsid protein;
f. the reference AAV capsid protein is a capsid protein of AAV6 and the 7-mer muscle-targeting peptide is inserted between D590 and P591 or S588 and T589 of the capsid protein;
g. the reference AAV capsid protein is a capsid protein of AAV7 and the 7-mer muscle-targeting peptide is inserted between N589 and T590 of the capsid protein;
h. the reference AAV capsid protein is a capsid protein of AAV8 and the 7-mer muscle-targeting peptide is inserted between N590 and T591 of the capsid protein;
i. the reference AAV capsid protein is a capsid protein of AAV9 and the 7-mer muscle-targeting peptide is inserted between Q588 and A589 of the capsid protein;
j. the reference AAV capsid protein is a capsid protein of AAVrh10 and the 7-mer muscle-targeting peptide is inserted between N590 and A591 of the capsid protein;
k. the reference AAV capsid protein is a capsid protein of AAVpo.1 and the 7-mer muscle-targeting peptide is inserted between N567 and S568 or between N569 and T570 of the capsid protein; or
l. the reference AAV capsid protein is a capsid protein of AAV12 and the 7-mer muscle-targeting peptide is inserted between N592 and A593 or between T594 and T595 of the capsid protein.

156. The rAAV of any one of claims 144 to 155, wherein the muscle targeting peptide is inserted into a site within VR VIII of a liver-toggle mutant capsid, optionally a liver-toggle mutant capsid as described in any one of claims 124 to 137.

157. The rAAV of claim 156, wherein the muscle targeting peptide is inserted into an amino acid position between 565 and 595 of the liver toggle mutant.

158. The rAAV of claim 157, wherein:
a. the reference AAV capsid protein is a capsid protein of AAV1 and the targeting peptide is inserted between D590 and P591 or between S588 and T589 of the liver-toggle mutant;
b. the reference AAV capsid protein is a capsid protein of AAV2 and the targeting peptide is inserted between R588 and Q589 or between N587 and R588 of the liver-toggle mutant;
c. the reference AAV capsid protein is a capsid protein of AAV3b and the targeting peptide is inserted between S586 and S587 or between N588 and T589 of the liver-toggle mutant;
d. the reference AAV capsid protein is a capsid protein of AAV4 and the targeting peptide is inserted between S584 and N585 or between S586 and N587 of the liver-toggle mutant;
e. the reference AAV capsid protein is a capsid protein of AAV5 and the targeting peptide is inserted between S575 and S576 or between T577 and T578 of the liver-toggle mutant;
f. the reference AAV capsid protein is a capsid protein of AAV6 and the targeting peptide is inserted between D590 and P591 or S588 and T589 of the liver-toggle mutant;
g. the reference AAV capsid protein is a capsid protein of AAV7 and the targeting peptide is inserted between N589 and T590 of the liver-toggle mutant;
h. the reference AAV capsid protein is a capsid protein of AAV8 and the targeting peptide is inserted between N590 and T591 of the liver-toggle mutant;
i. the reference AAV capsid protein is a capsid protein of AAV9 and the targeting peptide is inserted between Q588 and A589 of the liver-toggle mutant;
j. the reference AAV capsid protein is a capsid protein of AAVrh10 and the targeting peptide is inserted between N590 and A591 of the liver-toggle mutant;
k. the reference AAV capsid protein is a capsid protein of AAVpo.1 and the targeting peptide is inserted between N567 and S568 or between N569 and T570 of the liver-toggle mutant; or
l. the reference AAV capsid protein is a capsid protein of AAV12 and the targeting peptide is inserted between N592 and A593 or between T594 and T595 of the liver-toggle mutant.

159. The rAAV of claim 144, wherein the capsid protein has the sequence of SEQ ID NO:158.

160. The rAAV claim 144, wherein the rAAV capsid protein has the sequence of SEQ H) NO:159.

161. The rAAV of any one of claims 124 to 160, except when dependent on claims 77 to 115, in which the ERE comprises a constitutive promoter.

162. The rAAV of claim 161, wherein the constitutive promoter is the Rous sarcoma virus (RSV) LTR promoter (optionally with the RSV enhancer), the cytomegalovirus (CMV) promoter (optionally with the CMV enhancer), the SV40 promoter, the dihydrofolate reductase (DHFR) promoter, the β-actin promoter, the phosphoglycerol kinase 1 (PGK1) promoter (optionally the minimal PGK1 promoter), or the EF1 alpha promoter (optionally with intron).

163. The rAAV of any one of claims 124 to 160, except when dependent on claims 77 to 115, in which the ERE comprises an inducible promoter.

164. The rAAV of claim 163, wherein the inducible promoter is a tetracycline or rapamycin inducible promoter.

165. The rAAV of any one of claims 124 to 160, except when dependent on claims 77 to 115, in which the ERE comprises a muscle-specific promoter.

166. The rAAV of claim 165, wherein the muscle specific promoter is a desmin promoter (which is optionally a CpG depleted desmin promoter), a CKM promoter derivative or an MTM1 promoter.

167. The rAAV of any one of claims 161 to 166, where the promoter is a human promoter.

168. The rAAV of any one of claims 1 to 167 which comprises a rabbit globin poly A sequence 3′ to the MTM1 coding sequence, optionally wherein the rabbit globin poly A sequence has at least 90% sequence identity to SEQ ID NO:177.

169. The rAAV claim 168, wherein the rabbit globin poly A sequence has at least 95% sequence identity to SEQ ID NO:177.

170. The rAAV claim 169, wherein the rabbit globin poly A sequence has at least 98% sequence identity to SEQ ID NO: 177.

171. The rAAV claim 170, wherein the rabbit globin poly A sequence has at least 99% sequence identity to SEQ ID NO: 177.

172. The rAAV claim 171, wherein the rabbit globin poly A sequence has 100% sequence identity to SEQ ID NO:177.

173. The rAAV of any one of claims 1 to 172 whose genome comprises AAV-derived inverted terminal repeat sequences (ITRs).

174. The rAAV of claim 173, wherein the ITRs are derived from AAV serotype 2.

175. The rAAV of claim 173 or claim 174, which comprises a first ITR having at least 90% sequence identity to SEQ ID NO:178 and a second ITR having at least 90% sequence identity to SEQ ID NO: 179.

176. The rAAV of claim 175, wherein the first ITR has at least 95% sequence identity to SEQ ID NO: 178 and the second ITR has at least 95% sequence identity to SEQ ID NO:179.

177. The rAAV of claim 176, wherein the first ITR has at least 98% sequence identity to SEQ ID NO:178 and the second ITR has at least 98% sequence identity to SEQ ID NO:179.

178. The rAAV of claim 177, wherein the first ITR has at least 99% sequence identity to SEQ ID NO: 178 and the second ITR has at least 99% sequence identity to SEQ ID NO:179.

179. The rAAV of claim 178, wherein the first ITR 100% sequence identity to SEQ ID NO: 178 and the second ITR has 100% sequence identity to SEQ ID NO:179.

180. The rAAV of any one of claims 1 to 179, which comprises a heterologous splice acceptor sequence 5′ to the MTM1 coding sequence.

181. The rAAV of claim 180, wherein the heterologous splice acceptor sequence is derived from human beta globin exon 3.

182. The rAAV of claim 181, wherein the heterologous splice acceptor sequence comprises the nucleotide sequence of 180.

183. An rAAV comprising:
a. modified AAV capsid protein comprising at least one liver-toggle mutation and/or one muscle-targeting element, optionally wherein the modified capsid protein comprises the amino acid sequence of SEQ ID NO:158, SEQ ID NO:159, or SEQ ID NO:163;
b. a genome comprising:
i. a first ITR sequence;
ii. a hybrid expression regulatory element (ERE) comprising a CMV enhancer and a chicken beta actin promoter, optionally wherein the ERE comprises the nucleotide sequence of SEQ ID NO:173;
iii. an MTM1 coding sequence operably linked to the ERE; and
iv. a second ITR sequence.

184. The rAAV of claim 183, which further comprises a chimeric intron between the ERE and the MTM1 coding sequence, optionally wherein the chimeric intron comprises the nucleotide sequence of SEQ ID NO:176.

185. The rAAV of claim 183 or claim 184, which further comprises a splice acceptor site 5′ to the MTM1 coding sequence, optionally wherein the splice acceptor site comprises the nucleotide sequence of SEQ ID NO:180.

186. The rAAV of any one of claims 183 to 185, which further comprises a polyadenylation sequence 3′ to the MTM1 coding sequence, optionally wherein the polyadenylation sequence comprises the nucleotide sequence of SEQ ID NO:177.

187. The rAAV of any one of claims 183 to 186, wherein the MTM1 coding sequence is codon optimized for expression in human cells, optionally wherein the MTM1 coding sequence comprises the nucleotide sequence of SEQ ID NO: 167, SEQ ID NO:168, SEQ ID NO:169 or SEQ ID NO:170.

188. The rAAV of any one of claims 1 to 187 whose genome is self-complementary, optionally wherein the genome is fully self-complementary.

189. A pharmaceutical composition comprising the rAAV of any one of claims 1 to 188 and a pharmaceutically acceptable carrier.

190. The pharmaceutical composition of claim 189 which is in the form of a unit dose.

191. The pharmaceutical composition of claim 189 or claim 190 which comprises 1×10¹⁰ to 1×10¹⁶ genome copy numbers (GC) of the rAAV and/or in which the rAAV concentration is 1×10¹⁰ vg/ml to 1×10¹⁶ vg/ml.

192. The pharmaceutical composition of any one of claims 189 to 191 which is formulated for parenteral administration, for example systemic (e.g., intravenous), intramuscular or subcutaneous administration.

193. A host cell engineered to produce the rAAV of any one of claims 1 to 188.

194. The host cell of claim 193, which comprises a polynucleotide expressing one or more capsid proteins of the rAAV, a functional rep gene, and a recombinant nucleic acid vector comprising AAV ITRs and the MTM coding sequence operably linked to an expression regulatory element (ERE), optionally wherein the ERE is a hybrid ERE comprising a CMV enhancer and a chicken beta actin promoter.

195. A method for treating or ameliorating or preventing X-linked myotubular myopathy in a subject, comprising administering a therapeutically effective amount of the rAAV of any one of claims 1 to 188 or the pharmaceutical composition of any one of claims 189 to 192.

196. The method of claim 195, wherein the effective dose comprises 1×10¹⁰ to 1×10¹⁶ genome copy numbers (GC) of the rAAV.

197. The method of claim 195 or claim 196, wherein the effective dose is 1×10¹⁵ GC or less.

198. The method of claim 195 or claim 196, wherein the effective dose is 5×10¹⁴ GC or less.

199. The method of claim 195 or claim 196, wherein the effective dose is 1×10¹⁴ GC or less.

200. The method of claim 195 or claim 196, wherein the effective dose is 5×10¹³ GC or less.

201. The method of claim 195 or claim 196, wherein the effective dose is 1×10¹³ CC or less.

202. The method of any one of claims 195 to 201, wherein the administration is parenteral.

203. The method of claim 202, wherein the administration is systemic (e.g., intravenous).

204. The method of claim 202, wherein the administration is intramuscular.

205. The method of claim 202, wherein the administration is subcutaneous.

206. The rAAV of any one of claims 1 to 188 or the pharmaceutical composition of any one of claims 189 to 192, for use in treating and/or preventing X-linked myotubular myopathy.

207. The rAAV of any one of claims 1 to 188 or the pharmaceutical composition of any one of claims 189 to 192, for use in expressing myotubularin in a muscle cell.
